(19) United States
(12) Patent Application Publication
(10) Pub. No.: US 2024/0180900 A1
(43) Pub. Date: Jun. 6, 2024

(54) COMPOUNDS AND METHODS FOR THE TARGETED DEGRADATION OF ANDROGEN RECEPTOR

(71) Applicant: Arvinas Operations, Inc., New Haven, CT (US)

(72) Inventors: Ronald PECK, Cheshire, CT (US); Sarah Deborah CHIRNOMAS, Woodbridge, CT (US); Edward CHAN, Westlake Village, CA (US); Richard Walter GEDRICH, Guilford, CT (US); Elmer J. BERGHORN, Jr., Eden, NY (US); Jeanette Toni ALICEA, Newtown, CT (US); Lawrence B. SNYDER, Killingworth, CT (US); SangHyun LEE, Chesterbrook, PA (US); Taavi NEKLESA, Orange, CT (US)

(21) Appl. No.: 18/493,773

(22) Filed: Oct. 24, 2023

Related U.S. Application Data
(60) Provisional application No. 63/592,176, filed on Oct. 21, 2023.
(60) Provisional application No. 63/582,504, filed on Sep. 13, 2023.
(60) Provisional application No. 63/506,829, filed on Jun. 7, 2023.
(60) Provisional application No. 63/496,008, filed on Apr. 13, 2023.
(60) Provisional application No. 63/493,309, filed on Mar. 30, 2023.
(60) Provisional application No. 63/434,813, filed on Dec. 22, 2022.
(60) Provisional application No. 63/418,927, filed on Oct. 24, 2022.
(60) Provisional application No. 63/418,926, filed on Oct. 24, 2022.

(51) Int. Cl. A61K 31/496; A61K 31/573; A61K 31/58; A61P 35/00

(52) U.S. Cl. CPC A61K 31/496; A61K 31/573; A61K 31/58; A61P 35/00

(57) ABSTRACT

This disclosure pertains to methods of treating prostate cancer in a subject, including prostate cancer comprising at least one somatic AR tumor mutation, such as metastatic prostate cancer, castrate-resistant prostate cancer, metastatic castrate-resistant prostate cancer, progressive metastatic castrate-resistant prostate cancer, castrate-sensitive prostate cancer, metastatic castrate-sensitive prostate cancer, prostate cancer naïve to novel hormonal agents (NHAs), metastatic prostate cancer naïve NHAs, castrate-resistant prostate cancer naïve to NHAs, castrate-sensitive prostate cancer naïve to NHAs, metastatic castrate-resistant prostate cancer naïve to NHAs, and metastatic castrate-sensitive prostate cancer naïve to NHAs, wherein the method comprises administering to a subject in need thereof a therapeutically effective amount of Compound A, or a pharmaceutically acceptable salt thereof.

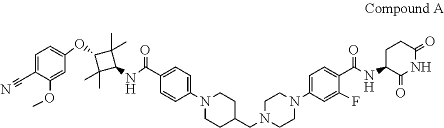 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

### CROSS REFERENCE TO RELATED APPLICATIONS

This application claims priority to, and the benefit of, U.S. Provisional Application No. 63/418,926, filed Oct. 24, 2022, U.S. Provisional Application No. 63/418,927, filed Oct. 24, 2022, U.S. Provisional Application No. 63/434,813, filed Dec. 22, 2022, U.S. Provisional Application No. 63/493,309, filed Mar. 30, 2023, U.S. Provisional Application No. 63/496,008, filed Apr. 13, 2023, U.S. Provisional Application No. 63/506,829, filed Jun. 7, 2023, U.S. Provisional Application No. 63/582,504, filed Sep. 13, 2023, and U.S. Provisional Application No. 63/592,176, filed Oct. 21, 2023, the contents of which are incorporated herein by reference in their entirety.

### INCOPORATION BY REFERENCE OF A SEQUENCE LISTING

The contents of the electronic sequence listing, file named “ARVN-016-001US_ST26.xml”, which was created on Oct. 20, 2023, and is 2,926 bytes in size, are hereby incorporated by reference in their entirety.

### TECHNICAL FIELD

The disclosure provides methods of using bifunctional compounds for treating prostate cancer.

### BACKGROUND OF THE DISCLOSURE

Most small molecule drugs bind enzymes or receptors in tight and well-defined pockets. On the other hand, protein-protein interactions are notoriously difficult to target using small molecules due to their large contact surfaces and the shallow grooves or flat interfaces involved. E3 ubiquitin ligases (of which hundreds are known in humans) confer substrate specificity for ubiquitination and are therefore attractive therapeutic targets. The development of ligands of E3 ligases has proven challenging, in part due to the fact that they must disrupt protein-protein interactions. However, recent developments have provided specific ligands which bind to these ligases.

One E3 ubiquitin ligase with therapeutic potential is cereblon. Cereblon is a protein that in humans is encoded by the CRBN gene. Thalidomide and its analogs, e.g., pomalidomide and lenalidomide, are known to bind cereblon. These agents bind to cereblon, altering the specificity of the complex to induce the ubiquitination and degradation of transcription factors essential for multiple myeloma growth. Indeed, higher expression of cereblon has been linked to an increase in efficacy of imide drugs in the treatment of multiple myeloma.

Androgen Receptor (AR) belongs to a nuclear hormone receptor family that is activated by androgens, such as testosterone and dihydrotestosterone (Pharmacol. Rev. 2006, 58(4), 782-97; Vitam. Horn. 1999, 55:309-52.). In the absence of androgens, AR is bound by Heat Shock Protein 90 (Hsp90) in the cytosol. When an androgen binds AR, its conformation changes to release AR from Hsp90 and to expose the Nuclear Localization Signal (NLS). The latter enables AR to translocate into the nucleus where AR acts as a transcription factor to promote gene expression responsible for male sexual characteristics (Endocr. Rev. 1987, 8(1):1-28; Mol. Endocrinol. 2002, 16(10), 2181-7). AR deficiency leads to Androgen Insensitivity Syndrome, formerly termed testicular feminization.

While AR is responsible for development of male sexual characteristics, it is also a well-documented oncogene in certain forms of cancers including prostate cancers (Endocr. Rev. 2004, 25(2), 276-308). A commonly measured target gene of AR activity is the secreted Prostate Specific Antigen (PSA) protein. The current treatment regimen for prostate cancer involves inhibiting the androgen-AR axis by two methods. The first approach relies on reduction of androgens, while the second strategy aims to inhibit AR function (Nat. Rev. Drug Discovery, 2013, 12, 823-824). Despite the development of effective targeted therapies, most patients develop resistance and the disease progresses. An alternative approach for the treatment of prostate cancer involves eliminating the AR protein.

Because AR is a critical driver of tumorigenesis in many forms of prostate cancers, its elimination should lead to a therapeutically beneficial response. There exists an ongoing need in the art for effective treatments for diseases, especially cancer, prostate cancer, and Kennedy's Disease.

However, non-specific effects, and the inability to target and modulate certain classes of proteins altogether, such as transcription factors, remain as obstacles to the development of effective anti-cancer agents. As such, small molecule therapeutic agents that leverage or potentiate cereblon's substrate specificity and, at the same time, are “tunable” such that a wide range of protein classes can be targeted and modulated with specificity would be very useful as a therapeutic.

Over 70 different somatic missense AR tumor mutation have been identified in patients with prostate cancer (Gottlieb, B., Hum. Mutat. 2004, 23: 527-533). The majority of these AR tumor mutations reside in the ligand binding domain. Without being bound by theory, AR tumor mutations in the ligand binding domain result in decreased ligand specificity, thereby enabling AR to function independently of androgen. Such AR tumor mutations provide tumor cells with the capability to proliferate in androgen-depleted environments, and thus are selected in response to therapies for prostate cancer that block or reduce androgen levels (e.g., luteinizing hormone-releasing hormone agonists). Accordingly, AR tumor mutations are observed with increased frequency in patients having advanced, androgen-independent tumors as compared to patients having early-stage prostate cancer (Taplin, M. E., et al. N. Engl. J. Med. (1995) 332: 1393-1398; Marcelli, M., et al. Cancer Res. (2000) 60: 944-949).

### SUMMARY

In one aspect, this application pertains to a method of treating prostate cancer in a subject comprising administering to the subject in need thereof a therapeutically effective amount of Compound A,

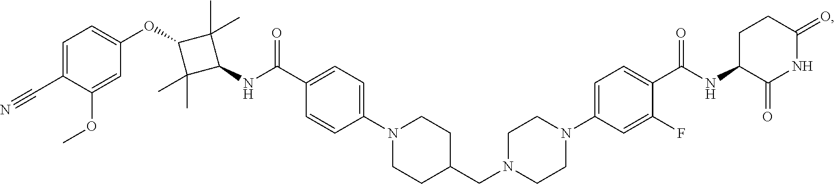 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

or a pharmaceutically acceptable salt thereof; wherein the prostate cancer is adenocarcinoma of the prostate, metastatic prostate cancer, castrate-resistant prostate cancer, metastatic castrate-resistant prostate cancer, or progressive metastatic castrate-resistant prostate cancer; and wherein the therapeutically effective amount of Compound A is about 5 mg to about 750 mg.

In one aspect, this application pertains to a method of treating prostate cancer in a subject comprising administering to the subject in need thereof a therapeutically effective amount of Compound A,

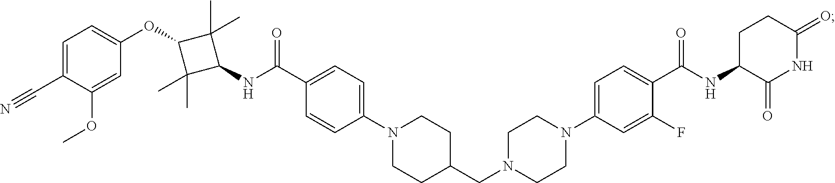 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

wherein the prostate cancer is adenocarcinoma of the prostate, metastatic prostate cancer, castrate-resistant prostate cancer, metastatic castrate-resistant prostate cancer, or progressive metastatic castrate-resistant prostate cancer; and wherein the therapeutically effective amount of Compound A is about 5 mg to about 750 mg.

In one aspect, this application pertains to a method of treating prostate cancer in a subject comprising administering to the subject in need thereof:
(i) a therapeutically effective amount of Compound A,

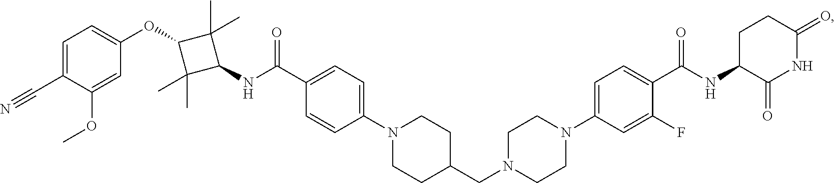 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

or a pharmaceutically acceptable salt thereof; and
(ii) a therapeutically effective amount of abiraterone acetate; wherein the prostate cancer is adenocarcinoma of the prostate, metastatic prostate cancer, castrate-resistant prostate cancer, metastatic castrate-resistant prostate cancer, or progressive metastatic castrate-resistant prostate cancer; and wherein the therapeutically effective amount of Compound A is about 5 mg to about 750 mg.

In one aspect, this application pertains to a method of treating prostate cancer in a subject comprising administering to the subject in need thereof:
(i) a therapeutically effective amount of Compound A,

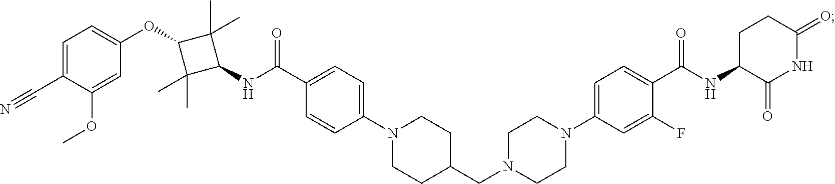 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

and
(ii) a therapeutically effective amount of abiraterone acetate; wherein the prostate cancer is adenocarcinoma of the prostate, metastatic prostate cancer, castrate-resistant prostate cancer, metastatic castrate-resistant prostate cancer, or progressive metastatic castrate-resistant prostate cancer; and wherein the therapeutically effective amount of Compound A is about 5 mg to about 750 mg.

In some embodiments, the prostate cancer is adenocarcinoma of the prostate.

In some embodiments, the prostate cancer is metastatic prostate cancer.

In some embodiments, the prostate cancer is castrate-resistant prostate cancer.

In some embodiments, the prostate cancer is metastatic castrate-resistant prostate cancer.

In some embodiments, the prostate cancer is progressive metastatic castrate-resistant prostate cancer.

In one aspect, this application pertains to a method of treating prostate cancer in a subject comprising administering to the subject in need thereof a therapeutically effective amount of Compound A,

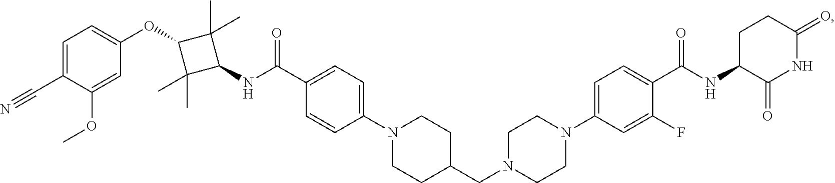 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

or a pharmaceutically acceptable salt thereof; wherein the prostate cancer is castrate-sensitive prostate cancer or metastatic castrate-sensitive prostate cancer; and wherein the therapeutically effective amount of Compound A is about 5 mg to about 750 mg.

In one aspect, this application pertains to a method of treating prostate cancer in a subject comprising administering to the subject in need thereof a therapeutically effective amount of Compound A,

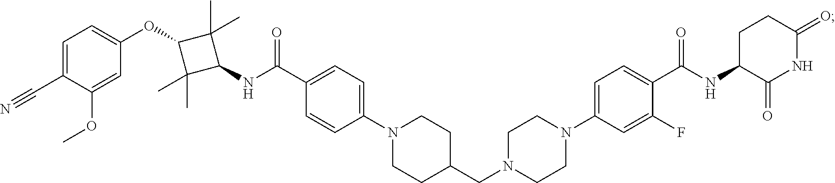 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

wherein the prostate cancer is castrate-sensitive prostate cancer or metastatic castrate-sensitive prostate cancer; and wherein the therapeutically effective amount of Compound A is about 5 mg to about 750 mg.

In one aspect, this application pertains to a method of treating prostate cancer in a subject comprising administering to the subject in need thereof:
(i) a therapeutically effective amount of Compound A,

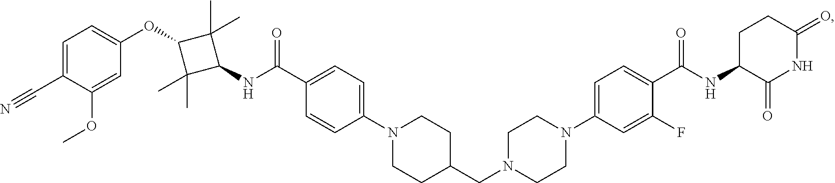 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

or a pharmaceutically acceptable salt thereof; and
(ii) a therapeutically effective amount of abiraterone acetate; wherein the prostate cancer is castrate-sensitive prostate cancer or metastatic castrate-sensitive prostate cancer; and wherein the therapeutically effective amount of Compound A is about 5 mg to about 750 mg.

In one aspect, this application pertains to a method of treating prostate cancer in a subject comprising administering to the subject in need thereof:
(i) a therapeutically effective amount of Compound A,

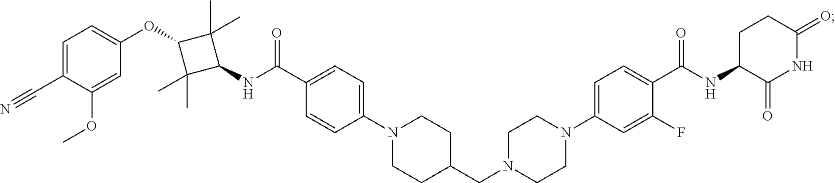 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

and
(ii) a therapeutically effective amount of abiraterone acetate; wherein the prostate cancer is castrate-sensitive prostate cancer or metastatic castrate-sensitive prostate cancer; and wherein the therapeutically effective amount of Compound A is about 5 mg to about 750 mg.

In some embodiments, the prostate cancer is castrate-sensitive prostate cancer.

In some embodiments, the prostate cancer is metastatic castrate-sensitive prostate cancer.

In one aspect, this application pertains to a method of treating prostate cancer naïve to novel hormonal agents (NHAs) in a subject comprising administering to the subject in need thereof a therapeutically effective amount of Compound A,

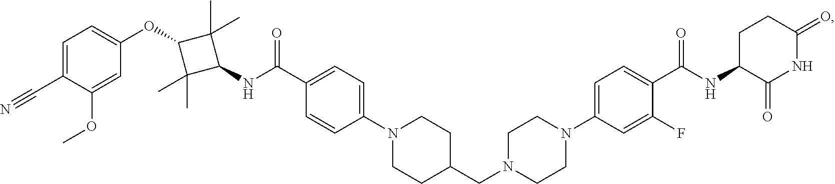 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

or a pharmaceutically acceptable salt thereof; and wherein the therapeutically effective amount of Compound A is about 5 mg to about 750 mg.

In one aspect, this application pertains to a method of treating prostate cancer naïve to novel hormonal agents (NHAs) in a subject comprising administering to the subject in need thereof a therapeutically effective amount of Compound A,

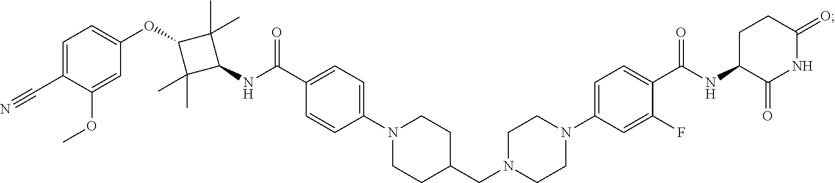 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

and wherein the therapeutically effective amount of Compound A is about 5 mg to about 750 mg.

In one aspect, this application pertains to a method of treating prostate cancer naïve to novel hormonal agents (NHAs) in a subject comprising administering to the subject in need thereof:
(i) a therapeutically effective amount of Compound A,

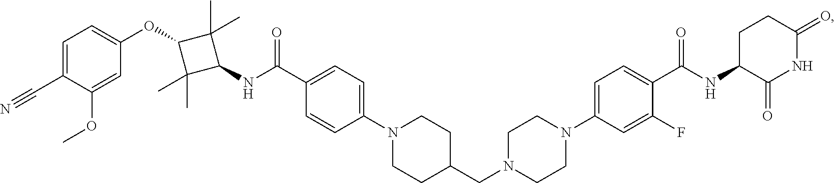 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

or a pharmaceutically acceptable salt thereof; and
(ii) a therapeutically effective amount of abiraterone acetate; and wherein the therapeutically effective amount of Compound A is about 5 mg to about 750 mg.

In one aspect, this application pertains to a method of treating prostate cancer naïve to novel hormonal agents (NHAs) in a subject comprising administering to the subject in need thereof:
(i) a therapeutically effective amount of Compound A,

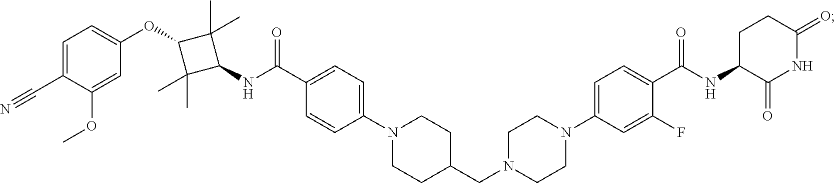 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

and

(ii) a therapeutically effective amount of abiraterone acetate; and wherein the therapeutically effective amount of Compound A is about 5 mg to about 750 mg.

In some embodiments, the prostate cancer naïve to NHAs is metastatic prostate cancer naïve to NHAs.

In some embodiments, the prostate cancer naïve to NHAs is castrate-resistant prostate cancer naïve to NHAs.

In some embodiments, the prostate cancer naïve to NHAs is castrate-sensitive prostate cancer naïve to NHAs.

In some embodiments, the prostate cancer naïve to NHAs is metastatic castrate-resistant prostate cancer naïve to NHAs.

In some embodiments, the prostate cancer naïve to NHAs is metastatic castrate-sensitive prostate cancer naïve to NHAs.

In some embodiments, the prostate cancer naïve to NHAs has not been previously treated with a second-generation antiandrogen.

In some embodiments, the prostate cancer naïve to NHAs has not been previously treated with an androgen biosynthesis inhibitor or an androgen receptor blocker.

In some embodiments, the prostate cancer naïve to NHAs has not been previously treated with abiraterone acetate.

In some embodiments, the prostate cancer naïve to NHAs has not been previously treated with an androgen receptor blocker selected from enzalutamide, darolutamide, and apalutamide.

In some embodiments, the prostate cancer naïve to NHAs has not been previously treated with an anti-cancer agent.

In some embodiments, the subject has not been previously administered an androgen biosynthesis inhibitor or an androgen receptor blocker.

In some embodiments, the subject has not been previously administered abiraterone acetate.

In some embodiments, the subject has not been previously administered an androgen receptor blocker selected from enzalutamide, darolutamide, and apalutamide.

In some embodiments, the subject has not been previously administered an anti-cancer agent.

In some embodiments, the therapeutically effective amount of Compound A is administered orally to the subject.

In some embodiments, the therapeutically effective amount of Compound A is administered to the subject once a day, twice a day, three times a day, or four times a day.

In some embodiments, the therapeutically effective amount of Compound A is administered to the subject once a day.

In some embodiments, the therapeutically effective amount of Compound A is about 100 mg to about 500 mg.

In some embodiments, the therapeutically effective amount of Compound A is about 5 mg to about 250 mg.

In some embodiments, the therapeutically effective amount of Compound A is about 250 mg to about 500 mg.

In some embodiments, the therapeutically effective amount of Compound A is about 500 mg to about 750 mg.

In some embodiments, the therapeutically effective amount of Compound A is about 100 mg.

In some embodiments, the therapeutically effective amount of Compound A is about 150 mg.

In some embodiments, the therapeutically effective amount of Compound A is about 200 mg.

In some embodiments, the therapeutically effective amount of Compound A is about 300 mg.

In some embodiments, the therapeutically effective amount of Compound A is about 320 mg.

In some embodiments, the therapeutically effective amount of Compound A is about 400 mg.

In some embodiments, the therapeutically effective amount of Compound A is about 480 mg.

In some embodiments, the therapeutically effective amount of Compound A is about 500 mg.

In some embodiments, the subject is in a fed state at the time of administration.

In some embodiments, the subject is in a fasted state at the time of administration.

In some embodiments, the prostate cancer comprises at least one somatic AR tumor mutation.

In some embodiments, the subject with prostate cancer comprises at least one somatic AR tumor mutation.

In some embodiments, the at least one somatic AR tumor mutation is an AR ligand binding domain missense mutation.

In some embodiments, the at least one somatic AR tumor mutation is selected from the group consisting of L702X, V716X, V731X, W742X, M750X, H875X, F877X, T878X, D880X, L882X, S889X, D891X, M895X, M896X, E898X, and any combination thereof, wherein “X” refers to an amino acid residue, other than the wild type residue at that position, selected from alanine (A); valine (V); leucine (L); isoleucine (I); phenylalanine (F); methionine (M); tryptophan (W); proline (P); glycine (G); serine (S); threonine (T); cysteine (C); asparagine (N); glutamine (Q); tyrosine (Y); lysine (K); arginine (R); histidine (H); aspartate (D); and glutamate (E).

In some embodiments, the at least one somatic AR tumor mutation is selected from the group consisting of L702H, V716M, V731M, W742L, W742C, H875Y, H875Q, H875L, F877L, T878A, T878S, D880E, L882I, S889G, D891H, D891Y, M896T, M896V, E898G, and any combination thereof.

In some embodiments, the at least one somatic AR tumor mutation is selected from the group consisting of L702H, M895V, W742C, S889G, M750V, M896V, T878A, F877L, D891H, H875Y, and any combination thereof.

In some embodiments, the at least one somatic AR tumor mutation is
(i) L702H;
(ii) T878A;
(iii) T878S;
(iv) H875Y;
(v) L702H and T878A;
(vi) L702H and T878S;
(vii) L702H and H875Y;
(viii) T878A and H875Y;
(ix) T878S and H875Y;
(x) L702H, T878A, and H875Y; or
(xi) L702H, T878S, and H875Y.

In some embodiments, the at least one somatic AR tumor mutation is L702H.

In some embodiments, the therapeutically effective amount of abiraterone acetate is about 250 mg to about 1500 mg.

In some embodiments, the therapeutically effective amount of abiraterone acetate is about 1000 mg.

In some embodiments, the therapeutically effective amount of abiraterone acetate is orally administered to the subject.

In some embodiments, the therapeutically effective amount of abiraterone acetate is administered to the subject once daily.

In some embodiments, the methods of this application further comprise administering a corticosteroid to the subject.

In some embodiments, the method of claim 59, wherein about 0.5 mg, about 1 mg, about 1.5 mg, about 2 mg, about 2.5 mg, about 3 mg, about 3.5 mg, about 4 mg, about 4.5 mg, or about 5 mg of the corticosteroid is administered to the subject.

In some embodiments, the corticosteroid is orally administered to the subject once daily.

In some embodiments, the corticosteroid is orally administered to the subject twice daily.

In some embodiments, the corticosteroid is prednisone, prednisolone, methylprednisolone, cortisone, cortisol, dexamethasone, betamethasone, triamcinolone, deflazacort, fludrocortisone acetate, deoxycorticosterone acetate, aldosterone, or beclomethasone.

In some embodiments, the corticosteroid is prednisone.

In some embodiments, the corticosteroid is prednisolone.

In some embodiments, the methods of this application comprise the subject discontinuing the use of a PPI prior to beginning administering Compound A, or a pharmaceutically acceptable salt thereof.

In some embodiments, the PPI is esomeprazole.

In one aspect, this application pertains to a method of treating prostate cancer having at least one somatic AR tumor mutation in a subject comprising administering to the subject in need thereof a therapeutically effective amount of Compound A,

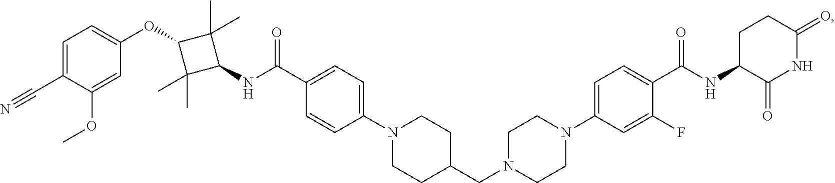 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

or a pharmaceutically acceptable salt thereof; wherein the prostate cancer having at least one somatic AR tumor mutation is adenocarcinoma of the prostate, metastatic prostate cancer, castrate-resistant prostate cancer, metastatic castrate-resistant prostate cancer, or progressive metastatic castrate-resistant prostate cancer; and wherein the therapeutically effective amount of Compound A is about 5 mg to about 750 mg.

In one aspect, this application pertains to a method of treating prostate cancer having at least one somatic AR tumor mutation in a subject comprising administering to the subject in need thereof a therapeutically effective amount of Compound A,

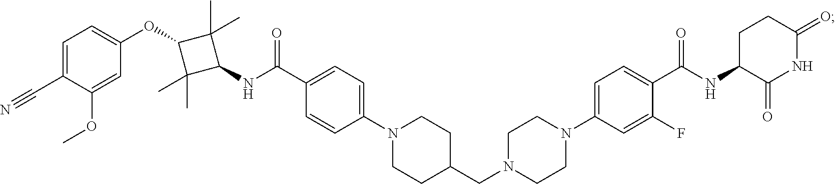 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

wherein the prostate cancer having at least one somatic AR tumor mutation is adenocarcinoma of the prostate, metastatic prostate cancer, castrate-resistant prostate cancer, metastatic castrate-resistant prostate cancer, or progressive metastatic castrate-resistant prostate cancer; and wherein the therapeutically effective amount of Compound A is about 5 mg to about 750 mg.

In one aspect, this application pertains to a method of treating prostate cancer having at least one somatic AR tumor mutation in a subject comprising administering to the subject in need thereof:
(i) a therapeutically effective amount of Compound A,

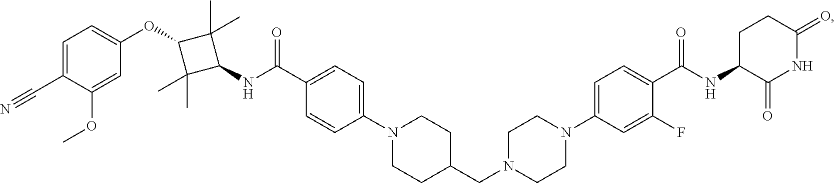 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

or a pharmaceutically acceptable salt thereof; and
(ii) a therapeutically effective amount of abiraterone acetate; wherein the prostate cancer having at least one somatic AR tumor mutation is adenocarcinoma of the prostate, metastatic prostate cancer, castrate-resistant prostate cancer, metastatic castrate-resistant prostate cancer, or progressive metastatic castrate-resistant prostate cancer; and wherein the therapeutically effective amount of Compound A is about 5 mg to about 750 mg.

In one aspect, this application pertains to a method of treating prostate cancer having at least one somatic AR tumor mutation in a subject comprising administering to the subject in need thereof:
(i) a therapeutically effective amount of Compound A,

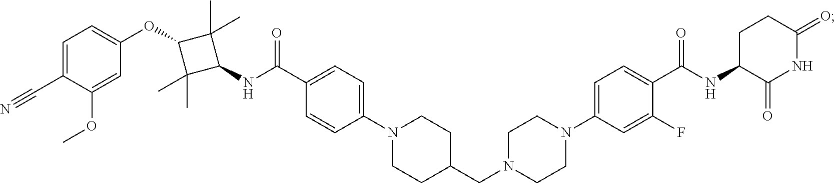 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

and
(ii) a therapeutically effective amount of abiraterone acetate; wherein the prostate cancer having at least one somatic AR tumor mutation is adenocarcinoma of the prostate, metastatic prostate cancer, castrate-resistant prostate cancer, metastatic castrate-resistant prostate cancer, or progressive metastatic castrate-resistant prostate cancer; and wherein the therapeutically effective amount of Compound A is about 5 mg to about 750 mg.

In some embodiments, the prostate cancer having at least one somatic AR tumor mutation is adenocarcinoma of the prostate.

In some embodiments, the prostate cancer having at least one somatic AR tumor mutation is metastatic prostate cancer.

In some embodiments, the prostate cancer having at least one somatic AR tumor mutation is castrate-resistant prostate cancer.

In some embodiments, the prostate cancer having at least one somatic AR tumor mutation is metastatic castrate-resistant prostate cancer.

In some embodiments, the prostate cancer having at least one somatic AR tumor mutation is progressive metastatic castrate-resistant prostate cancer.

In one aspect, this application pertains to a method of treating prostate cancer having at least one somatic AR tumor mutation in a subject comprising administering to the subject in need thereof a therapeutically effective amount of Compound A,

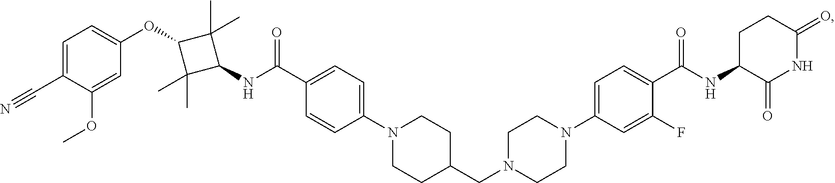 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

or a pharmaceutically acceptable salt thereof; wherein the prostate cancer having at least one somatic AR tumor mutation is castrate-sensitive prostate cancer or metastatic castrate-sensitive prostate cancer; and wherein the therapeutically effective amount of Compound A is about 5 mg to about 750 mg.

In one aspect, this application pertains to a method of treating prostate cancer having at least one somatic AR tumor mutation in a subject comprising administering to the subject in need thereof a therapeutically effective amount of Compound A,

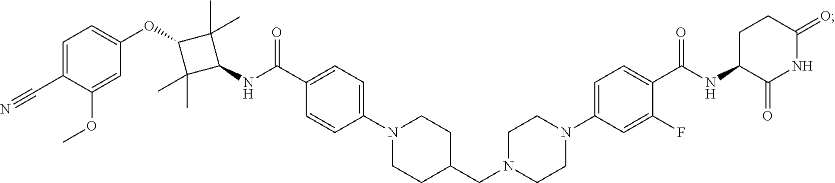 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

wherein the prostate cancer having at least one somatic AR tumor mutation is castrate-sensitive prostate cancer or metastatic castrate-sensitive prostate cancer; and wherein the therapeutically effective amount of Compound A is about 5 mg to about 750 mg.

In one aspect, this application pertains to a method of treating prostate cancer having at least one somatic AR tumor mutation in a subject comprising administering to the subject in need thereof:
(i) a therapeutically effective amount of Compound A,

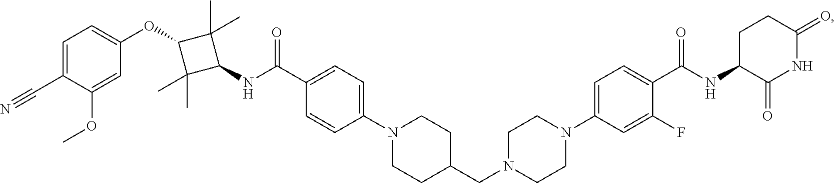 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

or a pharmaceutically acceptable salt thereof; and
(ii) a therapeutically effective amount of abiraterone acetate; wherein the prostate cancer having at least one somatic AR tumor mutation is castrate-sensitive prostate cancer or metastatic castrate-sensitive prostate cancer; and wherein the therapeutically effective amount of Compound A is about 5 mg to about 750 mg.

In one aspect, this application pertains to a method of treating prostate cancer having at least one somatic AR tumor mutation in a subject comprising administering to the subject in need thereof:
(i) a therapeutically effective amount of Compound A,

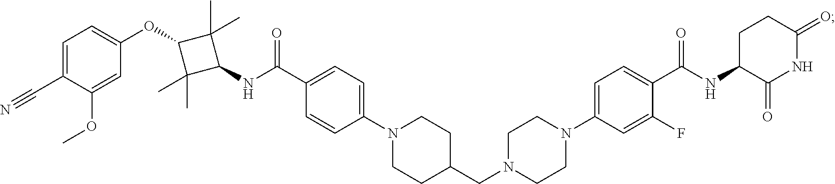 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

and
(ii) a therapeutically effective amount of abiraterone acetate; wherein the prostate cancer having at least one somatic AR tumor mutation is castrate-sensitive prostate cancer or metastatic castrate-sensitive prostate cancer; and wherein the therapeutically effective amount of Compound A is about 5 mg to about 750 mg.

In some embodiments, the prostate cancer having at least one somatic AR tumor mutation is castrate-sensitive prostate cancer.

In some embodiments, the prostate cancer having at least one somatic AR tumor mutation is metastatic castrate-sensitive prostate cancer.

In one aspect, this application pertains to a method of treating prostate cancer naïve to novel hormonal agents (NHAs) having at least one somatic AR tumor mutation in a subject comprising administering to the subject in need thereof a therapeutically effective amount of Compound A,

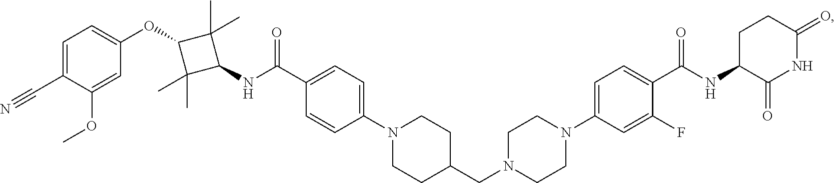 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

or a pharmaceutically acceptable salt thereof; and wherein the therapeutically effective amount of Compound A is about 5 mg to about 750 mg.

In one aspect, this application pertains to a method of treating prostate cancer naïve to novel hormonal agents (NHAs) having at least one somatic AR tumor mutation in a subject comprising administering to the subject in need thereof a therapeutically effective amount of Compound A,

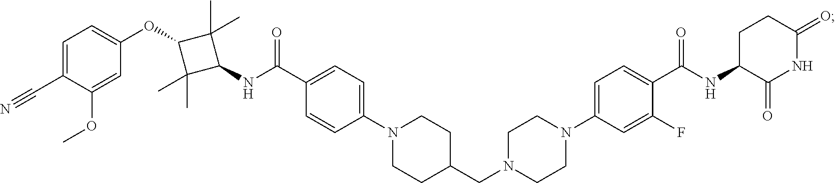 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

and wherein the therapeutically effective amount of Compound A is about 5 mg to about 750 mg.

In one aspect, this application pertains to a method of treating prostate cancer naïve to novel hormonal agents (NHAs) having at least one somatic AR tumor mutation in a subject comprising administering to the subject in need thereof:
(i) a therapeutically effective amount of Compound A,

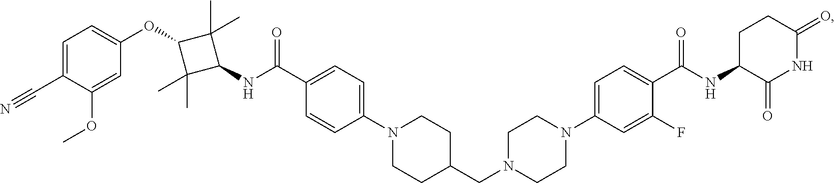 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

or a pharmaceutically acceptable salt thereof; and
(ii) a therapeutically effective amount of abiraterone acetate; and wherein the therapeutically effective amount of Compound A is about 5 mg to about 750 mg.

In one aspect, this application pertains to a method of treating prostate cancer naïve to novel hormonal agents (NHAs) having at least one somatic AR tumor mutation in a subject comprising administering to the subject in need thereof:
(i) a therapeutically effective amount of Compound A,

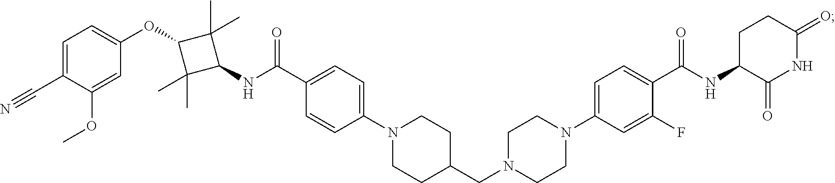 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

and
(ii) a therapeutically effective amount of abiraterone acetate; and wherein the therapeutically effective amount of Compound A is about 5 mg to about 750 mg.

In some embodiments, the prostate cancer naïve to NHAs having at least one somatic AR tumor mutation is metastatic prostate cancer naïve to NHAs.

In some embodiments, the prostate cancer naïve to NHAs having at least one somatic AR tumor mutation is castrate-resistant prostate cancer naïve to NHAs.

In some embodiments, the prostate cancer naïve to NHAs having at least one somatic AR tumor mutation is castrate-sensitive prostate cancer naïve to NHAs.

In some embodiments, the prostate cancer naïve to NHAs having at least one somatic AR tumor mutation is metastatic castrate-resistant prostate cancer naïve to NHAs.

In some embodiments, the prostate cancer naïve to NHAs having at least one somatic AR tumor mutation is metastatic castrate-sensitive prostate cancer naïve to NHAs.

In some embodiments, the at least one somatic AR tumor mutation is an AR ligand binding domain missense mutation.

In some embodiments, the at least one somatic AR tumor mutation is selected from the group consisting of L702X, V716X, V731X, W742X, M750X, H875X, F877X, T878X, D880X, L882X, S889X, D891X, M895X, M896X, E898X, and any combination thereof, wherein “X” refers to an amino acid residue, other than the wild type residue at that position, selected from alanine (A); valine (V); leucine (L); isoleucine (I); phenylalanine (F); methionine (M); tryptophan (W); proline (P); glycine (G); serine (S); threonine (T); cysteine (C); asparagine (N); glutamine (Q); tyrosine (Y); lysine (K); arginine (R); histidine (H); aspartate (D); and glutamate (E).

In some embodiments, the at least one somatic AR tumor mutation is selected from the group consisting of L702H, V716M, V731M, W742L, W742C, H875Y, H875Q, H875L, F877L, T878A, T878S, D880E, L882I, S889G, D891H, D891Y, M896T, M896V, E898G, and any combination thereof.

In some embodiments, the at least one somatic AR tumor mutation is selected from the group consisting of L702H, M895V, W742C, S889G, M750V, M896V, T878A, F877L, D891H, H875Y, and any combination thereof.

In some embodiments, the at least one somatic AR tumor mutation is selected from the group consisting of L702H, M895V, W742C, S889G, M750V, M896V, T878A, F877L, D891H, H875Y, and any combination thereof.

In some embodiments, the at least one somatic AR tumor mutation is
(i) L702H;
(ii) T878A;
(iii) T878S;
(iv) H875Y;
(v) L702H and T878A;
(vi) L702H and T878S;
(vii) L702H and H875Y;
(viii) T878A and H875Y;
(ix) T878S and H875Y;
(x) L702H, T878A, and H875Y; or
(xi) L702H, T878S, and H875Y.

In some embodiments, the at least one somatic AR tumor mutation is L702H.

In one aspect, this application pertains to a combined preparation of:
(a) Compound A,

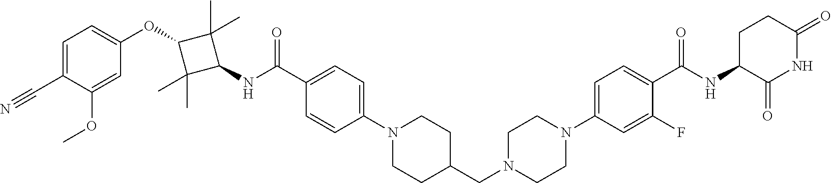 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

or a pharmaceutically acceptable salt thereof and
(b) abiraterone acetate; for simultaneous, separate or sequential use in a method of treating prostate cancer in a subject; wherein the prostate cancer is adenocarcinoma of the prostate, metastatic prostate cancer, castrate-resistant prostate cancer, metastatic castrate-resistant prostate cancer, progressive metastatic castrate-resistant prostate cancer, castrate-sensitive prostate cancer, or metastatic castrate-sensitive prostate cancer.

In some embodiments, the method comprises:
(i) oral, once daily administration of about 5 mg to about 750 mg of Compound A, or a pharmaceutically acceptable salt thereof; and
(ii) oral, once daily administration of about 250 mg to about 1500 mg abiraterone acetate.

In some embodiments, the method comprises:
(i) oral, once daily administration of about 5 mg to about 750 mg of Compound A; and (ii) oral, once daily administration of about 1000 mg abiraterone acetate.

In one aspect, this application pertains to a combined preparation of:
(a) Compound A,

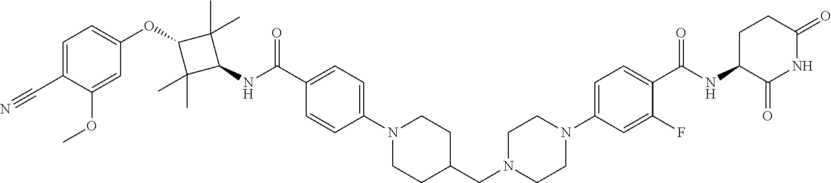 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

or a pharmaceutically acceptable salt thereof; and
(b) abiraterone acetate; for simultaneous, separate or sequential use in a method of treating prostate cancer naïve to novel hormonal agents (NHAs) in a subject.

In some embodiments, the method comprises:
(i) oral, once daily administration of about 5 mg to about 750 mg of Compound A, or a pharmaceutically acceptable salt thereof; and
(ii) oral, once daily administration of about 250 mg to about 1500 mg abiraterone acetate.

In some embodiments, the method comprises:
(i) oral, once daily administration of about 5 mg to about 750 mg of Compound A; and
(ii) oral, once daily administration of about 1000 mg abiraterone acetate.

In some embodiments, the prostate cancer naïve to novel hormonal agents (NHAs) is metastatic prostate cancer naïve to NHAs, castrate-resistant prostate cancer naïve to NHAs, castrate-sensitive prostate cancer naïve to NHAs, metastatic castrate-resistant prostate cancer naïve to NHAs, or metastatic castrate-sensitive prostate cancer naïve to NHAs. In one aspect, this application pertains to Compound A,

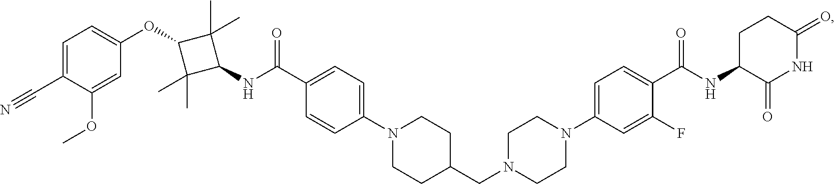 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

or a pharmaceutically acceptable salt thereof, for use in a method of treating prostate cancer in a subject, wherein the method comprises:
(i) oral, once daily administration of about 5 mg to about 750 mg of Compound A or the pharmaceutically acceptable salt thereof; and
(ii) oral, once daily administration of about 250 mg to about 1500 mg abiraterone acetate; wherein the prostate cancer is adenocarcinoma of the prostate, metastatic prostate cancer, castrate-resistant prostate cancer, metastatic castrate-resistant prostate cancer, progressive metastatic castrate-resistant prostate cancer, castrate-sensitive prostate cancer, or metastatic castrate-sensitive prostate cancer.

In one aspect, this application pertains to Compound A,

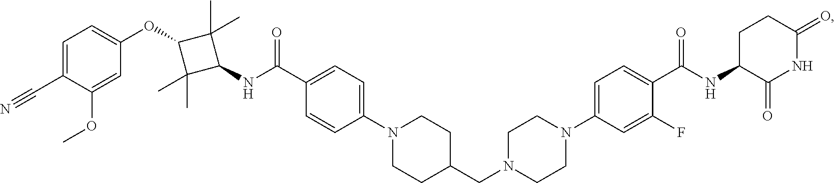 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

or a pharmaceutically acceptable salt thereof, for use in a method of treating prostate cancer in a subject, wherein the method comprises:
(i) oral, once daily administration of about 5 mg to about 750 mg of Compound A; and
(ii) oral, once daily administration of about 1000 mg abiraterone acetate; wherein the prostate cancer is adenocarcinoma of the prostate, metastatic prostate cancer, castrate-resistant prostate cancer, metastatic castrate-resistant prostate cancer, progressive metastatic castrate-resistant prostate cancer, castrate-sensitive prostate cancer, or metastatic castrate-sensitive prostate cancer.

In one aspect, this application pertains to Compound A,

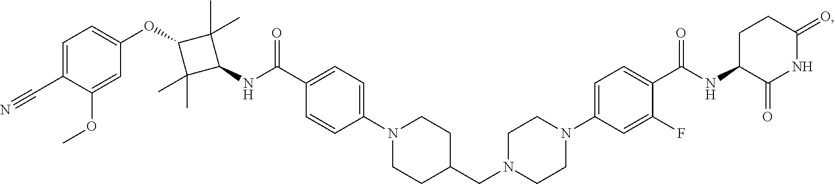 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

or a pharmaceutically acceptable salt thereof, for use in a method of treating prostate cancer naïve to novel hormonal agents (NHAs) in a subject, wherein the method comprises:
(i) oral, once daily administration of about 5 mg to about 750 mg of Compound A or the pharmaceutically acceptable salt thereof; and
(ii) oral, once daily administration of about 250 mg to about 1500 mg abiraterone acetate.

In one aspect, this application pertains to Compound A,

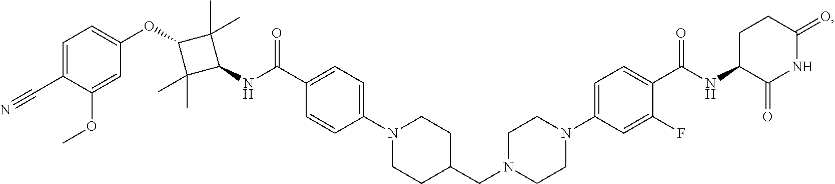 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

or a pharmaceutically acceptable salt thereof, for use in a method of treating prostate cancer naïve to novel hormonal agents (NHAs) in a subject, wherein the method comprises:
(i) oral, once daily administration of about 5 mg to about 750 mg of Compound A; and
(ii) oral, once daily administration of about 1000 mg abiraterone acetate.

In some embodiments, the prostate cancer naïve to novel hormonal agents (NHAs) is metastatic prostate cancer naïve to NHAs, castrate-resistant prostate cancer naïve to NHAs, castrate-sensitive prostate cancer naïve to NHAs, metastatic castrate-resistant prostate cancer naïve to NHAs, or metastatic castrate-sensitive prostate cancer naïve to NHAs.

In one aspect, this application pertains to a method of treating prostate cancer in a subject comprising administering to a subject in need thereof a therapeutically effective amount of Compound A,

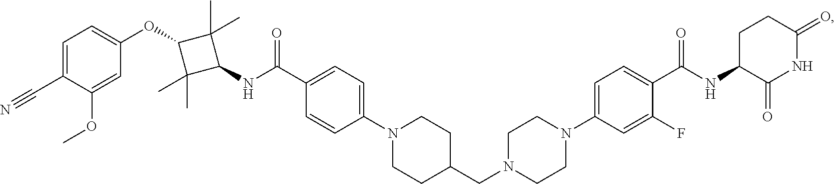 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

or a pharmaceutically acceptable salt thereof.

In one aspect, this application pertains to a method of treating prostate cancer in a subject comprising administering to a subject in need thereof a therapeutically effective amount of Compound A,

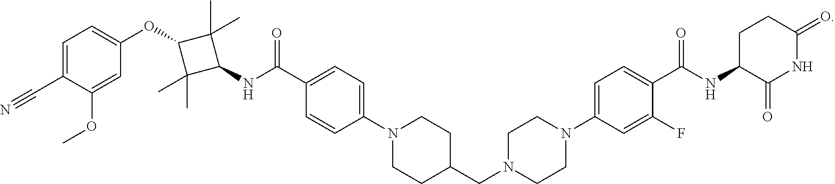 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

In one aspect, this application pertains to a method of treating prostate cancer in a subject comprising administering to a subject in need thereof a therapeutically effective amount of Compound A,

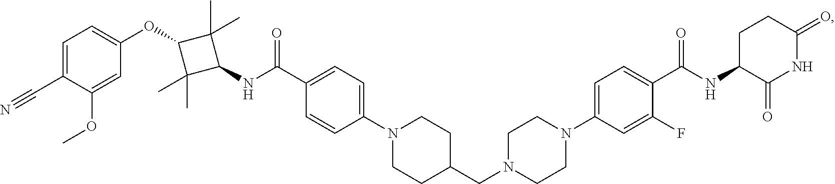 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

or a pharmaceutically acceptable salt thereof, wherein the subject is in a fed state at the time of administration.

In one aspect, this application pertains to a method of treating prostate cancer in a subject comprising administering to a subject in need thereof a therapeutically effective amount of Compound A,

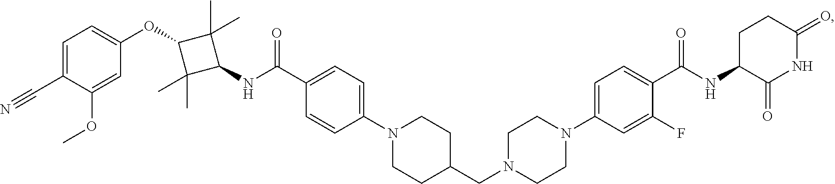 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

wherein the subject is in a fed state at the time of administration.

In one aspect, this application pertains to a method of treating: adenocarcinoma of the prostate, metastatic prostate cancer, castrate-resistant prostate cancer, metastatic castrate-resistant prostate cancer, progressive metastatic castrate-resistant prostate cancer, castrate-sensitive prostate cancer, metastatic castrate-sensitive prostate cancer, prostate cancer naïve to novel hormonal agents (NHA), metastatic prostate cancer naïve to novel hormonal agents (NHA), castrate-resistant prostate cancer naïve to novel hormonal agents (NHA), castrate-sensitive prostate cancer naïve to novel hormonal agents (NHA), metastatic castrate-resistant prostate cancer naïve to novel hormonal agents (NHA), or metastatic castrate-sensitive prostate cancer naïve to novel hormonal agents (NHA), in a subject comprising administering to a subject in need thereof a therapeutically effective amount of Compound A,

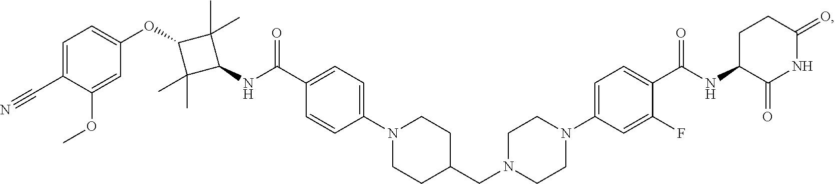 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

or a pharmaceutically acceptable salt thereof.

In one aspect, this application pertains to a method of treating: adenocarcinoma of the prostate, metastatic prostate cancer, castrate-resistant prostate cancer, metastatic castrate-resistant prostate cancer, progressive metastatic castrate-resistant prostate cancer, castrate-sensitive prostate cancer, metastatic castrate-sensitive prostate cancer, prostate cancer naïve to novel hormonal agents (NHA), metastatic prostate cancer naïve to novel hormonal agents (NHA), castrate-resistant prostate cancer naïve to novel hormonal agents (NHA), castrate-sensitive prostate cancer naïve to novel hormonal agents (NHA), metastatic castrate-resistant prostate cancer naïve to novel hormonal agents (NHA), or metastatic castrate-sensitive prostate cancer naïve to novel hormonal agents (NHA), in a subject comprising administering to a subject in need thereof a therapeutically effective amount of Compound A,

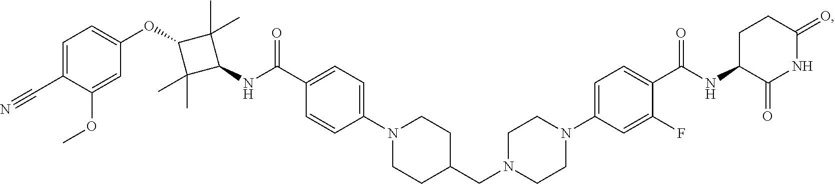 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

or a pharmaceutically acceptable salt thereof, wherein the subject is in a fed state at the time of administration.

In one aspect, this application pertains to a method of treating: adenocarcinoma of the prostate, metastatic prostate cancer, castrate-resistant prostate cancer, metastatic castrate-resistant prostate cancer, progressive metastatic castrate-resistant prostate cancer, castrate-sensitive prostate cancer, metastatic castrate-sensitive prostate cancer, prostate cancer naïve to novel hormonal agents (NHA), metastatic prostate cancer naïve to novel hormonal agents (NHA), castrate-resistant prostate cancer naïve to novel hormonal agents (NHA), castrate-sensitive prostate cancer naïve to novel hormonal agents (NHA), metastatic castrate-resistant prostate cancer naïve to novel hormonal agents (NHA), or metastatic castrate-sensitive prostate cancer naïve to novel hormonal agents (NHA), in a subject comprising administering to a subject in need thereof a therapeutically effective amount of Compound A,

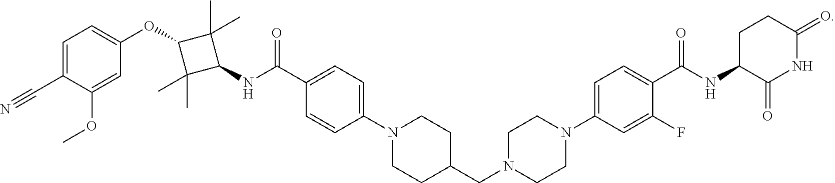 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

In one aspect, this application pertains to a method of treating: adenocarcinoma of the prostate, metastatic prostate cancer, castrate-resistant prostate cancer, metastatic castrate-resistant prostate cancer, progressive metastatic castrate-resistant prostate cancer, castrate-sensitive prostate cancer, metastatic castrate-sensitive prostate cancer, prostate cancer naïve to novel hormonal agents (NHA), metastatic prostate cancer naïve to novel hormonal agents (NHA), castrate-resistant prostate cancer naïve to novel hormonal agents (NHA), castrate-sensitive prostate cancer naïve to novel hormonal agents (NHA), metastatic castrate-resistant prostate cancer naïve to novel hormonal agents (NHA), or metastatic castrate-sensitive prostate cancer naïve to novel hormonal agents (NHA), in a subject comprising administering to a subject in need thereof a therapeutically effective amount of Compound A,

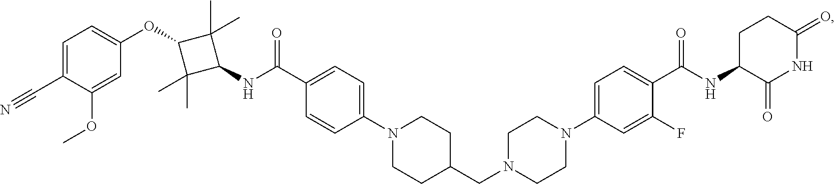 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

wherein the subject is in a fed state at the time of administration.

In one aspect, this application pertains to a method of treating prostate cancer naïve to novel hormonal agents (NHA), metastatic prostate cancer naïve to novel hormonal agents (NHA), castrate-resistant prostate cancer naïve to novel hormonal agents (NHA), castrate-sensitive prostate cancer naïve to novel hormonal agents (NHA), metastatic castrate-resistant prostate cancer naïve to novel hormonal agents (NHA), or metastatic castrate-sensitive prostate cancer naïve to novel hormonal agents (NHA), in a subject comprising administering to a subject in need thereof a therapeutically effective amount of Compound A,

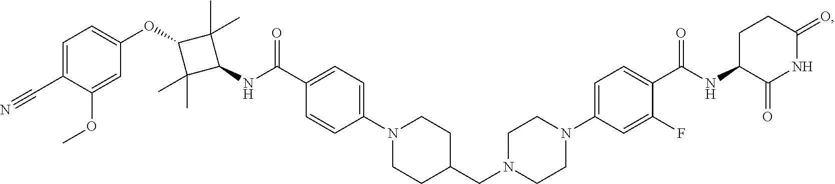 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

or a pharmaceutically acceptable salt thereof.

In one aspect, this application pertains to a method of treating prostate cancer naïve to novel hormonal agents (NHA), metastatic prostate cancer naïve to novel hormonal agents (NHA), castrate-resistant prostate cancer naïve to novel hormonal agents (NHA), castrate-sensitive prostate cancer naïve to novel hormonal agents (NHA), metastatic castrate-resistant prostate cancer naïve to novel hormonal agents (NHA), or metastatic castrate-sensitive prostate cancer naïve to novel hormonal agents (NHA), in a subject comprising administering to a subject in need thereof a therapeutically effective amount of Compound A,

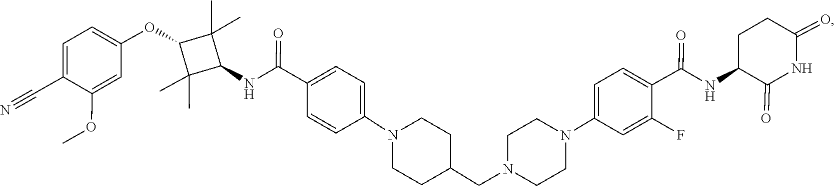 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

or a pharmaceutically acceptable salt thereof, wherein the subject is in a fed state at the time of administration.

In one aspect, this application pertains to a method of treating prostate cancer naïve to novel hormonal agents (NHA), metastatic prostate cancer naïve to novel hormonal agents (NHA), castrate-resistant prostate cancer naïve to novel hormonal agents (NHA), castrate-sensitive prostate cancer naïve to novel hormonal agents (NHA), metastatic castrate-resistant prostate cancer naïve to novel hormonal agents (NHA), or metastatic castrate-sensitive prostate cancer naïve to novel hormonal agents (NHA), in a subject comprising administering to a subject in need thereof a therapeutically effective amount of Compound A,

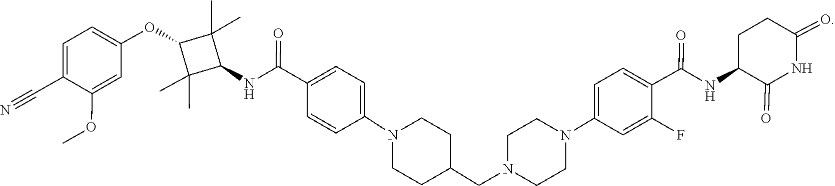 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

In one aspect, this application pertains to a method of treating prostate cancer naïve to novel hormonal agents (NHA), metastatic prostate cancer naïve to novel hormonal agents (NHA), castrate-resistant prostate cancer naïve to novel hormonal agents (NHA), castrate-sensitive prostate cancer naïve to novel hormonal agents (NHA), metastatic castrate-resistant prostate cancer naïve to novel hormonal agents (NHA), or metastatic castrate-sensitive prostate cancer naïve to novel hormonal agents (NHA), in a subject comprising administering to a subject in need thereof a therapeutically effective amount of Compound A,

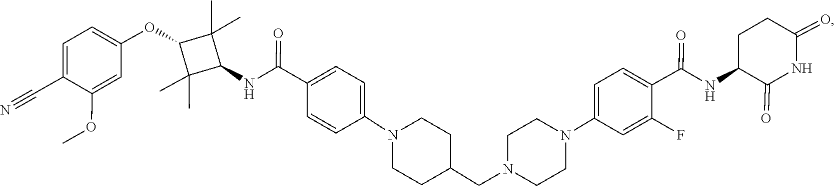 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

wherein the subject is in a fed state at the time of administration.

In one aspect, this application pertains to a method of treating prostate cancer naïve to novel hormonal agents (NHA), metastatic prostate cancer naïve to novel hormonal agents (NHA), castrate-resistant prostate cancer naïve to novel hormonal agents (NHA), castrate-sensitive prostate cancer naïve to novel hormonal agents (NHA), metastatic castrate-resistant prostate cancer naïve to novel hormonal agents (NHA), or metastatic castrate-sensitive prostate cancer naïve to novel hormonal agents (NHA), in a subject comprising administering to a subject in need thereof a therapeutically effective amount of Compound A,

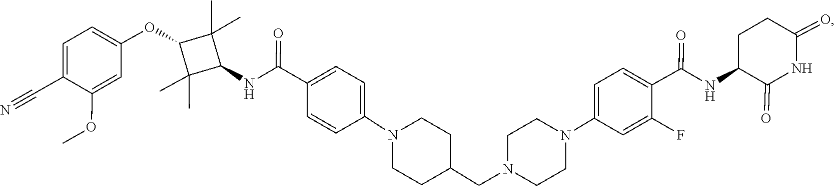 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

or a pharmaceutically acceptable salt thereof, where the subject with prostate cancer comprises at least one somatic AR tumor mutation.

In one aspect, this application pertains to a method of treating prostate cancer naïve to novel hormonal agents (NHA), metastatic prostate cancer naïve to novel hormonal agents (NHA), castrate-resistant prostate cancer naïve to novel hormonal agents (NHA), castrate-sensitive prostate cancer naïve to novel hormonal agents (NHA), metastatic castrate-resistant prostate cancer naïve to novel hormonal agents (NHA), or metastatic castrate-sensitive prostate cancer naïve to novel hormonal agents (NHA), in a subject comprising administering to a subject in need thereof a therapeutically effective amount of Compound A,

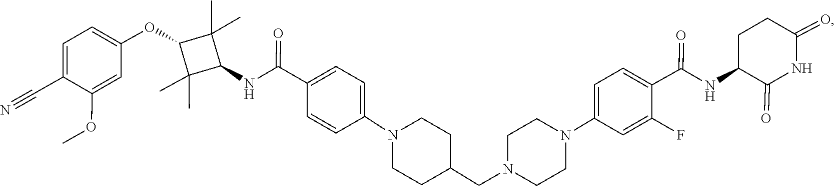 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

or a pharmaceutically acceptable salt thereof, wherein the subject is in a fed state at the time of administration, where the subject with prostate cancer comprises at least one somatic AR tumor mutation.

In one aspect, this application pertains to a method of treating prostate cancer naïve to novel hormonal agents (NHA), metastatic prostate cancer naïve to novel hormonal agents (NHA), castrate-resistant prostate cancer naïve to novel hormonal agents (NHA), castrate-sensitive prostate cancer naïve to novel hormonal agents (NHA), metastatic castrate-resistant prostate cancer naïve to novel hormonal agents (NHA), or metastatic castrate-sensitive prostate cancer naïve to novel hormonal agents (NHA), in a subject comprising administering to a subject in need thereof a therapeutically effective amount of Compound A,

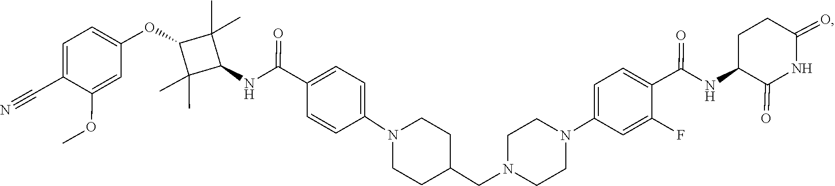 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

where the subject with prostate cancer comprises at least one somatic AR tumor mutation.

In one aspect, this application pertains to a method of treating prostate cancer naïve to novel hormonal agents (NHA), metastatic prostate cancer naïve to novel hormonal agents (NHA), castrate-resistant prostate cancer naïve to novel hormonal agents (NHA), castrate-sensitive prostate cancer naïve to novel hormonal agents (NHA), metastatic castrate-resistant prostate cancer naïve to novel hormonal agents (NHA), or metastatic castrate-sensitive prostate cancer naïve to novel hormonal agents (NHA), in a subject comprising administering to a subject in need thereof a therapeutically effective amount of Compound A,

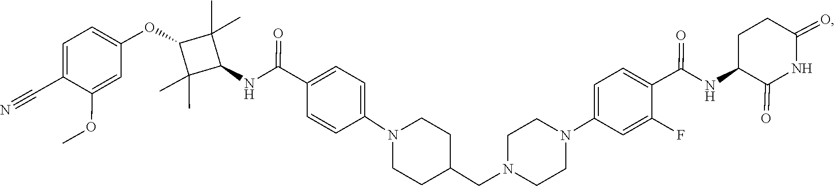 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

wherein the subject is in a fed state at the time of administration, where the subject with prostate cancer comprises at least one somatic AR tumor mutation.

In one aspect, this application pertains to a method of treating adenocarcinoma of the prostate in a subject comprising administering to the subject:
(i) a therapeutically effective amount of Compound A,

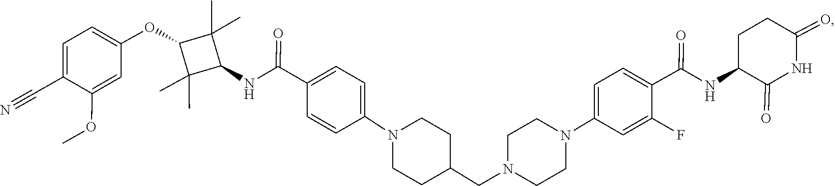 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

or a pharmaceutically acceptable salt thereof; and
(ii) a therapeutically effective amount of abiraterone acetate.

In one aspect, this application pertains to a method of treating adenocarcinoma of the prostate in a subject comprising administering to the subject:
(i) a therapeutically effective amount of Compound A,

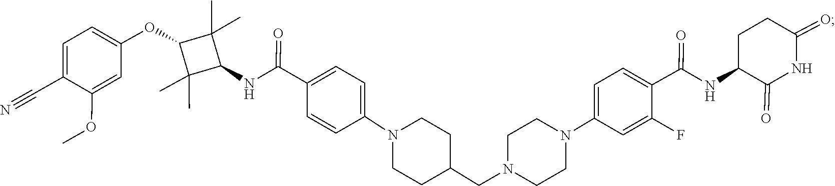 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

and
(ii) a therapeutically effective amount of abiraterone acetate.

In one aspect, this application pertains to a method of treating adenocarcinoma of the prostate in a subject comprising administering to the subject:
(i) a therapeutically effective amount of Compound A,

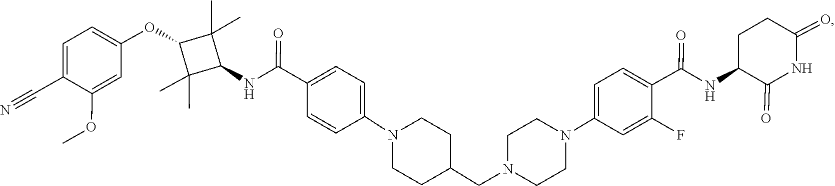 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

or a pharmaceutically acceptable salt thereof; and
(ii) a therapeutically effective amount of abiraterone acetate;

wherein the subject was not previously administered a novel hormonal agent (NHA). In some embodiments, the NHA is a second-generation antiandrogen. In some embodiments, the NHA is an androgen biosynthesis inhibitor. In some embodiments, the NHA is an androgen receptor blocker. In some embodiments, the NHA is abiraterone. In some embodiments, the NHA is abiraterone acetate. In some embodiments, the NHA is enzalutamide. In some embodiments, the NHA is darolutamide. In some embodiments, the NHA is apalutamide.

In one aspect, this application pertains to a method of treating adenocarcinoma of the prostate in a subject comprising administering to the subject:
(i) a therapeutically effective amount of Compound A,

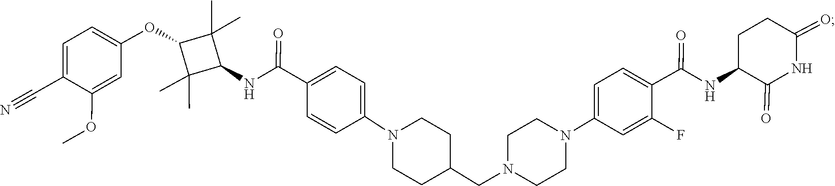 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

and
(ii) a therapeutically effective amount of abiraterone acetate;

wherein the subject was not previously administered a novel hormonal agent (NHA). In some embodiments, the NHA is a second-generation antiandrogen. In some embodiments, the NHA is an androgen biosynthesis inhibitor. In some embodiments, the NHA is an androgen receptor blocker. In some embodiments, the NHA is abiraterone. In some embodiments, the NHA is abiraterone acetate. In some embodiments, the NHA is enzalutamide. In some embodiments, the NHA is darolutamide. In some embodiments, the NHA is apalutamide.

In one aspect, this application pertains to a method of treating adenocarcinoma of the prostate in a subject comprising administering to the subject:
(i) a therapeutically effective amount of Compound A,

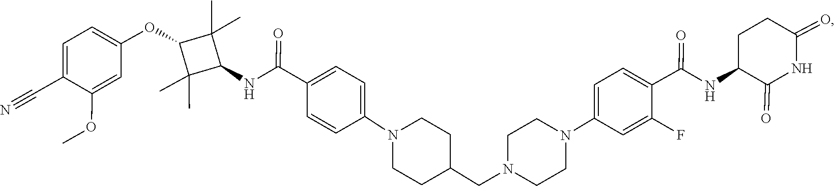 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

or a pharmaceutically acceptable salt thereof; and
(ii) a therapeutically effective amount of abiraterone acetate;
where the subject with adenocarcinoma of the prostate comprises at least one somatic AR tumor mutation.

In one aspect, this application pertains to a method of treating adenocarcinoma of the prostate in a subject comprising administering to the subject:
(i) a therapeutically effective amount of Compound A,

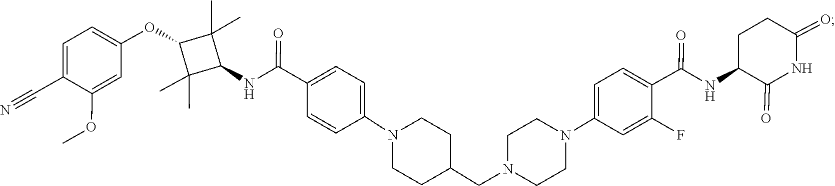 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

and
(ii) a therapeutically effective amount of abiraterone acetate;

where the subject with adenocarcinoma of the prostate comprises at least one somatic AR tumor mutation.

In one aspect, this application pertains to a method of treating metastatic prostate cancer in a subject comprising administering to the subject:
(i) a therapeutically effective amount of Compound A,

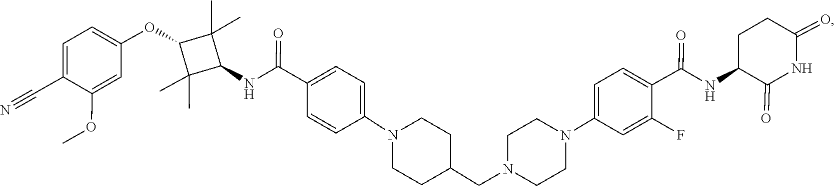 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

or a pharmaceutically acceptable salt thereof; and
(ii) a therapeutically effective amount of abiraterone acetate.

In one aspect, this application pertains to a method of treating metastatic prostate cancer in a subject comprising administering to the subject:
(i) a therapeutically effective amount of Compound A,

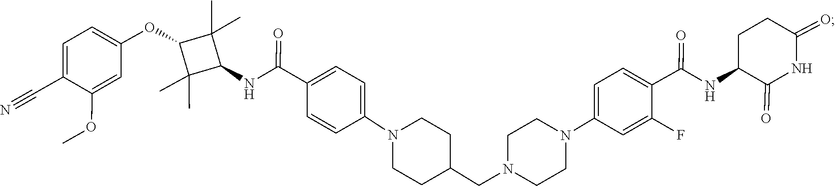 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

and
(ii) a therapeutically effective amount of abiraterone acetate.

In one aspect, this application pertains to a method of treating metastatic prostate cancer in a subject comprising administering to the subject:
(i) a therapeutically effective amount of Compound A,

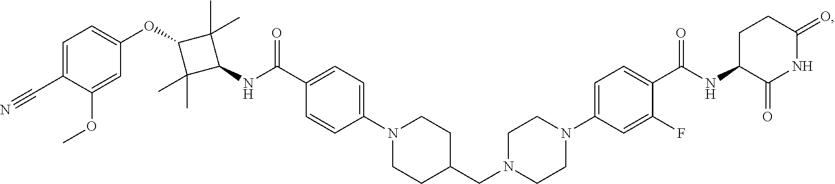 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

or a pharmaceutically acceptable salt thereof; and
(ii) a therapeutically effective amount of abiraterone acetate;
wherein the subject was not previously administered a novel hormonal agent (NHA). In some embodiments, the NHA is a second-generation antiandrogen. In some embodiments, the NHA is an androgen biosynthesis inhibitor. In some embodiments, the NHA is an androgen receptor blocker. In some embodiments, the NHA is abiraterone. In some embodiments, the NHA is abiraterone acetate. In some embodiments, the NHA is enzalutamide. In some embodiments, the NHA is darolutamide. In some embodiments, the NHA is apalutamide.

In one aspect, this application pertains to a method of treating metastatic prostate cancer in a subject comprising administering to the subject:
(i) a therapeutically effective amount of Compound A,

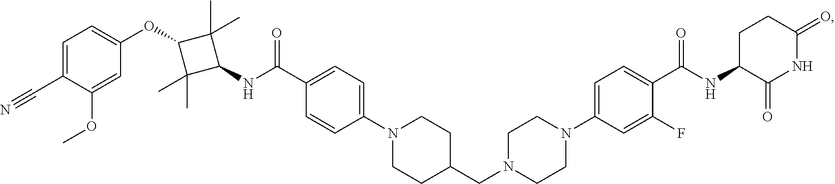 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

and
(ii) a therapeutically effective amount of abiraterone acetate;

wherein the subject was not previously administered a novel hormonal agent (NHA). In some embodiments, the NHA is a second-generation antiandrogen. In some embodiments, the NHA is an androgen biosynthesis inhibitor. In some embodiments, the NHA is an androgen receptor blocker. In some embodiments, the NHA is abiraterone. In some embodiments, the NHA is abiraterone acetate. In some embodiments, the NHA is enzalutamide. In some embodiments, the NHA is darolutamide. In some embodiments, the NHA is apalutamide.

In one aspect, this application pertains to a method of treating metastatic prostate cancer in a subject comprising administering to the subject:
(i) a therapeutically effective amount of Compound A,

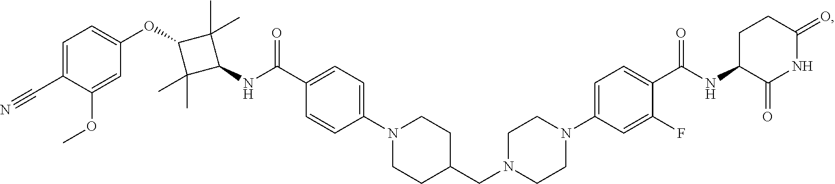 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

or a pharmaceutically acceptable salt thereof; and
(ii) a therapeutically effective amount of abiraterone acetate;
where the subject with metastatic prostate cancer comprises at least one somatic AR tumor mutation.

In one aspect, this application pertains to a method of treating metastatic prostate cancer in a subject comprising administering to the subject:
(i) a therapeutically effective amount of Compound A,

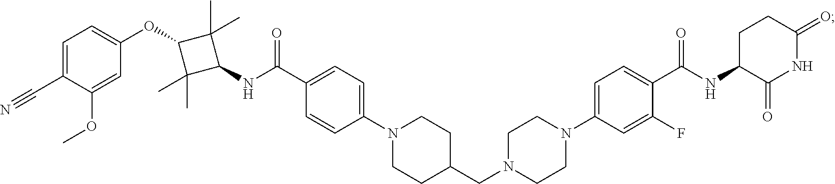 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

and
(ii) a therapeutically effective amount of abiraterone acetate;
where the subject with metastatic prostate cancer comprises at least one somatic AR tumor mutation.

In one aspect, this application pertains to a method of treating castrate-resistant prostate cancer in a subject comprising administering to the subject:
(i) a therapeutically effective amount of Compound A,

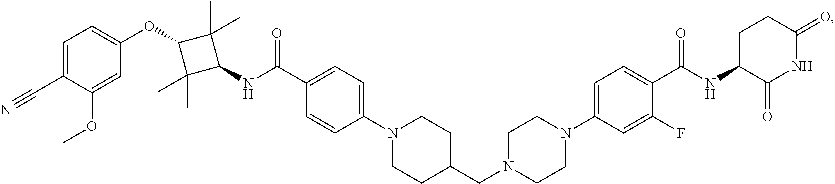 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

or a pharmaceutically acceptable salt thereof; and
(ii) a therapeutically effective amount of abiraterone acetate.

In one aspect, this application pertains to a method of treating castrate-resistant prostate cancer in a subject comprising administering to the subject:
(i) a therapeutically effective amount of Compound A,

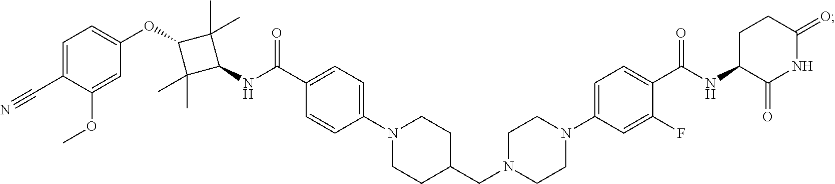 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

and
(ii) a therapeutically effective amount of abiraterone acetate.

In one aspect, this application pertains to a method of treating castrate-resistant prostate cancer in a subject comprising administering to the subject:
(i) a therapeutically effective amount of Compound A,

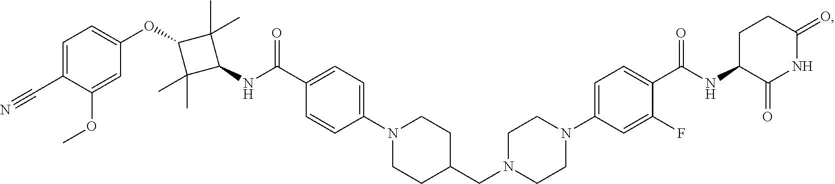 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

or a pharmaceutically acceptable salt thereof; and
(ii) a therapeutically effective amount of abiraterone acetate;
wherein the subject was not previously administered a novel hormonal agent (NHA). In some embodiments, the NHA is a second-generation antiandrogen. In some embodiments, the NHA is an androgen biosynthesis inhibitor. In some embodiments, the NHA is an androgen receptor blocker. In some embodiments, the NHA is abiraterone. In some embodiments, the NHA is abiraterone acetate. In some embodiments, the NHA is enzalutamide. In some embodiments, the NHA is darolutamide. In some embodiments, the NHA is apalutamide.

In one aspect, this application pertains to a method of treating castrate-resistant prostate cancer in a subject comprising administering to the subject:
(i) a therapeutically effective amount of Compound A,

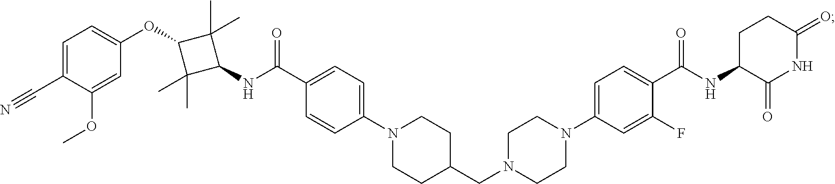 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

and
(ii) a therapeutically effective amount of abiraterone acetate;
wherein the subject was not previously administered a novel hormonal agent (NHA). In some embodiments, the NHA is a second-generation antiandrogen. In some embodiments, the NHA is an androgen biosynthesis inhibitor. In some embodiments, the NHA is an androgen receptor blocker. In some embodiments, the NHA is abiraterone. In some embodiments, the NHA is abiraterone acetate. In some embodiments, the NHA is enzalutamide. In some embodiments, the NHA is darolutamide. In some embodiments, the NHA is apalutamide.

In one aspect, this application pertains to a method of treating castrate-resistant prostate cancer in a subject comprising administering to the subject:
(i) a therapeutically effective amount of Compound A,

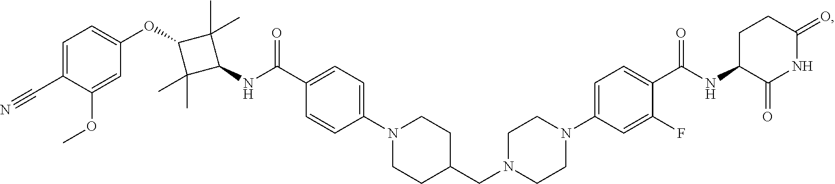 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

or a pharmaceutically acceptable salt thereof; and
(ii) a therapeutically effective amount of abiraterone acetate;
where the subject with castrate-resistant prostate cancer comprises at least one somatic AR tumor mutation.

In one aspect, this application pertains to a method of treating castrate-resistant prostate cancer in a subject comprising administering to the subject:
(i) a therapeutically effective amount of Compound A,

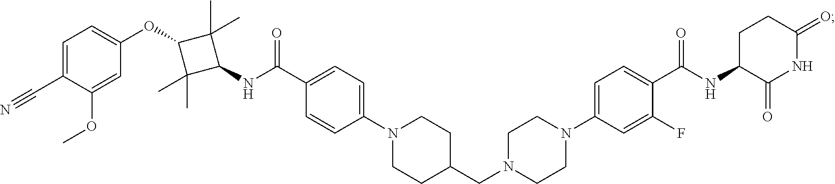 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

and
(ii) a therapeutically effective amount of abiraterone acetate;
where the subject with castrate-resistant prostate cancer comprises at least one somatic AR tumor mutation.

In one aspect, this application pertains to a method of treating metastatic castrate-resistant prostate cancer in a subject comprising administering to the subject:
(i) a therapeutically effective amount of Compound A,

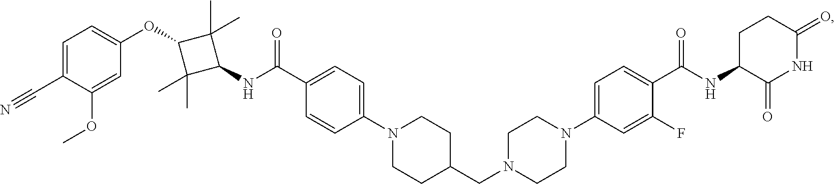 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

or a pharmaceutically acceptable salt thereof; and
(ii) a therapeutically effective amount of abiraterone acetate.

In one aspect, this application pertains to a method of treating metastatic castrate-resistant prostate cancer in a subject comprising administering to the subject:
(i) a therapeutically effective amount of Compound A,

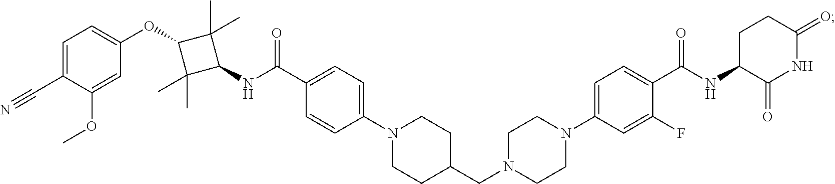 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

and
(ii) a therapeutically effective amount of abiraterone acetate.

In one aspect, this application pertains to a method of treating metastatic castrate-resistant prostate cancer in a subject comprising administering to the subject:
(i) a therapeutically effective amount of Compound A,

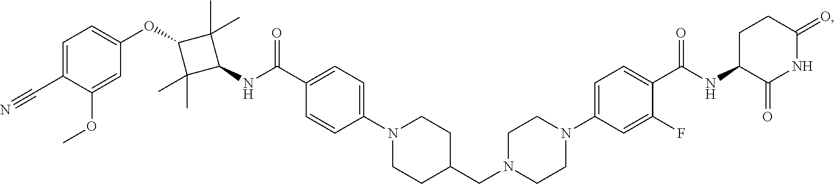 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

or a pharmaceutically acceptable salt thereof; and
(ii) a therapeutically effective amount of abiraterone acetate;
wherein the subject was not previously administered a novel hormonal agent (NHA). In some embodiments, the NHA is a second-generation antiandrogen. In some embodiments, the NHA is an androgen biosynthesis inhibitor. In some embodiments, the NHA is an androgen receptor blocker. In some embodiments, the NHA is abiraterone. In some embodiments, the NHA is abiraterone acetate. In some embodiments, the NHA is enzalutamide. In some embodiments, the NHA is darolutamide. In some embodiments, the NHA is apalutamide.

In one aspect, this application pertains to a method of treating metastatic castrate-resistant prostate cancer in a subject comprising administering to the subject:
(i) a therapeutically effective amount of Compound A,

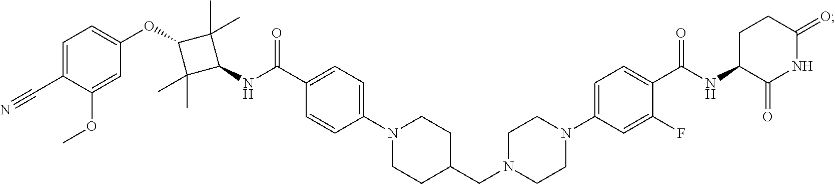 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

and
(ii) a therapeutically effective amount of abiraterone acetate;
wherein the subject was not previously administered a novel hormonal agent (NHA). In some embodiments, the NHA is a second-generation antiandrogen. In some embodiments, the NHA is an androgen biosynthesis inhibitor. In some embodiments, the NHA is an androgen receptor blocker. In some embodiments, the NHA is abiraterone. In some embodiments, the NHA is abiraterone acetate. In some embodiments, the NHA is enzalutamide. In some embodiments, the NHA is darolutamide. In some embodiments, the NHA is apalutamide.

In one aspect, this application pertains to a method of treating metastatic castrate-resistant prostate cancer in a subject comprising administering to the subject:
(i) a therapeutically effective amount of Compound A,

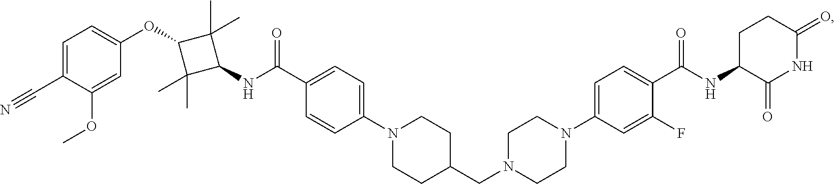 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

or a pharmaceutically acceptable salt thereof; and
(ii) a therapeutically effective amount of abiraterone acetate;
where the subject with metastatic castrate-resistant prostate cancer comprises at least one somatic AR tumor mutation.

In one aspect, this application pertains to a method of treating metastatic castrate-resistant prostate cancer in a subject comprising administering to the subject:
(i) a therapeutically effective amount of Compound A,

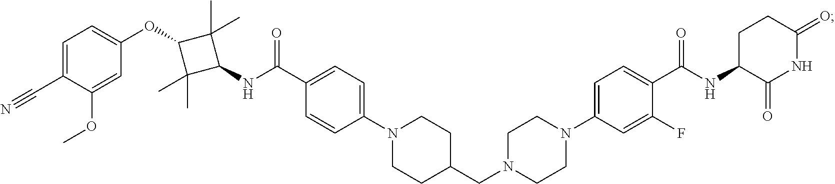 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

and
(ii) a therapeutically effective amount of abiraterone acetate;
where the subject with metastatic castrate-resistant prostate cancer comprises at least one somatic AR tumor mutation.

In one aspect, this application pertains to a method of treating progressive metastatic castrate-resistant prostate cancer in a subject comprising administering to the subject:
(i) a therapeutically effective amount of Compound A,

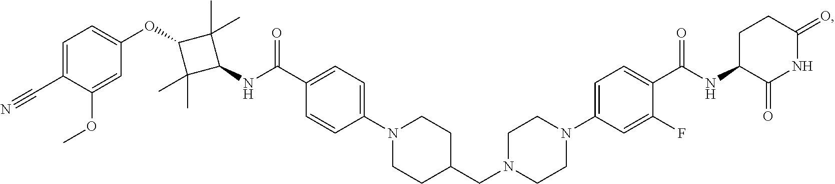 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

or a pharmaceutically acceptable salt thereof; and
(ii) a therapeutically effective amount of abiraterone acetate.

In one aspect, this application pertains to a method of treating progressive metastatic castrate-resistant prostate cancer in a subject comprising administering to the subject:
(i) a therapeutically effective amount of Compound A,

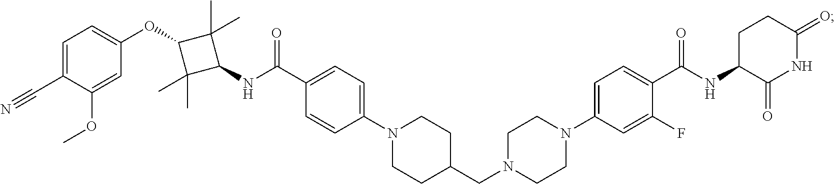 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

and
(ii) a therapeutically effective amount of abiraterone acetate.

In one aspect, this application pertains to a method of treating progressive metastatic castrate-resistant prostate cancer in a subject comprising administering to the subject:
(i) a therapeutically effective amount of Compound A,

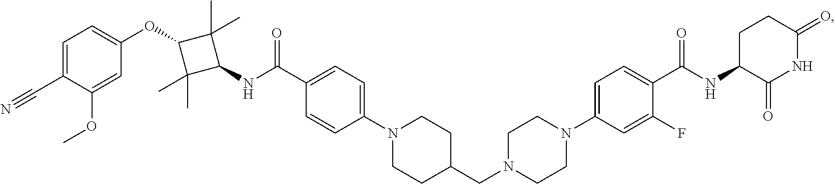 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

or a pharmaceutically acceptable salt thereof; and
(ii) a therapeutically effective amount of abiraterone acetate;
wherein the subject was not previously administered a novel hormonal agent (NHA). In some embodiments, the NHA is a second-generation antiandrogen. In some embodiments, the NHA is an androgen biosynthesis inhibitor. In some embodiments, the NHA is an androgen receptor blocker. In some embodiments, the NHA is abiraterone. In some embodiments, the NHA is abiraterone acetate. In some embodiments, the NHA is enzalutamide. In some embodiments, the NHA is darolutamide. In some embodiments, the NHA is apalutamide.

In one aspect, this application pertains to a method of treating progressive metastatic castrate-resistant prostate cancer in a subject comprising administering to the subject:
(i) a therapeutically effective amount of Compound A,

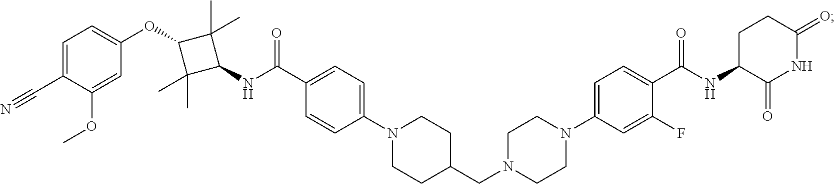 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

and
(ii) a therapeutically effective amount of abiraterone acetate;
wherein the subject was not previously administered a novel hormonal agent (NHA). In some embodiments, the NHA is a second-generation antiandrogen. In some embodiments, the NHA is an androgen biosynthesis inhibitor. In some embodiments, the NHA is an androgen receptor blocker. In some embodiments, the NHA is abiraterone. In some embodiments, the NHA is abiraterone acetate. In some embodiments, the NHA is enzalutamide. In some embodiments, the NHA is darolutamide. In some embodiments, the NHA is apalutamide.

In one aspect, this application pertains to a method of treating progressive metastatic castrate-resistant prostate cancer in a subject comprising administering to the subject:
(i) a therapeutically effective amount of Compound A,

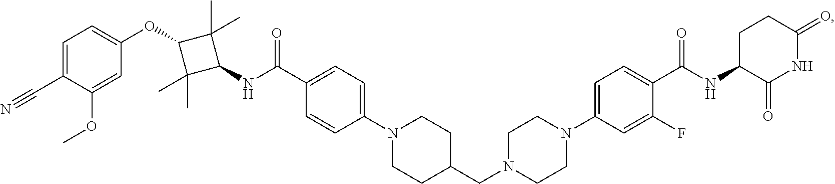 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

or a pharmaceutically acceptable salt thereof; and
(ii) a therapeutically effective amount of abiraterone acetate;
where the subject with progressive metastatic castrate-resistant prostate cancer comprises at least one somatic AR tumor mutation.

In one aspect, this application pertains to a method of treating progressive metastatic castrate-resistant prostate cancer in a subject comprising administering to the subject:
(i) a therapeutically effective amount of Compound A,

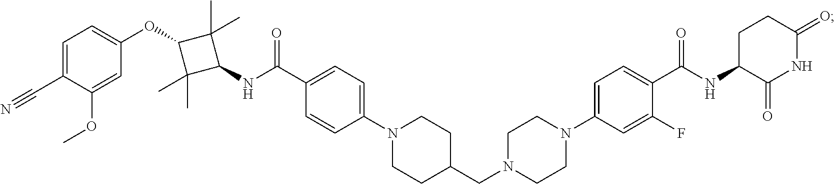 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

and
(ii) a therapeutically effective amount of abiraterone acetate;
where the subject with progressive metastatic castrate-resistant prostate cancer comprises at least one somatic AR tumor mutation.

In one aspect, this application pertains to a method of treating castrate-sensitive prostate cancer in a subject comprising administering to the subject:
(i) a therapeutically effective amount of Compound A,

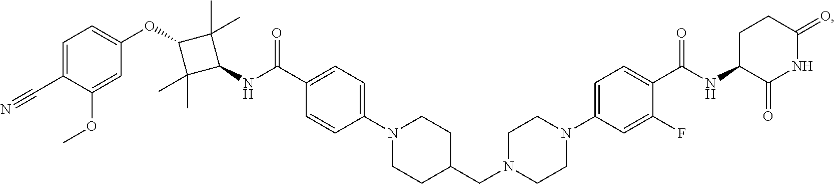 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

or a pharmaceutically acceptable salt thereof; and
(ii) a therapeutically effective amount of abiraterone acetate.

In one aspect, this application pertains to a method of treating castrate-sensitive prostate cancer in a subject comprising administering to the subject:
(i) a therapeutically effective amount of Compound A,

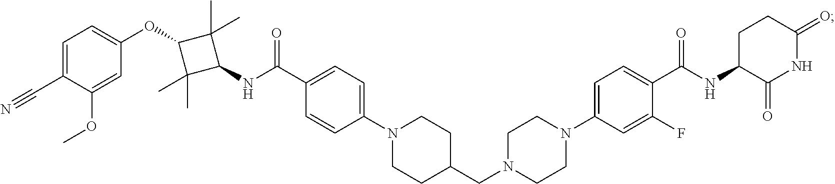 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

and
(ii) a therapeutically effective amount of abiraterone acetate.

In one aspect, this application pertains to a method of treating castrate-sensitive prostate cancer in a subject comprising administering to the subject:
(i) a therapeutically effective amount of Compound A,

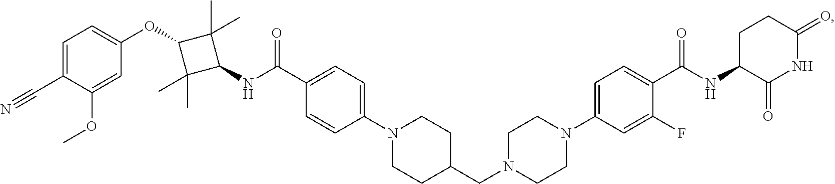 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

or a pharmaceutically acceptable salt thereof; and
(ii) a therapeutically effective amount of abiraterone acetate;
wherein the subject was not previously administered a novel hormonal agent (NHA). In some embodiments, the NHA is a second-generation antiandrogen. In some embodiments, the NHA is an androgen biosynthesis inhibitor. In some embodiments, the NHA is an androgen receptor blocker. In some embodiments, the NHA is abiraterone. In some embodiments, the NHA is abiraterone acetate. In some embodiments, the NHA is enzalutamide. In some embodiments, the NHA is darolutamide. In some embodiments, the NHA is apalutamide.

In one aspect, this application pertains to a method of treating castrate-sensitive prostate cancer in a subject comprising:
(i) a therapeutically effective amount of Compound A,

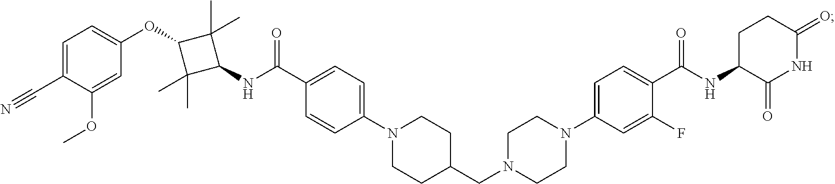 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

and
(ii) a therapeutically effective amount of abiraterone acetate;
wherein the subject was not previously administered a novel hormonal agent (NHA). In some embodiments, the NHA is a second-generation antiandrogen. In some embodiments, the NHA is an androgen biosynthesis inhibitor. In some embodiments, the NHA is an androgen receptor blocker. In some embodiments, the NHA is abiraterone. In some embodiments, the NHA is abiraterone acetate. In some embodiments, the NHA is enzalutamide. In some embodiments, the NHA is darolutamide. In some embodiments, the NHA is apalutamide.

In one aspect, this application pertains to a method of treating, castrate-sensitive prostate cancer in a subject comprising administering to the subject:
(i) a therapeutically effective amount of Compound A,

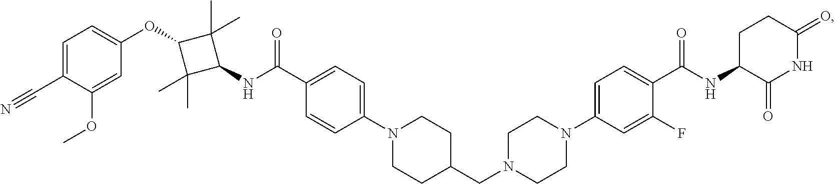 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

or a pharmaceutically acceptable salt thereof; and
(ii) a therapeutically effective amount of abiraterone acetate;
where the subject with castrate-sensitive prostate cancer comprises at least one somatic AR tumor mutation.

In one aspect, this application pertains to a method of treating castrate-sensitive prostate cancer in a subject comprising administering to the subject:
(i) a therapeutically effective amount of Compound A,

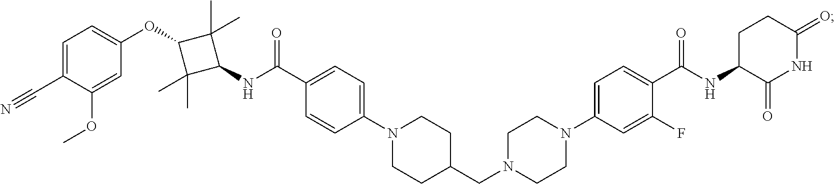 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

and
(ii) a therapeutically effective amount of abiraterone acetate;
where the subject with castrate-sensitive prostate cancer comprises at least one somatic AR tumor mutation.

In one aspect, this application pertains to a method of treating metastatic castrate-sensitive prostate cancer in a subject comprising administering to the subject:
(i) a therapeutically effective amount of Compound A,

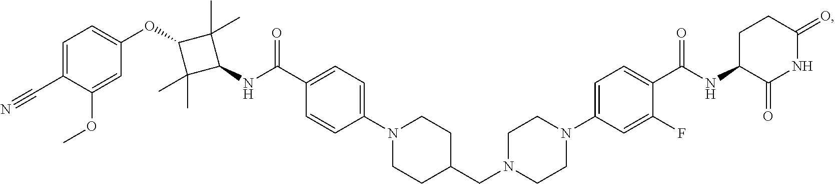 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

or a pharmaceutically acceptable salt thereof; and
(ii) a therapeutically effective amount of abiraterone acetate.

In one aspect, this application pertains to a method of treating metastatic castrate-sensitive prostate cancer in a subject comprising administering to the subject:
(i) a therapeutically effective amount of Compound A,

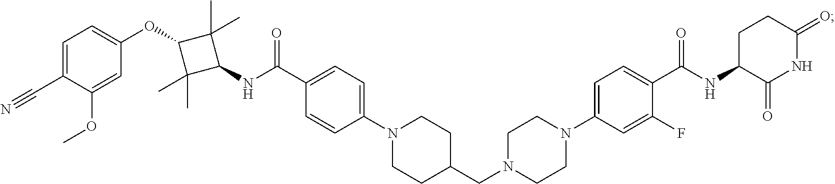 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

and
(ii) a therapeutically effective amount of abiraterone acetate.

In one aspect, this application pertains to a method of treating metastatic castrate-sensitive prostate cancer in a subject comprising administering to the subject:
(i) a therapeutically effective amount of Compound A,

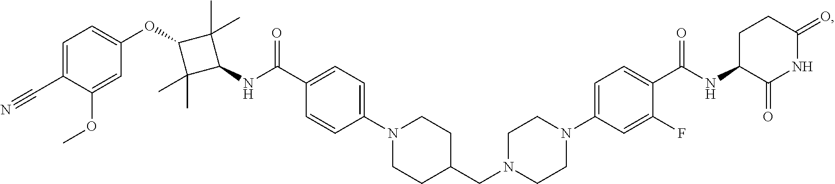 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

or a pharmaceutically acceptable salt thereof; and
(ii) a therapeutically effective amount of abiraterone acetate;
wherein the subject was not previously administered a novel hormonal agent (NHA). In some embodiments, the NHA is a second-generation antiandrogen. In some embodiments, the NHA is an androgen biosynthesis inhibitor. In some embodiments, the NHA is an androgen receptor blocker. In some embodiments, the NHA is abiraterone. In some embodiments, the NHA is abiraterone acetate. In some embodiments, the NHA is enzalutamide. In some embodiments, the NHA is darolutamide. In some embodiments, the NHA is apalutamide.

In one aspect, this application pertains to a method of treating metastatic castrate-sensitive prostate cancer in a subject comprising administering to the subject:
(i) a therapeutically effective amount of Compound A,

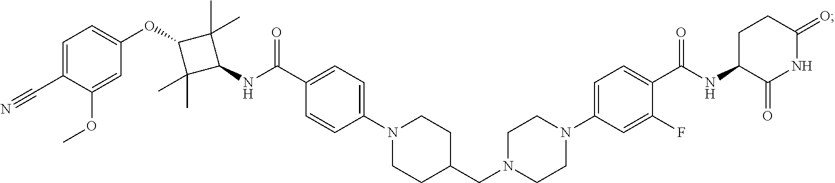 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

and
(ii) a therapeutically effective amount of abiraterone acetate;
wherein the subject was not previously administered a novel hormonal agent (NHA). In some embodiments, the NHA is a second-generation antiandrogen. In some embodiments, the NHA is an androgen biosynthesis inhibitor. In some embodiments, the NHA is an androgen receptor blocker. In some embodiments, the NHA is abiraterone. In some embodiments, the NHA is abiraterone acetate. In some embodiments, the NHA is enzalutamide. In some embodiments, the NHA is darolutamide. In some embodiments, the NHA is apalutamide.

In one aspect, this application pertains to a method of treating metastatic castrate-sensitive prostate cancer in a subject comprising administering to the subject:
(i) a therapeutically effective amount of Compound A,

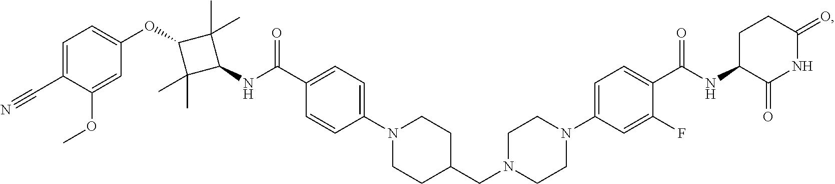 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

or a pharmaceutically acceptable salt thereof; and
(ii) a therapeutically effective amount of abiraterone acetate;
where the subject with metastatic castrate-sensitive prostate cancer comprises at least one somatic AR tumor mutation.

In one aspect, this application pertains to a method of treating metastatic castrate-sensitive prostate cancer in a subject comprising administering to the subject:
(i) a therapeutically effective amount of Compound A,

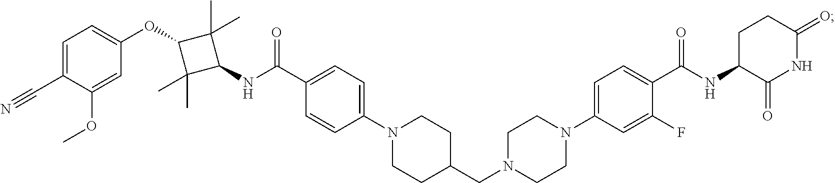 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

and
(ii) a therapeutically effective amount of abiraterone acetate;
where the subject with metastatic castrate-sensitive prostate cancer comprises at least one somatic AR tumor mutation.

In one aspect, this application pertains to a method of treating prostate cancer naïve to novel hormonal agents (NHA) in a subject comprising administering to the subject:
(i) a therapeutically effective amount of Compound A,

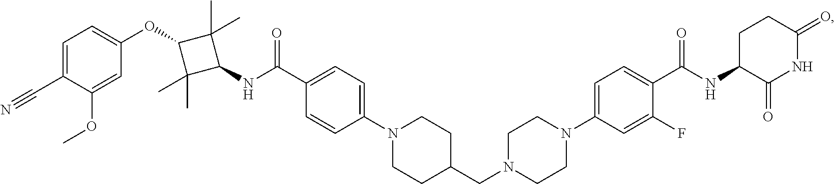 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

or a pharmaceutically acceptable salt thereof; and
(ii) a therapeutically effective amount of abiraterone acetate.

In one aspect, this application pertains to a method of treating prostate cancer naïve to novel hormonal agents (NHA) in a subject comprising administering to the subject:
(i) a therapeutically effective amount of Compound A,

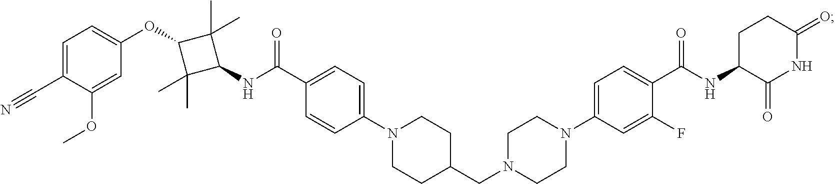 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

and
(ii) a therapeutically effective amount of abiraterone acetate.

In one aspect, this application pertains to a method of treating prostate cancer naïve to novel hormonal agents (NHA) in a subject comprising administering to the subject:
(i) a therapeutically effective amount of Compound A,

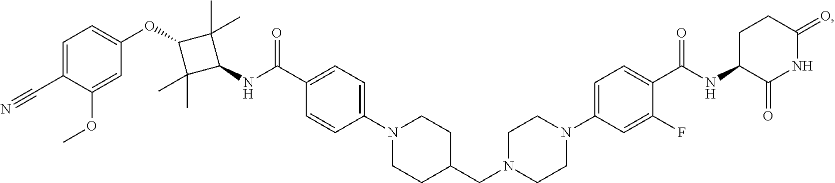 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

or a pharmaceutically acceptable salt thereof; and
(ii) a therapeutically effective amount of abiraterone acetate;
where the subject with prostate cancer naïve to novel hormonal agents (NHA) comprises at least one somatic AR tumor mutation.

In one aspect, this application pertains to a method of treating prostate cancer naïve to novel hormonal agents (NHA) in a subject comprising administering to the subject:
(i) a therapeutically effective amount of Compound A,

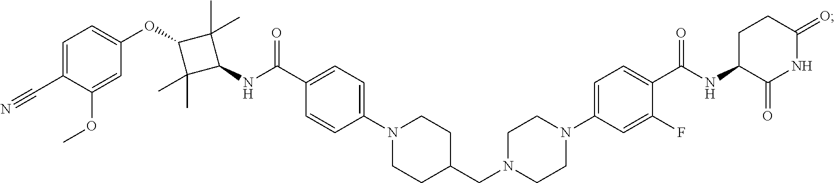 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

and
(ii) a therapeutically effective amount of abiraterone acetate;
where the subject with prostate cancer naïve to novel hormonal agents (NHA) comprises at least one somatic AR tumor mutation.

In one aspect, this application pertains to a method of treating metastatic prostate cancer naïve to novel hormonal agents (NHA) in a subject comprising administering to the subject:
(i) a therapeutically effective amount of Compound A,

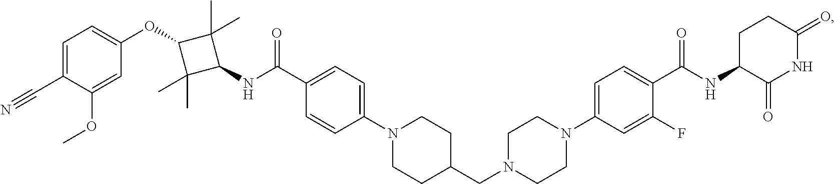 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

or a pharmaceutically acceptable salt thereof; and
(ii) a therapeutically effective amount of abiraterone acetate.

In one aspect, this application pertains to a method of treating metastatic prostate cancer naïve to novel hormonal agents (NHA) in a subject comprising administering to the subject:
(i) a therapeutically effective amount of Compound A,

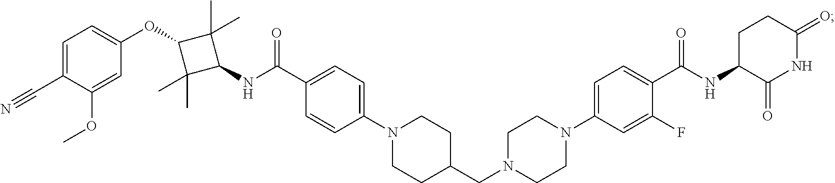 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

and
(ii) a therapeutically effective amount of abiraterone acetate.

In one aspect, this application pertains to a method of treating metastatic prostate cancer naïve to novel hormonal agents (NHA) in a subject comprising administering to the subject:
(i) a therapeutically effective amount of Compound A,

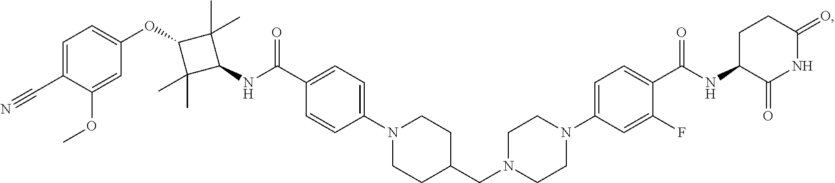 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

or a pharmaceutically acceptable salt thereof; and
(ii) a therapeutically effective amount of abiraterone acetate;
where the subject with metastatic prostate cancer naïve to novel hormonal agents (NHA) comprises at least one somatic AR tumor mutation.

In one aspect, this application pertains to a method of treating metastatic prostate cancer naïve to novel hormonal agents (NHA) in a subject comprising administering to the subject:
(i) a therapeutically effective amount of Compound A,

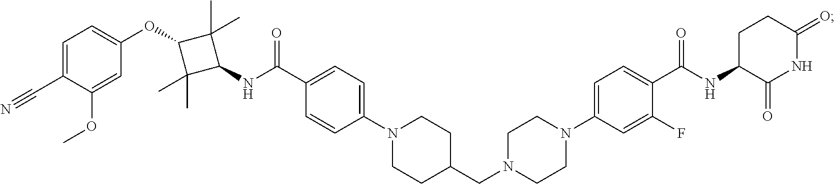 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

and
(ii) a therapeutically effective amount of abiraterone acetate;
where the subject with metastatic prostate cancer naïve to novel hormonal agents (NHA) comprises at least one somatic AR tumor mutation.

In one aspect, this application pertains to a method of treating castrate-resistant prostate cancer naïve to novel hormonal agents (NHA) in a subject comprising administering to the subject:
(i) a therapeutically effective amount of Compound A,

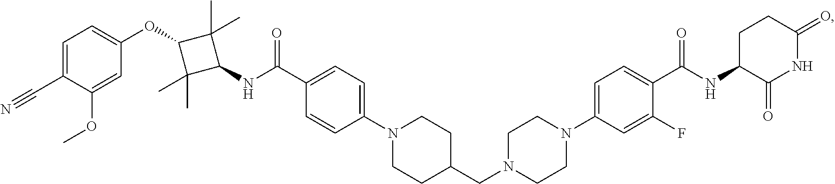 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

or a pharmaceutically acceptable salt thereof; and
(ii) a therapeutically effective amount of abiraterone acetate.

In one aspect, this application pertains to a method of treating castrate-resistant prostate cancer naïve to novel hormonal agents (NHA) in a subject comprising administering to the subject:
(i) a therapeutically effective amount of Compound A,

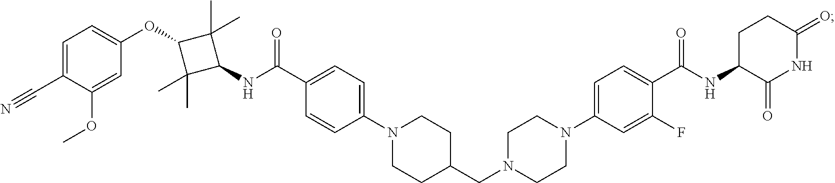 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

and
(ii) a therapeutically effective amount of abiraterone acetate.

In one aspect, this application pertains to a method of treating castrate-resistant prostate cancer naïve to novel hormonal agents (NHA), in a subject comprising administering to the subject:
(i) a therapeutically effective amount of Compound A,

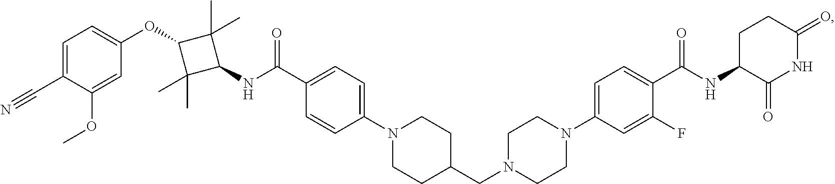 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

or a pharmaceutically acceptable salt thereof; and
(ii) a therapeutically effective amount of abiraterone acetate;
where the subject with castrate-resistant prostate cancer naïve to novel hormonal agents (NHA) comprises at least one somatic AR tumor mutation.

In one aspect, this application pertains to a method of treating castrate-resistant prostate cancer naïve to novel hormonal agents (NHA) in a subject comprising administering to the subject:
(i) a therapeutically effective amount of Compound A,

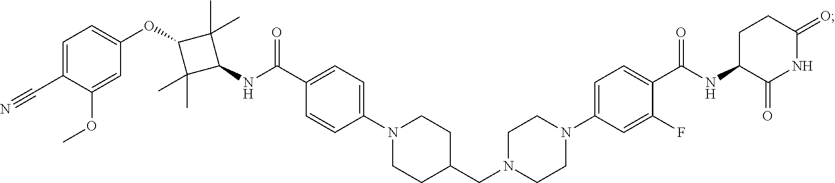 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

and
(ii) a therapeutically effective amount of abiraterone acetate;
where the subject with castrate-resistant prostate cancer naïve to novel hormonal agents (NHA) comprises at least one somatic AR tumor mutation.

In one aspect, this application pertains to a method of treating castrate-sensitive prostate cancer naïve to novel hormonal agents (NHA) in a subject comprising administering to the subject:
(i) a therapeutically effective amount of Compound A,

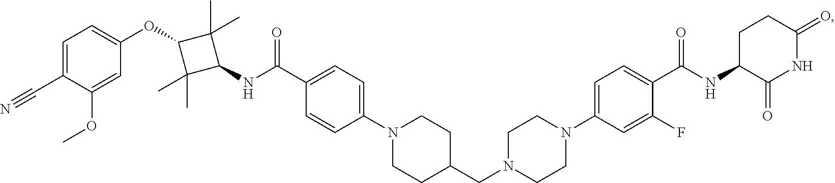 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

or a pharmaceutically acceptable salt thereof; and
(ii) a therapeutically effective amount of abiraterone acetate.

In one aspect, this application pertains to a method of treating castrate-sensitive prostate cancer naïve to novel hormonal agents (NHA) in a subject comprising administering to the subject:
(i) a therapeutically effective amount of Compound A,

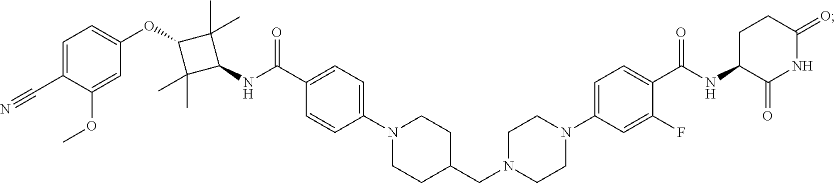 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

and
(ii) a therapeutically effective amount of abiraterone acetate.

In one aspect, this application pertains to a method of treating castrate-sensitive prostate cancer naïve to novel hormonal agents (NHA) in a subject comprising administering to the subject:
(i) a therapeutically effective amount of Compound A,

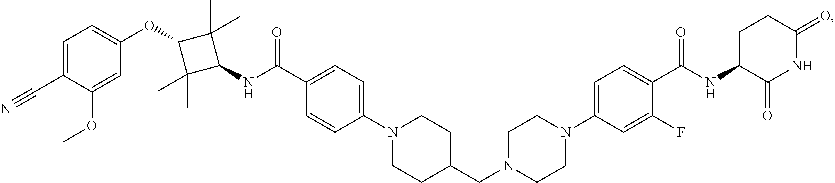 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

or a pharmaceutically acceptable salt thereof; and
(ii) a therapeutically effective amount of abiraterone acetate;
where the subject with castrate-sensitive prostate cancer naïve to novel hormonal agents (NHA) comprises at least one somatic AR tumor mutation.

In one aspect, this application pertains to a method of treating castrate-sensitive prostate cancer naïve to novel hormonal agents (NHA) in a subject comprising administering to the subject:
(i) a therapeutically effective amount of Compound A,

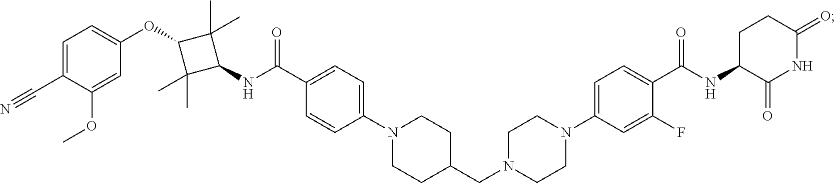 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

and
(ii) a therapeutically effective amount of abiraterone acetate;
where the subject with castrate-sensitive prostate cancer naïve to novel hormonal agents (NHA) comprises at least one somatic AR tumor mutation.

In one aspect, this application pertains to a method of treating metastatic castrate-resistant prostate cancer naïve to novel hormonal agents (NHA) in a subject comprising administering to the subject:
(i) a therapeutically effective amount of Compound A,

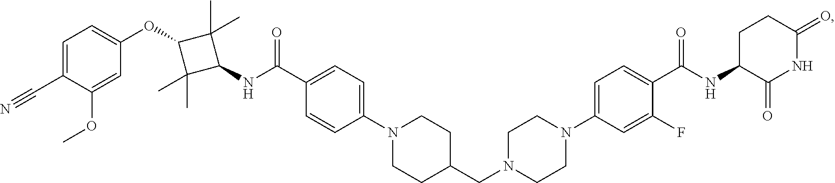 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

or a pharmaceutically acceptable salt thereof; and
(ii) a therapeutically effective amount of abiraterone acetate.

In one aspect, this application pertains to a method of treating metastatic castrate-resistant prostate cancer naïve to novel hormonal agents (NHA) in a subject comprising administering to the subject:
(i) a therapeutically effective amount of Compound A,

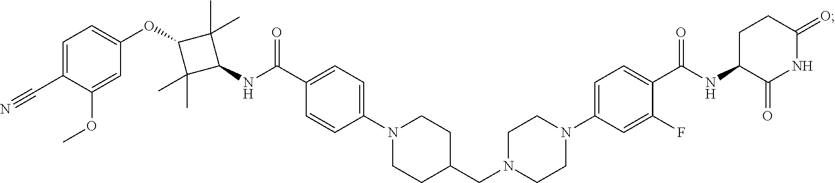 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

and
(ii) a therapeutically effective amount of abiraterone acetate.

In one aspect, this application pertains to a method of treating metastatic castrate-resistant prostate cancer naïve to novel hormonal agents (NHA) in a subject comprising administering to the subject:
(i) a therapeutically effective amount of Compound A,

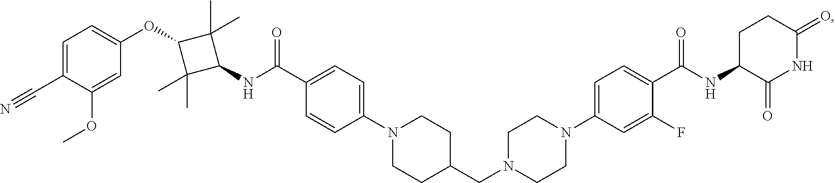 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

or a pharmaceutically acceptable salt thereof; and
(ii) a therapeutically effective amount of abiraterone acetate;
where the subject with metastatic castrate-resistant prostate cancer naïve to novel hormonal agents (NHA) comprises at least one somatic AR tumor mutation.

In one aspect, this application pertains to a method of treating metastatic castrate-resistant prostate cancer naïve to novel hormonal agents (NHA) in a subject comprising administering to the subject:
(i) a therapeutically effective amount of Compound A,

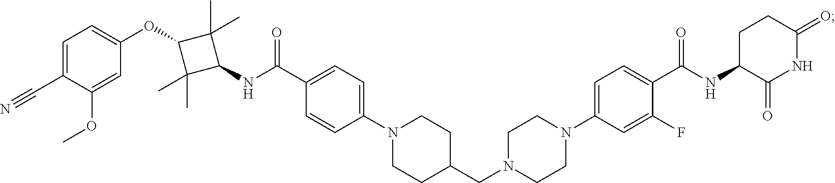 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

and
(ii) a therapeutically effective amount of abiraterone acetate;
where the subject with metastatic castrate-resistant prostate cancer naïve to novel hormonal agents (NHA) comprises at least one somatic AR tumor mutation.

In one aspect, this application pertains to a method of treating metastatic castrate-sensitive prostate cancer naïve to novel hormonal agents (NHA) in a subject comprising administering to the subject:
(i) a therapeutically effective amount of Compound A,

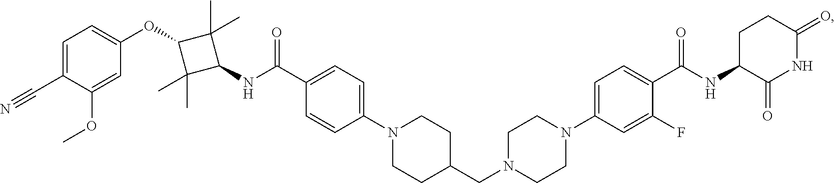 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

or a pharmaceutically acceptable salt thereof; and
(ii) a therapeutically effective amount of abiraterone acetate.

In one aspect, this application pertains to a method of treating metastatic castrate-sensitive prostate cancer naïve to novel hormonal agents (NHA) in a subject comprising administering to the subject:
(i) a therapeutically effective amount of Compound A,

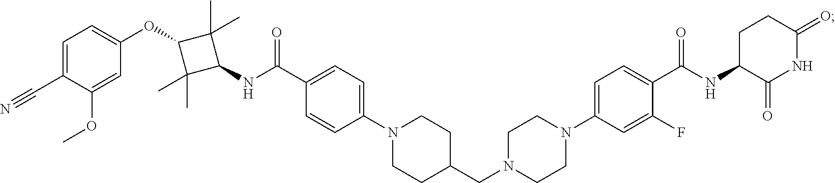 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

and
(ii) a therapeutically effective amount of abiraterone acetate.

In one aspect, this application pertains to a method of treating metastatic castrate-sensitive prostate cancer naïve to novel hormonal agents (NHA) in a subject comprising administering to the subject:
(i) a therapeutically effective amount of Compound A,

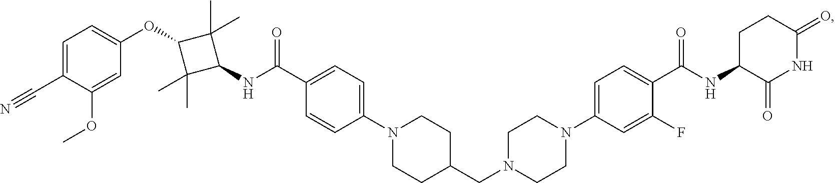 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

or a pharmaceutically acceptable salt thereof; and
(ii) a therapeutically effective amount of abiraterone acetate;
where the subject with metastatic castrate-sensitive prostate cancer naïve to novel hormonal agents (NHA) comprises at least one somatic AR tumor mutation.

In one aspect, this application pertains to a method of treating metastatic castrate-sensitive prostate cancer naïve to novel hormonal agents (NHA) in a subject comprising administering to the subject:
(i) a therapeutically effective amount of Compound A,

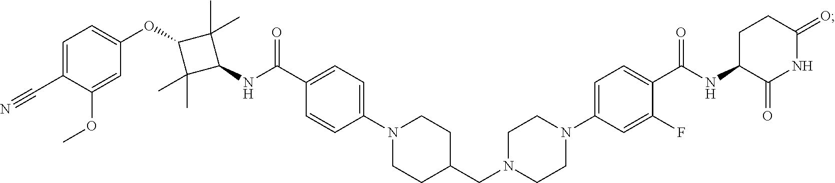 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

and
(ii) a therapeutically effective amount of abiraterone acetate;
where the subject with metastatic castrate-sensitive prostate cancer naïve to novel hormonal agents (NHA) comprises at least one somatic AR tumor mutation.

In one aspect, this application pertains to a method of treating prostate cancer in a subject that is not prostate cancer naïve to novel hormonal agents (NHA), optionally wherein the subject's prostate cancer has been treated previously with one, two, or more NHAs, comprising administering to a subject in need thereof a therapeutically effective amount of Compound A,

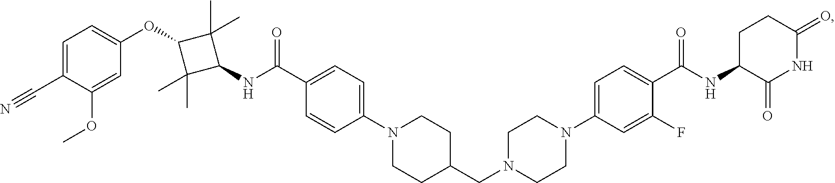 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

or a pharmaceutically acceptable salt thereof.

In one aspect, this application pertains to a method of treating prostate cancer in a subject that is not prostate cancer naïve to novel hormonal agents (NHA), optionally wherein the subject's prostate cancer has been treated previously with one, two, or more NHAs, comprising administering to a subject in need thereof a therapeutically effective amount of Compound A,

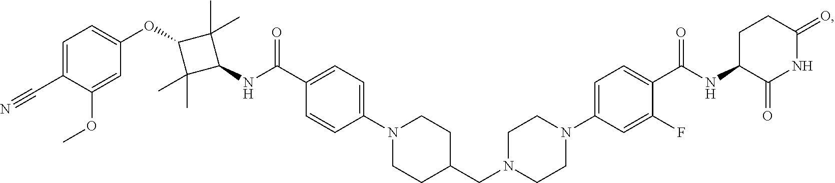 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

or a pharmaceutically acceptable salt thereof, wherein the subject is in a fed state at the time of administration.

In one aspect, this application pertains to a method of treating prostate cancer in a subject that is not prostate cancer naïve to novel hormonal agents (NHA), optionally wherein the subject's prostate cancer has been treated previously with one, two, or more NHAs, comprising administering to a subject in need thereof a therapeutically effective amount of Compound A,

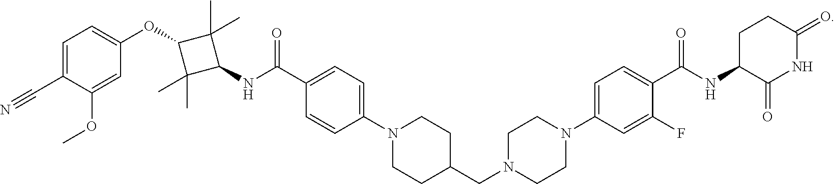 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

In one aspect, this application pertains to a method of treating prostate cancer in a subject that is not prostate cancer naïve to novel hormonal agents (NHA), optionally wherein the subject's prostate cancer has been treated previously with one, two, or more NHAs, comprising administering to a subject in need thereof a therapeutically effective amount of Compound A,

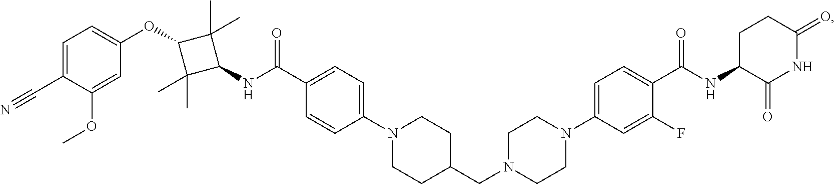 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

wherein the subject is in a fed state at the time of administration.

In some embodiments, the therapeutically effective amount of Compound A is administered orally to the subject.

In some embodiments, the therapeutically effective amount of Compound A is administered to the subject once a day, twice a day, three times a day, or four times a day.

In some embodiments, the therapeutically effective amount of Compound A is administered to the subject once a day.

In some embodiments, the therapeutically effective amount of Compound A is administered to the subject all at once or is administered in two, three, or four divided doses.

In some embodiments, the therapeutically effective amount of Compound A is about 1 mg to about 1000 mg.

In some embodiments, the therapeutically effective amount of Compound A is about 5 mg to about 750 mg.

In some embodiments, the therapeutically effective amount of Compound A is about 10 mg to about 500 mg.

In some embodiments, the therapeutically effective amount of Compound A is about 20 mg to about 250 mg.

In some embodiments, the therapeutically effective amount of Compound A is about 100 mg to about 500 mg.

In some embodiments, the therapeutically effective amount of Compound A is about 100 mg.

In some embodiments, the therapeutically effective amount of Compound A is 100 mg.

In some embodiments, the therapeutically effective amount of Compound A is about 150 mg.

In some embodiments, the therapeutically effective amount of Compound A is 150 mg.

In some embodiments, the therapeutically effective amount of Compound A is about 200 mg.

In some embodiments, the therapeutically effective amount of Compound A is 200 mg.

In some embodiments, the therapeutically effective amount of Compound A is about 300 mg.

In some embodiments, the therapeutically effective amount of Compound A is 300 mg.

In some embodiments, the therapeutically effective amount of Compound A is about 320 mg.

In some embodiments, the therapeutically effective amount of Compound A is 320 mg.

In some embodiments, the therapeutically effective amount of Compound A is about 400 mg.

In some embodiments, the therapeutically effective amount of Compound A is 400 mg.

In some embodiments, the therapeutically effective amount of Compound A is about 480 mg.

In some embodiments, the therapeutically effective amount of Compound A is 480 mg.

In some embodiments, the therapeutically effective amount of Compound A is about 500 mg.

In some embodiments, the therapeutically effective amount of Compound A is 500 mg.

In some embodiments, the subject is in a fed state at the time of administration.

In some embodiments, the subject is in a fasted state at the time of administration.

In some embodiments, the methods of the application further comprise the step of administering a PPI or H2 compound to the subject. In some embodiments, the methods of the application further comprise the step of administering a PPI or H2 compound to the subject prior to initiating administration of Compound A.

In some embodiments, the methods of the application further comprise the step of discontinuing the administration of the PPI or H2 compound to the subject prior to initiating administration of Compound A. In some embodiments, the administration of the PPI or H2 compound is discontinued in the subject beginning at a time point prior to initiating the administration of Compound A, wherein the time point is at least 10 hours.

In some embodiments, the methods of the application further comprise the step of reducing the administration of PPI or H2 compound to the subject prior to initiating administration of a therapeutically effective amount of Compound A. In some embodiments, the administration of the PPI or H2 compound is reduced in the subject at a time point prior to initiating the administration of Compound A, wherein the time point is at least 10 hours.

In some embodiments, the PPI or H2 compound is esomeprazole.

In some embodiments, the method further comprises administering an effective amount of at least one additional anti-cancer agent to the subject in need thereof.

In some embodiments, the additional anti-cancer agent is abiraterone, abiraterone acetate, estramustine, docetaxel, ketoconazole, goserelin, histrelin, triptorelin, buserelin, cyproterone, flutamide, bicalutamide, nilutamide, pamidronate, or zolendronate.

In some embodiments, the prostate cancer comprises at least one somatic AR tumor mutation.

In some embodiments, the subject with prostate cancer comprises at least one somatic AR tumor mutation.

In some embodiments, the at least one somatic AR tumor mutation is:
(i) selected from the group consisting of L702H, M895V, W742C, S889G, M750V, M896V, T878A, F877L, D891H, H875Y, and any combination thereof;
(ii) L702H;
(iii) T878A;
(iv) T878S;
(v) H875Y;
(vi) L702H and T878A;
(vii) L702H and T878S;
(viii) L702H and H875Y;
(ix) T878A and H875Y;
(x) T878S and H875Y;
(xi) L702H, T878A, and H875Y; or
(xii) L702H, T878S, and H875Y.

In one aspect, this application pertains to a method of treating prostate cancer in a subpopulation of prostate cancer subjects, comprising:
a. selecting a prostate cancer subject for treatment based on the subject's somatic AR tumor biomarker status; and
b. administering a therapeutically effective amount of Compound A,

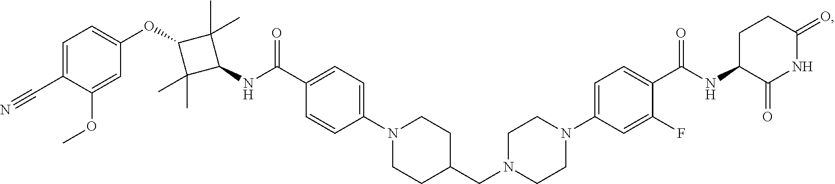 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

or a pharmaceutically acceptable salt thereof.

In one aspect, this application pertains to a method of treating prostate cancer in a subpopulation of prostate cancer subjects, comprising:
a. selecting a prostate cancer subject for treatment based on the subject's somatic AR tumor biomarker status; and
b. administering a therapeutically effective amount of Compound A,

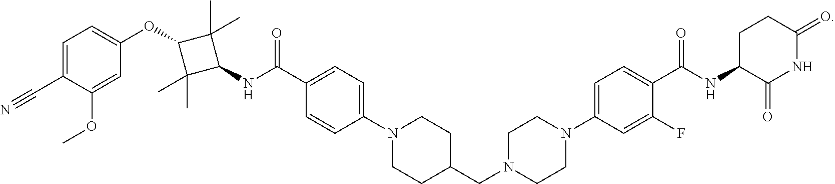 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

In some embodiments, the subject's somatic AR tumor biomarker status comprises at least one somatic AR tumor mutation.

In some embodiments, the at least one somatic AR tumor mutation is:
(i) selected from the group consisting of L702H, M895V, W742C, S889G, M750V, M896V, T878A, F877L, D891H, H875Y, and any combination thereof;
(ii) L702H;
(iii) T878A;
(iv) T878S;
(v) H875Y;
(vi) L702H and T878A;
(vii) L702H and T878S;
(viii) L702H and H875Y;
(ix) T878A and H875Y;
(x) T878S and H875Y;
(xi) L702H, T878A, and H875Y; or
(xii) L702H, T878S, and H875Y.

In some embodiments, the AR biomarker status of the subject is determined by ctDNA analysis, fluorescent in situ hybridization, immunohistochemistry, PCR analysis, or sequencing.

In some embodiments, the AR biomarker status of the subject is determined in a blood sample derived from the subject.

In some embodiments, the AR biomarker status of the subject is determined in a solid biopsy derived from the tumor of the subject.

In some embodiments, the subject is also undergoing ongoing androgen deprivation therapy (ADT).

In some embodiments, the ADT comprises the administration of a gonadotropin releasing hormone analog or inhibitor to the subject.

In some embodiments, the gonadotropin releasing hormone analog or inhibitor is leuprolide, goserelin, triptorelin, histrelin, or a pharmaceutically acceptable salt thereof.

In some embodiments, the subject has undergone an orchiectomy.

In some embodiments, the subject has a histological, pathological, or cytological confirmed diagnosis of adenocarcinoma of the prostate.

In some embodiments, prior to the administration of the therapeutically effective amount of Compound A, the subject experienced progression of prostate cancer on at least one prior approved systemic therapy for metastatic prostate cancer.

In some embodiments, prior to the administration of the therapeutically effective amount of Compound A, the subject experienced progression of prostate cancer on at least two prior approved systemic therapies for metastatic prostate cancer.

In some embodiments, the prior approved systemic therapy for metastatic prostate cancer is a second-generation androgen inhibitor.

In some embodiments, the second-generation androgen inhibitor is abiraterone, abiraterone acetate, enzalutamide, darolutamide, apalutamide, or a pharmaceutically acceptable salt thereof.

In some embodiments, the subject has an ECOG performance status of 0 or 1.

In some embodiments, the subject does not have a symptomatic brain metastasis requiring steroids above physiologic replacement doses.

In some embodiments, the subject does not have active inflammatory bowel disease.

In some embodiments, the subject does not have chronic diarrhea.

In some embodiments, the subject does not have diverticular disease.

In some embodiments, the subject has not previously undergone gastric resection.

In some embodiments, the subject has not previously undergone lap band surgery.

In some embodiments, the subject has not previously undergone radiation therapy within four weeks prior to the initial administration of the therapeutically effective amount of Compound A.

In some embodiments, the subject has not previously undergone radiation therapy where greater than about 25% of the subject's bone marrow was irradiated.

In some embodiments, the subject has not been administered an investigational drug within four weeks prior to the initial administration of the therapeutically effective amount of Compound A.

In some embodiments, the subject has metastatic castrate-resistant prostate cancer, wherein the subject's metastatic castrate-resistant prostate cancer shows radiographic evidence of metastatic disease.

In some embodiments, the subject has metastatic castrate-sensitive prostate cancer, wherein the subject's metastatic castrate-sensitive prostate cancer shows radiographic evidence of metastatic disease.

In some embodiments, the prostate cancer is adenocarcinoma of the prostate.

In some embodiments, the prostate cancer is metastatic prostate cancer.

In some embodiments, the prostate cancer is castrate-resistant prostate cancer.

In some embodiments, the prostate cancer is metastatic castrate-resistant prostate cancer.

In some embodiments, the metastatic castrate-resistant prostate cancer shows radiographic evidence of metastatic disease.

In some embodiments, the prostate cancer is progressive metastatic castrate-resistant prostate cancer.

In some embodiments, the prostate cancer is castrate-sensitive prostate cancer.

In some embodiments, the prostate cancer is metastatic castrate-sensitive prostate cancer.

In some embodiments, the metastatic castrate-sensitive prostate cancer shows radiographic evidence of metastatic disease.

In some embodiments, the prostate cancer is prostate cancer naïve to novel hormonal agents (NHA).

In some embodiments, the prostate cancer is metastatic prostate cancer naïve to novel hormonal agents (NHA).

In some embodiments, the prostate cancer is castrate-resistant prostate cancer naïve to novel hormonal agents (NHA).

In some embodiments, the prostate cancer is castrate-sensitive prostate cancer naïve to novel hormonal agents (NHA).

In some embodiments, the prostate cancer is metastatic castrate-resistant prostate cancer naïve to novel hormonal agents (NHA).

In some embodiments, the prostate cancer is progressive metastatic castrate-resistant prostate cancer naïve to novel hormonal agents (NHA).

In some embodiments, the prostate cancer is metastatic castrate-sensitive prostate cancer naïve to novel hormonal agents (NHA).

In some embodiments, the prostate cancer has not been previously treated with a second-generation antiandrogen. In some embodiments, the prostate cancer has not been previously treated with an androgen biosynthesis inhibitor or an androgen receptor blocker. In some embodiments, the prostate cancer has not been previously treated with an androgen biosynthesis inhibitor. In some embodiments, the prostate cancer has not been previously treated with an androgen receptor blocker. In some embodiments, the prostate cancer has not been previously treated with abiraterone acetate. In some embodiments, the prostate cancer has not been previously treated with an androgen receptor blocker selected from enzalutamide, darolutamide, and apalutamide.

In some embodiments, the subject has not been previously administered a second-generation antiandrogen. In some embodiments, the subject has not been previously administered an androgen biosynthesis inhibitor or an androgen receptor blocker. In some embodiments, the subject has not been previously administered an androgen biosynthesis inhibitor. In some embodiments, the subject has not been previously administered an androgen receptor blocker. In some embodiments, the subject has not been previously administered abiraterone acetate. In some embodiments, the subject has not been previously administered an androgen receptor blocker selected from enzalutamide, darolutamide, and apalutamide.

In some embodiments, the prostate cancer is metastatic prostate cancer naïve to novel hormonal agents (NHA). In some embodiments, the metastatic prostate cancer naïve to novel hormonal agents (NHA) has not been previously treated with one or more second-generation antiandrogens. In some embodiments, the metastatic prostate cancer naïve to novel hormonal agents (NHA) has not been previously treated with an androgen biosynthesis inhibitor or an androgen receptor blocker. In some embodiments, the metastatic prostate cancer naïve to novel hormonal agents (NHA) has not been previously treated with an androgen biosynthesis inhibitor. In some embodiments, the metastatic prostate cancer naïve to novel hormonal agents (NHA) has not been previously treated with an androgen receptor blocker. In some embodiments, the metastatic prostate cancer naïve to novel hormonal agents (NHA) has not been previously treated with abiraterone acetate. In some embodiments, the metastatic prostate cancer naïve to novel hormonal agents (NHA) has not been previously treated with an androgen receptor blocker selected from enzalutamide, darolutamide, and apalutamide.

In some embodiments, the prostate cancer is castrate-resistant prostate cancer naïve to novel hormonal agents (NHA). In some embodiments, the castrate-resistant prostate cancer naïve to novel hormonal agents (NHA) has not been previously treated with one or more second-generation antiandrogens. In some embodiments, the castrate-resistant prostate cancer naïve to novel hormonal agents (NHA) has not been previously treated with an androgen biosynthesis inhibitor or an androgen receptor blocker. In some embodiments, the castrate-resistant prostate cancer naïve to novel hormonal agents (NHA) has not been previously treated with an androgen biosynthesis inhibitor. In some embodiments, the castrate-resistant prostate cancer naïve to novel hormonal agents (NHA) has not been previously treated with an androgen receptor blocker. In some embodiments, the castrate-resistant prostate cancer naïve to novel hormonal agents (NHA) has not been previously treated with abiraterone acetate. In some embodiments, the castrate-resistant prostate cancer naïve to novel hormonal agents (NHA) has not been previously treated with an androgen receptor blocker selected from enzalutamide, darolutamide, and apalutamide.

In some embodiments, the prostate cancer is castrate-sensitive prostate cancer naïve to novel hormonal agents (NHA). In some embodiments, the castrate-sensitive prostate cancer naïve to novel hormonal agents (NHA) has not been previously treated with one or more second-generation antiandrogens. In some embodiments, the castrate-sensitive prostate cancer naïve to novel hormonal agents (NHA) has not been previously treated with an androgen biosynthesis inhibitor or an androgen receptor blocker. In some embodiments, the castrate-sensitive prostate cancer naïve to novel hormonal agents (NHA) has not been previously treated with an androgen biosynthesis inhibitor. In some embodiments, the castrate-sensitive prostate cancer naïve to novel hormonal agents (NHA) has not been previously treated with an androgen receptor blocker. In some embodiments, the castrate-sensitive prostate cancer naïve to novel hormonal agents (NHA) has not been previously treated with abiraterone acetate. In some embodiments, the castrate-sensitive prostate cancer naïve to novel hormonal agents (NHA) has not been previously treated with an androgen receptor blocker selected from enzalutamide, darolutamide, and apalutamide.

In some embodiments, the prostate cancer is metastatic castrate-resistant prostate cancer naïve to novel hormonal agents (NHA). In some embodiments, the metastatic castrate-resistant prostate cancer naïve to novel hormonal agents (NHA) has not been previously treated with one or more second-generation antiandrogens. In some embodiments, the metastatic castrate-resistant prostate cancer naïve to novel hormonal agents (NHA) has not been previously treated with an androgen biosynthesis inhibitor or an androgen receptor blocker. In some embodiments, the metastatic castrate-resistant prostate cancer naïve to novel hormonal agents (NHA) has not been previously treated with an androgen biosynthesis inhibitor. In some embodiments, the metastatic castrate-resistant prostate cancer naïve to novel hormonal agents (NHA) has not been previously treated with an androgen receptor blocker. In some embodiments, the metastatic castrate-resistant prostate cancer naïve to novel hormonal agents (NHA) has not been previously treated with abiraterone acetate. In some embodiments, the metastatic castrate-resistant prostate cancer naïve to novel hormonal agents (NHA) has not been previously treated with an androgen receptor blocker selected from enzalutamide, darolutamide, and apalutamide.

In some embodiments, the prostate cancer is metastatic castrate-sensitive prostate cancer naïve to novel hormonal agents (NHA). In some embodiments, the metastatic castrate-sensitive prostate cancer naïve to novel hormonal agents (NHA) has not been previously treated with one or more second-generation antiandrogens. In some embodiments, the metastatic castrate-sensitive prostate cancer naïve to novel hormonal agents (NHA) has not been previously treated with an androgen biosynthesis inhibitor or an androgen receptor blocker. In some embodiments, the metastatic castrate-sensitive prostate cancer naïve to novel hormonal agents (NHA) has not been previously treated with an androgen biosynthesis inhibitor. In some embodiments, the metastatic castrate-sensitive prostate cancer naïve to novel hormonal agents (NHA) has not been previously treated with an androgen receptor blocker. In some embodiments, the metastatic castrate-sensitive prostate cancer naïve to novel hormonal agents (NHA) has not been previously treated with abiraterone acetate. In some embodiments, the metastatic castrate-sensitive prostate cancer naïve to novel hormonal agents (NHA) has not been previously treated with an androgen receptor blocker selected from enzalutamide, darolutamide, and apalutamide.

In some embodiments, the second-generation antiandrogen is an androgen biosynthesis inhibitor or an androgen receptor blocker.

In some embodiments, the androgen biosynthesis inhibitor is abiraterone acetate.

In some embodiments, the androgen receptor blocker is selected from enzalutamide, darolutamide, and apalutamide.

In some embodiments, the prostate cancer is not prostate cancer naïve to novel hormonal agents (NHA), optionally wherein the subject's prostate cancer has been treated previously with one, two, or more NHAs.

### BRIEF DESCRIPTION OF THE DRAWINGS

The accompanying drawings, which are incorporated into and form a part of the specification, illustrate several embodiments of the present disclosure and, together with the description, serve to explain the principles of the disclosure. The drawings are only for the purpose of illustrating an embodiment of the disclosure and are not to be construed as limiting the disclosure. Further objects, features and advantages of the disclosure will become apparent from the following detailed description taken in conjunction with the accompanying figures showing illustrative embodiments of the disclosure, in which:

FIGS. 1A and 1B are illustrations of the general principle for the function of proteolysis targeting chimeric compounds. FIG. 1A represents exemplary proteolysis targeting chimeric compounds comprise a protein targeting moiety (PTM; darkly shaded rectangle), a cereblon ubiquitin ligase binding moiety (CLM; unshaded triangle), and a linker moiety (black line) coupling or tethering the PTM to the CLM. FIG. 1B illustrates the functional use of the proteolysis targeting chimeric compounds as described herein. Briefly, the CLM recognizes and binds to cereblon, an E3 Ubiquitin Ligase, and the PTM binds and recruits an intracellular target protein bringing it into close proximity to the cereblon E3 Ubiquitin Ligase. Typically, the cereblon E3 Ubiquitin Ligase is complexed with an E2 ubiquitin-conjugating protein, and either alone or via the E2 protein catalyzes attachment of ubiquitin (dark circles) to a lysine on the target protein via an isopeptide bond. The poly-ubiquitinated protein (far right) is then targeted for degradation by the proteosomal machinery of the cell.

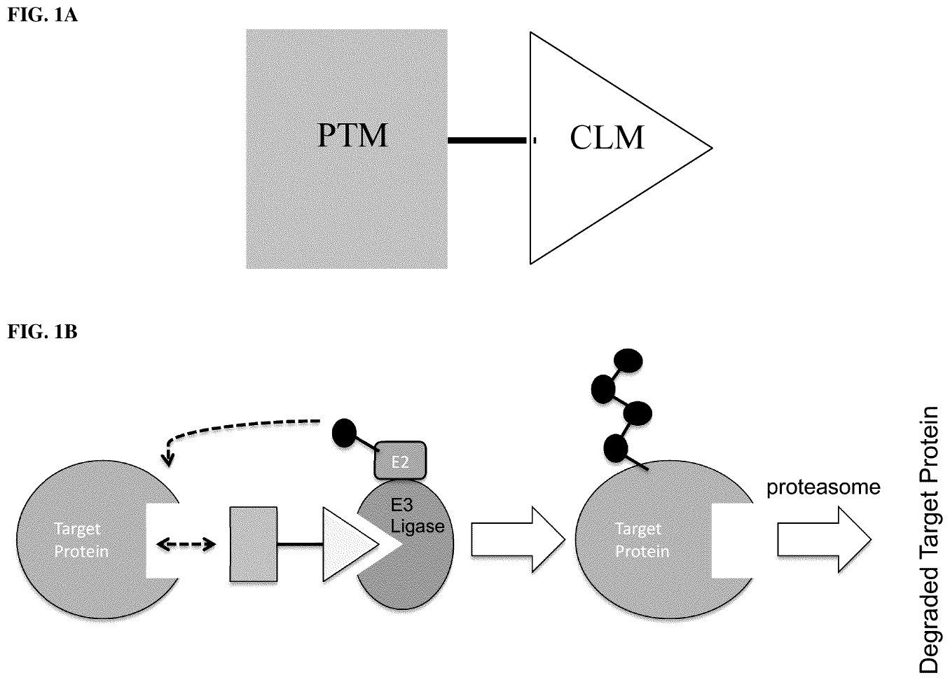

FIG. 2 is an analysis of Compound A in in vitro AR degradation assays conducted in VCaP cells and LNCaP cancer cells that harbor AR amplification and the AR mutation (T878A) mutation, respectively.

FIG. 3 is a graph showing Compound A's ability to degrade clinically relevant mutated forms AR.

FIG. 4 is an analysis of Compound A in in vitro cereblon neomorphic substrate degradation assays conducted in Ramos cells and SK-N-DZ cells.

FIG. 5 is a graph showing the effect of Compound A and enzalutamide in an in vivo VCaP tumor xenograft study in intact mice.

FIG. 6 shows the effects of Compound A and enzalutamide on the plasma PSA levels in samples from the intact VCaP tumor model.

FIG. 7 shows the best % PSA₅₀ change from baseline in patients with AR ligand binding domain (LBD) mutations, including PSA₅₀ responses in patients with co-occurring AR H875/T878/L702H mutations.

FIGS. 8A, 8B, and 8C show that Compound A inhibits PSA synthesis, blocks prostate cancer proliferation, and induces apoptosis in vertebral-cancer of the prostate (VCaP) cells. FIG. 8A shows the results of an experiment in which VCaP cells cultured in charcoal-stripped fetal bovine serum (CSS) supplemented with 0.1 nM R1881 (synthetic androgen) were treated with indicated concentrations of Compound A or enzalutamide for 48 hours. Intracellular prostate specific antigen (PSA) levels were determined using a PSA enzyme-linked immunosorbent assay (ELISA). FIG. 8B shows the results of an experiment in which VCaP cells were cultured in media containing CSS and supplemented with 0.1 nM R1881 and treated with indicated concentrations of Compound A or enzalutamide for 96 hours. The cell proliferation was determined using the CellTiter-Glo® assay. FIG. 8C shows the results of an experiment in which VCaP cells cultured in media containing CSS and supplemented with 0.1 nM R1881 were treated with indicated concentrations of Compound A for 72 hours. The CaspaseGlo® assay was carried out to measure the level of apoptosis in cells.

FIG. 9 shows the result of a cell proliferation assay was carried out in a high androgen environment condition in vitro to assess whether Compound A can overcome the effect of residual androgen. VCaP cells were grown in CS S-containing media in the presence of fixed concentrations of Compound A (300 nM) or enzalutamide (1,000 nM) and increasing concentrations of the synthetic androgen R1881. R1881 alone promoted the proliferation of VCaP cells, demonstrating that proliferation can be induced by activation of the AR signaling pathway. VCaP cells were cultured in media containing CSS, the indicated concentrations R1881 and either Compound A (300 nM) or enzalutamide (1,000 nM) for 5 days. Cell proliferation was determined using the CellTiter Glo® assay.

FIG. 10 shows the degradation of AR proteins harboring clinically relevant point mutations M896V, T878A, F877L, L702H, and H875Y. Plasmids expressing AR WT or the indicated AR mutants were stably transfected into HEK293 cells. Each cell line was treated with indicated concentrations of Compound A for 24 hours. AR levels were determined by AR ELISA without background subtraction. Plasmids expressing AR wild-type (WT) or the indicated AR mutants were stably transfected into HEK293 cells. Each cell line was treated with indicated concentrations of Compound A for 24 hours. AR levels were determined by AR ELISA without background subtraction.

FIG. 11 shows the AR degradation in castrated CB17/SCID male mice bearing VCaP xenografts treated with various single doses of Compound A (10, 3, 1, 0.3, 0.1, and 0.03 mg/kg). Tumors were harvested 16 hours post-dose and tumor AR level was established by western blotting. Antibody recognizing human mitochondria specific antibody (Mito.C) was employed as a loading control. The quantitation of AR levels from the western blot is shown (lower panel).

FIG. 12 shows tumor growth inhibition in castrated CB17/SCID male mice bearing VCaP xenografts treated with Compound A (3, 1, 0.3, or 0.1 mg/kg) or enzalutamide (20 mg/kg). Enza=enzalutamide; PO=oral; QD=once daily; scid=severe combined immune deficiency. Treatment groups (n=10 per group) were compared by one-way analysis of variance (ANOVA). Note: ns: not significant, *: p≤0.05, **: p≤0.01, ***: p≤0.001.

FIG. 13 shows the results of a 10-day prostate involution study in adult rats. Enzalutamide and Compound A were orally administered once daily at the indicated doses. After 10 days of treatment, the prostate tissue from each rat was isolated and weighed. Treatment groups (n=5 per group) were compared by one way ANOVA. Note: ns: not significant, *: p<0.05, **: p≤0.01.

FIG. 14 is a graph that shows the deep prostate-specific antigen (PSA) decline in a patient with metastatic castrate-resistant prostate cancer (mCRPC) with wild-type AR. These results support development in pre-novel hormonal agent (pre-NHA) settings, including metastatic castrate-sensitive prostate cancer (mCSPC). Evaluable analysis set include all Compound A treated participants with baseline PSA assessment and at least a C1D28 or later postbaseline PSA assessment. A PSA₉₀ and PSA₅₀ and PSA₃₀ response is defined as a =90% or 50% or 30% decline in PSA from baseline PSA value, respectively.

### SEQUENCE LISTING

All references to amino acid mutations in the Androgen Receptor are numbered relative to SEQ ID NO: 1, which is provided below:

| | |
|---|---|
| 1 | mevqlglgrv yprppsktyr gafqnlfqsv revignpgpr hpeaasaapp gasllllqqq |
| 61 | qqqqqqqqqq qqqqqqqqqq etsprqqqqq qgedgspqah rrgptgylvl deeqqpspqp |
| 121 | salechperg cvpepgaava askglpqqlp appdeddsaa pstlsllgpt fpglsscsad |
| 181 | lkdilseast mqllqqqqqe avsegsssgr areasgapts skdnylggts tisdnakelc |
| 241 | kavsvsmglg vealehlspg eqlrgdcmya pllgvppavr ptpcaplaec kgsllddsag |
| 301 | kstedtaeys pfkggytkgl egeslgcsgs aaagssgtle lpstlslyks galdeaaayq |
| 361 | srdyynfpla lagppppppp phpharikle npldygsawa aaaaqcrygd laslhgagaa |
| 421 | gpgsgspsaa assswhtlft aeegqlygpc gggggggggg gggggggggg gggeagavap |
| 481 | ygytrppqgl agqesdftap dvwypggmvs rvpypsptcv ksemgpwmds ysgpygdmrl |
| 541 | etardhvlpi dyyfppqktc licgdeasgc hygaltcgsc kvffkraaeg kqkylcasrn |
| 601 | dctidkfrrk ncpscrlrkc yeagmtlgar klkklgnlkl qeegeasstt spteettqkl |
| 661 | tvshiegyec qpiflnvlea iepgvvcagh dnnqpdsfaa llsslnelge rqlvhvvkwa |
| 721 | kalpgfrnlh vddqmavigy swmglmvfam gwrsftnvns rmlyfapdlv fneyrmhksr |
| 781 | mysqcvrmrh lsqefgwlqi tpqeflcmka lllfsiipvd glknqkffde lrmnyikeld |
| 841 | riiackrknp tscsrrfyql tklldsvqpi arelhqftfd llikshmvsv dfpemmaeii |
| 901 | svqvpkilsg kvkpiyfhtq |

### DETAILED DESCRIPTION

### Definitions

All references to amino acid mutations in the Androgen Receptor are numbered relative to SEQ ID NO: 1, which is provided herewith.

The term “Ubiquitin Ligase” refers to a family of proteins that facilitate the transfer of ubiquitin to a specific substrate protein, targeting the substrate protein for degradation. For example, cereblon is an E3 Ubiquitin Ligase protein that alone or in combination with an E2 ubiquitin-conjugating enzyme causes the attachment of ubiquitin to a lysine on a target protein, and subsequently targets the specific protein substrates for degradation by the proteasome. Thus, E3 ubiquitin ligase alone or in complex with an E2 ubiquitin conjugating enzyme is responsible for the transfer of ubiquitin to targeted proteins. In general, the ubiquitin ligase is involved in polyubiquitination such that a second ubiquitin is attached to the first; a third is attached to the second, and so forth. Polyubiquitination marks proteins for degradation by the proteasome. However, there are some ubiquitination events that are limited to mono-ubiquitination, in which only a single ubiquitin is added by the ubiquitin ligase to a substrate molecule. Mono-ubiquitinated proteins are not targeted to the proteasome for degradation but may instead be altered in their cellular location or function, for example, via binding other proteins that have domains capable of binding ubiquitin. Further complicating matters, different lysines on ubiquitin can be targeted by an E3 to make chains. The most common lysine is Lys48 on the ubiquitin chain. This is the lysine used to make polyubiquitin, which is recognized by the proteasome.

As used herein, “Compound”, “bifunctional compound”, or “Compound of the Disclosure”, as used herein, refers to the compounds disclosed by structure in the following tables and examples.

The term “substituted” or “optionally substituted” shall mean independently (i.e., where more than substituent occurs, each substituent is independent of another substituent) one or more substituents (independently up to five substituents, preferably up to three substituents, often 1 or 2 substituents on a moiety in a compound according to the present disclosure and may include substituents which themselves may be further substituted) at a carbon (or nitrogen) position anywhere on a molecule within context, and includes as substituents hydroxyl, thiol, carboxyl, cyano (CN), nitro (NO₂), halogen (preferably, 1, 2 or 3 halogens, especially on an alkyl, especially a methyl group such as a trifluoromethyl), an alkyl group (preferably, C₁-C₁₀, more preferably, C₁-C₆), aryl (especially phenyl and substituted phenyl for example benzyl or benzoyl), alkoxy group (preferably, C₁-C₆ alkyl or aryl, including phenyl and substituted phenyl), thioether (C₁-C₆ alkyl or aryl), acyl (preferably, C₁-C₆ acyl), ester or thioester (preferably, C₁-C₆ alkyl or aryl) including alkylene ester (such that attachment is on the alkylene group, rather than at the ester function which is preferably substituted with a C₁-C₆ alkyl or aryl group), preferably, C₁-C₆ alkyl or aryl, halogen (preferably, F or Cl), amine (including a five- or six-membered cyclic alkylene amine, further including a C₁-C₆ alkyl amine or a C₁-C₆ dialkyl amine which alkyl groups may be substituted with one or two hydroxyl groups) or an optionally substituted —N(C₀-C₆ alkyl)C(O)(O—C₁-C₆ alkyl) group (which may be optionally substituted with a polyethylene glycol chain to which is further bound an alkyl group containing a single halogen, preferably chlorine substituent), hydrazine, amido, which is preferably substituted with one or two C₁-C₆ alkyl groups (including a carboxamide which is optionally substituted with one or two C₁-C₆ alkyl groups), alkanol (preferably, C₁-C₆ alkyl or aryl), or alkanoic acid (preferably, C₁-C₆ alkyl or aryl). Substituents according to the present disclosure may include, for example —SiR₁R₂R₃ groups where each of R₁ and R₂ is as otherwise described herein and R₃ is H or a C₁-C₆ alkyl group, preferably R₁, R₂, R₃ in this context is a C₁-C₃ alkyl group (including an isopropyl or t-butyl group). Each of the above-described groups may be linked directly to the substituted moiety or alternatively, the substituent may be linked to the substituted moiety (preferably in the case of an aryl or heteraryl moiety) through an optionally substituted —(CH₂)ₘ— or alternatively an optionally substituted —(OCH₂)ₘ—, —(OCH₂CH₂)ₘ— or —(CH₂CH₂O)ₘ— group, which may be substituted with any one or more of the above-described substituents. Alkylene groups —(CH₂)ₘ— or —(CH₂)ₙ— groups or other chains such as ethylene glycol chains, as identified above, may be substituted anywhere on the chain. Preferred substituents on alkylene groups include halogen or C₁-C₆ (preferably C₁-C₃) alkyl groups, which may be optionally substituted with one or two hydroxyl groups, one or two ether groups (O—C₁-C₆ groups), up to three halo groups (preferably F), or a sidechain of an amino acid as otherwise described herein and optionally substituted amide (preferably carboxamide substituted as described above) or urethane groups (often with one or two C₀-C₆ alkyl substituents, which group(s) may be further substituted). In certain embodiments, the alkylene group (often a single methylene group) is substituted with one or two optionally substituted C₁-C₆ alkyl groups, preferably C₁-C₄ alkyl group, most often methyl or O-methyl groups or a sidechain of an amino acid as otherwise described herein. In the present disclosure, a moiety in a molecule may be optionally substituted with up to five substituents, preferably up to three substituents. Most often, in the present disclosure moieties which are substituted are substituted with one or two substituents.

The term “substituted” (each substituent being independent of any other substituent) shall also mean within its context of use C₁-C₆ alkyl, C₁-C₆ alkoxy, halogen, amido, carboxamido, sulfone, including sulfonamide, keto, carboxy, C₁-C₆ ester (oxyester or carbonylester), C₁-C₆ keto, urethane —O—C(O)—NR₁R₂ or —N(R₁)—C(O)—O—R₁, nitro, cyano and amine (especially including a C₁-C₆ alkylene-NR₁R₂, a mono- or di-C₁-C₆ alkyl substituted amines which may be optionally substituted with one or two hydroxyl groups). Each of these groups contain unless otherwise indicated, within context, between 1 and 6 carbon atoms. In certain embodiments, preferred substituents will include for example, —NH—, —NHC(O)—, —O—, ═O, —(CH₂)ₘ— (here, m and n are in context, 1, 2, 3, 4, 5 or 6), —S—, —S(O)—, SO₂— or —NH—C(O)—NH—, —(CH₂)ₙOH, —(CH₂)ₙSH, —(CH₂)ₙCOOH, C₁-C₆ alkyl, —(CH₂)ₙO—(C₁-C₆ alkyl), —(CH₂)ₙC(O)—(C₁-C₆ alkyl), —(CH₂)ₙOC(O)—(C₁-C₆ alkyl), —(CH₂)ₙC(O)O—(C₁-C₆ alkyl), —(CH₂)ₙNHC(O)—R₁, —(CH₂)ₙC(O)—NR₁R₂, —(OCH₂)ₙOH, —(CH₂O)ₙCOOH, C₁-C₆ alkyl, —(OCH₂)ₙO—(C₁-C₆ alkyl), —(CH₂O)ₙC(O)—(C₁-C₆ alkyl), —(OCH₂)ₙNHC(O)—R₁, —(CH₂O)ₙC(O)—NR₁R₂, —S(O)₂-R_{S}, —S(O)—R_{S} (R_{S} is C₁-C₆ alkyl or a —(CH₂)ₘ—NR₁R₂ group), NO₂, CN or halogen (F, Cl, Br, I, preferably F or Cl), depending on the context of the use of the substituent. R₁ and R₂ are each, within context, H or a C₁-C₆ alkyl group (which may be optionally substituted with one or two hydroxyl groups or up to three halogen groups, preferably fluorine). The term “substituted” shall also mean, within the chemical context of the compound defined and substituent used, an optionally substituted aryl or heteroaryl group or an optionally substituted heterocyclic group as otherwise described herein. Alkylene groups may also be substituted as otherwise disclosed herein, preferably with optionally substituted C₁-C₆ alkyl groups (methyl, ethyl or hydroxymethyl or hydroxyethyl is preferred, thus providing a chiral center), a sidechain of an amino acid group as otherwise described herein, an amido group as described hereinabove, or a urethane group O—C(O)—NR₁R₂ group where R₁ and R₂ are as otherwise described herein, although numerous other groups may also be used as substituents. Various optionally substituted moieties may be substituted with 3 or more substituents, preferably no more than 3 substituents and preferably with 1 or 2 substituents. It is noted that in instances where, in a compound at a particular position of the molecule substitution is required (principally, because of valency), but no substitution is indicated, then that substituent is construed or understood to be H, unless the context of the substitution suggests otherwise.

The term “aryl” or “aromatic”, in context, refers to a substituted (as otherwise described herein) or unsubstituted monovalent aromatic radical having a single ring (e.g., benzene, phenyl, benzyl) or condensed rings (e.g., naphthyl, anthracenyl, phenanthrenyl, etc.) and can be bound to the compound according to the present disclosure at any available stable position on the ring(s) or as otherwise indicated in the chemical structure presented. Other examples of aryl groups, in context, may include heterocyclic aromatic ring systems, “heteroaryl” groups having one or more nitrogen, oxygen, or sulfur atoms in the ring (moncyclic) such as imidazole, furyl, pyrrole, furanyl, thiene, thiazole, pyridine, pyrimidine, pyrazine, triazole, oxazole or fused ring systems such as indole, quinoline, indolizine, azaindolizine, benzofurazan, etc., among others, which may be optionally substituted as described above. Among the heteroaryl groups which may be mentioned include nitrogen-containing heteroaryl groups such as pyrrole, pyridine, pyridone, pyridazine, pyrimidine, pyrazine, pyrazole, imidazole, triazole, triazine, tetrazole, indole, isoindole, indolizine, azaindolizine, purine, indazole, quinoline, dihydroquinoline, tetrahydroquinoline, isoquinoline, dihydroisoquinoline, tetrahydroisoquinoline, quinolizine, phthalazine, naphthyridine, quinoxaline, quinazoline, cinnoline, pteridine, imidazopyridine, imidazotriazine, pyrazinopyridazine, acridine, phenanthridine, carbazole, carbazoline, pyrimidine, phenanthroline, phenacene, oxadiazole, benzimidazole, pyrrolopyridine, pyrrolopyrimidine and pyridopyrimidine; sulfur-containing aromatic heterocycles such as thiophene and benzothiophene; oxygen-containing aromatic heterocycles such as furan, pyran, cyclopentapyran, benzofuran and isobenzofuran; and aromatic heterocycles comprising 2 or more hetero atoms selected from among nitrogen, sulfur and oxygen, such as thiazole, thiadizole, isothiazole, benzoxazole, benzothiazole, benzothiadiazole, phenothiazine, isoxazole, furazan, phenoxazine, pyrazoloxazole, imidazothiazole, thienofuran, furopyrrole, pyridoxazine, furopyridine, furopyrimidine, thienopyrimidine and oxazole, among others, all of which may be optionally substituted.

The term “substituted aryl” refers to an aromatic carbocyclic group comprised of at least one aromatic ring or of multiple condensed rings at least one of which being aromatic, wherein the ring(s) are substituted with one or more substituents. For example, an aryl group can comprise a substituent(s) selected from: —(CH₂)ₙOH, —(CH₂)ₙ—O—(C₁-C₆)alkyl, —(CH₂)ₙ—O—(CH₂)ₙ—(C₁-C₆)alkyl, —(CH₂)ₙ—C(O)(C₀-C₆) alkyl, —(CH₂)ₙ—C(O)O(C₀-C₆)alkyl, —(CH₂)ₙ—OC(O)(C₀-C₆)alkyl, amine, mono- or di-(C₁-C₆ alkyl) amine wherein the alkyl group on the amine is optionally substituted with 1 or 2 hydroxyl groups or up to three halo (preferably F, Cl) groups, OH, COOH, C₁-C₆ alkyl, preferably CH₃, CF₃, OMe, OCF₃, NO₂, or CN group (each of which may be substituted in ortho-, meta- and/or para-positions of the phenyl ring, preferably para-), an optionally substituted phenyl group (the phenyl group itself is preferably substituted with a linker group attached to a ABM group, including a ULM group), and/or at least one of F, Cl, OH, COOH, CH₃, CF₃, OMe, OCF₃, NO₂, or CN group (in ortho-, meta- and/or para-positions of the phenyl ring, preferably para-), a naphthyl group, which may be optionally substituted, an optionally substituted heteroaryl, preferably an optionally substituted isoxazole including a methylsubstituted isoxazole, an optionally substituted oxazole including a methylsubstituted oxazole, an optionally substituted thiazole including a methyl substituted thiazole, an optionally substituted isothiazole including a methyl substituted isothiazole, an optionally substituted pyrrole including a methylsubstituted pyrrole, an optionally substituted imidazole including a methylimidazole, an optionally substituted benzimidazole or methoxybenzylimidazole, an optionally substituted oximidazole or methyloximidazole, an optionally substituted diazole group, including a methyldiazole group, an optionally substituted triazole group, including a methylsubstituted triazole group, an optionally substituted pyridine group, including a halo-(preferably, F) or methylsubstitutedpyridine group or an oxapyridine group (where the pyridine group is linked to the phenyl group by an oxygen), an optionally substituted furan, an optionally substituted benzofuran, an optionally substituted dihydrobenzofuran, an optionally substituted indole, indolizine or azaindolizine (2, 3, or 4-azaindolizine), an optionally substituted quinoline, and combinations thereof.

The term “heteroaryl” or “hetaryl” can mean but is in no way limited to an optionally substituted quinoline (which may be attached to the pharmacophore or substituted on any carbon atom within the quinoline ring), an optionally substituted indole (including dihydroindole), an optionally substituted indolizine, an optionally substituted azaindolizine (2, 3 or 4-azaindolizine) an optionally substituted benzimidazole, benzodiazole, benzoxofuran, an optionally substituted imidazole, an optionally substituted isoxazole, an optionally substituted oxazole (preferably methyl substituted), an optionally substituted diazole, an optionally substituted triazole, a tetrazole, an optionally substituted benzofuran, an optionally substituted thiophene, an optionally substituted thiazole (preferably methyl and/or thiol substituted), an optionally substituted isothiazole, an optionally substituted triazole (preferably a 1,2,3-triazole substituted with a methyl group, a triisopropylsilyl group, an optionally substituted —(CH₂)ₘ—O—C₁-C₆ alkyl group or an optionally substituted —(CH₂)ₘ—C(O)—O—C₁-C₆ alkyl group), an optionally substituted pyridine (2-, 3, or 4-pyridine) or a group according to the chemical structure:

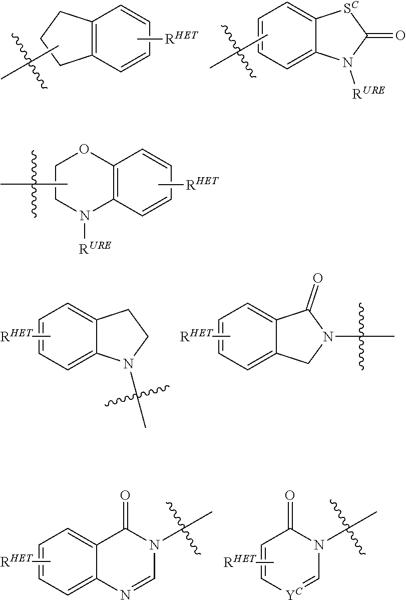

wherein
S^{c} is CHR^{SS}, NR^{URE} or O;
R^{HET} is H, CN, NO₂, halo (preferably Cl or F), optionally substituted C₁-C₆ alkyl (preferably substituted with one or two hydroxyl groups or up to three halo groups (e.g. CF₃), optionally substituted O(C₁-C₆ alkyl) (preferably substituted with one or two hydroxyl groups or up to three halo groups) or an optionally substituted acetylenic group —C≡C—Rₐ where Rₐ is H or a C₁-C₆ alkyl group (preferably C₁-C₃ alkyl);
R^{SS} is H, CN, NO₂, halo (preferably F or Cl), optionally substituted C₁-C₆ alkyl (preferably substituted with one or two hydroxyl groups or up to three halo groups), optionally substituted O—(C₁-C₆ alkyl) (preferably substituted with one or two hydroxyl groups or up to three halo groups) or an optionally substituted —C(O)(C₁-C₆ alkyl) (preferably substituted with one or two hydroxyl groups or up to three halo groups);
R^{URE} is H, a C₁-C₆ alkyl (preferably H or C₁-C₃ alkyl) or a —C(O)(C₁-C₆ alkyl), each of which groups is optionally substituted with one or two hydroxyl groups or up to three halogen, preferably fluorine groups, or an optionally substituted heterocycle, for example piperidine, morpholine, pyrrolidine, tetrahydrofuran, tetrahydrothiophene, piperidine, piperazine, each of which is optionally substituted, and

Y^{C} is N or C—R^{YC}, where R^{YC} is H, OH, CN, NO₂, halo (preferably Cl or F), optionally substituted C₁-C₆ alkyl (preferably substituted with one or two hydroxyl groups or up to three halo groups (e.g. CF₃), optionally substituted O(C₁-C₆ alkyl) (preferably substituted with one or two hydroxyl groups or up to three halo groups) or an optionally substituted acetylenic group —C≡C— Rₐ where Rₐ is H or a C₁-C₆ alkyl group (preferably C₁-C₃ alkyl).

The term “Heterocycle” refers to a cyclic group which contains at least one heteroatom, e.g., N, O or S, and may be aromatic (heteroaryl) or non-aromatic. Thus, the heteroaryl moieties are subsumed under the definition of heterocycle, depending on the context of its use. Exemplary heteroaryl groups are described hereinabove.

Exemplary heterocyclics include: azetidinyl, benzimidazolyl, 1,4-benzodioxanyl, 1,3-benzodioxolyl, benzoxazolyl, benzothiazolyl, benzothienyl, dihydroimidazolyl, dihydropyranyl, dihydrofuranyl, dioxanyl, dioxolanyl, ethyleneurea, 1,3-dioxolane, 1,3-dioxane, 1,4-dioxane, furyl, homopiperidinyl, imidazolyl, imidazolinyl, imidazolidinyl, indolinyl, indolyl, isoquinolinyl, isothiazolidinyl, isothiazolyl, isoxazolidinyl, isoxazolyl, morpholinyl, naphthyridinyl, oxazolidinyl, oxazolyl, pyridone, 2-pyrrolidone, pyridine, piperazinyl, N-methylpiperazinyl, piperidinyl, phthalimide, succinimide, pyrazinyl, pyrazolinyl, pyridyl, pyrimidinyl, pyrrolidinyl, pyrrolinyl, pyrrolyl, quinolinyl, tetrahydrofuranyl, tetrahydropyranyl, tetrahydroquinoline, thiazolidinyl, thiazolyl, thienyl, tetrahydrothiophene, oxane, oxetanyl, oxathiolanyl, thiane among others.

Heterocyclic groups can be optionally substituted with a member selected from the group consisting of alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, keto, thioketo, carboxy, carboxyalkyl, thioaryloxy, thioheteroaryloxy, thioheterocyclooxy, thiol, thioalkoxy, substituted thioalkoxy, aryl, aryloxy, heteroaryl, heteroaryloxy, heterocyclic, heterocyclooxy, hydroxyamino, alkoxyamino, nitro, —SO-alkyl, —SO-substituted alkyl, —SOaryl, —SO-heteroaryl, —SO₂-alkyl, —SO₂-substituted alkyl, —SO₂-aryl, oxo (═O), and —SO₂-heteroaryl. Such heterocyclic groups can have a single ring or multiple condensed rings. Examples of nitrogen heterocycles and heteroaryls include, but are not limited to, pyrrole, imidazole, pyrazole, pyridine, pyrazine, pyrimidine, pyridazine, indolizine, isoindole, indole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, piperidine, piperazine, indoline, morpholino, piperidinyl, tetrahydrofuranyl, and the like as well as N-alkoxy-nitrogen containing heterocycles. The term “heterocyclic” also includes bicyclic groups in which any of the heterocyclic rings is fused to a benzene ring or a cyclohexane ring or another heterocyclic ring (for example, indolyl, quinolyl, isoquinolyl, tetrahydroquinolyl, and the like).

The term “cycloalkyl” can mean but is in no way limited to univalent groups derived from monocyclic or polycyclic alkyl groups or cycloalkanes, as defined herein, e.g., saturated monocyclic hydrocarbon groups having from three to twenty carbon atoms in the ring, including, but not limited to, cyclopropyl, cyclobutyl, cyclopentyl, cyclohexyl, cycloheptyl and the like. The term “substituted cycloalkyl” can mean but is in no way limited to a monocyclic or polycyclic alkyl group and being substituted by one or more substituents, for example, amino, halogen, alkyl, substituted alkyl, carbyloxy, carbylmercapto, aryl, nitro, mercapto or sulfo, whereas these generic substituent groups have meanings which are identical with definitions of the corresponding groups as defined in this legend.

“Heterocycloalkyl” refers to a monocyclic or polycyclic alkyl group in which at least one ring carbon atom of its cyclic structure being replaced with a heteroatom selected from the group consisting of N, O, S and P. “Substituted heterocycloalkyl” refers to a monocyclic or polycyclic alkyl group in which at least one ring carbon atom of its cyclic structure being replaced with a heteroatom selected from the group consisting of N, O, S and P and the group is containing one or more substituents selected from the group consisting of halogen, alkyl, substituted alkyl, carbyloxy, carbylmercapto, aryl, nitro, mercapto and sulfo, whereas these generic substituent group have meanings which are identical with definitions of the corresponding groups as defined in this legend.

The term “spirocycle” or “spiro-fused cycloalkyl” refers to a polycyclic alkyl group containing at least two rings, in which two rings share exactly one ring atom. The term “spiroheterocycle” or “spiro-fused heterocycloalkyl” refers to spiro-fused cycloalkyl group in which at least one ring carbon atom of its cyclic structure is replaced with a heteroatom selected from the group consisting of N, O, S and P. Spiro-fused cycloalkyl and spiro-fused heterocycloalkyl groups may be further defined by their number of rings, e.g. bicyclic, tricyclic, tetracyclic, etc.

The term “bridged cycloalkyl” or refers to a polycyclic alkyl group containing at least two rings, in which two rings share at least three ring atoms. The term “bridged heterocycle” refers to bridged cycloalkyl groups in which at least one ring carbon atom of its cyclic structure is replaced with a heteroatom selected from the group consisting of N, O, S and P. Bridged cycloalkyl and spiro-fused heterocycloalkyl groups may be further defined by their number of rings, e.g., bicyclic, tricyclic, tetracyclic, etc.

“Halogen” or “halo” refers to fluorine (F), chlorine (Cl), bromine (Br), or iodine (I).

“C₁-C₆ alkyl” refers to a straight or branched chain saturated hydrocarbon containing 1-6 carbon atoms. Examples of a (C₁-C₆) alkyl group include, but are not limited to, methyl, ethyl, propyl, butyl, pentyl, hexyl, isopropyl, isobutyl, sec-butyl, tert-butyl, isopentyl, neopentyl, and isohexyl.

“Pharmaceutically acceptable salt”, as used herein with respect to a compound of the disclosure, means a salt form of the compound of the disclosure as well as hydrates of the salt form with one or more water molecules present. Such salt and hydrated forms retain the biological activity of the compound of the disclosure and are not biologically or otherwise undesirable, i.e., exhibit minimal, if any, toxicological effects. Representative “pharmaceutically acceptable salts” include, e.g., water-soluble and water-insoluble salts, such as the acetate, amsonate (4,4-diaminostilbene-2,2-disulfonate), benzenesulfonate, benzonate, bicarbonate, bisulfate, bitartrate, borate, bromide, butyrate, calcium, calcium edetate, camsylate, carbonate, chloride, citrate, clavulariate, dihydrochloride, edetate, edisylate, estolate, esylate, fumarate, gluceptate, gluconate, glutamate, glycollylarsanilate, hexafluorophosphate, hexylresorcinate, hydrabamine, hydrobromide, hydrochloride, hydroxynaphthoate, iodide, isothionate, lactate, lactobionate, laurate, magnesium, malate, maleate, mandelate, mesylate, methylbromide, methylnitrate, methylsulfate, mucate, napsylate, nitrate, N-methylglucamine ammonium salt, 3-hydroxy-2-naphthoate, oleate, oxalate, palmitate, pamoate (1,1-methene-bis-2-hydroxy-3-naphthoate, einbonate), pantothenate, phosphate/diphosphate, picrate, polygalacturonate, propionate, p-toluenesulfonate, salicylate, stearate, subacetate, succinate, sulfate, sulfosalicylate, suramate, tannate, tartrate, teoclate, tosylate, triethiodide, and valerate salts.

The term “isomer” refers to salts and/or compounds that have the same composition and molecular weight but differ in physical and/or chemical properties. The structural difference may be in constitution (geometric isomers) or in the ability to rotate the plane of polarized light (stereoisomers). With regard to stereoisomers, the salts of the compounds of the disclosure may have one or more asymmetric carbon atom and may occur as racemates, racemic mixtures and as individual enantiomers or diastereomers.

The compounds of the disclosure may exist in unsolvated as well as solvated forms such as, for example, hydrates.

“Solvate” means a solvent addition form that contains either a stoichiometric or non-stoichiometric amounts of solvent. Non-limiting examples of suitable solvates include ethanolate, methanolate, and the like. Some compounds have a tendency to trap a fixed molar ratio of solvent molecules in the crystalline solid state, thus forming a solvate. If the solvent is water, the solvate formed is a hydrate, when the solvent is alcohol, the solvate formed is an alcoholate. Hydrates are formed by the combination of one or more molecules of water with one of the substances in which the water retains its molecular state as H₂O, such combination being able to form one or more hydrate. In the hydrates, the water molecules are attached through secondary valencies by intermolecular forces, in particular hydrogen bridges. Solid hydrates contain water as so-called crystal water in stoichiometric ratios, where the water molecules do not have to be equivalent with respect to their binding state. Examples of hydrates are sesquihydrates, monohydrates, dihydrates or trihydrates. Equally suitable are the hydrates of salts of the compounds of the disclosure.

“Isotopic derivative”, as referred to herein, relates to a compound of the disclosure that is isotopically enriched or labelled (with respect to one or more atoms of the compound) with one or more stable isotopes. Thus, in this application, the compounds of the disclosure include, for example, compounds that are isotopically enriched or labelled with one or more atoms such as deuterium.

As used herein, “treating” describes the management and care of a subject for the purpose of combating a disease, condition, or disorder and includes decreasing or alleviating the symptoms or complications, or eliminating the disease, condition or disorder.

The term “treating”, as used herein, unless otherwise indicated, means reversing, alleviating, or inhibiting the progress of the disorder or condition to which such term applies, or one or more symptoms of such disorder or condition. The term “treatment”, as used herein, unless otherwise indicated, refers to the act of “treating” as defined immediately above. For example, the terms “treat”, “treating” and “treatment” can refer to a method of alleviating or abrogating a particular disorder and/or one or more of its attendant symptoms.

As used herein, “subject” means a human or animal (in the case of an animal, the subject can be a mammal). In one aspect, the subject is a human. In one aspect, the subject is a male.

Prostate cancer is the uncontrolled growth of cancerous cells in the prostate gland. In some embodiments, the prostate cancer is metastatic prostate cancer, castrate-resistant prostate cancer, metastatic castrate-resistant prostate cancer, castrate-sensitive prostate cancer, metastatic castrate-sensitive prostate cancer, prostate cancer naïve to novel hormonal agents (NHA), castrate-sensitive prostate cancer naïve to novel hormonal agents (NHA), castrate-resistant prostate cancer naïve to novel hormonal agents (NHA), metastatic prostate cancer naïve to novel hormonal agents (NHA), metastatic castrate-resistant prostate cancer naïve to novel hormonal agents (NHA), or metastatic castrate-sensitive prostate cancer naïve to novel hormonal agents (NHA).

Metastatic prostate cancer, or metastases, refers to prostate cancer that has spread beyond the prostate to other parts of the body, e.g., bones, lymph nodes, liver, lungs, brain.

Castrate-resistant prostate cancer or castration-resistant prostate cancer (or prostate cancer that is castrate- or castration-resistant) is a type of prostate cancer that keeps growing even when the amount of testosterone in the body is reduced to very low levels.

Metastatic castrate-resistant prostate cancer is a type of prostate cancer that has metastasized and continues to grow even when the amount of testosterone in the body is reduced to very low levels.

Castrate-sensitive prostate cancer or castration-sensitive prostate cancer (CSPC), or prostate cancer that is castrate- or castration-sensitive, is prostate cancer that can be controlled by reducing the amount of androgens (male hormones) in the body (e.g., through castration) and/or prostate cancer that requires androgens to grow and stops growing when androgens are not present. CSPC is also referred to as androgen-dependent prostate cancer, androgen-sensitive prostate cancer, or hormone-sensitive prostate cancer (HSPC).

Metastatic castrate-sensitive prostate cancer is a type of castrate-sensitive prostate cancer that has metastasized and requires androgens to grow or can be controlled by reducing the amount of androgens in the body (e.g., through castration).

Prostate cancer naïve to novel hormonal agents (NHA) is prostate cancer that has not been previously treated with second-generation antiandrogens such as androgen biosynthesis inhibitors or androgen receptor blockers. In some embodiments, the androgen biosynthesis inhibitor is abiraterone or abiraterone acetate. In some embodiments, the androgen receptor blocker is enzalutamide, darolutamide, or apalutamide.

Metastatic prostate cancer naïve to novel hormonal agents (NHA) is metastatic prostate cancer that has not been previously treated with second-generation antiandrogens such as androgen biosynthesis inhibitors or androgen receptor blockers. In some embodiments, the androgen biosynthesis inhibitor is abiraterone or abiraterone acetate. In some embodiments, the androgen receptor blocker is enzalutamide, darolutamide, or apalutamide.

Castrate-resistant prostate cancer naïve to novel hormonal agents (NHA) is castrate-resistant prostate cancer that has not been previously treated with second-generation antiandrogens such as androgen biosynthesis inhibitors or androgen receptor blockers. In some embodiments, the androgen biosynthesis inhibitor is abiraterone or abiraterone acetate. In some embodiments, the androgen receptor blocker is enzalutamide, darolutamide, or apalutamide.

Castrate-sensitive prostate cancer naïve to novel hormonal agents (NHA) is castrate-sensitive prostate cancer that has not been previously treated with second-generation antiandrogens such as androgen biosynthesis inhibitors or androgen receptor blockers. In some embodiments, the androgen biosynthesis inhibitor is abiraterone or abiraterone acetate). In some embodiments, the androgen receptor blocker is enzalutamide, darolutamide, or apalutamide.

Metastatic castrate-resistant prostate cancer naïve to novel hormonal agents (NHA) is metastatic castrate-resistant prostate cancer that has not been previously treated with second-generation antiandrogens such as androgen biosynthesis inhibitors or androgen receptor blockers. In some embodiments, the androgen biosynthesis inhibitor is abiraterone or abiraterone acetate. In some embodiments, the androgen receptor blocker is enzalutamide, darolutamide, or apalutamide.

Metastatic castrate-sensitive prostate cancer naïve to novel hormonal agents (NHA) is metastatic castrate-sensitive prostate cancer that has not been previously treated with second-generation antiandrogens such as androgen biosynthesis inhibitors or androgen receptor blockers. In some embodiments, the androgen biosynthesis inhibitor is abiraterone or abiraterone acetate). In some embodiments, the androgen receptor blocker is enzalutamide, darolutamide, or apalutamide.

As used herein, “preventing” describes stopping the onset of the symptoms or complications of the disease, condition or disorder.

“Administration” refers to introducing an agent, such as a compound of the disclosure into a subject. The related terms “administering” and “administration of” (and grammatical equivalents) refer both to direct administration, which may be administration to a subject by a medical professional or by self-administration by the subject, and/or to indirect administration, which may be the act of prescribing a drug. For example, a physician who instructs a patient to self-administer a drug and/or provides a patient with a prescription for a drug is administering the drug to the patient.

The terms “co-administration” and “co-administering” or “combination therapy” refer to both concurrent administration (administration of two or more therapeutic agents at the same time) and time varied administration (administration of one or more therapeutic agents at a time different from that of the administration of an additional therapeutic agent or agents), as long as the therapeutic agents are present in the patient to some extent, preferably at effective amounts, at the same time. In certain preferred aspects, one or more of the present compounds described herein, are co-administered in combination with at least one additional bioactive agent, especially including an anti-cancer agent. In particularly preferred aspects, the co-administration of compounds results in synergistic activity and/or therapy, including anticancer activity.

“Therapeutically effective amount”, as used herein means an amount of the free base of a compound of the disclosure, or the equivalent amount of a pharmaceutically acceptable salt of the compound of the disclosure, that is sufficient to treat, ameliorate, or prevent a specified disease (e.g., prostate cancer), disease symptom, disorder or condition, or to exhibit a detectable therapeutic or inhibitory effect. The effect can be detected by any assay method known in the art. The effective amount for a particular subject may depend upon the subject's body weight, size, and health; the nature and extent of the condition; and whether additional therapeutics are to be administered to the subject. Therapeutically effective amounts for a given situation can be determined by routine experimentation that is within the skill and judgment of the clinician.

“Cₘₐₓ”, as used herein, refers to the observed maximum (peak) plasma concentration of a specified compound in the subject after administration of a dose of that compound to the subject.

“AUC”, as used herein, refers to the total area under the plasma concentration-time curve, which is a measure of exposure to a compound of interest, and is the integral of the concentration-time curve after a single dose or at steady state. AUC is expressed in units of ng*H/mL (ng×H/mL), where “H” refers to hours.

“AUCₜₐᵤ”, as used herein, refers to the AUC from 0 hours to the end of a dosing interval.

“AUC₀₋₂₄” means the AUC from 0 hours to 24 hours after administration of a single dose.

“Controlled release” or “CR” as used herein with respect to an oral dosage form refers to where a compound of the disclosure is released from the dosage form according to a predetermined profile that may include when and where release occurs after oral administration and/or a specified rate of release over a specified time period

“Controlled release agent” as used herein with respect to an oral dosage form of the disclosure refers to one or more substances or materials that modulate release of a compound of the disclosure from the dosage form. Controlled release agents may be materials which are organic or inorganic, naturally occurring or synthetic, such as polymeric materials, triglycerides, derivatives of triglycerides, fatty acids and salts of fatty acids, talc, boric acid, colloidal silica, and combinations thereof.

“Enteric coating” as used herein with respect to a dosage form of the disclosure refers to a pH-dependent material that surrounds a core comprising a compound of the disclosure and which remains substantially intact in the acid environment of the stomach, but which dissolves in the pH environment of the intestines.

“Gastro-resistant” or “GR” as applied to a CR oral dosage form described herein means that release of a compound of the disclosure in the stomach of a subject shall not exceed 5%, 2.5%, 1% or 0.5% of the total amount of the compound of the disclosure in the dosage form.

“Oral dosage form” as used herein refers to a pharmaceutical drug product that contains a specified amount (dose) of a compound of the disclosure as the active ingredient, or a pharmaceutically acceptable salt and/or solvate thereof, and inactive components (excipients), formulated into a particular configuration that is suitable for oral administration, such as an oral tablet, liquid, or capsule. In some embodiments, the compositions are in the form of a tablet that can be scored.

The term “carrier”, as used in this disclosure, encompasses pharmaceutically acceptable excipients and diluents and means a material, composition or vehicle, such as a liquid or solid filler, diluent, excipient, solvent or encapsulating material, involved in carrying or transporting a pharmaceutical agent from one organ, or portion of the body, to another organ, or portion of the body of a subject.

The term “about” as part of a quantitative expression such as “about X”, includes any value that is 10% higher or lower than X, and also includes any numerical value that falls between X-10% and X+10%. Thus, for example, a weight of about 40 g includes a weight of between 36 to 44 g. When used herein to denote amino acid residues in the AR, the term “about” means any amino acid residue that is within 5 amino acid residues of what is specified. For example, when referring to a contiguous stretch of amino acid residues extending from about amino acid residue 560 to about amino acid residue 624 of the AR, this refers to a contiguous stretch of amino acid residues extending from amino acid residue 555, 556, 557, 558, 559, 560, 561, 562, 563, 564, or 565, to amino acid residue 619, 620, 621, 622, 623, 624, 625, 626, 627, 628, or 629 of the AR of SEQ ID NO: 1. In some embodiments, the term “about” means any amino acid residue that is within 3 amino acid residues of what is specified. In some embodiments, the term “about” means any amino acid residue that is within 1 amino acid residue of what is specified.

Compound A of the present disclosure refers to 4-(4-((1-(4-(((1R,3R)-3-(4-cyano-3-methoxyphenoxy)-2,2,4,4-tetramethylcyclobutyl)carbamoyl)phenyl)piperidin-4-yl)methyl)piperazin-1-yl)-N—((S)-2,6-dioxopiperidin-3-yl)-2-fluorobenzamide, which has the following structure:

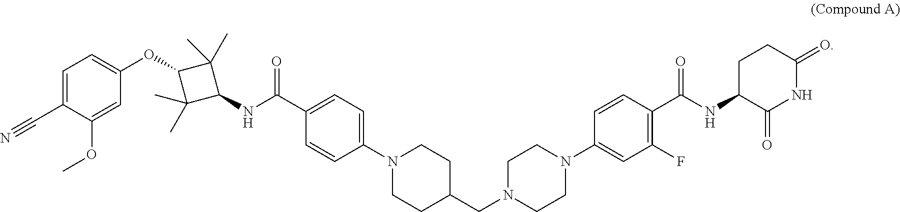 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

In some embodiments, Compound A can be prepared as described in US Patent Application Publication No. 2021/0196710 A1, which is incorporated herein by reference.

“Comprising” or “comprises” as applied to a particular dosage form, composition, use, method or process described or claimed herein means that the dosage form, composition, use, method, or process includes all of the recited elements in a specific description or claim but does not exclude other elements. “Consists essentially of” and “consisting essentially of” means that the described or claimed composition, dosage form, method, use, or process does not exclude other materials or steps that do not materially affect the recited physical, pharmacological, pharmacokinetic properties or therapeutic effects of the composition, dosage form, method, use, or process. “Consists of” and “consisting of” means the exclusion of more than trace elements of other ingredients and substantial method or process steps.

The ECOG Scale of Performance Status was developed by the Eastern Cooperative Oncology Group as standard criteria for measuring how the disease impacts a patient's daily living abilities. It describes a patient's level of functioning in terms of their ability to care for themself, daily activity, and physical ability (walking, working, etc.). Patients are categorized on a scale of 1-5:

Grade ECOG Performance Status
0 Fully active, able to carry on all pre-disease performance without restriction
1 Restricted in physically strenuous activity but ambulatory and able to carry out work of a light or sedentary nature, e.g., light housework, office work
2 Ambulatory and capable of all selfcare but unable to carry out any work activities; up and about more than 50% of waking hours
3 Capable of only limited selfcare; confined to bed or chair more than 50% of waking hours
4 Completely disabled; cannot carry on any selfcare; totally confined to bed or chair Dead

“Fasted condition” or “fasted state” as used to describe a subject means the subject has not eaten for at least 4 hours before a time point of interest, such as the time of administering a compound of the disclosure (preferably Compound A or a pharmaceutically acceptable salt thereof). In an embodiment, a subject in the fasted state has not eaten for at least any of 6, 8, 10 or 12 hours prior to administration of a compound of the disclosure (preferably Compound A or a pharmaceutically acceptable salt thereof).

“Fed condition” or “fed state” as used to describe a subject herein means the subject has eaten less than 4 hours before a time point of interest, such as the time of administering a compound of the disclosure (preferably Compound A or a pharmaceutically acceptable salt thereof). In an embodiment, a subject in the fed state has eaten within at least any of 3, 2, 1 or 0.5 hours prior to administration of a compound of the disclosure (preferably Compound A or a pharmaceutically acceptable salt thereof).

Corticosteroids are a class of steroid hormones that include both steroid hormones produced in humans and other vertebrates and synthetic derivatives or analogues of the same. In some embodiments, the corticosteroid is a glucocorticoid. In some embodiments, the corticosteroid is a mineralocorticoid. In some embodiments, the corticosteroid is prednisone, prednisolone, methylprednisolone, cortisone, cortisol, dexamethasone, betamethasone, triamcinolone, deflazacort, fludrocortisone acetate, deoxycorticosterone acetate, aldosterone, or beclometasone.

A “proton pump inhibitor”, or “PPI”, refers to a member of a class of compounds that reduces or eliminates the production of acid in the stomach. In some embodiments, the PPI is omeprazole, esomeprazole, lansoprazole, pantoprazole, dexlansoprazole, or rabeprazole.

An “H2 antagonist” or “H2 receptor blocker”, collectively referred to as “H2 compounds” herein, refers to a member of a class of compounds that reduces or prevents histamine-induced gastric acid secretion in the stomach. In some embodiments the H2 compound is rantidine, famotidine, nizatidine, or cimetidine.

“Stomach acid-modifying agents”, as used herein, refer collectively to PPIs, H2 compounds, and antacids, including those prescribed by a physician and those available over the counter.

As used herein, the term “CDK inhibitor” refers to a compound that inhibits the enzymes in humans referred to as cyclin-dependent kinases (CDK). In some embodiments, the CDK inhibitor is a CDK4/6 inhibitor. As used herein, the term “CDK4/6 inhibitor” refers to a compound that inhibits CDK 4 and/or 6. Examples of a CDK inhibitor include, without limitation, SHR6390, trilaciclib, lerociclib, AT7519M, dinaciclib, ribociclib, abemaciclib, palbociclib, or any pharmaceutically acceptable salt thereof. In some embodiments, the CDK inhibitor is palbociclib or a pharmaceutically acceptable salt thereof.

As used herein, the term “PARP inhibitor” refers to a compound that inhibits the enzymes in humans referred to as poly ADP ribose polymerase (PARP). Examples of a PARP inhibitor include, without limitation, olaparib, rucaparib, talazoparib, niraparib, veliparib, pamiparib, CEP 9722, E7016, 3-aminobenzamide, mefuparib, and AZD2281.

As used herein, the term “anti-cancer agent” is used to describe an anti-cancer agent, or a therapeutic agent administered concurrently with an anti-cancer agent (e.g., palonosetron), with which may be co-administered and/or co-formulated with a compound of the disclosure (preferably Compound A or a pharmaceutically acceptable salt thereof) to treat cancer, and the side effects associated with the cancer treatment. These agents include, for example, everolimus, trabectedin, abraxane, TLK 286, AV-299, DN-101, pazopanib, GSK690693, RTA 744, ON 0910.Na, AZD 6244 (ARRY-142886), AMN-107, TKI-258, GSK461364, AZD 1152, enzastaurin, vandetanib, ARQ-197, MK-0457, MLN8054, PHA-739358, R-763, AT-9263, a FLT-3 inhibitor, a VEGFR inhibitor, an EGFR TK inhibitor, an aurora kinase inhibitor, a PIK-1 modulator, a Bcl-2 inhibitor, an HDAC inhibitor, a c-MET inhibitor, a PARP inhibitor, a CDK inhibitor, an EGFR TK inhibitor, an IGFR-TK inhibitor, an anti-HGF antibody, a PI3 kinase inhibitor, an AKT inhibitor, an mTORC1/2 inhibitor, a JAK/STAT inhibitor, a checkpoint-1 or 2 inhibitor, a focal adhesion kinase inhibitor, a Map kinase (mek) inhibitor, a VEGF trap antibody, pemetrexed, erlotinib, dasatanib, nilotinib, decatanib, panitumumab, amrubicin, oregovomab, Lep-etu, nolatrexed, azd2171, batabulin, ofatumumab, zanolimumab, edotecarin, tetrandrine, rubitecan, tesmilifene, oblimersen, ticilimumab, ipilimumab, gossypol, Bio 111, 131-I-TM-601, ALT-110, BIO 140, CC 8490, cilengitide, gimatecan, IL13-PE38QQR, INO 1001, IPdRI KRX-0402, lucanthone, LY317615, neuradiab, vitespan, Rta 744, Sdx 102, talampanel, atrasentan, Xr 311, romidepsin, ADS-100380, sunitinib, 5-fluorouracil, vorinostat, etoposide, gemcitabine, doxorubicin, liposomal doxorubicin, 5′-deoxy-5-fluorouridine, vincristine, temozolomide, ZK-304709, seliciclib; PD0325901, AZD-6244, capecitabine, L-Glutamic acid, N-[4-[2-(2-amino-4,7-dihydro-4-oxo-1H-pyrrolo[2,3-d]pyrimidin-5-yl)ethyl]benzoyl]-, disodium salt, heptahydrate, camptothecin, PEG-labeled irinotecan, tamoxifen, toremifene citrate, anastrazole, exemestane, letrozole, DES(diethylstilbestrol), estradiol, estrogen, conjugated estrogen, bevacizumab, IMC-1C11, CHIR-258); 3-[5-(methylsulfonylpiperadinemethyl)-indolyl-quinolone, vatalanib, AG-013736, AVE-0005, goserelin acetate, leuprolide acetate, triptorelin pamoate, medroxyprogesterone acetate, hydroxyprogesterone caproate, megestrol acetate, raloxifene, bicalutamide, flutamide, nilutamide, megestrol acetate, CP-724714; TAK-165, HKI-272, erlotinib, lapatanib, canertinib, ABX-EGF antibody, erbitux, EKB-569, PKI-166, GW-572016, Ionafarnib, BMS-214662, tipifarnib; amifostine, NVP-LAQ824, suberoyl analide hydroxamic acid, valproic acid, trichostatin A, FK-228, SU11248, sorafenib, KRN951, aminoglutethimide, arnsacrine, anagrelide, L-asparaginase, Bacillus Calmette-Guerin (BCG) vaccine, adriamycin, bleomycin, buserelin, busulfan, carboplatin, carmustine, chlorambucil, cisplatin, cladribine, clodronate, cyproterone, cytarabine, dacarbazine, dactinomycin, daunorubicin, diethylstilbestrol, epirubicin, fludarabine, fludrocortisone, fluoxymesterone, flutamide, gleevec, gemcitabine, hydroxyurea, idarubicin, ifosfamide, imatinib, leuprolide, levamisole, lomustine, mechlorethamine, melphalan, 6-mercaptopurine, mesna, methotrexate, mitomycin, mitotane, mitoxantrone, nilutamide, octreotide, oxaliplatin, pamidronate, pentostatin, plicamycin, porfimer, procarbazine, raltitrexed, rituximab, streptozocin, teniposide, testosterone, thalidomide, thioguanine, thiotepa, tretinoin, vindesine, 13-cis-retinoic acid, phenylalanine mustard, uracil mustard, estramustine, altretamine, floxuridine, 5-deooxyuridine, cytosine arabinoside, 6-mecaptopurine, deoxycoformycin, calcitriol, valrubicin, mithramycin, vinblastine, vinorelbine, topotecan, razoxin, marimastat, COL-3, neovastat, BMS-275291, squalamine, endostatin, SU5416, SU6668, EMD121974, interleukin-12, IM862, angiostatin, vitaxin, droloxifene, idoxyfene, spironolactone, finasteride, cimitidine, trastuzumab, denileukin diftitox, gefitinib, bortezimib, paclitaxel, cremophor-free paclitaxel, docetaxel, epithilone B, BMS-247550, BMS-310705, droloxifene, 4-hydroxytamoxifen, pipendoxifene, ERA-923, arzoxifene, fulvestrant, acolbifene, lasofoxifene, idoxifene, TSE-424, HMR-3339, ZK186619, topotecan, PTK787/ZK 222584, VX-745, PD 184352, rapamycin, 40-O-(2-hydroxyethyl)-rapamycin, temsirolimus, AP-23573, RAD001, ABT-578, BC-210, LY294002, LY292223, LY292696, LY293684, LY293646, wortmannin, ZM336372, L-779,450, PEG-filgrastim, darbepoetin, erythropoietin, granulocyte colony-stimulating factor, zolendronate, prednisone, cetuximab, granulocyte macrophage colony-stimulating factor, histrelin, pegylated interferon alfa-2a, interferon alfa-2a, pegylated interferon alfa-2b, interferon alfa-2b, azacitidine, PEG-L-asparaginase, lenalidomide, gemtuzumab, hydrocortisone, interleukin-11, dexrazoxane, alemtuzumab, all-transretinoic acid, ketoconazole, interleukin-2, megestrol, immune globulin, nitrogen mustard, methylprednisolone, ibritgumomab tiuxetan, androgens, decitabine, hexamethylmelamine, bexarotene, tositumomab, arsenic trioxide, cortisone, editronate, mitotane, cyclosporine, liposomal daunorubicin, Edwina-asparaginase, strontium 89, casopitant, netupitant, an NK-1 receptor antagonist, palonosetron, aprepitant, diphenhydramine, hydroxyzine, metoclopramide, lorazepam, alprazolam, haloperidol, droperidol, dronabinol, dexamethasone, methylprednisolone, prochlorperazine, granisetron, ondansetron, dolasetron, tropisetron, pegfilgrastim, erythropoietin, epoetin alfa, darbepoetin alfa, and mixtures thereof. In some embodiments, the anti-cancer agent is selected from the group consisting of abiraterone, abiraterone acetate, estramustine, docetaxel, ketoconazole, goserelin, histrelin, triptorelin, buserelin, cyproterone, flutamide, bicalutamide, nilutamide, pamidronate, and zolendronate. In some embodiments, the anti-cancer agent is selected from the group consisting of FLT-3 inhibitor, androgen receptor inhibitor, VEGFR inhibitor, EGFR TK inhibitor, aurora kinase inhibitor, PIK-1 modulator, Bcl-2 inhibitor, HDAC inhibitor, c-Met inhibitor, PARP inhibitor, CDK 4/6 inhibitor, anti-HGF antibody, IGFR TK inhibitor, PI3 kinase inhibitor, AKT inhibitor, JAK/STAT inhibitor, checkpoint 1 inhibitor, checkpoint 2 inhibitor, focal adhesion kinase inhibitor, Map kinase inhibitor, VEGF trap antibody, and chemical castration agent.

In some embodiments, the anti-cancer agent is selected from the group consisting of temozolomide, capecitabine, irinotecan, tamoxifen, anastrazole, exemestane, letrozole, DES, Estradiol, estrogen, bevacizumab, goserelin acetate, leuprolide acetate, triptorelin pamoate, medroxyprogesterone acetate, hydroprogesterone caproate, raloxifene, megestrol acetate, carboplatin, cisplatin, dacarbazine, methotrexate, vinblastine, vinorelbine, topotecan, finasteride, arzoxifene, fulvestrant, prednisone, abiraterone, abiraterone acetate, enzalutamide, apalutamide, darolutamide, sipuleucel-T, pembrolizumab, nivolumab, cemiplimab, atezolizumab (Tecentriq), avelumab (Bavencio), durvalumab (Imfinzi), docetaxel (Taxotere), cabazitaxel (Jevtana), mitoxantrone (Novantrone), estramustine (Emcyt), docetaxel, ketoconazole, histrelin, triptorelin, buserelin, cyproterone, flutamide, bicalutamide, nilutamide, pamidronate, and zolendronate.

Abiraterone acetate, i.e.: [(3S,8R,9S,10R,13S,14S)-10,13-dimethyl-17-pyridin-3-yl-2,3,4,7,8,9,11,12,14,15-decahydro-1H-cyclopenta[a]phenanthren-3-yl] acetate, is a commercially available drug developed by Janssen and sold under the brand name Zytiga® that is indicated for use in combination with prednisone for the treatment of patients with metastatic castration-resistant prostate cancer. The structure of abiraterone acetate is:

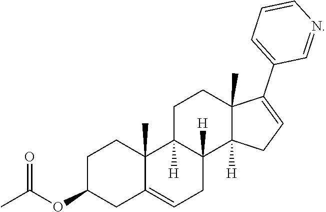

Abiraterone, i.e., (3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-ol, is the active metabolite of abiraterone acetate, and has the following structure:

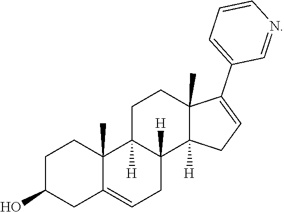

The articles “a” and “an” are used in this disclosure to refer to one or more than one (i.e., to at least one) of the grammatical object of the article. By way of example, “an element” means one element or more than one element.

The term “and/or” is used in this disclosure to mean either “and” or “or” unless indicated otherwise.

The terms “patient” and “subject” are used interchangeably herein, and refer to a mammal, e.g., a human, mouse, rat, guinea pig, dog, cat, horse, cow, pig, or non-human primate, such as a monkey, chimpanzee, baboon or rhesus.

In some embodiments, the subject is a human.

In some embodiments, the subject is a human who has been diagnosed with prostate cancer.

In some embodiments, the subject is a human who has been diagnosed with metastatic prostate cancer.

In some embodiments, the subject is a human who has been diagnosed with castrate-resistant prostate cancer.

In some embodiments, the subject is a human who has been diagnosed with metastatic castrate-resistant prostate cancer.

In some embodiments, the subject is a human who has been diagnosed with castrate-sensitive prostate cancer.

In some embodiments, the subject is a human who has been diagnosed with metastatic castrate-sensitive prostate cancer.

In some embodiments, the subject is a human who has been diagnosed with prostate cancer naïve to novel hormonal agents (NHA).

In some embodiments, the subject is a human who has been diagnosed with castrate-sensitive prostate cancer naïve to novel hormonal agents (NHA).

In some embodiments, the subject is a human who has been diagnosed with castrate-resistant prostate cancer naïve to novel hormonal agents (NHA)

In some embodiments, the subject is a human who has been diagnosed with metastatic prostate cancer naïve to novel hormonal agents (NHA).

In some embodiments, the subject is a human who has been diagnosed with metastatic castrate-resistant prostate cancer naïve to novel hormonal agents (NHA).

In some embodiments, the subject is a human who has been diagnosed with metastatic castrate-sensitive prostate cancer naïve to novel hormonal agents (NHA).

### Compounds of the Disclosure

In one aspect, the application pertains to a bifunctional or multifunctional compounds useful for regulating protein activity by inducing the degradation of a target protein. In some embodiments, the bifunctional compound comprises an E3 ubiquitin ligase binding moiety and a protein targeting moiety, preferably linked through a linker moiety, as otherwise described herein, wherein the E3 ubiquitin ligase binding moiety is coupled to the protein targeting moiety and wherein the E3 ubiquitin ligase binding moiety recognizes a ubiquitin pathway protein (e.g., a ubiquitin ligase, preferably an E3 ubiquitin ligase) and the protein targeting moiety recognizes the target protein such that degradation of the target protein will occur when the target protein is placed in proximity to the ubiquitin ligase, thus resulting in degradation/inhibition of the effects of the target protein and the control of protein levels. In certain embodiments, the bifunctional compound comprises a CLM coupled, e.g., linked covalently, directly, or indirectly, to a chemical linker L, and a PTM, which can be depicted as:

PTM-L-CLM

The CLM recognizes and binds to cereblon, an E3 Ubiquitin Ligase. The PTM is a small molecule protein binding moiety that binds and recruits an intracellular target protein or polypeptide bringing it into close proximity to the CLM to effect the degradation of the target protein, resulting in target protein ubiquitination. In certain embodiments, the PTM is an AR binding moiety (ABM).

In any of the compounds described herein, the PTM comprises the following chemical structure:

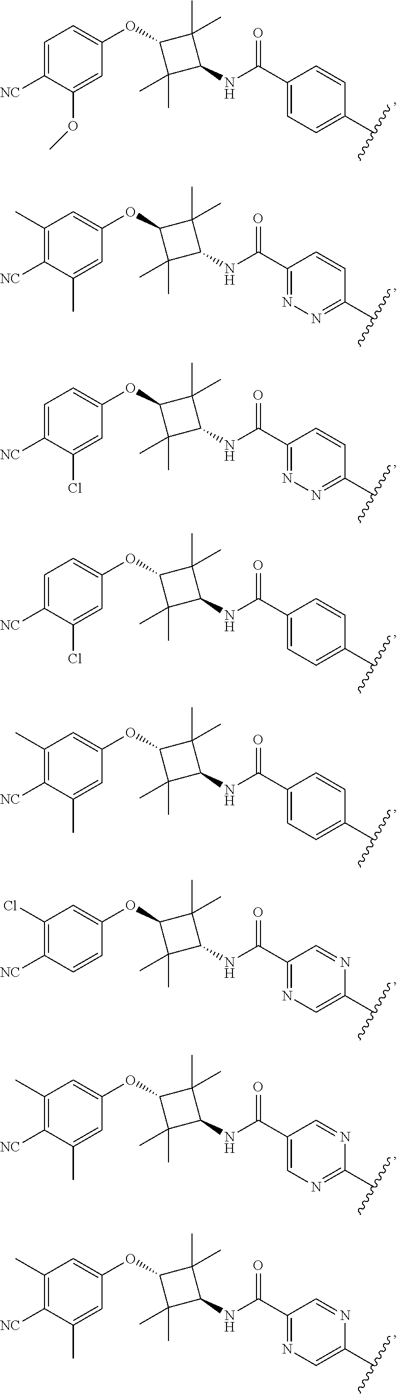

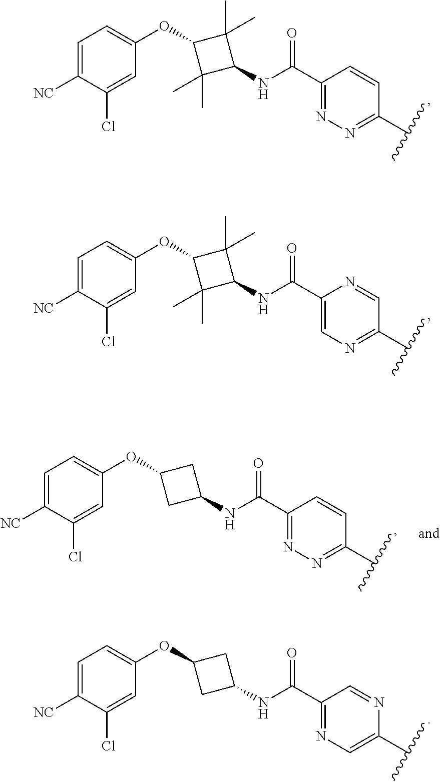 [SMILES: *c1ccc(C(=O)N[C@H]2C(C)(C)[C@H](Oc3ccc(C#N)c(Cl)c3)C2(C)C)nn1 ; *c1cnc(C(=O)N[C@H]2C(C)(C)[C@H](Oc3ccc(C#N)c(Cl)c3)C2(C)C)cn1 ; *c1ccc(C(=O)N[C@H]2C[C@H](Oc3ccc(C#N)c(Cl)c3)C2)nn1 ; *c1cnc(C(=O)N[C@H]2C[C@H](Oc3ccc(C#N)c(Cl)c3)C2)cn1]

In any of the compounds described herein, the L comprises the following chemical structure:

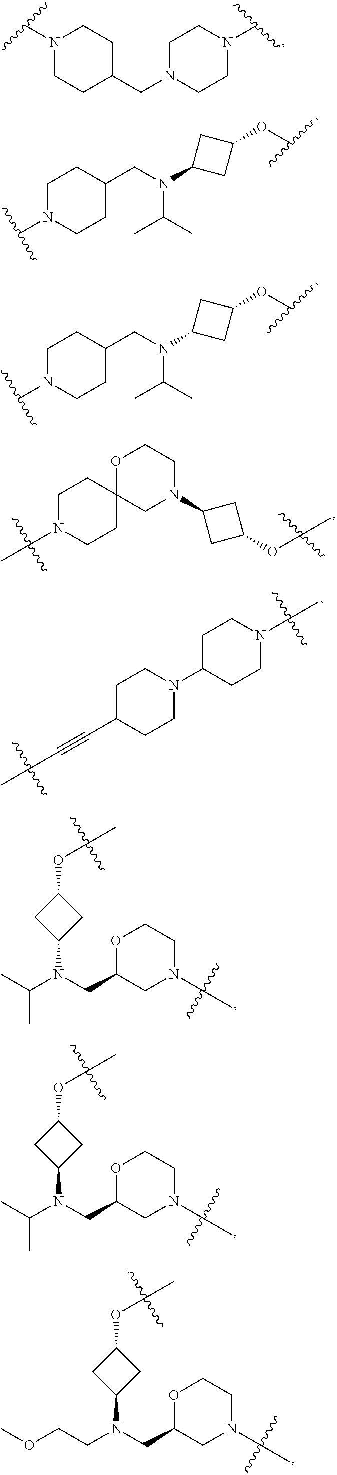

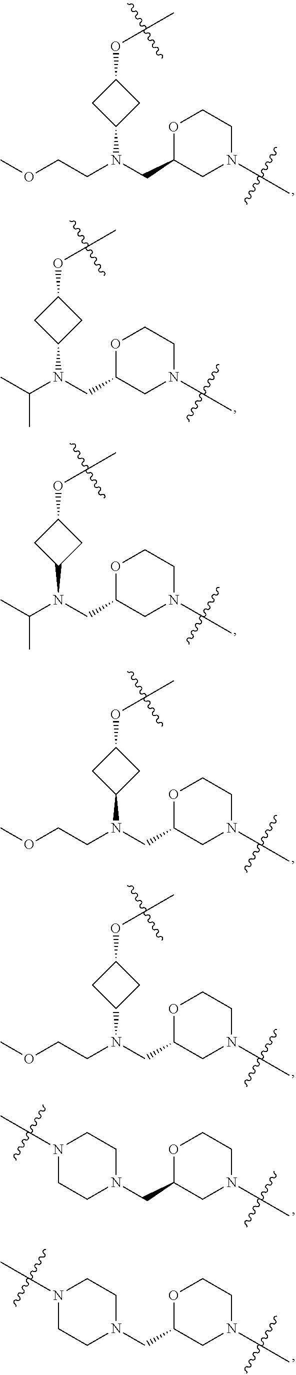

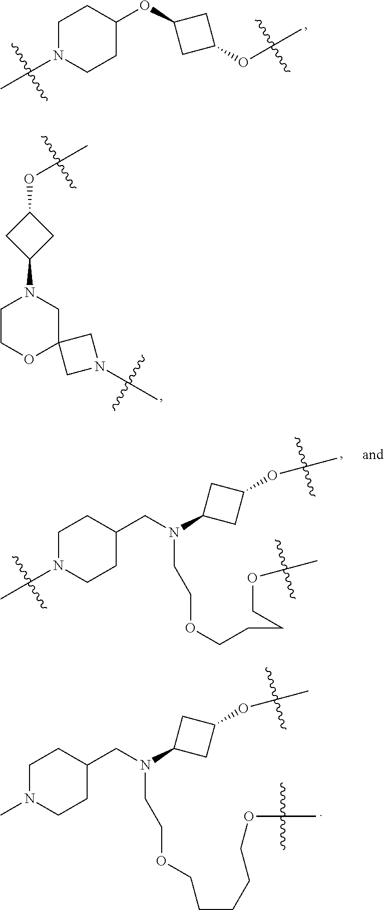 [SMILES: *O[C@H]1C[C@H](OC2CCN(*)CC2)C1 ; *O[C@H]1C[C@H](N2CCOC3(CN(*)C3)C2)C1 ; *OCCCCOCCN(CC1CCN(*)CC1)[C@H]1C[C@H](O*)C1 ; *OCCCCCOCCN(CC1CCN(C)CC1)[C@H]1C[C@H](O*)C1]

In any of the compounds described herein, the CLM comprises the following chemical structures:

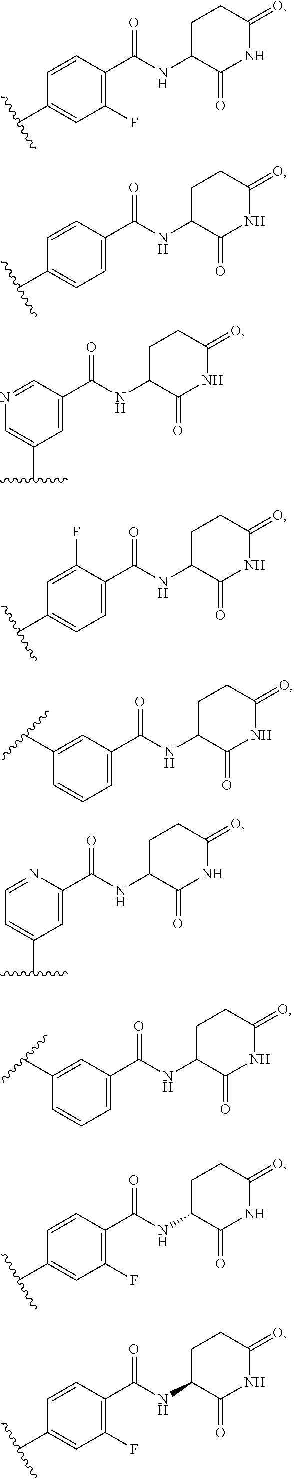

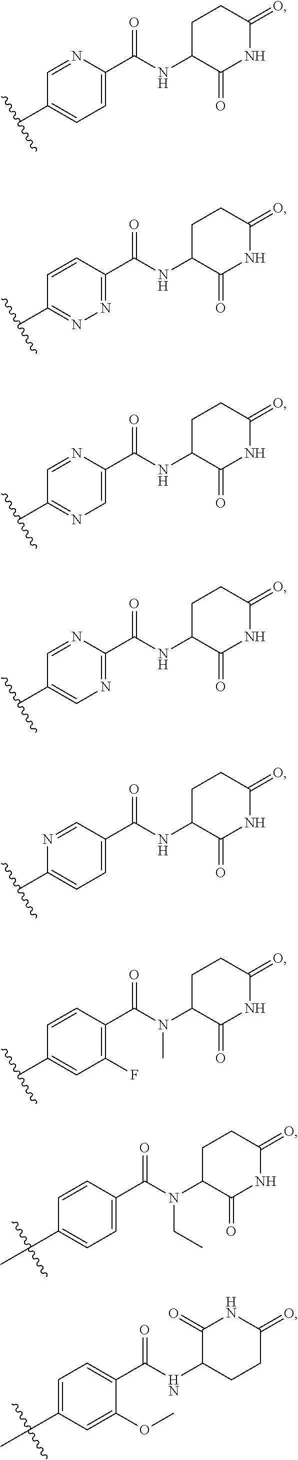

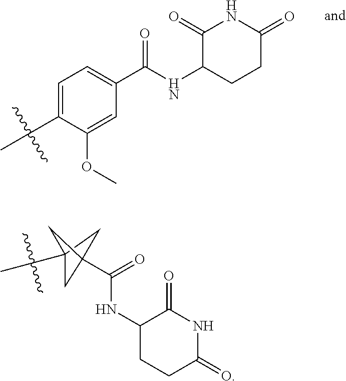 [SMILES: *c1ccc(C(=O)NC2CCC(=O)NC2=O)cc1OC ; *C12CC(C(=O)NC3CCC(=O)NC3=O)(C1)C2]

In another aspect, the application pertains to a bifunctional compound having the structure:

ABM-L-CLM,
or a pharmaceutically acceptable salt, thereof,
wherein:
(a) ABM is an androgen receptor (AR) binding moiety having the structure:

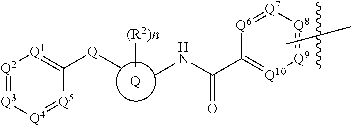

wherein:
   Q¹, Q² Q³ Q⁴, and Q⁵ are each independently CR¹, or N;

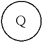

is 4-6 membered cycloalkyl, C₆-C₁₀ aryl, 4-6 membered heterocycloalkyl, or 4-6 membered heteroaryl, wherein the heterocycloalkyl or heteroaryl comprises 0-4 heteroatoms;
Q⁶, Q⁷, Q⁸, Q⁹, and Q¹⁰ are each independently CR³, or N;
Each R¹ is independently selected from the group consisting of H, optionally substituted linear or branched C₁-C₆ alkyl, cyano, halogen, and optionally substituted linear or branched C₁-C₆ alkoxy, wherein the alkyl or alkoxy group is optionally substituted with one or more halo;
Each R² is independently selected from the group consisting of optionally substituted linear or branched C₁-C₆ alkyl, cyano, halogen, and optionally substituted linear or branched C₁-C₆ alkoxy, wherein the alkyl or alkoxy group is optionally substituted with one or more halo;
Each R³ is independently selected from the group consisting of H, optionally substituted linear or branched C₁-C₆ alkyl, cyano, halogen, and optionally substituted linear or branched C₁-C₆ alkoxy, wherein the alkyl or alkoxy group is optionally substituted with one or more halo; and
   n is 0, 1, 2, 3, or 4;
(b) L is a chemical linking moiety having the structure:

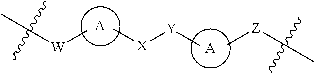

wherein:
   the ABM is linked to W, and the CLM is linked to Z or
   the ABM is linked to Z, and the CLM is linked to W;
   W is absent or

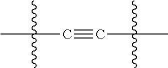 [SMILES: *C#C*]

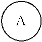

is 4-7 membered cycloalkyl, 4-7 membered heterocycle, or spiro-bicyclic heterocycloalkyl, wherein each ring in the spiro-bicycle is 4-7 membered;
X is —CH₂— or absent;
Y is —NR⁶—, —O—, or absent;

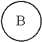

is 4-7 membered cycloalkyl or 4-7 membered heterocycle;
Z is —C(R⁷)₂—, —NR⁷—, —O—, or absent;
R⁶ is H, linear or branched Ci-6 alkyl, linear or branched Ci-6 alkoxy-C₁₋₆ alkyl, or

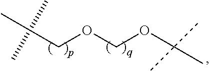

wherein

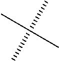

indicates a bond to Y, and

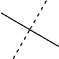

indicates a bond to

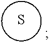

each R⁷ is independently selected from the group consisting of H, linear or branched Ci-6 alkyl, and linear or branched C₁₋₆ alkoxy;
p is 1, 2, 3, or 4; and
q is 1, 2, 3, 4, or 5;
(c) CLM is cereblon E3 ubiquitin ligase binding moiety having the structure:

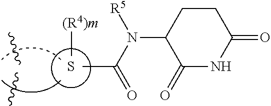

Wherein:

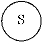

is C₆-C₁₀ aryl, 4-7 membered heteroaryl, or bridged bicyclic cycloalkyl;

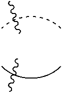

indicates that linking moiety L is connected to ring S by one or two covalent bonds;
Each R⁴ is independently selected from the group consisting of optionally substituted linear or branched C₁-C₆ alkyl, cyano, halogen, and optionally substituted linear or branched C₁-C₆ alkoxy, wherein the alkyl or alkoxy group is optionally substituted with one or more halo;
R⁵ is H, optionally substituted linear or branched C₁-C₆ alkyl, or optionally substituted linear or branched C₁-C₆ alkoxy, wherein the alkyl or alkoxy group is optionally substituted with one or more halo; and
m is 0, 1, 2, 3, or 4.

In another aspect, the application pertains to a compound of Formula (I):

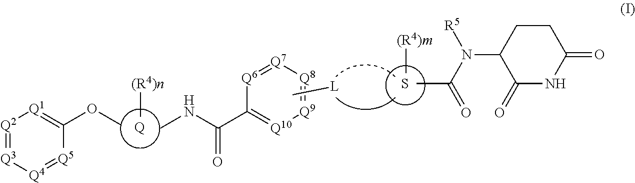

or a pharmaceutically acceptable salt, thereof,
wherein all variables are as defined herein.

In some embodiments, when

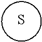

is pyridyl,

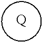

is tetramethylcyclobutyl, Q² is CR¹, and Q⁴ is CR¹, then R¹ is not chloro.

In some embodiments, the compound of Formula (I) is not N-(4-(3-chloro-4-cyanophenoxy)cyclohexyl)-6-(4-((4-(4-((2,6-dioxopiperidin-3-yl)carbamoyl)phenyl)piperazin-1-yl)methyl)piperidin-1-yl)pyridazine-3-carboxamide.

In some embodiments, the compound of Formula (I) is not

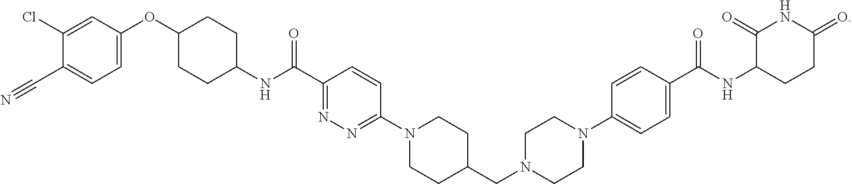 [SMILES: N#Cc1ccc(OC2CCC(NC(=O)c3ccc(N4CCC(CN5CCN(c6ccc(C(=O)NC7CCC(=O)NC7=O)cc6)CC5)CC4)nn3)CC2)cc1Cl]

In some embodiments, L is

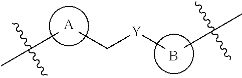

wherein:

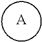

is 4-7 membered cycloalkyl or 4-7 membered heterocycle;
   Y is —NR⁶—, —O—, or absent;
   R⁶ is H, linear or branched C₁₋₆ alkyl, or linear or branched C₁₋₆ alkoxy; and

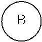

is 4-7 membered cycloalkyl or 4-7 membered heterocycle.

In some embodiments, L is

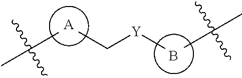

wherein:

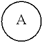

is 4-7 membered cycloalkyl or 4-7 membered heterocycle;
   Y is —NR⁶— or —O—;
   R⁶ is H, linear or branched C₁₋₆ alkyl, or linear or branched C₁₋₆ alkoxy; and

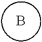

is 4-7 membered cycloalkyl or 4-7 membered heterocycle.

In some embodiments, L is

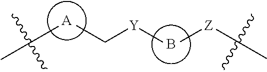

Wherein:

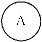

is piperidinyl or morpholinyl;
   Y is —NR⁶— or —O—;
   R₆ is

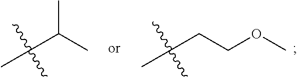

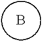

is cyclobutyl; and
   Z is —O—.

In some embodiments, L is

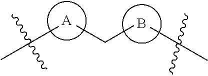

Wherein:

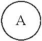

is piperidinyl or morpholinyl; and

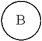

is piperazinyl.

In some embodiments,

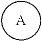

is piperidinyl.

In some embodiments, the compound is a compound of Formula (Ib) or a pharmaceutically acceptable salt thereof:
wherein all variables are as defined herein.

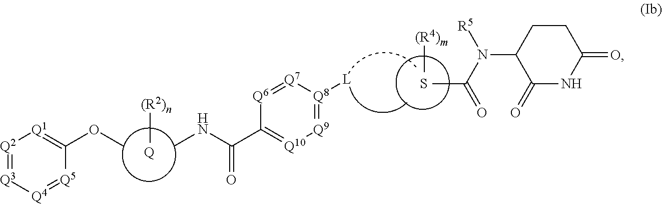

In some embodiments, the compound is a compound of Formula (Ic) or a pharmaceutically acceptable salt thereof:

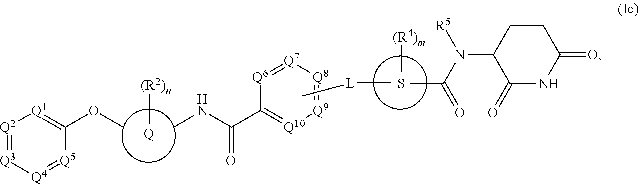

wherein all variables are as defined herein.

In some embodiments, Q¹-Q⁵ are each CR¹. In some embodiments, between 1-3 of Q¹-Q⁵ is N. In some embodiments, exactly 1 of Q¹-Q⁵ is N. In some embodiments, exactly 2 of Q¹-Q⁵ is N. In some embodiments, exactly 3 of Q¹-Q⁵ is N.

In some embodiments, Q⁶-Q¹⁰ are each CR¹. In some embodiments, between 1-3 of Q6-Q¹⁰ is N. In some embodiments, exactly 1 of Q⁶-Q¹⁰ is N. In some embodiments, exactly 2 of Q⁶-Q¹⁰ is N. In some embodiments, exactly 3 of Q⁶-Q¹⁰ is N.

In some embodiments, Q¹ is CH, Q² is C(CH₃), Q³ is C(CN), Q⁴ is C(CH₃), and Q⁵ is CH.

In some embodiments, Q¹ is CH, Q² is C(OCH₃), Q³ is C(CN), Q⁴ is CH, and Q⁵ is CH.

In some embodiments, Q¹ is CH, Q² is C(Cl), Q³ is C(CN), Q⁴ is CH, and Q⁵ is CH.

In some embodiments, R¹ is selected from the group consisting of H, CN and CH₃. In some embodiments, R¹ is selected from the group consisting of H, CN and OCH³. In some embodiments, R¹ is selected from the group consisting of H, CN and Cl. In some embodiments, at least one R₁ is CF₃.

In some embodiments,

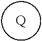

is 4-6 membered cycloalkyl. In some embodiments,

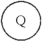

is cyclobutyl or cyclohexyl. In some embodiments,

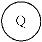

is cyclobutyl. In some embodiments,

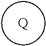

is cyclopentyl. In some embodiments,

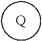

is cyclohexyl.

In some embodiments, n is 0. In some embodiments, n is 1. In some embodiments, n is 2. In some embodiments, n is 3. In some embodiments, n is 4.

In some embodiments, R² is linear or branched C₁-C₆ alkyl. In some embodiments, R² is methyl. In some embodiments, R² is ethyl. In some embodiments, R² is propyl. In some embodiments, R² is n-propyl. In some embodiments, R² is isopropyl. In some embodiments, R² is butyl. In some embodiments, R² is n-butyl. In some embodiments, R² is isobutyl. In some embodiments, R² is sec-butyl. In some embodiments, R² is tert-butyl. In some embodiments, R² is pentyl. In some embodiments, R² is hexyl.

In some embodiments,

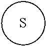

is phenyl, pyridinyl, pyridazinyl, pyrimidinyl, or pyrazinyl. In some embodiments,

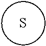

is phenyl In some embodiments,

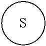

is pyridinyl. In some embodiments,

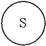

is pyridazinyl. In some embodiments,

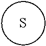

is pyrimidinyl. In some embodiments,

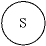

is pyrazinyl.

In some embodiments, each R⁴ is independently selected from the group consisting of F, methoxy, ethoxy, methyl, and ethyl. In some embodiments, each R⁴ is independently selected from the group consisting of F, methoxy, and methyl. In some embodiments R⁴ is F.

In some embodiments, m is 0, 1, or 2. In some embodiments, m is 0. In some embodiments, m is 1. In some embodiments, m is 2.

In one aspect, the application pertains to a compound of Formula (Id) or a pharmaceutically acceptable salt thereof:

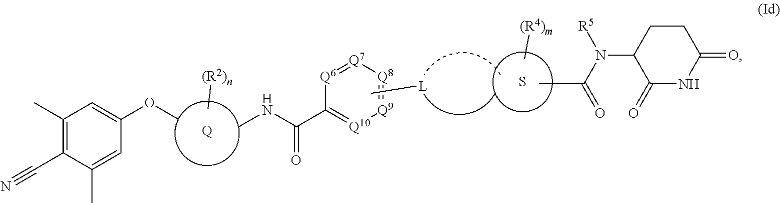

wherein all variables are as defined herein.

In one aspect, the application pertains to a compound of Formula (Ie) or a pharmaceutically acceptable salt thereof:

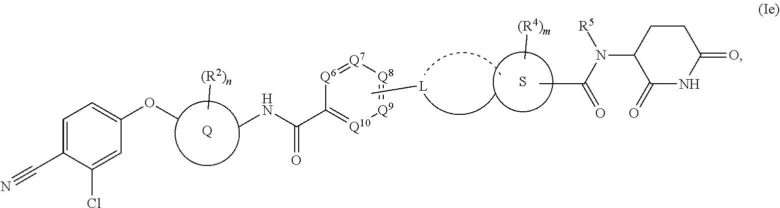

wherein all variables are as defined herein.

In one aspect, the application pertains to a compound of Formula (If) or a pharmaceutically acceptable salt thereof:

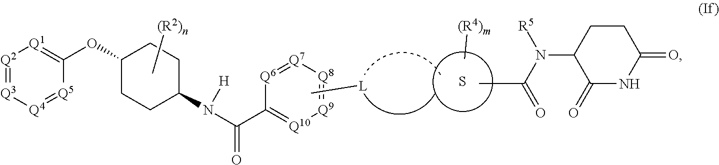

wherein all variables are as defined herein.

In one aspect, the application pertains to a compound of Formula (Ig) or a pharmaceutically acceptable salt thereof:

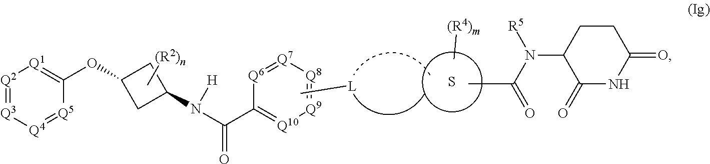

wherein all variables are as defined herein.

In one aspect, the application pertains to a compound of Formula (Ih) or a pharmaceutically acceptable salt thereof:

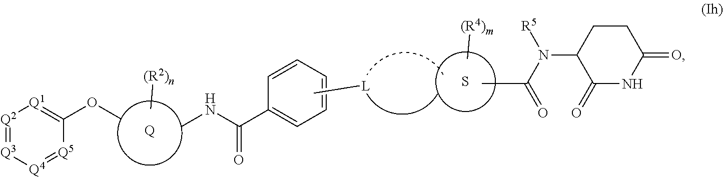

wherein all variables are as defined herein.

In one aspect, the application pertains to a compound of Formula (Ii) or a pharmaceutically acceptable salt thereof:

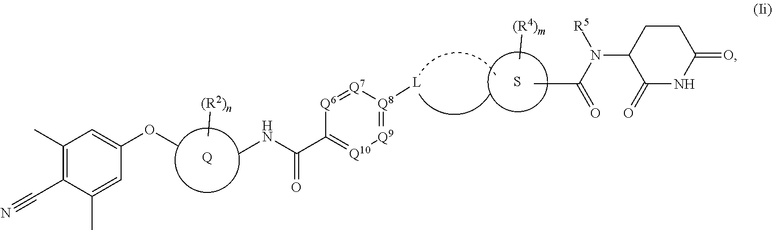

wherein all variables are as defined herein.

In one aspect, the application pertains to a compound of Formula (Ij) or a pharmaceutically acceptable salt thereof:

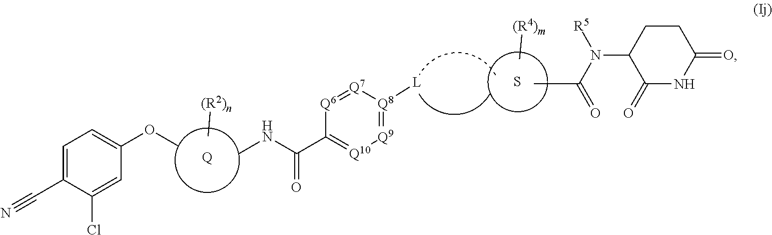

wherein all variables are as defined herein.

In one aspect, the application pertains to a compound of Formula (Ik) or a pharmaceutically acceptable salt thereof:

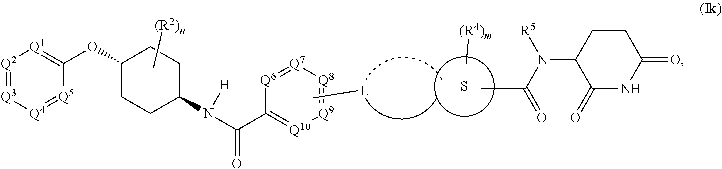

wherein all variables are as defined herein.

In one aspect, the application pertains to a compound of Formula (Il) or a pharmaceutically acceptable salt thereof:

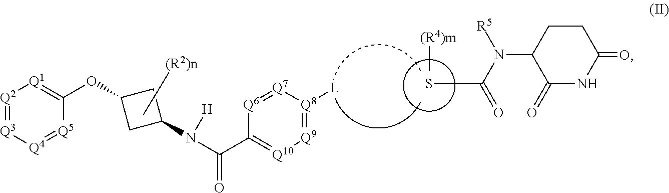

wherein all variables are as defined herein.

In one aspect, the application pertains to a compound of Formula (Im) or a pharmaceutically acceptable salt thereof:

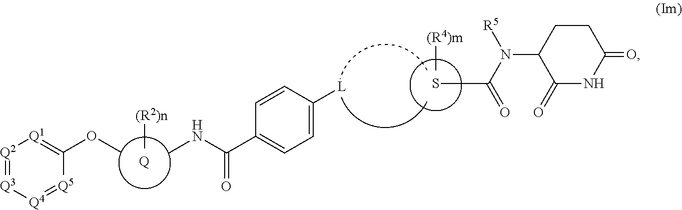

wherein all variables are as defined herein.

In one aspect, the application pertains to a compound of Formula (Io) or a pharmaceutically acceptable salt thereof:

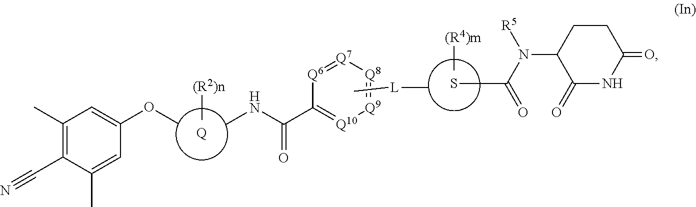

wherein all variables are as defined herein.

In one aspect, the application pertains to a compound of Formula (Ip) or a pharmaceutically acceptable salt thereof:

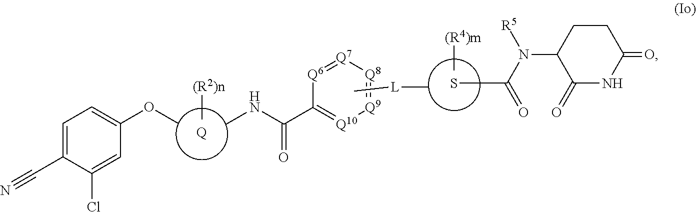

wherein all variables are as defined herein.

In one aspect, the application pertains to a compound of Formula (Iq) or a pharmaceutically acceptable salt thereof:

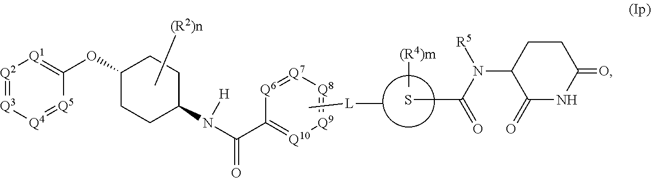

wherein all variables are as defined herein.

In one aspect, the application pertains to a compound of Formula (Ir) or a pharmaceutically acceptable salt thereof:

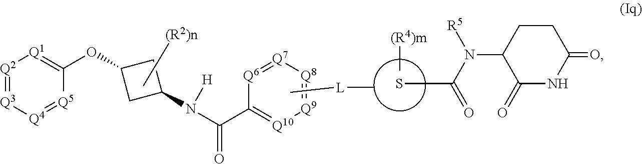

wherein all variables are as defined herein.

In one aspect, the application pertains to a compound of Formula (Is) or a pharmaceutically acceptable salt thereof:

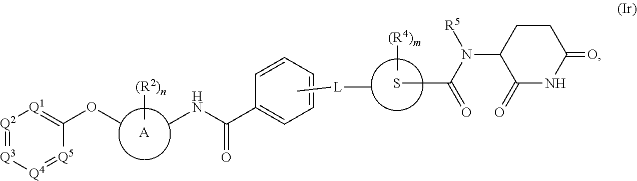

wherein all variables are as defined herein.

In some embodiments, the compound of the disclosure is Compound A, i.e.: 4-(4-((1-(4-(((1R,3R)-3-(4-cyano-3-methoxyphenoxy)-2,2,4,4-tetramethylcyclobutyl)carbamoyl)phenyl)piperidin-4-yl)methyl)piperazin-1-yl)-N—((S)-2,6-dioxopiperidin-3-yl)-2-fluorobenzamide, which has the following structure:

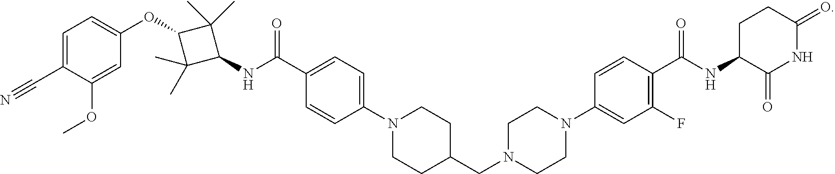 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

A compound of the disclosure may be synthesized using standard synthetic methods and procedures for the preparation of organic molecules and functional group transformations and manipulations, including the use of protective groups, as can be obtained from the relevant scientific literature or from standard reference textbooks in the field in view of this disclosure. Although not limited to any one or several sources, recognized reference textbooks of organic synthesis include: Smith, M. B.; March, J. March's Advanced Organic Chemistry: Reactions, Mechanisms, and Structure, 5^{th} ed.; John Wiley & Sons: New York, 2001; and Greene, T. W.; Wuts, P. G. M. Protective Groups in Organic Synthesis, 3^{rd}; John Wiley & Sons: New York, 1999. The synthetic methods described in U.S. Patent Application Publication No. 2018/0099940 and International Publication No. 2018/144649 are incorporated herein by reference in their entireties.

In some embodiments, the compounds of the disclosure may be prepared according to the procedures and methods disclosed herein, including, for example, where indicated in Table 1. Other bifunctional compounds of the disclosure can be prepared using similar methods from common intermediates or derivatives thereof.

### Methods of Ubiquitinating/Degrading a Target Protein in a Cell

The present disclosure provides a method of ubiquitinating/degrading a target protein in a cell. The method comprises administering a bifunctional composition comprising an E3 ubiquitin ligase binding moiety and a protein targeting moiety, preferably linked through a linker moiety, as otherwise described herein, wherein the E3 ubiquitin ligase binding moiety is coupled to the protein targeting moiety and wherein the E3 ubiquitin ligase binding moiety recognizes a ubiquitin pathway protein (e.g., an ubiquitin ligase, preferably an E3 ubiquitin ligase) and the protein targeting moiety recognizes the target protein such that degradation of the target protein will occur when the target protein is placed in proximity to the ubiquitin ligase, thus resulting in degradation/inhibition of the effects of the target protein and the control of protein levels. The control of protein levels afforded by the present disclosure provides treatment of a disease state or condition, which is modulated through the target protein by lowering the level of that protein in the cells of a patient.

In one aspect, this application provides a compound of Formula (I), or a pharmaceutically acceptable salt thereof, that degrades the androgen receptor (AR) protein. In some embodiments, the AR that is degraded by the compound of Formula (I) is wild type AR. In some embodiments, the AR that is degraded by the compound of Formula (I) is a mutant form of AR.

As understood by the skilled artisan, AR has a modular structure comprising three functional domains: the N-terminal transcriptional regulation domain, the DNA-binding domain, and the ligand binding domain (MacLean H E, et al J. Steroid Biochem Mol Biol. (1997) 62:233-42). The DNA-binding domain is linked to the ligand-binding domain via a hinge. The AR ligand binding domain refers to the functional domain of human AR that folds to form a hydrophobic pocket that binds to the AR cognate hormone ligand (e.g., androgen).

Moreover, it is understood in the art that AR is 920 amino acid residues in length, wherein the N-terminal transcriptional regulation domain extends from amino acid residue 1 to about amino acid residue 559, the DNA-binding domain extends from about amino acid residue 560 to about amino acid residue 624, the hinge extends from about amino acid residue 625 to about amino acid residue 676, and the ligand binding domain extends from about amino acid residue 677 to about amino acid residue 920. A suitable AR reference sequence is set forth by SEQ ID NO: 1 and identified in the UniProt database as P10275 (ANDR_HUMAN). The gene encoding AR (“the AR gene”) is approximately 90 kb and has chromosomal coordinates 67544021-67730619 according to human reference genome GRCh38.p13. The AR gene contains 8 exons, with exon 1 encoding the N-terminal transcriptional regulation domain; exon 2-3 encoding the DNA-binding domain; and exons 4-8 encoding the hinge and ligand binding domain (Jenster, et al (1992) J. Steroid Biochem. Mol. Biol. 41:671-75).

In some embodiments, the subject has a prostate cancer comprising at least one somatic AR tumor mutation in a functional domain of AR. In some embodiments, the at least one somatic AR tumor mutation is an insertion, deletion, or substitution of one or more amino acid residues in an AR functional domain as compared to an AR reference sequence (e.g., SEQ ID NO: 1). In some embodiments, the at least one somatic AR tumor mutation is a substitution of one or more amino acid residues in an AR functional domain as compared to an AR reference sequence (e.g., SEQ ID NO: 1). In some embodiments, the at least one somatic AR tumor mutation is an insertion, deletion, or substitution of one or more amino acid residues in the AR ligand binding domain as compared to an AR reference sequence (e.g., SEQ ID NO: 1). In some embodiments, the at least one somatic AR tumor mutation is a substitution of one or more amino acid residues in the AR ligand binding domain as compared to an AR reference sequence (e.g., SEQ ID NO: 1). In some embodiments, the at least one somatic AR tumor mutation is an insertion, deletion or substitution of one or more amino acid residues selected from amino acid residues 677-920 as compared to an AR reference sequence, wherein the AR reference sequence is set forth by SEQ ID NO: 1. In some embodiments, the at least one somatic AR tumor mutation is a substitution of one or more amino acid residues selected from amino acid residues 677-920 as compared to an AR reference sequence, wherein the AR reference sequence is set forth by SEQ ID NO: 1.

In some embodiments, the alteration (e.g., substitution) of an amino acid residue in the AR ligand binding domain provides a mutant AR having reduced ligand specificity and/or enhanced cofactor recruitment. Without being bound by theory, a mutant AR having a reduced ligand specificity and/or enhanced cofactor recruitment has increased potency for triggering the AR signaling pathway, thereby conferring a growth advantage on a tumor cell comprising the mutant AR.

In some embodiments, the prostate cancer comprises cancer cells characterized by expression of at least one somatic AR tumor mutation described herein. Methods to identify a cancer characterized by expression of somatic mutations are known in the art, and include, e.g., obtaining a biological sample from the subject, harvesting the biological sample to obtain genetic material (e.g., genomic DNA or RNA), and performing sequencing analysis, RNA-sequencing analysis, or real-time polymerase chain reaction (RT-PCR). For example, in some embodiments, genomic DNA is first obtained (using any standard technique) from cancerous tissue obtained from the subject, cDNA is prepared, and amplification is performed (e.g., using a polymerase chain reaction) to provide the cDNA in sufficient quantity for sequence analysis, and sequencing is performed using, e.g., next generation sequencing. Genomic DNA or RNA is typically extracted from biological samples such as tissues removed from the subject, e.g., by tissue biopsy. In some embodiments, the biological sample is a tissue biopsy sample (e.g., a prostate biopsy sample), wherein sequence analysis of genomic DNA or RNA is performed to identify the presence of somatic mutations in AR (e.g., a somatic mutation resulting in a substitution of an amino acid residue in the AR ligand binding domain). In some embodiments, the biological sample comprises plasma obtained from the subject is used to detect somatic AR tumor mutations present in circulating tumor DNA, e.g., using PCR-based amplification, followed by gene sequencing.

In some embodiments, the mutant form of AR that is degraded by the compound of Formula (I) or a pharmaceutically acceptable salt thereof, comprises at least one AR somatic tumor mutation.

In some embodiments, the at least one somatic AR tumor mutation is selected from any one or any combination of L702X, V716X, V731X, W742X, M750X, H875X, F877X, T878X, D880X, L882X, S889X, D891X, M895X, M896X, and E898X, wherein “X” refers to any amino acid residue other than the wild type residue at that position. In some embodiments, the at least one somatic AR tumor mutation is selected from any one or any combination of L702X, V716X, V731X, W742X, M750X, H875X, F877X, T878X, D880X, L882X, S889X, D891X, M895X, M896X, and E898X, wherein “X” refers to an amino acid residue, other than the wild type residue at that position, selected from alanine (A); valine (V); leucine (L); isoleucine (I); phenylalanine (F); methionine (M); tryptophan (W); proline (P); glycine (G); serine (S); threonine (T); cysteine (C); asparagine (N); glutamine (Q); tyrosine (Y); lysine (K); arginine (R); histidine (H); aspartate (D); and glutamate (E).

In some embodiments, the at least one somatic AR tumor mutation is selected from any one or any combination of L702X, T878X, H875X, W742X, F877X, V716X, D891X, M750X, M895X, M896X, and S889X, wherein “X” refers to any amino acid residue other than the wild type residue at that position. In some embodiments, the at least one somatic AR tumor mutation is selected from any one or any combination of L702X, T878X, H875X, W742X, F877X, V716X, D891X, M750X, M895X, M896X, and S889X, wherein “X” refers to an amino acid residue, other than the wild type residue at that position, selected from alanine (A); valine (V); leucine (L); isoleucine (I); phenylalanine (F); methionine (M); tryptophan (W); proline (P); glycine (G); serine (S); threonine (T); cysteine (C); asparagine (N); glutamine (Q); tyrosine (Y); lysine (K); arginine (R); histidine (H); aspartate (D); and glutamate (E).

In some embodiments, the at least one somatic AR tumor mutation is selected from any one or any combination of L702X, T878X, and H875X, wherein “X” refers to any amino acid residue other than the wild type residue at that position. In some embodiments, the at least one somatic AR tumor mutation is selected from any one or any combination of L702X, T878X, and H875X, wherein “X” refers to an amino acid residue, other than the wild type residue at that position, selected from alanine (A); valine (V); leucine (L); isoleucine (I); phenylalanine (F); methionine (M); tryptophan (W); proline (P); glycine (G); serine (S); threonine (T); cysteine (C); asparagine (N); glutamine (Q); tyrosine (Y); lysine (K); arginine (R); histidine (H); aspartate (D); and glutamate (E).

In some embodiments, the at least one somatic AR tumor mutation is L702X, wherein “X” refers to any amino acid residue other than the wild type residue at that position. In some embodiments, the at least one somatic AR tumor mutation is L702X, wherein “X” refers to an amino acid residue, other than the wild type residue at that position, selected from alanine (A); valine (V); leucine (L); isoleucine (I); phenylalanine (F); methionine (M); tryptophan (W); proline (P); glycine (G); serine (S); threonine (T); cysteine (C); asparagine (N); glutamine (Q); tyrosine (Y); lysine (K); arginine (R); histidine (H); aspartate (D); and glutamate (E). In some embodiments, the at least one somatic AR tumor mutation is L702H.

In some embodiments, the at least one somatic AR tumor mutation is T878X, wherein “X” refers to any amino acid residue other than the wild type residue at that position. In some embodiments, the at least one somatic AR tumor mutation is T878X, wherein “X” refers to an amino acid residue, other than the wild type residue at that position, selected from alanine (A); valine (V); leucine (L); isoleucine (I); phenylalanine (F); methionine (M); tryptophan (W); proline (P); glycine (G); serine (S); threonine (T); cysteine (C); asparagine (N); glutamine (Q); tyrosine (Y); lysine (K); arginine (R); histidine (H); aspartate (D); and glutamate (E). In some embodiments, the at least one somatic AR tumor mutation is T878X, wherein “X” refers to an amino acid residue, other than the wild type residue at that position, selected from alanine (A) and serine (S). In some embodiments, the at least one somatic AR tumor mutation is T878A. In some embodiments, the at least one somatic AR tumor mutation is T878A.

In some embodiments, the at least one somatic AR tumor mutation is H875X, wherein “X” refers to any amino acid residue other than the wild type residue at that position. In some embodiments, the at least one somatic AR tumor mutation is H875X, wherein “X” refers to an amino acid residue, other than the wild type residue at that position, selected from alanine (A); valine (V); leucine (L); isoleucine (I); phenylalanine (F); methionine (M); tryptophan (W); proline (P); glycine (G); serine (S); threonine (T); cysteine (C); asparagine (N); glutamine (Q); tyrosine (Y); lysine (K); arginine (R); histidine (H); aspartate (D); and glutamate (E). In some embodiments, the at least one somatic AR tumor mutation is H875Y.

In some embodiments, the at least one somatic AR tumor mutation is not selected from any one or any combination of L702X, T878X, and H875X, wherein “X” refers to any amino acid residue other than the wild type residue at that position. In some embodiments, the at least one somatic AR tumor mutation is not selected from any one or any combination of L702X, T878X, and H875X, wherein “X” refers to an amino acid residue, other than the wild type residue at that position, selected from alanine (A); valine (V); leucine (L); isoleucine (I); phenylalanine (F); methionine (M); tryptophan (W); proline (P); glycine (G); serine (S); threonine (T); cysteine (C); asparagine (N); glutamine (Q); tyrosine (Y); lysine (K); arginine (R); histidine (H); aspartate (D); and glutamate (E).

In some embodiments, the at least one somatic AR tumor mutation is not L702X, wherein “X” refers to any amino acid residue other than the wild type residue at that position. In some embodiments, the at least one somatic AR tumor mutation is not L702X, wherein “X” refers to an amino acid residue, other than the wild type residue at that position, selected from alanine (A); valine (V); leucine (L); isoleucine (I); phenylalanine (F); methionine (M); tryptophan (W); proline (P); glycine (G); serine (S); threonine (T); cysteine (C); asparagine (N); glutamine (Q); tyrosine (Y); lysine (K); arginine (R); histidine (H); aspartate (D); and glutamate (E). In some embodiments, the at least one somatic AR tumor mutation is not L702H.

In some embodiments, the at least one somatic AR tumor mutation is not T878X, wherein “X” refers to any amino acid residue other than the wild type residue at that position. In some embodiments, the at least one somatic AR tumor mutation is not T878X, wherein “X” refers to an amino acid residue, other than the wild type residue at that position, selected from alanine (A); valine (V); leucine (L); isoleucine (I); phenylalanine (F); methionine (M); tryptophan (W); proline (P); glycine (G); serine (S); threonine (T); cysteine (C); asparagine (N); glutamine (Q); tyrosine (Y); lysine (K); arginine (R); histidine (H); aspartate (D); and glutamate (E). In some embodiments, the at least one somatic AR tumor mutation is not T878X, wherein “X” refers to an amino acid residue, other than the wild type residue at that position, selected from alanine (A) and serine (S). In some embodiments, the at least one somatic AR tumor mutation is not T878A. In some embodiments, the at least one somatic AR tumor mutation is not T878A.

In some embodiments, the at least one somatic AR tumor mutation is not H875X, wherein “X” refers to any amino acid residue other than the wild type residue at that position. In some embodiments, the at least one somatic AR tumor mutation is not H875X, wherein “X” refers to an amino acid residue, other than the wild type residue at that position, selected from alanine (A); valine (V); leucine (L); isoleucine (I); phenylalanine (F); methionine (M); tryptophan (W); proline (P); glycine (G); serine (S); threonine (T); cysteine (C); asparagine (N); glutamine (Q); tyrosine (Y); lysine (K); arginine (R); histidine (H); aspartate (D); and glutamate (E). In some embodiments, the at least one somatic AR tumor mutation is not H875Y.

In some embodiments, the at least one somatic AR tumor mutation is selected from any one or any combination of L702H, V716M, V731M, W742L, W742C, H875Y, H875Q, H875L, F877L, T878A, T878S, D880E, L882I, S889G, D891H, D891Y, M896T, M896V, and E898G.

In some embodiments, the at least one somatic AR tumor mutation is selected from any one or any combination of L702H, T878A, H875Y, W742C, W742L, F877L, T878S, V716M, D891H, M750V, M750T, M895X, M896X, and S889G. In some embodiments, the at least one somatic AR tumor mutation is selected from any one or any combination of E666K, Q799E, Q793E, Q118K, Y447N, S532Y, G751C, Q825E, L702H, T878A, H875Y, H875L, Q825E, W742C, W742L, F877L, T878S, V716M, D891H, M750V, M750T, and S889G. In some embodiments, the at least one somatic AR tumor mutation is selected from any one or any combination of E666K, Q799E, Q793E, Q118K, Y447N, S532Y, G751C, Q825E, T878A, H875Y, H875L, Q825E, W742C, W742L, F877L, T878S, V716M, D891H, M750V, M750T, and S889G.

In some embodiments, the at least one somatic AR tumor mutation is selected from any one or any combination of T878A, H875Y, H875L, Q825E, W742C, W742L, F877L, T878S, V716M, D891H, M750V, M750T, M895X, M896X, and S889G. In some embodiments, the at least one somatic AR tumor mutation is selected from any one or any combination of H875Y, H875L, T878A, F877L, V716M, T878S, W742C, and W742L. In some embodiments, the at least one somatic AR tumor mutation is selected from any one or any combination of H875Y, H875L, Q825E, T878A, F877L, V716M, T878S, W742C, and W742L. In some embodiments, the at least one somatic AR tumor mutation is L702H. In some embodiments, the at least one somatic AR tumor mutation is T878A. In some embodiments, the at least one somatic AR tumor mutation is H875Y. In some embodiments, the at least one somatic AR tumor mutation is H875L. In some embodiments, the at least one somatic AR tumor mutation is Q825E. In some embodiments, the at least one somatic AR tumor mutation is W742C. In some embodiments, the at least one somatic AR tumor mutation is W742L. In some embodiments, the at least one somatic AR tumor mutation is F877L. In some embodiments, the at least one somatic AR tumor mutation is T878S. In some embodiments, the at least one somatic AR tumor mutation is V716M. In some embodiments, the at least one somatic AR tumor mutation is D891H. In some embodiments, the at least one somatic AR tumor mutation is M750V. In some embodiments, the at least one somatic AR tumor mutation is M750T. In some embodiments, the at least one somatic AR tumor mutation is S889G.

In some embodiments, the at least one somatic AR tumor mutation is selected from L702H, T878A, H875Y, W742C, W742L, F877L, T878S, V716M, D891H, M750V, M750T, and S889G.

In some embodiments, the at least one somatic AR tumor mutation is selected from L702H, M895V, W742C, S889G, M750V, M896V, T878A, T878S, F877L, D891H, H875Y, and any combination thereof.

In some embodiments, the mutant form of AR that is degraded by the compound of Formula (I) or a pharmaceutically acceptable salt thereof, comprises at least two AR somatic tumor mutations.

In some embodiments, the at least two somatic AR tumor mutation are selected from L702H, M895V, W742C, S889G, M750V, M896V, T878A, T878S, F877L, D891H, H875Y, and any combination thereof.

In some embodiments, the mutant form of AR that is degraded by the compound of Formula (I) comprises at least two AR somatic tumor mutations.

In some embodiments, the at least two somatic AR tumor mutations are selected from L702X, T878X, H875X, W742X, F877X, V716X, D891X, M750X, M895X, M896X, and S889X, wherein “X” refers to any amino acid residue other than the wild type residue at that position. In some embodiments, the at least two somatic AR tumor mutations are selected from L702X, T878X, H875X, W742X, F877X, V716X, D891X, M750X, M895X, M896X, and S889X, wherein “X” refers to an amino acid residue, other than the wild type residue at that position, selected from alanine (A); valine (V); leucine (L); isoleucine (I); phenylalanine (F); methionine (M); tryptophan (W); proline (P); glycine (G); serine (S); threonine (T); cysteine (C); asparagine (N); glutamine (Q); tyrosine (Y); lysine (K); arginine (R); histidine (H); aspartate (D); and glutamate (E).

In some embodiments, the at least two somatic AR tumor mutations are selected from L702X, V716X, V731X, W742X, M750X, H875X, F877X, T878X, D880X, L882X, S889X, D891X, M895X, M896X, and E898X, wherein “X” refers to any amino acid residue other than the wild type residue at that position. In some embodiments, the at least two somatic AR tumor mutations are selected from L702X, V716X, V731X, W742X, M750X, H875X, F877X, T878X, D880X, L882X, S889X, D891X, M895X, M896X, and E898X, wherein “X” refers to an amino acid residue, other than the wild type residue at that position, selected from alanine (A); valine (V); leucine (L); isoleucine (I); phenylalanine (F); methionine (M); tryptophan (W); proline (P); glycine (G); serine (S); threonine (T); cysteine (C); asparagine (N); glutamine (Q); tyrosine (Y); lysine (K); arginine (R); histidine (H); aspartate (D); and glutamate (E).

In some embodiments, the at least two somatic AR tumor mutations are selected from H875X, Q825X, T878X, F877X, V716X, T878X, W742X, D891X, M750X, M895X, M896X, and S889X, wherein “X” refers to any amino acid residue, other than the wild type residue at that position. In some embodiments, the at least two somatic AR tumor mutations are selected from H875X, Q825X, T878X, F877X, V716X, T878X, W742X, D891X, M750X, M895X, M896X, and S889X wherein “X” refers to an amino acid residue, other than the wild type residue at that position, selected from alanine (A); valine (V); leucine (L); isoleucine (I); phenylalanine (F); methionine (M); tryptophan (W); proline (P); glycine (G); serine (S); threonine (T); cysteine (C); asparagine (N); glutamine (Q); tyrosine (Y); lysine (K); arginine (R); histidine (H); aspartate (D); and glutamate (E).

In some embodiments, the at least two somatic AR tumor mutations are selected from L702H, T878A, H875Y, W742C, W742L, F877L, T878S, V716M, D891H, M750V, M750T, M895X, M896X, and S889G. In some embodiments, the at least two somatic AR tumor mutations are selected from E666K, Q799E, Q793E, Q118K, Y447N, S532Y, G751C, Q825E, L702H, T878A, H875Y, H875L, Q825E, W742C, W742L, F877L, T878S, V716M, D891H, M750V, M750T, and S889G. In some embodiments, the at least two somatic AR tumor mutations are selected from E666K, Q799E, Q793E, Q118K, Y447N, S532Y, G751C, Q825E, T878A, H875Y, H875L, Q825E, W742C, W742L, F877L, T878S, V716M, D891H, M750V, M750T, and S889G.

In some embodiments, the at least two somatic AR tumor mutation are selected from L702H, V716M, V731M, W742L, W742C, H875Y, H875Q, H875L, F877L, T878A, T878S, D880E, L882I, S889G, D891H, D891Y, M896T, M896V, and E898G.

In some embodiments, the at least two somatic AR tumor mutations are selected from T878A, H875Y, H875L, Q825E, W742C, W742L, F877L, T878S, V716M, D891H, M750V, M750T, M895X, M896X, and S889G. In some embodiments, the at least two somatic AR tumor mutations are selected from H875Y, H875L, T878A, F877L, V716M, T878S, W742C, and W742L. In some embodiments, the at least two somatic AR tumor mutations are selected from H875Y, H875L, Q825E, T878A, F877L, V716M, T878S, W742C, and W742L.

In some embodiments, the at least two somatic AR tumor mutation are selected from L702H, H875Y, T878A, F877L, V716M, T878S, W742C, and W742L.

In some embodiments, the at least two somatic AR tumor mutations are selected from the following groups of mutations:
L702H and H875Y;
L702H, T878A, and H875Y;
T878A, F877L, L702H, and V716M;
T878S and H875Y;
T878S and W742C;
W742C and W742L; and
L702H and T878A.

In some embodiments, the at least two somatic AR tumor mutations are selected from the following groups of mutations:
L702H and H875Y;
L702H, T878A, and H875Y;
H875L and Q825E;
T878A, F877L, and V716M;
T878A, L702H, M750T, and D891H;
T878S and H875Y;
T878A and T878S;
T878S and W742C;
W742C and W742L; and
L702H and T878A.

In some embodiments, the at least two somatic AR tumor mutations are selected from the following groups of mutations:
T878A, and H875Y;
H875L and Q825E;
T878A, F877L, and V716M;
T878A, M750T, and D891H;
T878S and H875Y;
T878A and T878S;
T878S and W742C;
W742C and W742L.

In some embodiments, the somatic AR tumor mutation(s) is/are selected from the following groups of mutation(s):
L702H;
T878A;
T878S;
H875Y;
L702H and T878A;
L702H and T878S;
L702H and H875Y;
T878A and H875Y;
T878S and H875Y;
L702H, T878A, and H875Y; and
L702H, T878S, and H875Y.

In some embodiments, the somatic AR tumor mutation is not selected from one of the following groups of mutation(s):
L702H;
T878A;
T878S;
H875Y;
L702H and T878A;
L702H and T878S;
L702H and H875Y;
T878A and H875Y;
T878S and H875Y;
L702H, T878A, and H875Y; and
L702H, T878S, and H875Y.

In some embodiments, the somatic AR tumor mutation is an AR ligand binding domain (LBD) missense mutation(s) that is not selected from one of the following groups of mutation(s):
L702H;
T878A;
T878S;
H875Y;
L702H and T878A;
L702H and T878S;
L702H and H875Y;
T878A and H875Y;
T878S and H875Y;
L702H, T878A, and H875Y; and
L702H, T878S, and H875Y.

In some embodiments, the present disclosure is directed to a method of treating a patient in need for a disease state or condition modulated through a protein where the degradation of that protein will produce a therapeutic effect in that patient, the method comprising administering to a patient in need an effective amount of a compound of Formula (I) or a pharmaceutically acceptable salt thereof, optionally in combination with another anti-cancer agent. The disease state or condition may be a disease caused by a microbial agent or other exogenous agent such as a virus, bacteria, fungus, protozoa, or other microbe or may be a disease state caused by overexpression of a protein, which leads to a disease state and/or condition.

### Methods of Treatment

In one aspect, the present application pertains to a method of treating cancer comprising administering to a subject in need thereof a therapeutically effective amount of a compound of the disclosure, preferably Compound A, or a pharmaceutically acceptable salt, enantiomer, stereoisomer, solvate, or isotopic derivative thereof.

In one aspect, the present application pertains to a method of preventing cancer comprising administering to a subject in need thereof a therapeutically effective amount of a compound of the disclosure, preferably Compound A, or a pharmaceutically acceptable salt, enantiomer, stereoisomer, solvate, polymorph, or isotopic derivative thereof.

In one aspect, the present application pertains to a method of treating and/or preventing cancer comprising administering to a subject in need thereof a therapeutically effective amount of Compound A, or a pharmaceutically acceptable salt, enantiomer, stereoisomer, solvate, or isotopic derivative thereof.

In one aspect, the present application pertains to a method of treating and/or preventing cancer comprising administering to a subject in need thereof a therapeutically effective amount of Compound A, or a pharmaceutically acceptable salt, enantiomer, stereoisomer, solvate, polymorph, isotopic derivative, or prodrug thereof, in combination with one or more additional anti-cancer agents.

In one aspect, the present application pertains to a method of treating and/or preventing cancer comprising administering to a subject in need thereof a therapeutically effective amount of Compound A, or a pharmaceutically acceptable salt thereof.

In one aspect, the present application pertains to a method of treating and/or preventing cancer comprising administering to a subject in need thereof a therapeutically effective amount of Compound A, or a pharmaceutically acceptable salt thereof, in combination with one or more additional anti-cancer agents.

The methods of treating cancer described herein result in a reduction in tumor size. Alternatively, or in addition, the cancer is metastatic cancer, and this method of treatment includes inhibition of metastatic cancer cell invasion.

In some embodiments, the cancer is prostate cancer.

In some embodiments, the cancer is adenocarcinoma of the prostate.

In some embodiments, the cancer is metastatic prostate cancer.

In some embodiments, the cancer is castrate-resistant prostate cancer.

In some embodiments, the cancer is metastatic castrate-resistant prostate cancer (mCRPC).

In some embodiments, the cancer is progressive mCRPC.

In some embodiments, the prostate cancer is castrate-sensitive prostate cancer.

In some embodiments, the prostate cancer is metastatic castrate-sensitive prostate cancer.

In some embodiments, the prostate cancer is prostate cancer naïve to novel hormonal agents (NHA).

In some embodiments, the prostate cancer is metastatic prostate cancer naïve to novel hormonal agents (NHA).

In some embodiments, the prostate cancer is castrate-resistant prostate cancer naïve to novel hormonal agents (NHA).

In some embodiments, the prostate cancer is castrate-sensitive prostate cancer naïve to novel hormonal agents (NHA).

In some embodiments, the prostate cancer is metastatic castrate-resistant prostate cancer naïve to novel hormonal agents (NHA).

In some embodiments, the prostate cancer is metastatic castrate-sensitive prostate cancer naïve to novel hormonal agents (NHA).

In some embodiments, the prostate cancer is not prostate cancer naïve to novel hormonal agents (NHA). In some embodiments, the prostate cancer that is not prostate cancer naïve to novel hormonal agents (NHA) is also metastatic prostate cancer, castrate-resistant prostate cancer, metastatic castrate-resistant prostate cancer, castrate-sensitive prostate cancer, or metastatic castrate-sensitive prostate cancer.

In some embodiments, the subject has a histological, pathological, or cytological confirmed diagnosis of adenocarcinoma of the prostate.

In some embodiments, the subject suffering from prostate cancer (e.g., mCRPC) is also undergoing ongoing androgen deprivation therapy (ADT). In some embodiments, ADT comprises the administration of a gonadotropin releasing hormone analog or inhibitor to the subject. In some embodiments, the gonadotropin releasing hormone analog or inhibitor is leuprolide, goserelin, triptorelin, histrelin, leuprolide mesylate, or a pharmaceutically acceptable salt thereof. In some embodiments, the gonadotropin releasing hormone analog or inhibitor is leuprolide. In some embodiments, the gonadotropin releasing hormone analog or inhibitor is leuprolide mesylate. In some embodiments, the gonadotropin releasing hormone analog or inhibitor is goserelin. In some embodiments, the gonadotropin releasing hormone analog or inhibitor is goserelin acetate. In some embodiments, the gonadotropin releasing hormone analog or inhibitor is triptorelin. In some embodiments, the gonadotropin releasing hormone analog or inhibitor is triptorelin acetate. In some embodiments, the gonadotropin releasing hormone analog or inhibitor is triptorelin pamoate. In some embodiments, the gonadotropin releasing hormone analog or inhibitor is histrelin. In some embodiments, the gonadotropin releasing hormone analog or inhibitor is histrelin acetate. In some embodiments, the subject has undergone an orchiectomy.

In some embodiments, prior to the administration of the therapeutically effective amount of a bifunctional compound of the present disclosure, the subject experienced progression of prostate cancer on at least one prior approved systemic therapy for metastatic prostate cancer. In some embodiments, prior to the administration of the therapeutically effective amount of a bifunctional compound of the present disclosure, the subject experienced progression of prostate cancer on at least two prior approved systemic therapies for metastatic prostate cancer. In some embodiments, at least one of the prior approved systemic therapies for metastatic prostate cancer is a second-generation androgen inhibitor. In some embodiments, prior to the administration of the therapeutically effective amount of a bifunctional compound of the present disclosure, the subject experienced progression of prostate cancer on at least two prior approved systemic therapies for metastatic prostate cancer, at least one of which is a second-generation androgen inhibitor. In some embodiments, the second-generation androgen inhibitor is abiraterone, abiraterone acetate, enzalutamide, darolutamide, apalutamide, or a pharmaceutically acceptable salt thereof. In some embodiments, the second-generation androgen inhibitor is abiraterone. In some embodiments, the second-generation androgen inhibitor is abiraterone acetate. In some embodiments, the second-generation androgen inhibitor is enzalutamide. In some embodiments, the second-generation androgen inhibitor is darolutamide. In some embodiments, the second-generation androgen inhibitor is apalutamide.

In some embodiments, prior to the administration of the therapeutically effective amount of a bifunctional compound of the present disclosure, the subject received at least one but not more than three prior second-generation anti-androgen agents (e.g., enzalutamide, abiraterone acetate, abiraterone, darolutamide, or apalutamide). In some embodiments, the subject received one prior second-generation anti-androgen agent. In some embodiments, the subject received two prior second-generation anti-androgen agents. In some embodiments, the subject received three prior second-generation anti-androgen agents.

In some embodiments, prior to the administration of the therapeutically effective amount of a bifunctional compound of the present disclosure, the subject received no more than two prior chemotherapy regimens. In some embodiments, the subject received two prior chemotherapy regimens. In some embodiments, the subject received one prior chemotherapy regimen. In some embodiments, the subject has not received a prior chemotherapy regimen.

In some embodiments, prior to the administration of the therapeutically effective amount of a bifunctional compound of the present disclosure (preferably Compound A) for the treatment of prostate cancer naïve to novel hormonal agents (NHAs), the subject received no more than two prior chemotherapy regimens. In some embodiments, the subject received two prior chemotherapy regimens. In some embodiments, the subject received one prior chemotherapy regimen. In some embodiments, the subject has not received a prior chemotherapy regimen.

In some embodiments, the subject has an ECOG performance status of 0 or 1. In some embodiments, the subject has an ECOG performance status of 1. In some embodiments, the subject has an ECOG performance status of 0.

In some embodiments, the subject does not have a symptomatic brain metastasis requiring steroids above physiologic replacement doses.

In some embodiments, the subject does not have active inflammatory bowel disease.

In some embodiments, the subject does not have chronic diarrhea.

In some embodiments, the subject does not have diverticular disease.

In some embodiments, the subject has not previously undergone gastric resection.

In some embodiments, the subject has not previously undergone lap band surgery.

In some embodiments, the subject has not previously undergone radiation therapy within four weeks prior to the initial the administration of a compound of the present disclosure.

In some embodiments, the subject has not previously undergone radiation therapy where greater than about 25% of the patient's bone marrow was irradiated.

In some embodiments, the subject has not been administered an investigational drug within four weeks prior to the initial the administration of a compound of the present disclosure.

In some embodiments, the subject has not received a systemic anti-cancer therapy within 2 weeks of the first administration of the therapeutically effective amount of a bifunctional compound of the present disclosure. In some embodiments, the subject has not received a systemic anti-cancer therapy within 2 weeks of the first administration of the therapeutically effective amount of a bifunctional compound of the present disclosure, except for agents to maintain castration status.

In some embodiments, the subject has not received bicalutamide within 6 weeks of the first administration of the therapeutically effective amount of a bifunctional compound of the present disclosure.

In some embodiments, the subject has not received mitomycin C within 6 weeks of the first administration of the therapeutically effective amount of a bifunctional compound of the present disclosure.

In some embodiments, the subject has not received nitrosoureas within 6 weeks of the first administration of the therapeutically effective amount of a bifunctional compound of the present disclosure.

In some embodiments, the subject has not received abiraterone or abiraterone acetate within 4 weeks of the first administration of the therapeutically effective amount of a bifunctional compound of the present disclosure.

In some embodiments, the subject suffering from prostate cancer (e.g., mCRPC) will have a different response to treatment with a compound of the disclosure (preferably Compound A) or a pharmaceutically acceptable salt, depending on the AR biomarker status of the subject, i.e., whether the subject has one or more somatic tumor mutations to AR.

In some embodiments, the subject with prostate cancer comprises at least one somatic AR tumor mutation.

In some embodiments, the subject with prostate cancer comprises at least the somatic AR tumor mutation of L702.

In some embodiments, the subject with prostate cancer comprises at least the somatic AR tumor mutation of L702H.

In some embodiments, the subject with prostate cancer comprises at least the somatic AR tumor mutation of M895.

In some embodiments, the subject with prostate cancer comprises at least the somatic AR tumor mutation of M895V.

In some embodiments, the subject with prostate cancer comprises at least the somatic AR tumor mutation of W742.

In some embodiments, the subject with prostate cancer comprises at least the somatic AR tumor mutation of W742C.

In some embodiments, the subject with prostate cancer comprises at least the somatic AR tumor mutation of S889.

In some embodiments, the subject with prostate cancer comprises at least the somatic AR tumor mutation of S889G.

In some embodiments, the subject with prostate cancer comprises at least the somatic AR tumor mutation of M750.

In some embodiments, the subject with prostate cancer comprises at least the somatic AR tumor mutation of M750V.

In some embodiments, the subject with prostate cancer comprises at least the somatic AR tumor mutation of M896.

In some embodiments, the subject with prostate cancer comprises at least the somatic AR tumor mutation of M896V.

In some embodiments, the subject with prostate cancer comprises at least the somatic AR tumor mutation of T878.

In some embodiments, the subject with prostate cancer comprises at least the somatic AR tumor mutation of T878A.

In some embodiments, the subject with prostate cancer comprises at least the somatic AR tumor mutation of T878S.

In some embodiments, the subject with prostate cancer comprises at least the somatic AR tumor mutation of F877.

In some embodiments, the subject with prostate cancer comprises at least the somatic AR tumor mutation of F877L.

In some embodiments, the subject with prostate cancer comprises at least the somatic AR tumor mutation of D891.

In some embodiments, the subject with prostate cancer comprises at least the somatic AR tumor mutation of D891H.

In some embodiments, the subject with prostate cancer comprises at least the somatic AR tumor mutation of H875.

In some embodiments, the subject with prostate cancer comprises at least the somatic AR tumor mutation of H875Y.

In some embodiments, the subject with prostate cancer comprises at least two somatic AR tumor mutations.

In some embodiments, the subject with prostate cancer comprises at least the two somatic AR tumor mutations of T878A/D891H.

In some embodiments, the subject with prostate cancer comprises at least the two somatic AR tumor mutations of T878A/S889G.

In some embodiments, the subject with prostate cancer comprises at least the two somatic AR tumor mutations of F877L/T878A.

In some embodiments, the subject with prostate cancer comprises at least the two somatic AR tumor mutations of L702H and T878A.

In some embodiments, the subject with prostate cancer comprises two somatic AR tumor mutations of L702H and T878A.

In some embodiments, the subject with prostate cancer comprises at least the two somatic AR tumor mutations of L702H and T878S.

In some embodiments, the subject with prostate cancer comprises two somatic AR tumor mutations of L702H and T878S.

In some embodiments, the subject with prostate cancer comprises at least the two somatic AR tumor mutations of L702H and H875Y.

In some embodiments, the subject with prostate cancer comprises two somatic AR tumor mutations of L702H and H875Y.

In some embodiments, the subject with prostate cancer comprises at least the two somatic AR tumor mutations of T878A and H875Y.

In some embodiments, the subject with prostate cancer comprises two somatic AR tumor mutations of T878A and H875Y.

In some embodiments, the subject with prostate cancer comprises at least the two somatic AR tumor mutations of T878S and H875Y.

In some embodiments, the subject with prostate cancer comprises two somatic AR tumor mutations of T878S and H875Y.

In some embodiments, the subject with prostate cancer comprises at least the three somatic AR tumor mutations of L702H, T878A, and H875Y.

In some embodiments, the subject with prostate cancer comprises three somatic AR tumor mutations of L702H, T878A, and H875Y.

In some embodiments, the subject with prostate cancer comprises at least the three somatic AR tumor mutations of L702H, T878S, and H875Y.

In some embodiments, the subject with prostate cancer comprises three somatic AR tumor mutations of L702H, T878S, and H875Y.

In some embodiments, the subject with prostate cancer comprises at least one somatic AR tumor mutation that is not L702H, T878A, T878S, or H875Y.

In some embodiments, the subject with prostate cancer comprises one somatic AR tumor mutation that is not L702H, T878A, T878S, or H875Y.

In some embodiments, the subject with prostate cancer comprises at least two somatic AR tumor mutation that are not L702H, T878A, T878S, or H875Y.

In some embodiments, the subject with prostate cancer comprises two somatic AR tumor mutation that are not L702H, T878A, T878S, or H875Y.

In some embodiments, the subject with prostate cancer comprises at least three somatic AR tumor mutation that are not L702H, T878A, T878S, or H875Y.

In some embodiments, the subject with prostate cancer comprises three somatic AR tumor mutation that are not L702H, T878A, T878S, or H875Y.

In one aspect, the application pertains to treating prostate cancer with a compound of the disclosure (preferably Compound A or a pharmaceutically acceptable salt thereof) in combination with another anti-cancer agent. In some embodiments, the prostate cancer treated with the combination of a compound of the disclosure (preferably Compound A or a pharmaceutically acceptable salt thereof) and another anti-cancer agent is adenocarcinoma of the prostate. In some embodiments, the prostate cancer treated with the combination of a compound of the disclosure (preferably Compound A or a pharmaceutically acceptable salt thereof) and another anti-cancer agent is metastatic prostate cancer. In some embodiments, the prostate cancer treated with the combination of a compound of the disclosure (preferably Compound A or a pharmaceutically acceptable salt thereof) and another anti-cancer agent is castrate-resistant or castration-resistant prostate cancer. In some embodiments, the prostate cancer treated with the combination of a compound of the disclosure (preferably Compound A or a pharmaceutically acceptable salt thereof) and another anti-cancer agent is metastatic castrate-resistant prostate cancer. In some embodiments, the prostate cancer treated with the combination of a compound of the disclosure (preferably Compound A or a pharmaceutically acceptable salt thereof) and another anti-cancer agent is progressive mCRPC. In some embodiments, the prostate cancer treated with the combination of a compound of the disclosure (preferably Compound A or a pharmaceutically acceptable salt thereof) and another anti-cancer agent is castrate-sensitive or castration-sensitive prostate cancer. In some embodiments, the prostate cancer treated with the combination of a compound of the disclosure (preferably Compound A or a pharmaceutically acceptable salt thereof) and another anti-cancer agent is metastatic castrate-sensitive prostate cancer. In some embodiments, the prostate cancer treated with the combination of a compound of the disclosure (preferably Compound A or a pharmaceutically acceptable salt thereof) and another anti-cancer agent is prostate cancer naïve to novel hormonal agents (NHA). In some embodiments, the prostate cancer treated with the combination of a compound of the disclosure (preferably Compound A or a pharmaceutically acceptable salt thereof) and another anti-cancer agent is metastatic prostate cancer naïve to novel hormonal agents (NHA). In some embodiments, the prostate cancer treated with the combination of a compound of the disclosure (preferably Compound A or a pharmaceutically acceptable salt thereof) and another anti-cancer agent is castrate-resistant prostate cancer naïve to novel hormonal agents (NHA). In some embodiments, the prostate cancer treated with the combination of a compound of the disclosure (preferably Compound A or a pharmaceutically acceptable salt thereof) and another anti-cancer agent is castrate-sensitive prostate cancer naïve to novel hormonal agents (NHA). In some embodiments, the prostate cancer treated with the combination of a compound of the disclosure (preferably Compound A or a pharmaceutically acceptable salt thereof) and another anti-cancer agent is metastatic castrate-resistant prostate cancer naïve to novel hormonal agents (NHA). In some embodiments, the prostate cancer treated with the combination of a compound of the disclosure (preferably Compound A or a pharmaceutically acceptable salt thereof) and another anti-cancer agent is metastatic castrate-sensitive prostate cancer naïve to novel hormonal agents (NHA). In some embodiments, the other anti-cancer agent is abiraterone, abiraterone acetate, estramustine, docetaxel, ketoconazole, goserelin, histrelin, triptorelin, buserelin, cyproterone, flutamide, bicalutamide, nilutamide, pamidronate, zolendronate, or a pharmaceutically acceptable salt thereof. In some embodiments, the other anti-cancer agent is abiraterone or a pharmaceutically acceptable salt thereof. In some embodiments, the other anti-cancer agent is abiraterone acetate or a pharmaceutically acceptable salt thereof. In some embodiments, the other anti-cancer agent is abiraterone acetate.

In some embodiments, the application pertains to treating prostate cancer with a compound of the disclosure (preferably Compound A or a pharmaceutically acceptable salt thereof) in combination with another anti-cancer agent, wherein the subject was not previously administered a novel hormonal agent (NHA). In some embodiments, the NHA is an androgen biosynthesis inhibitor. In some embodiments, the NHA is an androgen receptor blocker. In some embodiments, the NHA is abiraterone. In some embodiments, the NHA is abiraterone acetate. In some embodiments, the NHA is enzalutamide. In some embodiments, the NHA is darolutamide. In some embodiments, the NHA is apalutamide.

In some embodiments, the prostate cancer treated with the combination of a compound of the disclosure (preferably Compound A or a pharmaceutically acceptable salt thereof) and another anti-cancer agent, wherein the subject was not previously administered a novel hormonal agent (NHA) is adenocarcinoma of the prostate.

In some embodiments, the prostate cancer treated with the combination of a compound of the disclosure (preferably Compound A or a pharmaceutically acceptable salt thereof) and another anti-cancer agent, wherein the subject was not previously administered a novel hormonal agent (NHA) is metastatic prostate cancer.

In some embodiments, the prostate cancer treated with the combination of a compound of the disclosure (preferably Compound A or a pharmaceutically acceptable salt thereof) and another anti-cancer agent, wherein the subject was not previously administered a novel hormonal agent (NHA) is castrate-resistant or castration-resistant prostate cancer.

In some embodiments, the prostate cancer treated with the combination of a compound of the disclosure (preferably Compound A or a pharmaceutically acceptable salt thereof) and another anti-cancer agent, wherein the subject was not previously administered a novel hormonal agent (NHA) is metastatic castrate-resistant prostate cancer.

In some embodiments, the prostate cancer treated with the combination of a compound of the disclosure (preferably Compound A or a pharmaceutically acceptable salt thereof) and another anti-cancer agent, wherein the subject was not previously administered a novel hormonal agent (NHA) is progressive mCRPC.

In some embodiments, the prostate cancer treated with the combination of a compound of the disclosure (preferably Compound A or a pharmaceutically acceptable salt thereof) and another anti-cancer agent, wherein the subject was not previously administered a novel hormonal agent (NHA) is castrate-sensitive or castration-sensitive prostate cancer.

In some embodiments, the prostate cancer treated with the combination of a compound of the disclosure (preferably Compound A or a pharmaceutically acceptable salt thereof) and another anti-cancer agent, wherein the subject was not previously administered a novel hormonal agent (NHA) is metastatic castrate-sensitive prostate cancer.

In some embodiments, the other anti-cancer agent is abiraterone, abiraterone acetate, estramustine, docetaxel, ketoconazole, goserelin, histrelin, triptorelin, buserelin, cyproterone, flutamide, bicalutamide, nilutamide, pamidronate, zolendronate, or a pharmaceutically acceptable salt thereof.

In some embodiments, the other anti-cancer agent is abiraterone or a pharmaceutically acceptable salt thereof.

In some embodiments, the other anti-cancer agent is abiraterone acetate or a pharmaceutically acceptable salt thereof.

In some embodiments, the other anti-cancer agent is abiraterone acetate.

In one aspect, treating cancer results in a reduction in size of a tumor. A reduction in size of a tumor may also be referred to as “tumor regression.” Preferably, after treatment, tumor size is reduced by 5% or greater relative to its size prior to treatment; more preferably, tumor size is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75% or greater. Size of a tumor may be measured by any reproducible means of measurement. In a preferred aspect, size of a tumor may be measured as a diameter of the tumor.

In another aspect, treating cancer results in a reduction in tumor volume. Preferably, after treatment, tumor volume is reduced by 5% or greater relative to its volume prior to treatment; more preferably, tumor volume is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75% or greater. Tumor volume may be measured by any reproducible means of measurement.

In another aspect, treating cancer results in a decrease in number of tumors. Preferably, after treatment, tumor number is reduced by 5% or greater relative to number prior to treatment; more preferably, tumor number is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75%. Number of tumors may be measured by any reproducible means of measurement. In a preferred aspect, number of tumors may be measured by counting tumors visible to the naked eye or at a specified magnification. In a preferred aspect, the specified magnification is 2×, 3×, 4×, 5×, 10×, or 50×.

In another aspect, treating cancer results in a decrease in number of metastatic lesions in other tissues or organs distant from the primary tumor site. Preferably, after treatment, the number of metastatic lesions is reduced by 5% or greater relative to number prior to treatment; more preferably, the number of metastatic lesions is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75%. The number of metastatic lesions may be measured by any reproducible means of measurement. In a preferred aspect, the number of metastatic lesions may be measured by counting metastatic lesions visible to the naked eye or at a specified magnification. In a preferred aspect, the specified magnification is 2×, 3×, 4×, 5×, 10×, or 50×.

In another aspect, treating cancer results in an increase in average survival time of a population of treated subjects in comparison to a population receiving carrier alone. Preferably, the average survival time is increased by more than 30 days; more preferably, by more than 60 days; more preferably, by more than 90 days; and most preferably, by more than 120 days. An increase in average survival time of a population may be measured by any reproducible means. In a preferred aspect, an increase in average survival time of a population may be measured, for example, by calculating for a population the average length of survival following initiation of treatment with an active agent or compound of the disclosure. In another preferred aspect, an increase in average survival time of a population may also be measured, for example, by calculating for a population the average length of survival following completion of a first round of treatment with an active agent or compound of the disclosure.

In another aspect, treating cancer results in an increase in average survival time of a population of treated subjects in comparison to a population of untreated subjects. Preferably, the average survival time is increased by more than 30 days; more preferably, by more than 60 days; more preferably, by more than 90 days; and most preferably, by more than 120 days. An increase in average survival time of a population may be measured by any reproducible means. In a preferred aspect, an increase in average survival time of a population may be measured by calculating for a population the average length of survival following initiation of treatment with an active agent or compound of the disclosure. In another preferred aspect, an increase in average survival time of a population may be measured by calculating for a population the average length of survival following completion of a first round of treatment with a compound of the disclosure (preferably Compound A or a pharmaceutically acceptable salt thereof).

In another aspect, treating cancer results in a decrease in tumor growth rate. Preferably, after treatment, tumor growth rate is reduced by at least 5% relative to growth rate prior to treatment; more preferably, tumor growth rate is reduced by at least 10%; more preferably, reduced by at least 20%; more preferably, reduced by at least 30%; more preferably, reduced by at least 40%; more preferably, reduced by at least 50%; even more preferably, reduced by at least 50%; and most preferably, reduced by at least 75%. Tumor growth rate may be measured by any reproducible means of measurement. In a preferred aspect, tumor growth rate is measured according to a change in tumor diameter per unit time.

In another aspect, treating cancer results in a decrease in tumor regrowth. Preferably, after treatment, tumor regrowth is less than 5%; more preferably, tumor regrowth is less than 10%; more preferably, less than 20%; more preferably, less than 30%; more preferably, less than 40%; more preferably, less than 50%; even more preferably, less than 50%; and most preferably, less than 75%. Tumor regrowth may be measured by any reproducible means of measurement. In a preferred aspect, tumor regrowth is measured by measuring an increase in the diameter of a tumor after a prior tumor shrinkage that followed treatment. In another preferred aspect, a decrease in tumor regrowth is indicated by failure of tumors to reoccur after treatment has stopped.

In some embodiments, the subject is also administered a stomach acid-modifying reagent, a PPI, or an H2 compound. In some embodiments, the subject initiates, continues, or maintains the same level of treatment with a stomach acid-modifying agent while being treated with a compound of Formula (I) (e.g., Compound A).

In some embodiments, subjects should not initiate, continue, or maintain the same level of treatment with a stomach acid-modifying agent while being treated with a compound of Formula (I) (e.g., Compound A). In some embodiments, the stomach acid-modifying agent is a PPI. In some embodiments, the PPI is esomeprazole. In some embodiments, the stomach acid-modifying agent is an H2 compound. In some embodiments, the stomach acid-modifying agent is an antacid.

In some embodiments, subjects should discontinue the treatment with or use of a stomach acid-modifying agent beginning at a time point prior to initiating the administration of a compound of Formula (I) (e.g., Compound A). In some embodiments, the time point is at least 1 hour. In some embodiments, the time point is at least 2 hours. In some embodiments, the time point is at least 3 hours. In some embodiments, the time point is at least 4 hours. In some embodiments, the time point is at least 5 hours. In some embodiments, the time point is at least 6 hours. In some embodiments, the time point is at least 7 hours. In some embodiments, the time point is at least 8 hours. In some embodiments, the time point is at least 9 hours. In some embodiments, the time point is at least 10 hours. In some embodiments, the time point is at least 11 hours. In some embodiments, the time point is at least 12 hours. In some embodiments, the time point is at least 24 hours. In some embodiments, the time point is at least 36 hours. In some embodiments, the time point is at least 48 hours. In some embodiments, the time point is at least 60 hours. In some embodiments, the time point is at least 72 hours. In some embodiments, the stomach acid-modifying agent is a PPI. In some embodiments, the PPI is esomeprazole. In some embodiments, the stomach acid-modifying agent is an H2 compound. In some embodiments, the stomach acid-modifying agent is an antacid.

In some embodiments, subjects should reduce the level of treatment with or use of a stomach acid-modifying agent beginning at a time point prior to initiating the administration of a compound of Formula (I) (e.g., Compound A). In some embodiments, the time point is at least 1 hour. In some embodiments, the time point is at least 2 hours. In some embodiments, the time point is at least 3 hours. In some embodiments, the time point is at least 4 hours. In some embodiments, the time point is at least 5 hours. In some embodiments, the time point is at least 6 hours. In some embodiments, the time point is at least 7 hours. In some embodiments, the time point is at least 8 hours. In some embodiments, the time point is at least 9 hours. In some embodiments, the time point is at least 10 hours. In some embodiments, the time point is at least 11 hours. In some embodiments, the time point is at least 12 hours. In some embodiments, the time point is at least 24 hours. In some embodiments, the time point is at least 36 hours. In some embodiments, the time point is at least 48 hours. In some embodiments, the time point is at least 60 hours. In some embodiments, the time point is at least 72 hours. In some embodiments, the stomach acid-modifying agent is a PPI. In some embodiments, the PPI is esomeprazole. In some embodiments, the stomach acid-modifying agent is an H2 compound. In some embodiments, the stomach acid-modifying agent is an antacid.

In some embodiments, subjects should not initiate, continue, or maintain the same level of treatment with a PPI or H2 compound while being treated with a compound of Formula (I) (e.g., Compound A). In some embodiments, the PPI is esomeprazole.

In some embodiments, subjects should not initiate, continue, or maintain the same level of treatment with a PPI while being treated with a compound of Formula (I) (e.g., Compound A). In some embodiments, the PPI is esomeprazole.

In some embodiments, subjects should not initiate, continue, or maintain the same level of treatment with esomeprazole while being treated with Compound A.

In some embodiments, the present invention provides packaged dosage forms of a compound of Formula (I) (e.g., Compound A) with printed instructions to avoid, reduce, or discontinue administering or the use of a PPI or H2 compound prior to administering or using a compound of Formula (I) (e.g., Compound A). In some embodiments, the PPI is esomeprazole.

In some embodiments, the present invention provides packaged dosage forms of Compound A with printed instructions to avoid, reduce, or discontinue administering or the use of a PPI prior to administering or using Compound A. In some embodiments, the PPI is esomeprazole.

A positive food effect was observed in humans being administered or using Compound A. For example, eating a high fat meal before being administered or using Compound A significantly increased the observed Cₘₐₓ of Compound A by -4-fold and the extent of systemic exposure by ˜3-fold compared to humans that did not eat high fat meal before being administered or using Compound A. In some embodiments, Compound A is administered or used orally with food, i.e., the subject is in a fed state.

In some embodiments, the concomitant use of PPIs with Compound A should be avoided.

In some embodiments, if PPI treatment or usage is required and there are no alternatives available, Compound A is administered or used orally with food, i.e., the subject is in a fed state, for example, by eating a moderate-fat meal (400 to 800 calories, approximately 35% fat) is recommended before Compound A is administered or used.

In some embodiments, H2 blockers (e.g., cimetidine, famotidine) or local antacids (e.g., aluminum hydroxide, calcium carbonate, bismuth subsalicylate) may be used. In some embodiments, Compound A is administered or used ≥2 hours before or 10 to 12 hours after H2 blockers are administered or used. In some embodiments, Compound A is administered or used ≥2 hours before or after antacids are administered or used.

The dosages of the compound of the disclosure for any of the methods and uses described herein vary depending on the agent, the age, weight, and clinical condition of the recipient subject, and the experience and judgment of the clinician or practitioner administering the therapy, among other factors affecting the selected dosage.

The therapeutically effective amount of the compound of the disclosure may be administered one or more times over a day for up to 30 or more days, followed by 1 or more days of non-administration of the compound. This type of treatment schedule, i.e., administration of a compound of the disclosure on consecutive days followed by non-administration of the compound on consecutive days may be referred to as a treatment cycle. A treatment cycle may be repeated as many times as necessary to achieve the intended affect.

In some embodiments, the therapeutically effective amount of the compound of the disclosure is 0.01, 0.05, 0.1, 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500, 505, 510, 515, 520, 525, 530, 535, 540, 545, 550, 555, 560, 565, 570, 575, 580, 585, 590, 595, 600, 605, 610, 615, 620, 625, 630, 635, 640, 645, 650, 655, 660, 665, 670, 675, 680, 685, 690, 695, 700, 705, 710, 715, 720, 725, 730, 735, 740, 745, 750, 755, 760, 765, 770, 775, 780, 785, 790, 795, 800, 805, 810, 815, 820, 825, 830, 835, 840, 845, 850, 855, 860, 865, 870, 875, 880, 885, 890, 895, 900, 905, 910, 915, 920, 925, 930, 935, 940, 945, 950, 955, 960, 965, 970, 975, 980, 985, 990, 995, or 1,000 mg administered once, twice, three times, four times, or more daily for one, two, three, four, five, six, seven, eight, nine, ten, eleven, twelve, thirteen, fourteen, fifteen, twenty, twenty-five, thirty consecutive days, or, once, twice, three times, four times, or more daily, in single or divided doses, for 2 months, 3 months, 4 months, 5 months, 6 months, or longer.

In some embodiments, the therapeutically effective amount of the compound of the disclosure is about 10 to about 40 mg, about 20 to about 50 mg, about 30 to about 60 mg, about 40 to about 70 mg, about 50 to about 80 mg, about 60 to about 90 mg, about 70 to about 100 mg, about 80 to about 110 mg, about 90 to about 120 mg, about 100 to about 130 mg, about 110 to about 140 mg, about 120 to about 150 mg, about 130 to about 160 mg, about 140 to about 170 mg, about 150 to about 180 mg, about 160 to about 190 mg, about 170 to about 200 mg, about 180 to about 210 mg, about 190 to about 220 mg, about 200 to about 230 mg, about 210 to about 240 mg, about 220 to about 250 mg, about 230 to about 260 mg, about 240 to about 270 mg, about 250 to about 280 mg, about 260 to about 290 mg, about 270 to about 300 mg, about 280 to about 310 mg, about 290 to about 320 mg, about 300 to about 330 mg, about 310 to about 340 mg, about 320 to about 350 mg, about 330 to about 360 mg, about 340 to about 370 mg, about 350 to about 380 mg, about 360 to about 390 mg, about 370 to about 400 mg, about 380 to about 410 mg, about 390 to about 420 mg, about 400 to about 430 mg, about 410 to about 440 mg, about 420 to about 450 mg, about 430 to about 460 mg, about 440 to about 470 mg, about 450 to about 480 mg, about 460 to about 490 mg, about 470 to about 500 mg, about 480 to about 510 mg, about 490 to about 520 mg, about 500 to about 530 mg, about 510 to about 540 mg, about 520 to about 550 mg, about 530 to about 560 mg, about 540 to about 570 mg, about 550 to about 580 mg, about 560 to about 590 mg, about 570 to about 600 mg, about 580 to about 610 mg, about 590 to about 620 mg, about 600 to about 630 mg, about 610 to about 640 mg, about 620 to about 650 mg, about 630 to about 660 mg, about 640 to about 670 mg, about 650 to about 680 mg, about 660 to about 690 mg, about 670 to about 700 mg, about 680 to about 710 mg, about 690 to about 720 mg, about 700 to about 730 mg, about 710 to about 740 mg, about 720 to about 750 mg, about 730 to about 760 mg, about 740 to about 770 mg, about 750 to about 780 mg, about 760 to about 790 mg, about 770 to about 800 mg, about 780 to about 810 mg, about 790 to about 820 mg, about 800 to about 830 mg, about 810 to about 840 mg, about 820 to about 850 mg, about 830 to about 860 mg, about 840 to about 870 mg, about 850 to about 880 mg, about 860 to about 890 mg, about 870 to about 900 mg, about 880 to about 910 mg, about 890 to about 920 mg, about 900 to about 930 mg, about 910 to about 940 mg, about 920 to about 950 mg, about 930 to about 960 mg, about 940 to about 970 mg, about 950 to about 980 mg, about 960 to about 990 mg, or about 970 to about 1,000 mg administered once, twice, three times, four times, or more daily in single or divided doses (which dose may be adjusted for the patient's weight in kg, body surface area in m², and/or age in years).

In some embodiments, the therapeutically effective amount of the compound of the disclosure is about 5 mg to about 250 mg.

In some embodiments, the therapeutically effective amount of the compound of the disclosure is about 250 mg to about 500 mg.

In some embodiments, the therapeutically effective amount of the compound of the disclosure is about 500 mg to about 750 mg.

In some embodiments, the therapeutically effective amount of the compound of the disclosure is about 70 mg to about 1000 mg administered once, twice, three times, four times, or more daily in single or divided doses (which dose may be adjusted for the patient's weight in kg, body surface area in m², and/or age in years).

In some embodiments, the therapeutically effective amount of the compound of the disclosure is about 70 mg, 105 mg, 140 mg, 175 mg, 210 mg, 245 mg, 280 mg, 315 mg, 350 mg, 385 mg, 420 mg, 455 mg, 490 mg, 525 mg, 560 mg, 595 mg, 630 mg, 665 mg, or 700 mg administered once, twice, three times, four times, or more daily in single or divided doses (which dose may be adjusted for the patient's weight in kg, body surface area in m², and/or age in years).

In some embodiments, the therapeutically effective amount of Compound A is about 1 mg to about 1000 mg.

In some embodiments, the therapeutically effective amount of Compound A is about 5 mg to about 750 mg. [0624] [0609]

In some embodiments, the therapeutically effective amount of Compound A is about 10 mg to about 500 mg.

In some embodiments, the therapeutically effective amount of Compound A is about 20 mg to about 250 mg.

In some embodiments, the therapeutically effective amount of Compound A is about 100 mg to about 500 mg.

In some embodiments, the therapeutically effective amount of the compound of the disclosure is about 100 mg, about 150 mg, or about 300 mg.

In some embodiments, the therapeutically effective amount of the compound of the disclosure is about 100 mg.

In some embodiments, the therapeutically effective amount of the compound of the disclosure is 100 mg.

In some embodiments, the therapeutically effective amount of the compound of the disclosure is 100 mg, administered orally and once daily.

In some embodiments, the therapeutically effective amount of the compound of the disclosure is about 150 mg.

In some embodiments, the therapeutically effective amount of the compound of the disclosure is 150 mg.

In some embodiments, the therapeutically effective amount of the compound of the disclosure is 150 mg, administered orally and once daily.

In some embodiments, the therapeutically effective amount of Compound A is about 200 mg.

In some embodiments, the therapeutically effective amount of Compound A is 200 mg.

In some embodiments, the therapeutically effective amount of the compound of the disclosure is 150 mg, administered orally and once daily.

In some embodiments, the therapeutically effective amount of the compound of the disclosure is about 300 mg.

In some embodiments, the therapeutically effective amount of the compound of the disclosure is 300 mg.

In some embodiments, the therapeutically effective amount of the compound of the disclosure is 300 mg, administered orally and once daily.

In some embodiments, the therapeutically effective amount of Compound A is about 320 mg.

In some embodiments, the therapeutically effective amount of Compound A is 320 mg.

In some embodiments, the therapeutically effective amount of the compound of the disclosure is 320 mg, administered orally and once daily.

In some embodiments, the therapeutically effective amount of Compound A is about 400 mg.

In some embodiments, the therapeutically effective amount of Compound A is 400 mg.

In some embodiments, the therapeutically effective amount of the compound of the disclosure is 400 mg, administered orally and once daily.

In some embodiments, the therapeutically effective amount of Compound A is about 480 mg.

In some embodiments, the therapeutically effective amount of Compound A is 480 mg.

In some embodiments, the therapeutically effective amount of the compound of the disclosure is 480 mg, administered orally and once daily.

In some embodiments, the therapeutically effective amount of Compound A is about 500 mg.

In some embodiments, the therapeutically effective amount of Compound A is 500 mg.

In some embodiments, the therapeutically effective amount of the compound of the disclosure is 500 mg, administered orally and once daily.

The therapeutically effective amount of the compound of the disclosure can also range from about 0.01 mg/kg per day to about 100 mg/kg per day. In an aspect, therapeutically effective amount of the compound of the disclosure can range from about 0.05 mg/kg per day to about 10 mg/kg per day. In an aspect, therapeutically effective amount of the compound of the disclosure can range from about 0.075 mg/kg per day to about 5 mg/kg per day. In an aspect, therapeutically effective amount of the compound of the disclosure can range from about 0.10 mg/kg per day to about 1 mg/kg per day. In an aspect, therapeutically effective amount of the compound of the disclosure can range from about 0.20 mg/kg per day to about 0.70 mg/kg per day.

In some embodiments, the therapeutically effective amount of the compound of the disclosure is about 0.10 mg/kg per day, about 0.15 mg/kg per day, about 0.20 mg/kg per day, about 0.25 mg/kg per day, about 0.30 mg/kg per day, about 0.35 mg/kg per day, about 0.40 mg/kg per day, about 0.45 mg/kg per day, about 0.50 mg/kg per day, about 0.55 mg/kg per day, about 0.60 mg/kg per day, about 0.65 mg/kg per day, about 0.70 mg/kg per day, about 0.75 mg/kg per day, about 0.80 mg/kg per day, about 0.85 mg/kg per day, about 0.90 mg/kg per day, about 0.95 mg/kg per day, or about 1.00 mg/kg per day.

In some embodiments, the therapeutically effective amount of the compound of the disclosure is about 1.05 mg/kg per day, about 1.10 mg/kg per day, about 1.15 mg/kg per day, about 1.20 mg/kg per day, about 1.25 mg/kg per day, about 1.30 mg/kg per day, about 1.35 mg/kg per day, about 1.40 mg/kg per day, about 1.45 mg/kg per day, about 1.50 mg/kg per day, about 1.55 mg/kg per day, about 1.60 mg/kg per day, about 1.65 mg/kg per day, about 1.70 mg/kg per day, about 1.75 mg/kg per day, about 1.80 mg/kg per day, about 1.85 mg/kg per day, about 1.90 mg/kg per day, about 1.95 mg/kg per day, or about 2.00 mg/kg per day.

In some embodiments, the therapeutically effective amount of the compound of the disclosure is about 2 mg/kg per day, about 2.5 mg/kg per day, about 3 mg/kg per day, about 3.5 mg/kg per day, about 4 mg/kg per day, about 4.5 mg/kg per day, about 5 mg/kg per day, about 5.5 mg/kg per day, about 6 mg/kg per day, about 6.5 mg/kg per day, about 7 mg/kg per day, about 7.5 mg/kg per day, about 8.0 mg/kg per day, about 8.5 mg/kg per day, about 9.0 mg/kg per day, about 9.5 mg/kg per day, or about 10 mg/kg per day.

In some embodiments, the therapeutically effective amount of the compound of the disclosure is administered to the subject once daily. In some embodiments, this daily dose of a compound of the compound of the disclosure may administered to the subject all at once. In some embodiments, this daily dose of the compound of the disclosure may administered to the subject in two portions (i.e., a divided dose). In some embodiments, this daily dose of the compound of the disclosure may administered to the subject in three divided doses. In some embodiments, this daily dose of the compound of the disclosure may administered to the subject in four divided doses. In some embodiments, this daily dose of the compound of the disclosure may be administered to the subject in five or more divided doses. In some embodiments, these portions or divided doses are administered to the subject at regular intervals throughout the day, for example, every 12 hours, every 8 hours, every 6 hours, every 5 hours, every 4 hours, etc.

The therapeutically effective amount of the compound of the disclosure can be estimated initially either in cell culture assays or in animal models, usually rats, mice, rabbits, dogs, or pigs. The animal model may also be used to determine the appropriate concentration range and route of administration. Such information can then be used to determine useful doses and routes for administration in humans. Therapeutic/prophylactic efficacy and toxicity may be determined by standard pharmaceutical procedures in cell cultures or experimental animals, e.g., ED₅₀ (the dose therapeutically effective in 50% of the population) and LD₅₀ (the dose lethal to 50% of the population). The dose ratio between toxic and therapeutic effects is the therapeutic index, and it can be expressed as the ratio, LD₅₀/ED₅₀. Pharmaceutical compositions that exhibit large therapeutic indices are preferred. The dosage may vary within this range depending upon the dosage form employed, sensitivity of the patient, and the route of administration.

Dosage and administration are adjusted to provide sufficient levels of the compound of the disclosure or to maintain the desired effect. Factors which may be taken into account include the severity of the disease state, general health of the subject, age, weight, and gender of the subject, diet, time and frequency of administration, drug combination(s), reaction sensitivities, and tolerance/response to therapy. Long-acting pharmaceutical compositions may be administered every 3 to 4 days, every week, once every two weeks, or monthly depending on half-life and clearance rate of the particular formulation.

In some embodiments, for the methods of treating prostate cancer with the combination of the compound of the disclosure and another anti-cancer agent, the therapeutically effective amount of the compound of the disclosure is described herein, and the therapeutically effective amount of the anti-cancer agent is 0.01, 0.05, 0.1, 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500, 505, 510, 515, 520, 525, 530, 535, 540, 545, 550, 555, 560, 565, 570, 575, 580, 585, 590, 595, 600, 605, 610, 615, 620, 625, 630, 635, 640, 645, 650, 655, 660, 665, 670, 675, 680, 685, 690, 695, 700, 705, 710, 715, 720, 725, 730, 735, 740, 745, 750, 755, 760, 765, 770, 775, 780, 785, 790, 795, 800, 805, 810, 815, 820, 825, 830, 835, 840, 845, 850, 855, 860, 865, 870, 875, 880, 885, 890, 895, 900, 905, 910, 915, 920, 925, 930, 935, 940, 945, 950, 955, 960, 965, 970, 975, 980, 985, 990, 995, or 1,000 mg administered once, twice, three times, four times, or more daily for one, two, three, four, five, six, seven, eight, nine, ten, eleven, twelve, thirteen, fourteen, fifteen, or thirty consecutive days, or, once, twice, three times, four times, or more daily, in single or divided doses, for 2 months, 3 months, 4 months, 5 months, 6 months, or longer.

In some embodiments, for the methods of treating prostate cancer with the combination of the compound of the disclosure and abiraterone, a pharmaceutically acceptable salt of abiraterone, abiraterone acetate, or a pharmaceutically acceptable salt of abiraterone acetate, the therapeutically effective amount of the compound of the disclosure is described herein, and the therapeutically effective amount of abiraterone, pharmaceutically acceptable salt of abiraterone, abiraterone acetate, or a pharmaceutically acceptable salt of abiraterone acetate, is 0.01, 0.05, 0.1, 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500, 505, 510, 515, 520, 525, 530, 535, 540, 545, 550, 555, 560, 565, 570, 575, 580, 585, 590, 595, 600, 605, 610, 615, 620, 625, 630, 635, 640, 645, 650, 655, 660, 665, 670, 675, 680, 685, 690, 695, 700, 705, 710, 715, 720, 725, 730, 735, 740, 745, 750, 755, 760, 765, 770, 775, 780, 785, 790, 795, 800, 805, 810, 815, 820, 825, 830, 835, 840, 845, 850, 855, 860, 865, 870, 875, 880, 885, 890, 895, 900, 905, 910, 915, 920, 925, 930, 935, 940, 945, 950, 955, 960, 965, 970, 975, 980, 985, 990, 995, or 1,000 mg administered once, twice, three times, four times, or more daily for one, two, three, four, five, six, seven, eight, nine, ten, eleven, twelve, thirteen, fourteen, fifteen, thirty consecutive days, or, once, twice, three times, four times, or more daily, in single or divided doses, for 2 months, 3 months, 4 months, 5 months, 6 months, or longer. In some embodiments, for the methods of treating prostate cancer with the combination of the compound of the disclosure (preferably Compound A) and abiraterone acetate, the therapeutically effective amount of a compound of the disclosure is described herein, and the therapeutically effective amount of abiraterone acetate is about 250 mg, about 500 mg, about 750 mg, about 1,000 mg, about 1,250 mg, or about 1,500 mg administered once daily.

In some embodiments, for the methods of treating prostate cancer with the combination of the compound of the disclosure (preferably Compound A) and abiraterone acetate, the therapeutically effective amount of a compound of the disclosure is described herein, and the therapeutically effective amount of abiraterone acetate is about 125 mg, about 250 mg, about 375 mg, about 500 mg, about 625 mg, about 750 mg, about 875 mg, about 1,000 mg, about 1,125 mg, about 1,250 mg, about 1,375 mg, or about 1,500 mg administered once daily.

In some embodiments, for the methods of treating prostate cancer with the combination of the compound of the disclosure (preferably Compound A) and abiraterone acetate, the therapeutically effective amount of a compound of the disclosure is described herein, and the therapeutically effective amount of abiraterone acetate is about 125 mg, about 250 mg, about 375 mg, about 500 mg, about 625 mg, about 750 mg, about 875 mg, about 1,000 mg, about 1,125 mg, about 1,250 mg, about 1,375 mg, or about 1,500 mg administered twice daily.

In some embodiments, for the methods of treating prostate cancer with the combination of the compound of the disclosure (preferably Compound A) and abiraterone acetate, the therapeutically effective amount of a compound of the disclosure is described herein, and the therapeutically effective amount of abiraterone acetate is about 125 mg, about 250 mg, about 375 mg, about 500 mg, about 625 mg, about 750 mg, about 875 mg, about 1,000 mg, about 1,125 mg, about 1,250 mg, about 1,375 mg, or about 1,500 mg administered three times daily.

In some embodiments, for the methods of treating prostate cancer with the combination of the compound of the disclosure (preferably Compound A) and abiraterone acetate, the therapeutically effective amount of the compound of the disclosure (preferably Compound A) is described herein, and the therapeutically effective amount of abiraterone acetate is 1,000 mg administered orally once daily for one, two, three, four, five, six, seven, eight, nine, ten, eleven, twelve, thirteen, fourteen, fifteen, thirty, or more consecutive days, in single or divided doses. In some embodiments, the abiraterone acetate is administered in combination with a corticosteroid, administered orally, once or twice daily. In some embodiments, the abiraterone acetate is administered in combination with 5 mg of prednisone or prednisolone administered orally, twice daily.

In some embodiments, for the methods of treating prostate cancer with the combination of the compound of the disclosure (preferably Compound A) and abiraterone acetate, the therapeutically effective amount of the compound of the disclosure (preferably Compound A) is described herein, and the therapeutically effective amount of abiraterone acetate is 1,000 mg administered orally once daily for one, two, three, four, five, six, seven, eight, nine, ten, eleven, twelve, thirteen, fourteen, fifteen, twenty, twenty-five, thirty, or more consecutive days, in single or divided doses. In some embodiments, the abiraterone acetate is administered in combination with 5 mg of prednisone administered orally, twice daily. In some embodiments, the combination of the compound of the disclosure (preferably Compound A) and abiraterone acetate is administered to the subject in need thereof in the fasted state. In some embodiments, the subject does not eat for at least two hours before, and at least one hour after, the administration of the combination of the compound of the disclosure (preferably Compound A) and abiraterone acetate.

In some embodiments, the compound of the disclosure (preferably Compound A) and abiraterone acetate are administered to the subject simultaneously. In some embodiments, the compound of the disclosure (preferably Compound A) and abiraterone acetate are administered to the subject sequentially.

In some embodiments, the compound of the disclosure (preferably Compound A) and the anti-cancer agent are administered to the subject in temporal proximity.

In some embodiments, “temporal proximity” means that administration of the compound of the disclosure occurs within a time period before or after the administration of anti-cancer agent, such that the therapeutic effect of the compound of the disclosure overlaps with the therapeutic effect of the anti-cancer agent. In some embodiments, the therapeutic effect of the compound of the disclosure completely overlaps with the therapeutic effect of the anti-cancer agent. In some embodiments, “temporal proximity” means that administration of the compound of the disclosure occurs within a time period before or after the administration of anti-cancer agent, such that there is a synergistic effect between the compound of the disclosure and the anti-cancer agent. In some embodiments, the anti-cancer agent is abiraterone acetate.

“Temporal proximity” may vary according to various factors, including but not limited to, the age, gender, weight, genetic background, medical condition, disease history, and treatment history of the subject to which the therapeutic agents are to be administered; the disease or condition to be treated or ameliorated; the therapeutic outcome to be achieved; the dosage, dosing frequency, and dosing duration of the therapeutic agents; the pharmacokinetics and pharmacodynamics of the therapeutic agents; and the route(s) through which the therapeutic agents are administered. In some embodiments, “temporal proximity” means within 15 minutes, within 30 minutes, within an hour, within two hours, within four hours, within six hours, within eight hours, within 12 hours, within 18 hours, within 24 hours, within 36 hours, within 2 days, within 3 days, within 4 days, within 5 days, within 6 days, within a week, within 2 weeks, within 3 weeks, within 4 weeks, with 6 weeks, or within 8 weeks. In some embodiments, multiple administration of one therapeutic agent can occur in temporal proximity to a single administration of another therapeutic agent. In some embodiments, temporal proximity may change during a treatment cycle or within a dosing regimen.

In some embodiments, the compound of the disclosure (preferably Compound A) and abiraterone, a pharmaceutically acceptable salt of abiraterone, abiraterone acetate, or a pharmaceutically acceptable salt of abiraterone acetate are administered to the subject simultaneously.

In some embodiments, the compound of the disclosure (preferably Compound A) and abiraterone, a pharmaceutically acceptable salt of abiraterone, abiraterone acetate, or a pharmaceutically acceptable salt of abiraterone acetate are administered to the subject simultaneously in separate formulations (e.g., in separate tablets). In some embodiments, the compound of the disclosure (preferably Compound A) and abiraterone, a pharmaceutically acceptable salt of abiraterone, abiraterone acetate, or a pharmaceutically acceptable salt of abiraterone acetate are administered to the subject simultaneously in the same formulation (e.g., in a single tablet).

In some embodiments, the compound of the disclosure (preferably Compound A) and abiraterone, a pharmaceutically acceptable salt of abiraterone, abiraterone acetate, or a pharmaceutically acceptable salt of abiraterone acetate are administered to the subject sequentially.

In some embodiments, the compound of the disclosure (preferably Compound A) and abiraterone, a pharmaceutically acceptable salt of abiraterone, abiraterone acetate, or a pharmaceutically acceptable salt of abiraterone acetate are administered to the subject in temporal proximity.

In some embodiments, the compound of the disclosure (preferably Compound A) is administered to the subject before abiraterone, a pharmaceutically acceptable salt of abiraterone, abiraterone acetate, or a pharmaceutically acceptable salt of abiraterone acetate, is administered to the subject, wherein the subject is in a fasted state.

In some embodiments, the compound of the disclosure (preferably Compound A) is administered to the subject before abiraterone, a pharmaceutically acceptable salt of abiraterone, abiraterone acetate, or a pharmaceutically acceptable salt of abiraterone acetate, is administered to the subject, wherein the subject is in a fed state.

In some embodiments, the compound of the disclosure (preferably Compound A) is administered to the subject after abiraterone, a pharmaceutically acceptable salt of abiraterone, abiraterone acetate, or a pharmaceutically acceptable salt of abiraterone acetate, is administered to the subject, wherein the subject is in a fasted state.

In some embodiments, the compound of the disclosure (preferably Compound A) is administered to the subject after abiraterone, a pharmaceutically acceptable salt of abiraterone, abiraterone acetate, or a pharmaceutically acceptable salt of abiraterone acetate, is administered to the subject, wherein the subject is in a fed state.

### Pharmaceutical Compositions

In some embodiments, the compound of the disclosure (preferably Compound A) is formulated for oral administration. For example, in some embodiments, the compound of the disclosure (preferably Compound A) is formulated as a tablet that comprises zero, one, two, or more of each of the following: emulsifier; surfactant, binder; disintegrant, glidant; and lubricant.

In some embodiments, the emulsifier is hypromellose.

In some embodiments, the surfactant is vitamin E polyethylene glycol succinate.

In some embodiments, the binder (also referred to herein as a filler) is selected from the group consisting of microcrystalline cellulose, lactose monohydrate, sucrose, glucose, and sorbitol.

In some embodiments, the disintegrant is croscarmellose sodium.

In some embodiments, the glidant refers to a substance used to promote powder flow by reducing interparticle cohesion. In some embodiments, in the dosage forms of the disclosure, the glidant is selected from the group consisting of silicon dioxide, silica colloidal anhydrous, starch, and talc.

In some embodiments, the lubricant refers to a substance that prevents ingredients from sticking and/or clumping together in the machines used in preparation of the dosage forms of the disclosure. In some embodiments, in the dosage forms of the disclosure, the lubricant is selected from the group consisting of magnesium stearate, sodium stearyl fumarate, stearic acid, and vegetable stearin.

The pharmaceutical compositions containing the compound of the disclosure (preferably Compound A) may be manufactured in a manner that is generally known, e.g., by means of conventional mixing, dissolving, granulating, dragee-making, levigating, emulsifying, encapsulating, entrapping, or lyophilizing processes. Pharmaceutical compositions may be formulated in a conventional manner using one or more pharmaceutically acceptable carriers comprising excipients and/or auxiliaries that facilitate processing of the compound of the disclosure into preparations that can be used pharmaceutically. Of course, the appropriate formulation is dependent upon the route of administration chosen.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the compound of the disclosure (preferably Compound A) in the required amount in an appropriate solvent with one or a combination of ingredients enumerated above, as required, followed by filtered sterilization. Generally, dispersions are prepared by incorporating the active agent or compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible pharmaceutically acceptable carrier. They can be enclosed in gelatin capsules or compressed into tablets. For the purpose of oral therapeutic administration, the compound of the disclosure can be incorporated with excipients and used in the form of tablets, troches, or capsules. Oral compositions can also be prepared using a fluid carrier for use as a mouthwash, wherein the agent or compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate; a glidant such as colloidal silicon dioxide; a sweetening agent such as sucrose or saccharin; or a flavoring agent such as peppermint, methyl salicylate, or orange flavoring.

For administration by inhalation, the agents or compounds are delivered in the form of an aerosol spray from pressured container or dispenser, which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active agents or compounds are formulated into ointments, salves, gels, or creams as generally known in the art.

In one aspect, the compound of the disclosure (preferably Compound A) is prepared with pharmaceutically acceptable carriers that will protect the agent or compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active agent or compound calculated to produce the desired therapeutic effect in association with the required pharmaceutical carrier. The specification for the dosage unit forms of the application are dictated by and directly dependent on the unique characteristics of the compound of the disclosure and the particular therapeutic effect to be achieved.

The pharmaceutical compositions can be included in a container, pack, or dispenser together with instructions for administration.

Illustrative modes of administration for the compound of the disclosure (preferably Compound A) includes systemic or local administration such as oral, nasal, parenteral, transdermal, subcutaneous, vaginal, buccal, rectal or topical administration modes. In some embodiments, the compound of the disclosure is administered orally. In some embodiments, the compound of the disclosure is administered as a tablet, capsule, caplet, solution, suspension, syrup, granule, bead, powder, or pellet.

Illustrative pharmaceutical compositions are tablets and gelatin capsules comprising a salt of the compound of the disclosure (preferably Compound A) and a pharmaceutically acceptable carrier, such as a) a diluent, e.g., purified water, triglyceride oils, such as hydrogenated or partially hydrogenated vegetable oil, or mixtures thereof, corn oil, olive oil, sunflower oil, safflower oil, fish oils, such as EPA or DHA, or their esters or triglycerides or mixtures thereof, omega-3 fatty acids or derivatives thereof, lactose, dextrose, sucrose, mannitol, sorbitol, cellulose, sodium, saccharin, glucose and/or glycine; b) a lubricant, e.g., silica, talcum, stearic acid, its magnesium or calcium salt, sodium oleate, sodium stearate, magnesium stearate, sodium benzoate, sodium acetate, sodium chloride and/or polyethylene glycol; for tablets also; c) a binder, e.g., magnesium aluminum silicate, starch paste, gelatin, tragacanth, methylcellulose, sodium carboxymethylcellulose, magnesium carbonate, natural sugars such as glucose or beta-lactose, corn sweeteners, natural and synthetic gums such as acacia, tragacanth or sodium alginate, waxes and/or polyvinylpyrrolidone, if desired; d) a disintegrant, e.g., starches, agar, methyl cellulose, bentonite, xanthan gum, algic acid or its sodium salt, or effervescent mixtures; e) absorbent, colorant, flavorant and sweetener; f) an emulsifier or dispersing agent, such as Tween 80, Labrasol, HPMC, DOSS, caproyl 909, labrafac, labrafil, peceol, transcutol, capmul MCM, capmul PG-12, captex 355, gelucire, vitamin E TGPS or other acceptable emulsifier; and/or g) an agent that enhances absorption of the salt such as cyclodextrin, hydroxypropyl-cyclodextrin, PEG400, and/or PEG200.

For preparing pharmaceutical compositions from the compound of the disclosure, or a salt or hydrate thereof, inert, pharmaceutically acceptable carriers can be either solid or liquid. Solid form preparations include powders, tablets, dispersible granules, capsules, cachets and suppositories. The powders and tablets may be comprised of from about 5 to about 95 percent active ingredient. Suitable solid carriers are known in the art, e.g., magnesium carbonate, magnesium stearate, talc, sugar or lactose. Tablets, powders, cachets and capsules can be used as solid dosage forms suitable for oral administration. Examples of pharmaceutically acceptable carriers and methods of manufacture for various compositions may be found in A. Gennaro (ed.), Remington's Pharmaceutical Sciences, 18th Edition, (1990), Mack Publishing Co., Easton, Pa.

Liquid form preparations include solutions, suspensions and emulsions. For example, water or water-propylene glycol solutions for parenteral injection or addition of sweeteners and opacifiers for oral solutions, suspensions and emulsions. Liquid form preparations may also include solutions for intranasal administration.

Liquid, particularly injectable, compositions can, for example, be prepared by dissolution, dispersion, etc. For example, the disclosed salt is dissolved in or mixed with a pharmaceutically acceptable solvent such as, for example, water, saline, aqueous dextrose, glycerol, ethanol, and the like, to thereby form an injectable isotonic solution or suspension. Proteins such as albumin, chylomicron particles, or serum proteins can be used to solubilize the disclosed compounds.

Parental injectable administration is generally used for subcutaneous, intramuscular or intravenous injections and infusions. Injectables can be prepared in conventional forms, either as liquid solutions or suspensions or solid forms suitable for dissolving in liquid prior to injection.

Aerosol preparations suitable for inhalation may include solutions and solids in powder form, which may be in combination with a pharmaceutically acceptable carrier, such as an inert compressed gas, e.g., nitrogen.

Also included are solid form preparations that are intended to be converted, shortly before use, to liquid form preparations for either oral or parenteral administration. Such liquid forms include solutions, suspensions and emulsions.

Depending on the intended mode of administration, the disclosed compositions can be in solid, semi-solid or liquid dosage form, such as, for example, injectables, tablets, suppositories, pills, time-release capsules, elixirs, tinctures, emulsions, syrups, powders, liquids, suspensions, or the like, sometimes in unit dosages and consistent with conventional pharmaceutical practices. Likewise, they can also be administered in intravenous (both bolus and infusion), intraperitoneal, intrathecal, subcutaneous or intramuscular form, and all using forms well known to those skilled in the pharmaceutical arts.

Pharmaceutical compositions can be prepared according to conventional mixing, granulating or coating methods, respectively, and the present pharmaceutical compositions can contain from about 0.1% to about 99%, from about 5% to about 90%, or from about 1% to about 20% of the disclosed free base or salt by weight or volume.

The pharmaceutical compositions containing the compound of the disclosure (preferably Compound A) may further comprising one or more additional anti-cancer agents, including any of those disclosed herein.

All amounts of any component of an oral dosage form described herein, e.g., a tablet, that are indicated based on % w/w refer to the total weight of the oral dosage form, unless otherwise indicated.

### EXAMPLES

The disclosure is further illustrated by the following examples, which are not to be construed as limiting this disclosure in scope or spirit to the specific procedures herein described. It is to be understood that the examples are provided to illustrate certain embodiments and that no limitation to the scope of the disclosure is intended thereby. It is to be further understood that resort may be had to various other embodiments, modifications, and equivalents thereof which may suggest themselves to those skilled in the art without departing from the spirit of the present disclosure and/or scope of the appended claims.

### Example 1—Degradation of AR in Wild-Type LNCaP and VCaP Cells

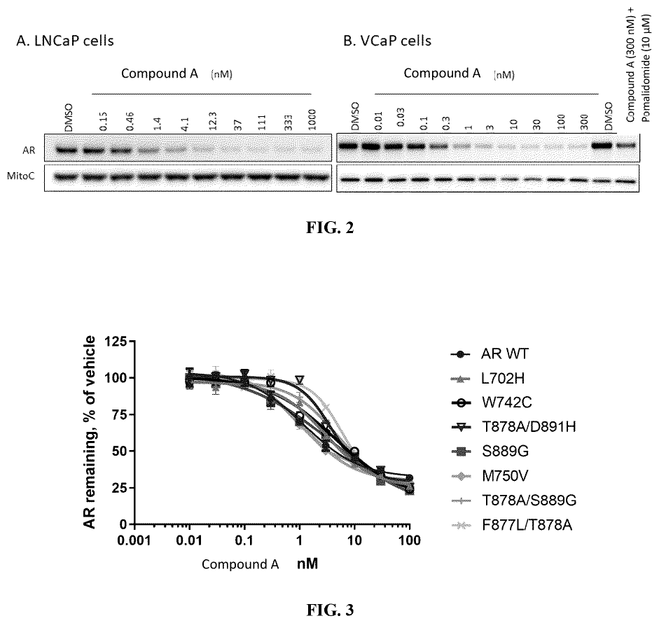

To determine the pharmacological effect of Compound A in vitro, AR degradation assays were conducted in VCaP (vertebral-cancer of the prostate) cancer cells and LNCaP (lymph node carcinoma of the prostate) cancer cells that harbor AR amplification and the AR mutation (T878A) mutation, respectively. (FIG. 2) After 24 hours of treatment, Compound A effectively degraded the AR in LNCaP (Dₘₐₓ ≥91% with DC₅₀ of ≤1.3 nM) and VCaP (Dₘₐₓ ≥94% with DC₅₀ of <1 nM). A degradation time course experiment also performed in VCaP cells demonstrated that >50% degradation of AR is achieved within 2 hours of Compound A treatment of VCaP cells and near complete elimination of AR from the cell is observed by 6 hours. Since Compound A is thought to engage the cereblon protein at the same binding site as immunomodulatory imide drugs (IMiDs), such as thalidomide, lenalidomide and pomalidomide, an excess amount of pomalidomide would compete for the binding of Compound A to the cereblon protein. Consistent with the PROTAC's mode of action, pomalidomide (10 μM) blocked Compound A mediated degradation. In addition, a poly-ubiquitination assay was conducted that demonstrated that ubiquitination of the AR is significantly increased with Compound A treatment. These data support the proposed mode of action of Compound A in degrading AR via the engagement of E3 ligase cereblon, ubiquitination of AR and degradation via the proteasome.

### Example 2—Degradation of AR Proteins Harboring Clinically Relevant Point Mutants

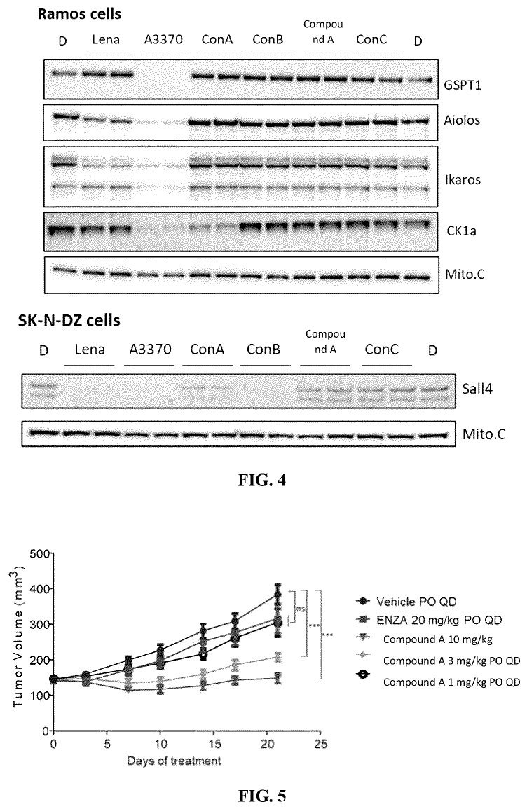

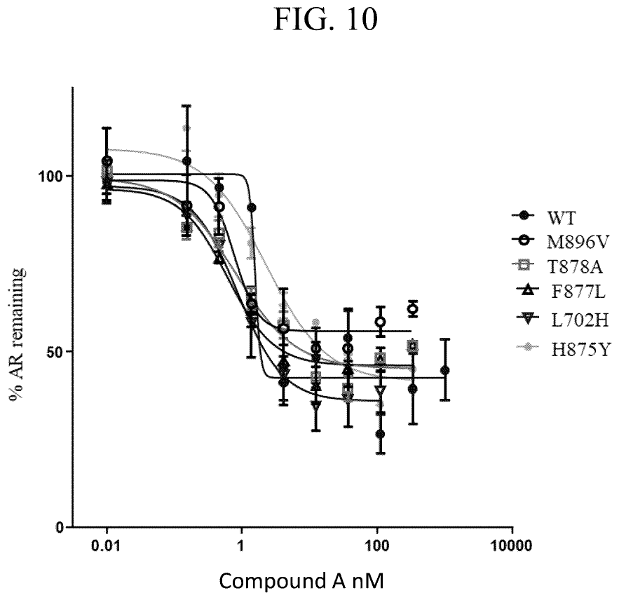

To assess the ability of Compound A to degrade variants of the AR, vectors expressing wild-type AR or mutant AR proteins harboring clinically observed mutations in the ligand binding domain (13 with single amino acid changes or 4 with 2 amino acid changes) (Azad A. A., Volik S. V., Wyatt A. W., et al. Androgen receptor gene aberrations in circulating cell-free DNA: biomarkers of therapeutic resistance in castration-resistant prostate cancer. Clin Cancer Res. 2015; 21:2315-2324; Robinson D., Van Allen E. M., Wu Y. M., et al. Integrative clinical genomics of advanced prostate cancer. Cell. 2015; 161:1215-1228; Ledet E. M., Lilly M. B., Sonpavde G., et al. Comprehensive analysis of AR alterations in circulating tumor DNA from patients with advanced prostate cancer. Oncologist. 2020 April; 25(4):327-333) were stably transfected into HEK293 or T-REx™-293 cells, which do not express endogenous AR (Table 1, FIG. 3, and FIG. 10). Compound A-mediated degradation of all the clinically relevant AR point mutants tested was comparable to that observed for wild-type AR.

TABLE 1
Compound A Half-Maximal Degradation Concentrations (DC₅₀) for
Wild-Type and Mutant AR Expressed in T-Rex-293 Cell Lines

| | Average | SEM | |
|---|---|---|---|
| AR Variant | DC₅₀ (nM) | (nM) | n |
| WT | 1.5 | 0.4 | 7 |
| L702H | 2.4 | 0.4 | 6 |
| V716M | 0.4 | 0.2 | 3 |
| W742C | 2.7 | 1.6 | 2 |
| W742L | 2.3 | 0.5 | 2 |
| M750T | 0.6 | 0.3 | 2 |
| M750V | 1.4 | 0.2 | 2 |
| T878S | 0.9 | 0.4 | 2 |
| S889G | 3.1 | 0.8 | 2 |
| D891H* | 3.9 | 0.2 | 2 |
| H875Y/T878A | 7.7 | 0.5 | 2 |
| F877L/T878A | 7.0 | 1.1 | 2 |
| T878A/D891H | 3.4 | 0.4 | 2 |
| T878A/S889G | 3.6 | 1.2 | 2 |

SEM = standard error of the mean.
AR degradation by Compound A was characterized by ELISA for all AR variants except AR D891H (asterisk) which was characterized by western blot. For western blot analyses, AR levels were quantitated by densitometry using Bio-Rad ImageLab software. AR band intensity was normalized to corresponding glyceraldehyde-3-phosphate dehydrogenase (GAPDH) bands. For wild-type AR and all mutants, the percent AR remaining relative to vehicle control was calculated. Graphing and curve fitting using a four-parameter logistic (4PL) model was performed using GraphPad PRISM. Half-maximal degradation concentration (DC₅₀) values were calculated using Nonlinear Fit.

### Example 3—In Vitro Evaluation of Potential for Degradation of Neosubstrates of Cereblon

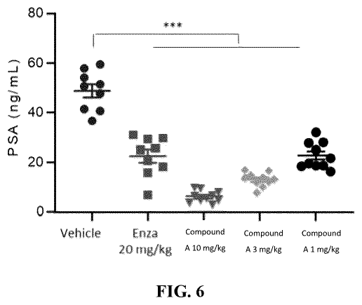

Treatment of Ramos cells and SK-N-DZ cells with 1 μM of Compound A did not lead to degradation of GSPT1, Aiolos, Ikaros, CK1α, and SALL4 (FIG. 4). Other PROTAC compound controls (Con A-D), A3370 (known neomorphic substrate degrader) and 10 mM lenalidomide were also tested. Compound A engages the cereblon protein at same site as IMiDs. Lenalidomide and A3370 induced CBN neosubstrate degradation as expected. Since Compound A does not lead to degradation of known cereblon neomorphic substrates, potential liabilities ascribed to the IMiD class (thalidomide, pomalidomide, lenalidomide) might not apply to Compound A.

### Example 4—Compound a Prostate Tumor Growth Inhibition in the Intact (Non-Castrated) Mouse VCaP Model

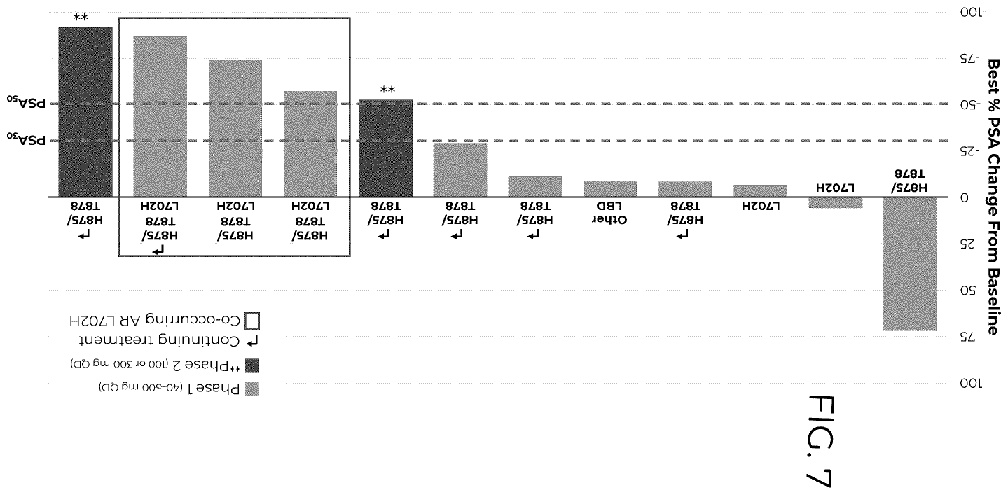

To mimic higher androgen levels found in an incomplete testosterone suppression environment, intact (non-castrated) mice were employed for a VCaP tumor xenograft study. Published data for enzalutamide has demonstrated very modest activity, or no activity at all, in the intact VCaP setting (Asangani, I. A., Dommeti, V. L., Wang X., et al. Therapeutic targeting of BET bromodomain proteins in castration-resistant prostate cancer. Nature. 2014; 510:278-282; Li, J., Alyamani, M., Zhang, A., et al. Aberrant corticosteroid metabolism in tumor cells enables GR takeover in enzalutamide resistant prostate cancer. Elife. 2017; 6, 17 pages). The mice were treated with vehicle, enzalutamide (20 mg/kg, “ENZA”) or Compound A (10, 3, and 1 mg/kg/day) orally, once daily (QD) for 21 days. As shown in FIG. 5, Compound A at 10, 3, and 1 mg/kg/day demonstrated 98%, 74%, and 34% tumor growth inhibition (TGI), respectively, whereas limited efficacy was observed in mice treated with enzalutamide (TGI=27%).

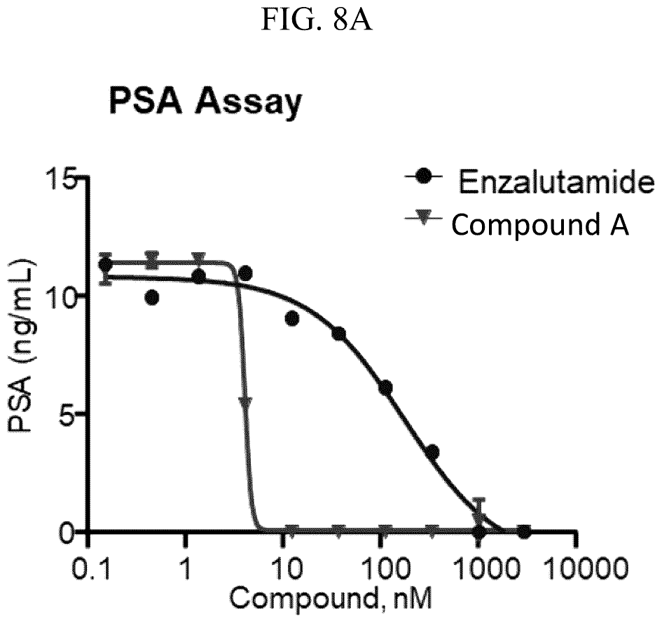

Plasma PSA levels are used as a clinical biomarker of disease progression and are thought to be closely related with the degree of AR activity (Catalona, W. J., Richie, J. P., Ahmann, F. R., et al. Comparison of digital rectal examination and serum prostate specific antigen in the early detection of prostate cancer: results of a multicenter clinical trial of 6,630 men. The Journal of Urology. 1994; 151(5):1283-1290. Cleutjens, K. B., van Eekelen, C. C., van der Korput, H. A., et al. Two androgen response regions cooperate in steroid hormone regulated activity of the prostate-specific antigen promoter. J Biol Chem. 1996; 271(11):6379-6388.). Plasma PSA levels upon Compound A treatment were evaluated by ELISA in samples from the intact VCaP tumor model. As shown in FIG. 6, all 3 doses of Compound A reduced PSA levels in a dose-dependent manner in tumor-bearing mice. In particular, 3 mg/kg and 10 mg/kg of Compound A reduced PSA levels more robustly than 20 mg/kg of enzalutamide. Consistently, the level of PSA reduction by Compound A correlates with TGI shown in FIG. 5.

These TGI and PSA data further demonstrate that Compound A inhibits AR signaling in the presence of high androgen concentrations.

### Example 5—Compound a Food/Proton Pump Inhibitor (PPI) Effect in Healthy Volunteers

Healthy volunteers received a single oral dose of Compound A in a fasting state, in a fed state, and co-administered with the PPI esomeprazole in the fed state. The primary endpoints were AUC_{inf} and Cₘₐₓ. Additional parameters measured included AUCₗₐₛₜ and Tₘₐₓ. A single oral dose of Compound A was generally well tolerated by healthy male participants, with no meaningful difference in the safety profile whether administered in the fasting state, fed state, or in combination with esomeprazole.

The primary PK parameters exhibited less variability under fed state compared to fasted state. Food prolonged absorption and significantly increased the peak concentration and the extent of systemic exposure of Compound A.

Comparable variability was shown between administration of Compound A alone and co-administration of Compound A and esomeprazole. Co-administration slightly decreased the peak concentration and extent of systemic exposure of Compound A.

This phase 1, open-label study included a crossover, fed/fasted cohort and a fixed-sequence PPI cohort. For the fed/fasted cohort, healthy male volunteers received a single oral dose of Compound A 200 mg in a fasted state during treatment A and in a fed state, following a high-calorie/high-fat meal, during treatment B. Participants were randomized to a dosing sequence of treatments AB or BA. For the PPI cohort, a single oral dose of Compound A 300 mg was administered alone in a fed state during treatment C, and during treatment D, participants received esomeprazole 40 mg for 5 days prior to a single oral dose of Compound A 300 mg on day 5 in a fed state. Participants were assigned to a fixed-dosing sequence of treatments CD. There was a 24-day washout period between Compound A treatments. PK blood samples were collected predose and at selected times up to 312 hours post-Compound A dose. Primary endpoints were area under the plasma concentration-time curve from the time of dosing extrapolated to infinity (AUCinf) and maximum plasma concentration (Cₘₐₓ) of Compound A. Secondary endpoints included additional PK parameters and safety and tolerability.

A total of 30 volunteers were enrolled. Analysis populations were food effects (n=14), PPI effects (n=16), and safety (n=30). The median time to Cₘₐₓ (Tₘₐₓ) was delayed by ≈4 hours in fed vs fasted states (12 vs 7.75 hours), and Cₘₐₓ and AUC_{inf} increased 3.9-fold and 3.1-fold, respectively (Table 2). Interindividual variability in PK was lower under fed vs fasted conditions. Compound A in combination with esomeprazole vs Compound A alone resulted in slight reductions in Cₘₐₓ and AUC_{inf} (≈20% and 11%, respectively; Table 2), and median Tₘₐₓ was delayed by 3 hours (10 vs 7 hours).

Food improved absorption, increased the peak concentration and extent of systemic exposure of Compound A, as well as decreased PK variability compared with fasting. Coadministration with esomeprazole slightly decreased the peak concentration and extent of systemic exposure of Compound A when given with food.

The effect of food and a PPI on the PK of Compound A was evaluated in healthy participants. PK results are presented in Tables 2 and 3 and are summarized here.

After single administration of 200 mg Compound A under fed (high-fat meal) and fasted conditions, median peak plasma concentrations were reached at 12 hours and 7.75 hours, respectively. Food significantly increased Compound A Cₘₐₓ (142 ng/mL vs 36 ng/mL) by ˜4-fold and extent of systemic exposure (AUC_{inf} of 10,590 hr*ng/mL vs 3,457 hr*ng/mL) by ˜3-fold. Food also reduced PK variability of Compound A. Under both fed and fasted conditions, Compound A has a mean T1/2 of approximately 60 to 76 hours.

Relative bioavailability was assessed for the effect of food on the PK of Compound A; results indicate that food effect on the 200 mg dose led to higher relative bioavailability (ratio of Compound A fed to fasted: 306.34% for AUC_{inf}, 313.03% for AUCₗₐₛₜ, and 394.46% for Cₘₐₓ). The 90% CIs for peak and systemic exposure ratios were entirely above the upper bound of the conventional equivalence range of 80% to 125%, indicating a clinically relevant effect of food on Compound A exposure. Intra-subject variability was low for AUC and Cₘₐₓ.

Following a single dose of 300 mg Compound A alone or with multiple doses of 40 mg esomeprazole under moderate-fat fed conditions, Compound A was slowly absorbed with median peak plasma levels being reached at 7 and 10 hours, respectively. Coadministration of Compound A with esomeprazole slightly decreased the Cₘₐₓ (by ˜20%) and AUC_{inf} (by ˜11%) of Compound A. Relative bioavailability was assessed for the effect of PPI on the PK of Compound A; results show the ratio of Compound A with esomeprazole to Compound A alone as 89.20% for AUC_{inf}, 88.10% for AUCₗₐₛₜ, and 80.16% for Cₘₐₓ. Results indicate a small effect, but not clinically significant, of esomeprazole on Compound A exposure. Intra-subject variability was low for AUC and Cₘₐₓ.

TABLE 2
Food and PPI Effects on Compound A PK
Food Effects on Compound A PK

| | Geometric LS Mean | | | | | | |
|---|---|---|---|---|---|---|---|
| | B: | | A: | | | | |
| | Compound A 200 | | Compound A 200 | | Geometric | | |
| | mg alone, fed | | mg alone, fasted | | LS Mean Ratio | | Intra- |
| | (Test) | | (Reference) | | (Test/Reference) | | participant |
| PK Parameter | n | LS Mean | n | LS Mean | Estimate (%) | 90% CI | CV % |
| AUC_{inf} (h*ng/mL) | 14 | 10590 | 14 | 3457 | 306.3 | (260.1-360.8) | 24.7 |
| AUCₗₐₛₜ (h*ng/mL) | 14 | 10340 | 14 | 3302 | 313.0 | (264.2-370.9) | 25.6 |
| Cₘₐₓ (ng/mL) | 14 | 142 | 14 | 36 | 394.5 | (323.0-481.7) | 30.3 |

PPI Effects on Compound A PK

| | Geometric LS Mean | | | | | | |
|---|---|---|---|---|---|---|---|
| | D: | | | | | | |
| | Compound A 300 | | C: | | | | |
| | mg with | | Compound A 300 | | Geometric | | |
| | esomeprazole, fed | | mg alone, fed | | LS Mean Ratio | | Intra- |
| | (Test) | | (Reference) | | (Test/Reference) | | participant |
| PK Parameter | n | LS Mean | n | LS Mean | Estimate (%) | 90% CI | CV % |
| AUC_{inf} (h*ng/mL) | 16 | 9716 | 16 | 10890 | 89.2 | (80.4-99.0) | 16.9 |
| AUCₗₐₛₜ (h*ng/mL) | 16 | 9369 | 16 | 10630 | 88.1 | (79.5-97.7) | 16.8 |
| Cₘₐₓ (ng/mL) | 16 | 117 | 16 | 146 | 80.2 | (70.8-90.8) | 20.3 |

AUCₗₐₛₜ = area under the curve from the time of dosing to the time of the last measurable (positive) concentration;
AUC_{inf} = area under the concentration-time curve from time zero, extrapolated to infinity;
CV = coefficient of variation; LS = least square.

TABLE 3
Food and PPI Effects: Summary of Pharmacokinetic
Parameters of Compound A in Study

| | 200 mg Compound A, Fasted | 200 mg Compound A, Fed |
|---|---|---|
| Pharmacokinetic | Geometric Mean | Geometric Mean |
| Parameters | (Geometric CV %) [n] | (Geometric CV %) [n] |
| AUCₗₐₛₜ (ng*hr/mL) | 3302 (48.0) [n = 14] | 10340 (35.8) [n = 14] |
| AUC_{inf}_{_{—}}_{Obs} (ng*hr/mL) | 3457 (46.2) [n = 14] | 10590 (35.8) [n = 14] |
| Cₘₐₓ (ng/mL) | 36.09 (60.2) [n = 14] | 142.3 (33.5) [n = 14] |
| Tₘₐₓ (hr) | 7.754 (5.00, 24.03) [n = 14] | 11.997 (5.99, 24.13) [n = 14] |
| T_{1/2} (hr) | 65.297 (12.1) [n = 14] | 58.651 (12.7) [n = 14] |
| CL/F_Obs (L/hr) | 57.85 (46.2) [n = 14] | 18.89 (35.8) [n = 14] |
| V_{z}/F (L) | 5450 (46.7) [n = 14] | 1598 (40.3) [n = 14] |
| | 300 mg Compound A Alone | 40 mg Esomeprazole + 300 mg |
| Pharmacokinetic | Geometric Mean | Compound A Geometric Mean |
| Parameters | (Geometric CV %) [n] | (Geometric CV %) [n] |
| AUCₗₐₛₜ (ng*hr/mL) | 10630 (39.8) [n = 16] | 9369 (39.2) [n = 16] |
| AUC_{inf}_{_{—}}_{Obs} (ng*hr/mL) | 10890 (39.9) [n = 16] | 9716 (39.2) [n = 16] |
| Cₘₐₓ (ng/mL) | 145.5 (35.6) [n = 16] | 116.6 (39.9) [n = 16] |
| Tₘₐₓ (hr) | 7.001 (5.97, 24.00) [n = 16] | 10.003 (6.00, 24.00) [n = 16] |
| T_{1/2} (hr) | 58.382 (10.7) [n = 16] | 65.461 (11.5) [n = 16] |
| CL/F_Obs (L/hr) | 27.54 (39.9) [n = 16] | 30.88 (39.2) [n = 16] |
| V_{z}/F (L) | 2320 (40.0) [n = 16] | 2916 (41.6) [n = 16] |

AUC_{inf}_{_{—}}_{Obs} = area under the concentration-time curve extrapolated to infinity, based on the last observed concentration; Cₘₐₓ = maximum concentration; Tₘₐₓ = time to maximum plasma concentration; T_{1/2} = half-life of elimination; V_{z}/F = apparent volume of distribution during the terminal elimination phase after extravascular administration.

### Example 6—In Vivo Degradation of AR

Data from the Phase 1/2 dose escalation and expansion trial shows Compound A was well-tolerated with promising activity in a heavily pre-treated, post-NHA, all-comers patient population, including in patients with a L702H mutation. Patients in the Phase 1 studies were administered from 40 mg to 500 mg Compound A once daily. Patients in the Phase 2 studies were administered 100 mg or 300 mg Compound A once daily.

Efficacy:

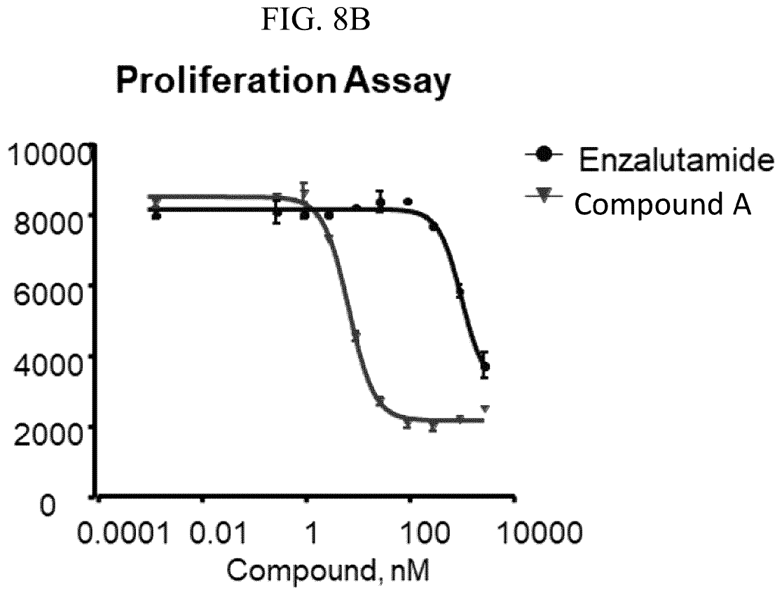

In the Phase 1 and interim Phase 2 trial, AR LBD mutations were present in 28% (13 of 47) of patients' tumors. 42% of patients with AR LBD mutations achieved a reduction of PSA of 50% or greater (PSA₅₀), including L702H mutations. In the three patients with co-occurring AR T878/H875/L702H mutations, 100% (3/3) achieved PSA₅₀. (FIG. 7; AR: androgen receptor; LBD: ligand-binding domain; PSA: prostate-specific antigen; PSA₃₀: best PSA declines ≥30%; PSA₅₀: best PSA declines ≥50%; H875: H875Y; T878: T878A or T878S.)

As the trial progressed, 41% of patients (7 of 17) with AR LBD mutations achieved a reduction of PSA of 50% or greater (PSA₅₀). 50% of patients (4 of 8) with tumors harboring L702H mutations achieved a reduction of PSA of 50% or greater.

Safety:

Compound A was well tolerated to date and the majority of treatment-related adverse events (TRAEs) in the Phase 1 dose escalation data and the Phase 2 interim dose expansion data were Grade 1/2. Low rates of Grade 2 and Grade 3 TRAs were observed. There have been no Grade ≥14 TRAEs.

There was no treatment discontinuation due to TRAE in Phase 1. In Phase 2, there was one treatment discontinuation and two dose reductions due to a TRAE

TABLE 4
Phase 1 and Phase 2 Trials for Compound A: Enrolling
Post-NHA, Heavily Pretreated Patient Population

| | | | Phase 2 | |
|---|---|---|---|---|
| | Phase 1 | | (interim data)* | |
| Parameters | (n = 34) | | (n = 13) | |
| Median age (range), yr | 70 | (59-86) | 67 | (60-76) |

ECOG performance status, n (%)

| 0 | 20 | (59) | 9 | (69) |
|---|---|---|---|---|
| 1 | 14 | (41) | 4 | (31) |
| Visceral disease^{†}, | 23 | (68) | 7 | (54) |
| n (%) | | | | |
| Measurable disease at | 15 | (44) | 3 | (23) |
| baseline, n (%) | | | | |
| Median duration of tx (wks) | 10.8 | (3.9-38.1) | 8.0 | (2.3-15.9) |
| Median no. of prior systemic | 4 | | 5 | |
| anti-cancer regimens | | | | |
| (all setting) | | | | |
| Prior radiotherapy, n (%) | 19 | (61) | 12 | (92) |

Type of prior therapy, n (%)

| | | | | |
|---|---|---|---|---|
| Novel hormonal agent | 34 | (100) | 13 | (100)** |
| Abiraterone acetate alone | 8 | (24) | 4 | (31) |
| Enzalutamide/Darolutamide/ | 8 | (23) | 6 | (46) |
| Apalutamide alone | | | | |
| Combination NHA | 18 | (53) | 2 | (15) |
| Chemotherapy | 24 | (71) | 4 | (31) |

*Phase 2 enrollment ongoing (April 2023 data cutoff date);
**Prior NHA data on one subject was entered into the database after the data cutoff

TABLE 5
AR LBD Mutation Status in Phase 1/2 Patients

| | | |
|---|---|---|
| | Phase 1 | Phase 2 (interim data)^{†} |
| Parameters | (n = 34) | (n = 13) |

AR LBD Mutation Status, n (%)

| | | | | |
|---|---|---|---|---|
| Mutant | 10 | (29) | 3 | (23) |
| H875Y/T878X^{††} without L702H | 4 | (12) | 3 | (23) |
| H875Y/T878X^{††} with L702H | 3 | (9) | 0 | (0) |
| L702H alone | 2 | (6) | 0 | (0) |
| Other LBD missense mutations^{†††} | 1 | (3) | 0 | (0) |
| Wild type | 23 | (68) | 9 | (69) |
| Unknown/Missing | 1 | (3) | 1 | (8) |

^{††}T878X = T878A or T878S.
^{†††}Includes wild type, amplified, and other non-LBD mutations.

### Example 7—AR Degradation and Poly-Ubiquitination

To determine the pharmacological effect of Compound A in vitro, AR degradation assays were conducted in VCaP cells and LNCaP prostate cancer cells that harbor AR amplification and the AR mutation (T878A) mutation, respectively. After 24 hours of treatment, Compound A effectively degraded the AR in LNCaP (Dₘₐₓ ≥91% with DC₅₀ of ≤1.3 nM) and VCaP (Dₘₐₓ ≥94% with DC₅₀ of <1 nM). A degradation time course experiment also performed in VCaP cells demonstrated that >50% degradation of AR is achieved within 2 hours of Compound A treatment of VCaP cells and near complete elimination of AR from the cell is observed by 6 hours. Since Compound A is thought to engage the cereblon protein at the same binding site as ImiDs, such as thalidomide, lenalidomide, and pomalidomide, an excess amount of pomalidomide would compete for the binding of Compound A to the cereblon protein. Consistent with the PROTAC's mode of action, pomalidomide (10 μM) blocked Compound A mediated degradation. In addition, a poly-ubiquitination assay was conducted that demonstrated that ubiquitination of the AR is significantly increased with Compound A treatment. These data support the proposed mode of action of Compound A in degrading AR via the engagement of E3 ligase cereblon, ubiquitination of AR, and degradation via the proteasome.

### Example 8—Compound a Inhibits PSA Synthesis, Blocks Prostate Cancer Cell Proliferation, and Induces Apoptosis in VCaP Cells

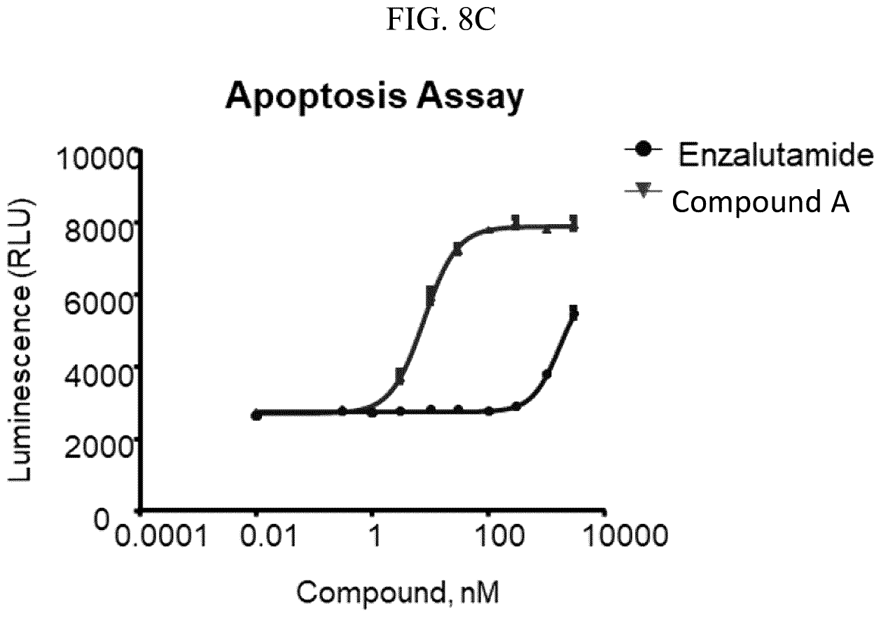

To evaluate the pharmacological effect of Compound A in vitro, PSA, cell proliferation, and apoptosis assays were carried out in VCaP cells. As shown in FIG. 8A, FIG. 8B, and FIG. 8C, Compound A was more potent than enzalutamide at inhibiting PSA expression (IC₅₀: 4 nM vs 175 nM), suppressing cellular proliferation (IC₅₀: 7 nM vs 1,071 nM), and inducing apoptosis (EC₅₀: 8 nM vs 1,661 nM) in VCaP cells. These data demonstrate that Compound A inhibits AR-dependent cellular signaling more potently than enzalutamide.

### Example 9—Effects of Compound A in Inhibiting AR Signaling in a High Androgen Environment In Vitro

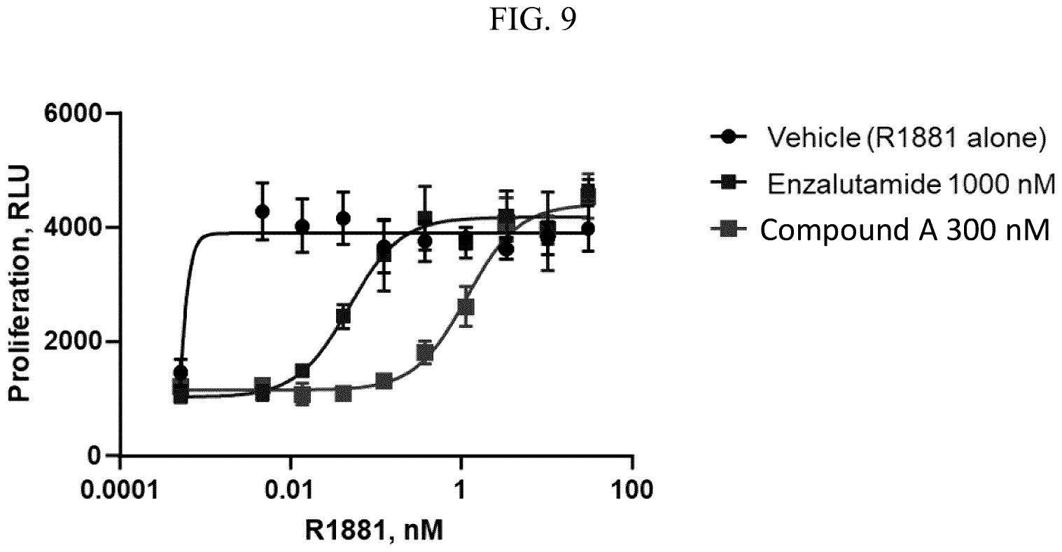

It has been suggested that residual androgen production contributes to the growth of CRPC (Chen C. D., Welsbie D. S., Tran C., et al. Molecular determinants of resistance to antiandrogen therapy. Nat Med. 2004; 10:33-39; Page, S. T., Lin D. W., Mostaghel E. A., et al. Persistent intraprostatic androgen concentrations after medical castration in healthy men. J Clin Endocrinol Metab. 2006; 91:3850-3856.) To evaluate whether Compound A can overcome the effect of residual androgen, a cell proliferation assay was carried out in a high androgen environment condition in vitro. VCaP cells were grown in CSS-containing media in the presence of fixed concentrations of Compound A (300 nM) or enzalutamide (1,000 nM) and increasing concentrations of the synthetic androgen R1881. R1881 alone promoted the proliferation of VCaP cells, demonstrating that proliferation can be induced by activation of the AR signaling pathway FIG. 9). Compound A displayed more potent antiproliferative effects than enzalutamide across a broad range of R1881 concentrations (0.01 to 1 nM), indicating that Compound A is a more potent inhibitor of AR signaling in a high androgen environment.

### Example 10—Dose-Dependent AR Degradation

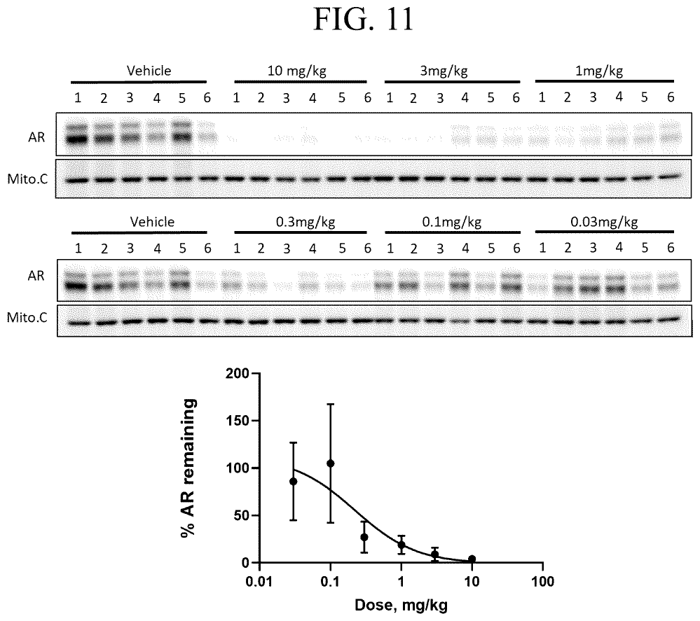

Castration eliminates testosterone produced by the testes. Upon castration, prostate tumor cells implanted in the mouse start to produce testosterone on their own, enabling their growth. The effect of Compound A on AR degradation in vivo was evaluated in castrated mice bearing VCaP xenografts. To recapitulate human CRPC, when the implanted tumors reached a volume of approximately 100 mm³, the mice were surgically castrated. Three weeks after castration, mice were administered a single PO dose of Compound A at 10, 3, 1, 0.3, 0.1, 0.03, or 0 (vehicle) mg/kg via gavage. Sixteen hours after treatment, the tumor tissues were collected, and lysates were prepared to assess AR expression by western blotting. At Compound A doses of 10, 3, 1, 0.3, 0.1, and 0.03 mg/kg, AR was reduced on average by 96%, 91%, 81%, 73%, -5%, and 14%, respectively (FIG. 11), compared to vehicle control. Thus, robust AR reduction was observed in vivo upon oral dosing of Compound A at 1 mg/kg or higher.

### Example 11—Compound A Prostate Tumor Growth Inhibition in the Castrated Mouse VCaP Model

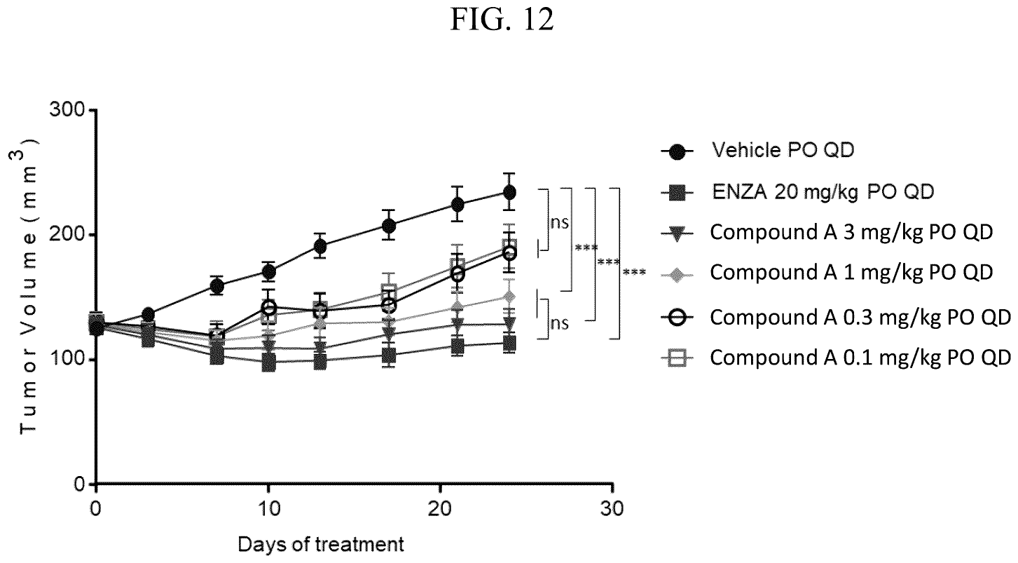

The anti-tumor growth effects of Compound A in vivo were evaluated in the castrated mouse VCaP xenograft model. Briefly, 6- to 8-week-old male CB17/scid mice were implanted with 3×10⁶ VCaP tumor cells in the flank region. Once the tumors reached a volume of approximately 100 mm³, the mice were surgically castrated. When tumors began to grow again and reached about 150 mm³ in castrated animals (approximately 2 to 4 weeks after castration), the mice in each arm of the study (n=10) were treated with 3, 1, 0.3, 0.1, or 0 (vehicle) mg/kg/day Compound A, or 20 mg/kg dose of enzalutamide QD for 24 days, via oral gavage. This dose of enzalutamide has been reported to exceed clinical exposure (U.S. FDA, 2012). All treatments were well tolerated including no effect on body weight. As shown in FIG. 9, FIG. 10, and FIG. 11, treatment with Compound A at 3 mg/kg, 1 mg/kg, and enzalutamide at 20 mg/kg resulted in significant TGI relative to the vehicle control group (100%, 81%, 111%, respectively). Compound A treatment groups at 0.3 mg/kg (TGI=49%) and 0.1 mg/kg (TGI=45%) exhibited a modest and statistically insignificant TGI relative to the vehicle control group. These efficacy data are consistent with the in vivo AR degradation data shown in FIG. 12.

### Example 12—Compound A Effect on Prostate Involution in Rat

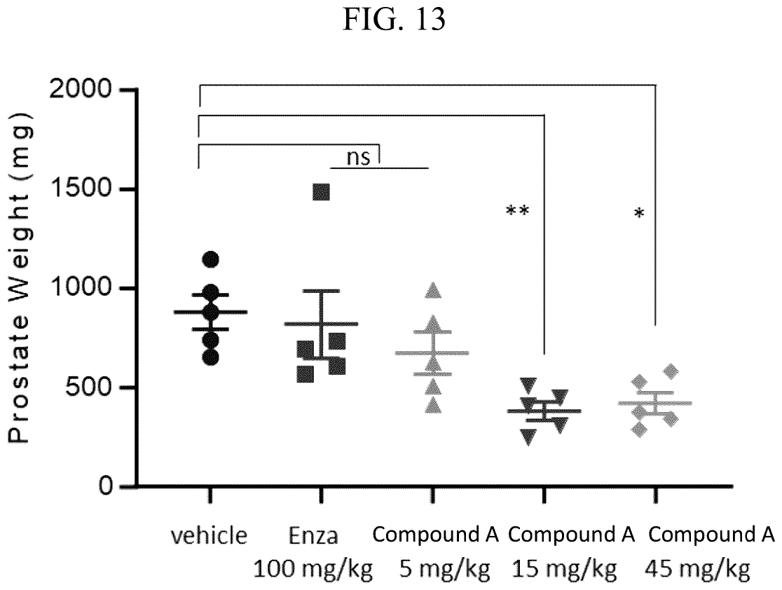

To substantiate the on-target mechanism of Compound A, a prostate involution study was conducted in rats. Intact (non-castrated) adult male Sprague-Dawley rats were treated orally with enzalutamide (100 mg/kg/day) or Compound A (5, 15, or 45 mg/kg/day) for 10 days. Twenty-four hours after the last dose, the rats were euthanized, and the prostate organs were isolated and weighed. Mean (±SEM) prostate weights for the vehicle, enzalutamide 100 mg/kg, Compound A 5 mg/kg, 15 mg/kg and 45 mg/kg treated groups were 880±87, 818±170, 673+105, 384±47, and 422±56 mg, respectively. Compared with enzalutamide, Compound A reduced prostate weights to a greater degree at all doses tested (FIG. 13). These data suggest that Compound A inhibits AR function more than enzalutamide in a high androgen environment, which correlates with the in vitro and in vivo data described herein.

### Example 13—Selectivity Screening

Compound A was tested at 1 μM in a screening study to assess the interaction of Compound A with a panel of 44 targets, including receptors, ion channels, transporters, and enzymes. Any effect greater than 50% of the positive control for a target was considered to represent an interaction of the test compound with the target. As expected, Compound A demonstrated binding to AR at 98.6% of positive control testosterone. No interaction with any other target was identified.

### Example 14—Preliminary PK Data of Compound A

The preliminary PK data of Compound A is presented in Table 6 and summarized in this section.

A chiral bioanalytical method by LC-MS/MS has been developed to characterize the PK of Compound A. Pharmacokinetic analyses were performed using noncompartmental analysis methods.

Following oral administration of Compound A, median Tₘₐₓ was reached at approximately 8 hours post-dose. Mean steady state peak/trough ratio is less than 2, which indicates minimal fluctuation in plasma concentrations once steady state is achieved. Steady state occurs within 3 weeks into treatment.

Preliminary PK results indicated dose related increases in Cₘₐₓ and AUC₀₋₂₄ for Compound A on Day 21 up to 320 mg QD. Accumulation occurred between Day 1 and Day 21 with the average degree of exposure accumulation ranging from approximately 5- to 8-fold. Highest exposures were achieved at 320 mg QD on Day 21. Increasing dose beyond 320 mg QD did not result in higher exposures; instead, at higher doses (400 mg, 480 mg, and 500 mg QD), exposure plateau was evident and a decrease in steady state exposures was observed. Due to continuous QD dosing of mCRPC participants, the terminal phase TI/2 of Compound A could not be determined. Compound A has an effective mean TI/2 of approximately 83 to 111 hours.

Exposure at the 100 mg QD dose (N=6) with mean steady-state Compound A AUC₀₋₂₄=4,610 ng*hr/mL on Cycle 1 Day 21 achieved the minimally efficacious preclinical exposure associated with tumor growth inhibition in the VCaP tumor xenograft mouse model (AUC_{inf}=3,930 ng*hr/mL at 1 mg/kg/day dose; data not included).

TABLE 6
Preliminary Pharmacokinetic Data of Compound A

| | | Cₘₐₓ Day 1 | Cₘₐₓ Day 21 | AUC₀₋₂₄ Day 1 | AUC₀₋₂₄ Day 21 |
|---|---|---|---|---|---|
| QD Dose of | | (ng/mL) | (ng/mL) | (h*ng/mL) | (h*ng/mL) |
| Compound A | | Mean (CV %) [n] | Mean (CV %) [n] | Mean (CV %) [n] | Mean (CV %) [n] |
| 20 | mg^{a} | 11.1 ( ) [1] | 44.1 ( ) [1] | 178 ( ) [1] | 997 ( ) [1] |
| 40 | mg^{a} | 19.1 ( ) [1] | 60.3 ( ) [1] | 314 ( ) [1] | 1,300 ( ) [1] |
| 80 | mg^{a} | 22.2 ( ) [1] | 139 ( ) [1] | 432 ( ) [1] | 3,110 ( ) [1] |
| 100 | mg | 45.1 (51%) [7] | 233 (16%) [6] | 816 (49%) [7] | 4,610 (18%) [6] |
| 160 | mg | 125 (74%) [4] | 445 (35%) [4] | 2,050 (60%) [4] | 8,450 (39%) [4] |
| 200 | mg | 110 (52%) [13] | 559 (46%) [13] | 1,830 (52%) [12] | 11,500 (46%) [13] |
| 300 | mg | 119 (48%) [4] | 672 (42%) [3] | 2,220 (44%) [4] | 14,900 (45%) [3] |
| 320 | mg | 223 (31%) [3] | 1,070 (35%) [3] | 3,990 (21%) [3] | 23,100 (37%) [3] |
| 400 | mg | 232 (64%) [3] | 526 (62%) [3] | 3,350 (48%) [3] | 11,900 (65%) [3] |
| 480 | mg | 145 (97%) [3] | 644 (31%) [3] | 2,540 (102%) [3] | 12,900 (28%) [3] |
| 500 | mg | 203 (63%) [2] | 703 (37%) [2] | 3590 (66%) [2] | 15,000 (45%) [2] |

AUC₀₋₂₄ = area under the concentration-time curve from time zero to 24 hours; Cₘₐₓ = maximum concentration; CV = coefficient of variation; h = hour(s); QD = once daily.
^{a}For lower dose groups when n = 1, Mean represents individual values and CV % was not determined.

### Example 15—Safety in Humans

Safety in Healthy Volunteers

A Phase 1, open-label, multi-cohort study in healthy male participants to evaluate the effect of food and a PPI (esomeprazole) on the single-dose PK and safety of Compound A at doses of 200 mg and 300 mg, respectively.

The study consisted of 2 independent cohorts: an open-label, randomized, 2-period, crossover Fed/Fasted Cohort to determine food effects; and an open-label, 2-period, fixed sequence PPI Cohort to evaluate interactions with esomeprazole.

In the 14 healthy participants treated in the Fed/Fasted Cohort, TEAEs were reported by 5 (35.7%) participants. All TEAEs were Grade 1 in severity. The most frequently reported event was headache, occurring in 4 (28.6%) participants. A total of 3 (21.4%) participants experienced at least one TEAE considered related to Compound A treatment by the Investigator. The TRAEs were headache (2 participants), dizziness (1 participant), and fatigue (1 participant) and all were Grade 1.

In the 16 healthy participants treated in the PPI Cohort, TEAEs were reported by 8 (50.0%) participants. The most frequently reported event was COVID-19, occurring in 3 (18.8%) participants. Most TEAEs were Grade 1 in severity, while 2 (12.5%) participants experienced Grade 2 events of pain in the extremity and CRP increased, respectively. There were no TEAEs of Grade 3 or higher. Two (12.5%) participants experienced at least 1 TEAE considered related to Compound A treatment by the investigator (1 participant experienced Grade 1 hypoesthesia oral and Grade 1 oral discomfort; 1 participant experienced Grade 1 headache and Grade 2 CRP increased). The Grade 2 CRP increased was considered to be possibly related to Compound A and esomeprazole.

There were no deaths, SAEs, or participant discontinuations due to AEs in this study. Overall, there were no clinically significant findings in the safety assessments for laboratory results, vital signs, physical examinations, 12-lead ECGs, or C-SSRS assessments.

Safety in mCRPC Participants

An ongoing, open-label, Phase 1/2 study in male participants with mCRPC. The Phase 1 dose escalation investigated Compound A doses of 20 mg, 40 mg, 80 mg, 100 mg, 160 mg, 200 mg, 320 mg, 400 mg, 480 mg, and 500 mg administered QD. No DLTs have been reported in Part A (Phase 1 dose escalation) of this study. Doses of 100 mg QD and 300 mg QD were selected for the Phase 2 cohort expansion.

All Causality Adverse Events

TEAEs regardless of attribution to Compound A were reported for 44 of the 47 (93.6%) participants treated with Compound A in Phases 1 and 2. Most TEAEs were mild to moderate in severity. Table 7 (Phase 1) and Table 8 (Phase 2) present the most common TEAE PTs reported by ≥10% of participants; the TEAEs are ordered in decreasing order of frequency by SOC, then PT. The most commonly reported TEAE PTs in Phase 1 were fatigue, blood alkaline phosphatase increased, blood creatinine increased, nausea, and diarrhea; all reported by ≥20% of participants. The most common TEAE PTs in Phase 2, reported in ≥20% of participants, were anemia, dysgeusia, diarrhea, hematuria, and blood alkaline phosphatase increased.

The only Grade ≥3 TEAL that occurred in more than one participant (including events that occurred in fewer than 1000 of participants) was diarrhea, which was reported by 2 participants as Grade 3 events in Phase 1.

TABLE 7
All-causality TEAEs Reported for ≥10% of
Participants^{a} (by SOC and PT) in Phase
1 Study (All Dose Levels Combined, Safety Analysis Set)

| | All Phase 1 Dose Levels | | | | | | |
|---|---|---|---|---|---|---|---|
| | Combined (N = 34) | | | | | | |
| System Organ Class | Number (%) of Participants | | | | | | |
| Preferred Term | Any Grade | | Grade 3 | | Grade 4 | | Grade 5 |
| Participants with any | 33 | (97.1) | 10 | (29.4) | 2 | (5.9) | 1 (2.9) |
| TEAEs | | | | | | | |
| Investigations | 19 | (55.9) | 1 | (2.9) | 1 | (2.9) | 0 |

| Blood alkaline | 8 | (23.5) | 0 | | 0 | 0 |
|---|---|---|---|---|---|---|
| phosphatase | | | | | | |
| increased | | | | | | |
| Blood creatinine | 7 | (20.6) | 0 | | 0 | 0 |
| increased | | | | | | |
| Aspartate | 5 | (14.7) | 1 | (2.9) | 0 | 0 |
| aminotransferase | | | | | | |
| increased | | | | | | |

| Neutrophil count | 4 | (11.8) | 0 | 0 | | 0 |
|---|---|---|---|---|---|---|
| decreased | | | | | | |
| Lymphocyte count | 4 | (11.8) | 0 | 1 | (2.9) | 0 |
| decreased | | | | | | |

| General disorders and | 16 | (47.1) | 1 | (2.9) | 0 | 0 |
|---|---|---|---|---|---|---|
| administration site | | | | | | |
| conditions | | | | | | |
| Fatigue | 12 | (35.3) | 1 | (2.9) | 0 | 0 |
| Gastrointestinal | 15 | (44.1) | 2 | (5.9) | 0 | 0 |
| disorders | | | | | | |
| Nausea | 7 | (20.6) | 0 | | 0 | 0 |
| Diarrhoea | 7 | (20.6) | 2 | (5.9) | 0 | 0 |
| Constipation | 4 | (11.8) | 0 | | 0 | 0 |
| Metabolism and | 15 | (44.1) | 1 | (2.9)^{b} | 0 | 0 |
| nutrition disorders | | | | | | |

| Decreased appetite | 5 | (14.7) | 0 | 0 | | 0 |
|---|---|---|---|---|---|---|
| Hypophosphataemia | 4 | (11.8) | 0 | 0 | | 0 |
| Hyponatraemia | 4 | (11.8) | 0 | 0 | | 0 |
| Skin and subcutaneous | 10 | (29.4) | 0 | 0 | | 0 |
| tissue disorders | | | | | | |
| Rash maculo-papular | 4 | (11.8) | 0 | 0 | | 0 |
| Alopecia | 4 | (11.8) | 0 | 0 | | 0 |
| Blood and lymphatic | 7 | (20.6) | 0 | 1 | (2.9)^{c} | 0 |
| system disorders | | | | | | |

| Anaemia | 6 | (17.6) | 0 | | 0 | 0 |
|---|---|---|---|---|---|---|
| Psychiatric disorders | 7 | (20.6) | 0 | | 0 | 0 |
| Insomnia | 5 | (14.7) | 0 | | 0 | 0 |
| Respiratory, thoracic | 6 | (17.6) | 2 | (5.9)^{d} | 0 | 0 |
| and mediastinal | | | | | | |
| disorders | | | | | | |
| Dyspnoea | 4 | (11.8) | 1 | (2.9) | 0 | 0 |

| | | | | | |
|---|---|---|---|---|---|
| Cough | 4 | (11.8) | 0 | 0 | 0 |

MedDRA: 25.0. TEAE is defined as an AE occurring on or after the first dose and within 30 days of last dose for participants who already came off treatment. For ongoing participants, any AE occurring on or after the first dose is considered a TEAE.
Participants with multiple events under the same SOC or PT are counted once only at the highest CTCAE grade
^{a}The ≥10% of participants threshold was based on incidence in “Any Grade” category.
One participant had Grade 3 hypokalemia and Grade 3 hypomagnesemia, PTs that did not meet the ≥10% threshold.
One participant had Grade 4 febrile neutropenia, a PT that did not meet the ≥10% threshold.
One participant had Grade 3 hypoxia, a PT that did not meet the ≥10% threshold.

TABLE 8
All-causality TEAEs Reported for >10% of
Participants^{a} (by SOC and PT) in Phase 2 Study
(Dose Levels Combined, Safety Analysis Set)

| | Phase 2 Dose Levels | | | | | |
|---|---|---|---|---|---|---|
| | Combined (N = 13) | | | | | |
| System Organ Class | Number (%) of Participants | | | | | |
| Preferred Term | Any Grade | | Grade 3 | | Grade 4 | Grade 5 |
| Participants with | 11 | (84.6) | 2 | (15.4) | 0 | 0 |
| any TEAEs | | | | | | |
| Gastrointestinal | 7 | (53.8) | 1 | (7.7) | 0 | 0 |
| disorders | | | | | | |
| Diarrhoea | 3 | (23.1) | 0 | | 0 | 0 |
| Vomiting | 2 | (15.4) | 0 | | 0 | 0 |
| Nausea | 2 | (15.4) | 0 | | 0 | 0 |
| Abdominal pain | 2 | (15.4) | 1 | (7.7) | 0 | 0 |
| Renal and urinary | 6 | (46.2) | 0 | | 0 | 0 |
| disorders | | | | | | |
| Haematuria | 3 | (23.1) | 0 | | 0 | 0 |
| Proteinuria | 2 | (15.4) | 0 | | 0 | 0 |
| Investigations | 5 | (38.5) | 0 | | 0 | 0 |
| Blood alkaline | 3 | (23.1) | 0 | | 0 | 0 |
| phosphatase | | | | | | |
| increased | | | | | | |
| Blood and lymphatic | 4 | (30.8) | 0 | | 0 | 0 |
| system disorders | | | | | | |
| Anaemia | 4 | (30.8) | 0 | | 0 | 0 |
| Nervous system | 3 | (23.1) | 0 | | 0 | 0 |
| disorders | | | | | | |
| Dysgeusia | 3 | (23.1) | 0 | | 0 | 0 |
| General disorders and | 2 | (15.4) | 0 | | 0 | 0 |
| administration site | | | | | | |
| conditions | | | | | | |
| Fatigue | 2 | (15.4) | 0 | | 0 | 0 |
| Skin and subcutaneous | 2 | (15.4) | 0 | | 0 | 0 |
| tissue disorders | | | | | | |
| Alopecia | 2 | (15.4) | 0 | | 0 | 0 |
| Vascular disorders | 2 | (15.4) | 1 | (7.7) | 0 | 0 |
| Hypertension | 2 | (15.4) | 1 | (7.7) | 0 | 0 |

MedDRA: 25.0. TEAE is defined as an AE occurring on or after the first dose and within 30 days of last dose for participants who already came off treatment. For ongoing participants, any AE occurring on or after the first dose is considered a TEAE.
Participants with multiple events under the same SOC or PT are counted once only at the highest CTCAE grade.
b. Determination for meeting ≥10% threshold was based on incidence in “Any Grade” category.

Treatment-Related Adverse Events

TEAEs that were considered at least potentially related to Compound A treatment were reported for 25 of 47 (53.2%) participants in Phases 1 and 2 combined (data not shown). In Phase 1, TRAEs reported in ≥10% of participants are fatigue (7 [20.6%] participants) and nausea and diarrhea (4 [11.8%] participants each). In Phase 2, TRAEs reported in ≥10% of participants are nausea, abdominal pain, dysgeusia, and alopecia, each reported by 2 (15.4%) participants. There was 1 TRAE of Grade 3 fatigue and 1 TRAE of Grade 3 aspartate aminotransferase increased in Phase 1, and 1 TRAE of Grade 3 abdominal pain in Phase 2. There were no Grade 4 or 5 TRAEs in Phase 1 of the Study. Most TRAEs were mild to moderate in severity.

Adverse Events Leading to Treatment Discontinuation, Dose Interruption, or Dose Reduction

No participant discontinued study treatment due to a TEAE in Phase 1. One participant in Phase 2 discontinued treatment due to a Grade 3 non-serious TEAE of abdominal pain; it was considered related to Compound A.

Six (17.6%) participants interrupted study treatment due to 1 or more TEAEs in Phase 1; one participant's TEAEs were considered related to Compound A (Grade 3 aspartate aminotransferase increased and Grade 2 blood creatine phosphokinase increased). Three (23.1%) participants interrupted study treatment due to 1 or more TEAEs in Phase 2. Two of these participants' TEAEs were considered related to Compound A (one participant with Grade 1 nausea and one participant with Grade 2 diarrhea, vomiting, and dehydration).

Two (5.9%) participants experienced TEAEs leading to dose reduction in Phase 1; both participants' events were considered related to Compound A (Grade 2 diarrhea and Grade 3 fatigue, 1 participant each). One (7.7%) participant experienced a TEAE leading to dose reduction in Phase 2 (Grade 1 diarrhea). It was considered not related to Compound A.

All Causality Serious Adverse Events

From the beginning of the study until data cut-off, treatment-emergent SAEs regardless of attribution to Compound A were reported for 11 of 34 (32.40%) participants in Phase 1; no SAEs were reported in Phase 2. No treatment-related SAEs were reported in either phase. No individual SAL PT was reported in more than one participant.

All SAEs were Grade ≥3 except for one SA of Grade 2 lethargy. There was one Grade 4 SAL (febrile neutropenia). The investigator and Sponsor assessed the event of febrile neutropenia as unrelated to Compound A. This event was attributed to the participant's chemotherapy, viral infection contracted while neutropenic, or a combination of both by the investigator. There was one Grade 5 SAL (brain stem stroke; unrelated to Compound A). Additional details of this death are provided below.

The overall incidence of SAEs combining Phases 1 and 2 is 11 of 47 (23.4) participants. All causality SAEs are presented for Phases 1 and 2 combined in Error! Reference source not found.

TABLE 9
All Causality Treatment Emergent SAEs (by SOC and
PT) Reported in Phases 1 and 2 of Study (Combined,
All Dose Levels Combined, Safety Analysis Set)

| | All Dose Levels and Phases 1 | | | | | |
|---|---|---|---|---|---|---|
| | and 2 Combined^{a} | | | | | |
| | (N = 47) | | | | | |
| System Organ Class | Number of Participants (%) | | | | | |
| Preferred Term | Any Grade | | Grade 3 | | Grade 4 | Grade 5 |
| Participants with any | 11 | (23.4)^{b} | 8 | (17.0) | 1 (2.1) | 1 (2.1) |
| serious TEAEs | | | | | | |
| Nervous system disorders | 4 | (8.5) | 2 | (4.3) | 0 | 1 (2.1) |
| Tremor | 1 | (2.1) | 1 | (2.1) | 0 | 0 |
| Spinal cord compression | 1 | (2.1) | 1 | (2.1) | 0 | 0 |
| Lethargy^{b} | 1 | (2.1) | 0 | | 0 | 0 |
| Brain stem stroke | 1 | (2.1) | 0 | | 0 | 1 (2.1) |
| Renal and urinary | 3 | (6.4) | 3 | (6.4) | 0 | 0 |
| disorders | | | | | | |
| Urinary tract obstruction | 1 | (2.1) | 1 | (2.1) | 0 | 0 |
| Haematuria | 1 | (2.1) | 1 | (2.1) | 0 | 0 |
| Cystitis noninfective | 1 | (2.1) | 1 | (2.1) | 0 | 0 |
| Infections and | 2 | (4.3) | 2 | (4.3) | 0 | 0 |
| infestations | | | | | | |
| Soft tissue infection | 1 | (2.1) | 1 | (2.1) | 0 | 0 |
| Pneumonia | 1 | (2.1) | 1 | (2.1) | 0 | 0 |
| Respiratory, thoracic and | 2 | (4.3) | 2 | (4.3) | 0 | 0 |
| mediastinal disorders | | | | | | |
| Hypoxia | 1 | (2.1) | 1 | (2.1) | 0 | 0 |
| Dyspnoea | 1 | (2.1) | 1 | (2.1) | 0 | 0 |
| Blood and lymphatic | 1 | (2.1) | 0 | | 1 (2.1) | 0 |
| system disorders | | | | | | |
| Febrile neutropenia | 1 | (2.1) | 0 | | 1 (2.1) | 0 |
| Gastrointestinal | 1 | (2.1) | 1 | (2.1) | 0 | 0 |
| disorders | | | | | | |
| Diarrhoea | 1 | (2.1) | 1 | (2.1) | 0 | 0 |

MedDRA:25.0. TEAE is defined as an AE occurring on or after the first dose and within 30 days of last dose for participants who already came off treatment. For ongoing participants, any AE occurring on or after the first dose is considered a TEAE.
c. No SAEs were reported in Phase 2; percentages are based on the total N for Phases 1 and 2.
One participant experienced Grade 2 lethargy.

Five deaths have been reported from the study start until the data cut-off. No deaths were considered related to Compound A.

Of the 5 deaths, 4 occurred more than 30 days after the last dose of Compound A and are not discussed further. The one death that occurred within 30 days of the last dose was due to a TEAE. This Phase 1 participant in the 100 mg QD dose group had a brain stem stroke and died 9 days later. The event of brain stem stroke was assessed as not related to Compound A treatment by the investigator and the Sponsor; it was attributed to the participant's known underlying history of hypertension, hyperlipidemia, and diabetes mellitus type 2 by the investigator. The last dose of Compound A was taken 1 day before the stroke occurred; study medication was held upon hospital admission and never resumed.

### Example 16—Deep PSA Decline

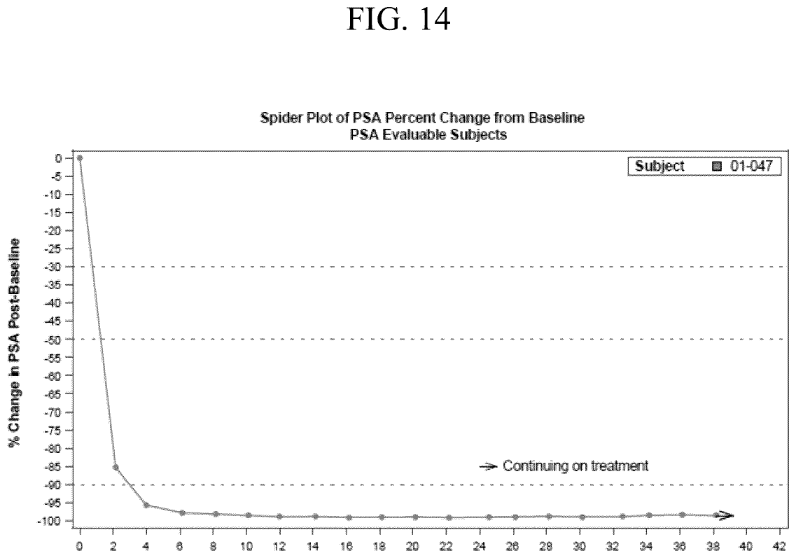

Deep PSA decline in patient with mCRPC with wild-type AR supports development in pre-NHA settings, including mCSPC (FIG. 14). Evaluable analysis set include all Compound A treated participants with baseline PSA assessment and at least a C1D28 or later postbaseline PSA assessment. A PSA₉₀ and PSA₅₀ and PSA₃₀ response is defined as =90% or 50% or 30% decline in PSA from baseline PSA value.

### Example 17—Compound A in Clinical Setting

TABLE 10
Treatment-Related Adverse Events (TRAEs) Greater than 10%

| | | |
|---|---|---|
| | Any Grade | Grade 3+ |
| TRAE ≥10% n (%) | (n = 84) | (n = 84) |

| | | | |
|---|---|---|---|
| Any TRAE | 55 | (66) | 7 (8) |
| Fatigue | 24 | (29) | 2 (2) |
| Nausea | 12 | (14) | 0 (0) |
| Diarrhea | 9 | (11) | 1 (1) |
| Vomiting | 9 | (11) | 0 (0) |
| Decreased appetite | 9 | (11) | 0 (0) |
| Alopecia | 8 | (10) | 0 (0) |
| Anemia | 3 | (4) | 0 (0) |
| Weight decrease | 1 | (1) | 0 (0) |
| Aspartate Aminotransferase increase | 6 | (7) | 1 (1) |

### ENUMERATED EMBODIMENTS

The aspects of the present disclosure are further described with reference to the following embodiments:

A. A method of treating prostate cancer in a subject comprising administering to a subject in need thereof a therapeutically effective amount of Compound A,

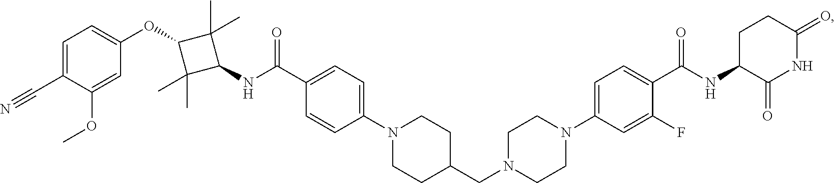 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

pharmaceutically acceptable salt thereof.
B. A method of treating prostate cancer in a subject comprising administering to a subject in need thereof a therapeutically effective amount of Compound A,

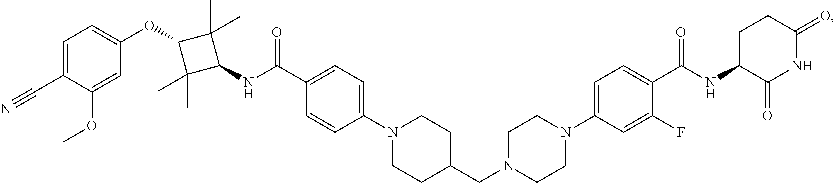 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

C. The method of embodiment B or C, wherein the therapeutically effective amount of Compound A is administered orally to the subject.
D. The method of any one of embodiments A-C, wherein the therapeutically effective amount of Compound A is administered to the subject once a day.
E. The method of any one of embodiments A-D, wherein the therapeutically effective amount of Compound A is administered to the subject all at once or is administered in two, three, or four divided doses.
F. The method of any one of embodiments A-E, wherein the therapeutically effective amount of Compound A is about 1 mg to about 1000 mg.
G. The method of any one of embodiments A-F, wherein the therapeutically effective amount of Compound A is about 5 mg to about 750 mg.
H. The method of any one of embodiments A-F, wherein the therapeutically effective amount of Compound A is about 10 mg to about 500 mg.
I. The method of any one of embodiments A-F, wherein the therapeutically effective amount of Compound A is about 20 mg to about 250 mg.
J. The method of any one of embodiments A-F, wherein the therapeutically effective amount of Compound A is about 100 mg.
K. The method of any one of embodiments A-F, wherein the therapeutically effective amount of Compound A is about 150 mg.
L. The method of any one of embodiments A-F, wherein the therapeutically effective amount of Compound A is about 300 mg.
M. The method of any one of embodiments A-L, wherein the subject is in a fed state at the time of administration.
N. The method of any one of embodiments A-L, wherein the subject is in a fasted state at the time of administration.
O. The method of any one of embodiments A-N, further comprising administering an effective amount of at least one additional anti-cancer agent to the subject in need thereof.
P. The method of embodiment 0, wherein the additional anti-cancer agent is abiraterone, abiraterone acetate, estramustine, docetaxel, ketoconazole, goserelin, histrelin, triptorelin, buserelin, cyproterone, flutamide, bicalutamide, nilutamide, pamidronate, or zolendronate.
Q. The method of any one of embodiments A-P, where the prostate cancer comprises at least one somatic AR tumor mutation.
R. The method of any one of embodiments A-P, where the subject with prostate cancer comprises at least one somatic AR tumor mutation.
S. The method of embodiment Q or R, wherein the at least one somatic AR tumor mutation is:
(i) selected from the group consisting of L702H, M895V, W742C, S889G, M750V, M896V, T878A, F877L, D891H, H875Y, and any combination thereof,
(ii) L702H;
(iii) T878A;
(iv) T878S;
(v) H875Y;
(vi) L702H and T878A;
(vii) L702H and T878S;
(viii) L702H and H875Y;
(ix) T878A and H875Y;
(x) T878S and H875Y;
(xi) L702H, T878A, and H875Y; or
(xii) L702H, T878S, and H875Y.
   T. A method of treating prostate cancer in a subpopulation of prostate cancer subjects, comprising:
(a) selecting a prostate cancer subject for treatment based on the subject's somatic AR tumor biomarker status; and
(b) administering a therapeutically effective amount of Compound A,

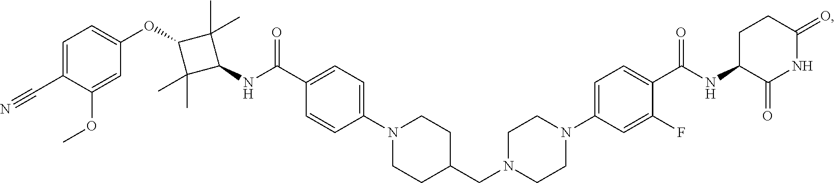 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

or a pharmaceutically acceptable salt thereof.
U. A method of treating prostate cancer in a subpopulation of prostate cancer subjects, comprising:
(a) selecting a prostate cancer subject for treatment based on the subject's somatic AR tumor biomarker status; and
(b) administering a therapeutically effective amount of Compound A,

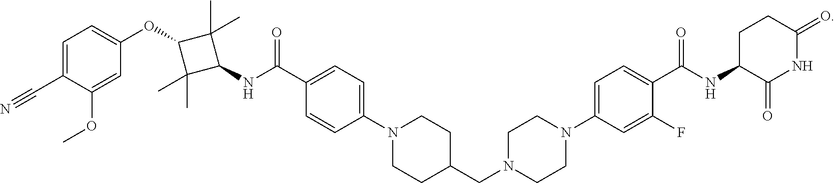 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

The method of any one of the preceding claims, where the subject's somatic AR tumor biomarker status comprises at least one somatic AR tumor mutation.
V. The method of embodiment T or U, wherein the at least one somatic AR tumor mutation is:
(i) selected from the group consisting of L702H, M895V, W742C, S889G, M750V, M896V, T878A, F877L, D891H, H875Y, and any combination thereof,
(ii) L702H;
(iii) T878A;
(iv) T878S;
(v) H875Y;
(vi) L702H and T878A;
(vii) L702H and T878S;
(viii) L702H and H875Y;
(ix) T878A and H875Y;
(x) T878S and H875Y;
(xi) L702H, T878A, and H875Y; or
(xii) L702H, T878S, and H875Y.
   W. The method of any one of embodiments T, U, or V, wherein the AR biomarker status of the subject is determined by ctDNA analysis, fluorescent in situ hybridization, immunohistochemistry, PCR analysis, or sequencing; or is determined in a blood sample derived from the subject.
   X. The method of any one of embodiments T, U, or V, wherein the AR biomarker status of the subject is determined in a solid biopsy derived from the tumor of the subject.
   Y. The method of any one of embodiments A-X, wherein the subject is also undergoing ongoing androgen deprivation therapy (ADT).
   Z. The method of embodiment Y, wherein the ADT comprises the administration of a gonadotropin releasing hormone analog or inhibitor to the subject.
   AA. The method of embodiment Z, wherein the gonadotropin releasing hormone analog or inhibitor is leuprolide, goserelin, triptorelin, histrelin, or a pharmaceutically acceptable salt thereof.
   BB. The method of any one of embodiments A-AA, wherein the subject has undergone an orchiectomy.
   CC. The method of any one of embodiments A-BB, wherein the subject has a histological, pathological, or cytological confirmed diagnosis of adenocarcinoma of the prostate.
   DD. The method of any one of embodiments A-CC, wherein, prior to the administration of the therapeutically effective amount of Compound A, the subject experienced progression of prostate cancer on at least one prior approved systemic therapy for metastatic prostate cancer.
   EE. The method of any one of embodiments A-DD, wherein, prior to the administration of the therapeutically effective amount of Compound A, the subject experienced progression of prostate cancer on at least two prior approved systemic therapies for metastatic prostate cancer.
   FF. The method of embodiment EE, wherein the prior approved systemic therapy for metastatic prostate cancer is a second-generation androgen inhibitor.
   GG. The method of embodiment FF, wherein the second-generation androgen inhibitor is abiraterone, abiraterone acetate, enzalutamide, darolutamide, apalutamide, or a pharmaceutically acceptable salt thereof.
   HH. The method of any one of embodiments A-GG, wherein the subject has an ECOG performance status of 0 or 1.
   II. The method of any one of embodiments A-HH, wherein the subject does not have a symptomatic brain metastasis requiring steroids above physiologic replacement doses.
   JJ. The method of any one of embodiments A-II, wherein the subject does not have active inflammatory bowel disease.
   KK. The method of any one of embodiments A-JJ, wherein the subject does not have chronic diarrhea.
   LL. The method of any one of embodiments A-KK, wherein the subject does not have diverticular disease.
   MM. The method of any one of embodiments A-LL, wherein the subject has not previously undergone gastric resection.
   NN. The method of any one of embodiments A-MM, wherein the subject has not previously undergone lap band surgery.
   OO. The method of any one of embodiments A-NN, wherein the subject has not previously undergone radiation therapy within four weeks prior to the initial administration of the therapeutically effective amount of Compound A.
   PP. The method of any one of embodiments A-GO, wherein the subject has not previously undergone radiation therapy where greater than about 25% of the subject's bone marrow was irradiated.
   QQ. The method of any one of embodiments A-PP, wherein the subject has not been administered an investigational drug within four weeks prior to the initial administration of the therapeutically effective amount of Compound A.
   RR. The method of any one of embodiments A-QQ, wherein the prostate cancer is adenocarcinoma of the prostate.
   SS. The method of any one of embodiments A-QQ, wherein the prostate cancer is metastatic prostate cancer.
   TT. The method of any one of embodiments A-QQ, wherein the prostate cancer is castrate-resistant prostate cancer.
   UU. The method of any one of embodiments A-QQ, wherein the prostate cancer is metastatic castrate-resistant prostate cancer.
   VV. The method of any one of embodiments A-QQ, wherein the prostate cancer is progressive metastatic castrate-resistant prostate cancer.
   WW. The method of any one of embodiments A-QQ, wherein the prostate cancer is castrate-sensitive prostate cancer.
   XX. The method of any one of embodiments A-QQ, wherein the prostate cancer is metastatic castrate-sensitive prostate cancer.
   YY. The method of any one of embodiments A-QQ, wherein the prostate cancer is prostate cancer naïve to novel hormonal agents (NHA).
   ZZ. The method of embodiment YY, wherein the prostate cancer naïve to novel hormonal agents (NHA) has not been previously treated with a second-generation antiandrogen.
   AAA. The method of any one of embodiments A-QQ, wherein the prostate cancer is metastatic prostate cancer naïve to novel hormonal agents (NHA).
   BBB. The method of embodiment AAA, wherein the metastatic prostate cancer naïve to novel hormonal agents (NHA) has not been previously treated with a second-generation antiandrogen.
   CCC. The method of any one of embodiments A-QQ, wherein the prostate cancer is castrate-resistant prostate cancer naïve to novel hormonal agents (NHA).
   DDD. The method of embodiment CCC, wherein the castrate-resistant prostate cancer naïve to novel hormonal agents (NHA) has not been previously treated with a second-generation antiandrogen.
   EEE. The method of any one of embodiments A-QQ, wherein the prostate cancer is castrate-sensitive prostate cancer naïve to novel hormonal agents (NHA).
   FFF. The method of embodiment EEE, wherein the castrate-sensitive prostate cancer naïve to novel hormonal agents (NHA) has not been previously treated with a second-generation antiandrogen.
   GGG. The method of any one of embodiments A-QQ, wherein the prostate cancer is metastatic castrate-resistant prostate cancer naïve to novel hormonal agents (NHA).
   HHH. The method of embodiment GGG, wherein the metastatic castrate-resistant prostate cancer naïve to novel hormonal agents (NHA) has not been previously treated with a second-generation antiandrogen.
   III. The method of any one of embodiments A-QQ, wherein the prostate cancer is metastatic castrate-sensitive prostate cancer naïve to novel hormonal agents (NHA).
   JJJ. The method of embodiment III, wherein the metastatic castrate-sensitive prostate cancer naïve to novel hormonal agents (NHA) has not been previously treated with a second-generation antiandrogen.
   LLL. The method of any one of embodiments A-JJJ, wherein the prostate cancer has not been previously treated with an androgen biosynthesis inhibitor or an androgen receptor blocker.
   MMM. The method of any one of embodiments A-LLL, wherein the prostate cancer has not been previously treated with abiraterone acetate.
   NNN. The method of any one of embodiments A-MMM, wherein the prostate cancer has not been previously treated with an androgen receptor blocker selected from enzalutamide, darolutamide, and apalutamide.
   OOO. The method of any one of embodiments A-JJJ, wherein the subject has not been previously administered an androgen biosynthesis inhibitor or an androgen receptor blocker.
   PPP. The method of any one of embodiments A-JJJ, wherein the subject has not been previously administered abiraterone acetate.
   QQQ. The method of any one of embodiments A-JJJ, wherein the subject has not been previously administered an androgen receptor blocker selected from enzalutamide, darolutamide, and apalutamide.
   RRR. A method of treating prostate cancer in a subject comprising administering to a subject in need thereof a therapeutically effective amount of Compound A,
   or a

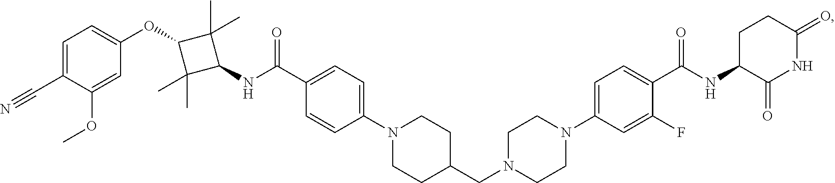 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

pharmaceutically acceptable salt thereof, wherein the therapeutically effective amount of Compound A is about 5 mg to about 750 mg.
SSS. A method of treating prostate cancer in a subject comprising administering to a subject in need thereof a therapeutically effective amount of Compound A,

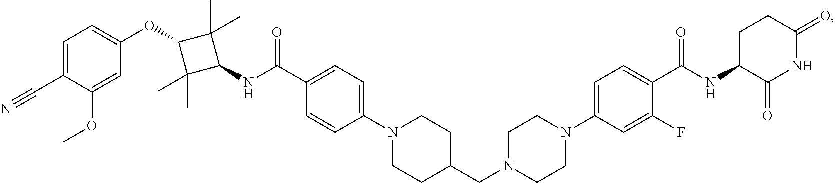 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

wherein the therapeutically effective amount of Compound A is about 5 mg to about 750 mg.
TTT. The method of Embodiment RRR or SSS, wherein the prostate cancer is adenocarcinoma of the prostate.
UUU. The method of Embodiment RRR or SSS, wherein the prostate cancer is metastatic prostate cancer.
VVV. The method of Embodiment RRR or SSS, wherein the prostate cancer is castrate-resistant prostate cancer.
WWW. The method of Embodiment RRR or SSS, wherein the prostate cancer is metastatic castrate-resistant prostate cancer.
XXX. The method of Embodiment RRR or SSS, wherein the prostate cancer is progressive metastatic castrate-resistant prostate cancer.
YYY. The method of Embodiment RRR or SSS, wherein the prostate cancer is castrate-sensitive prostate cancer.
ZZZ. The method of Embodiment RRR or SSS, wherein the prostate cancer is metastatic castrate-sensitive prostate cancer.
AAAA. A method of treating prostate cancer in a subject comprising administering to a subject in need thereof:
(i) a therapeutically effective amount of Compound A,

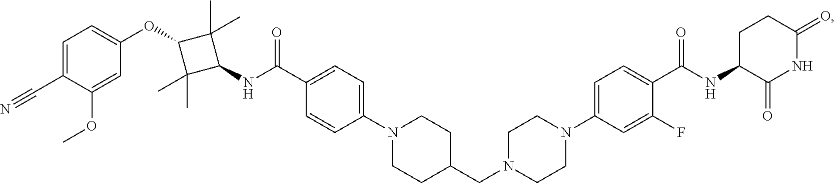 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

or a pharmaceutically acceptable salt thereof, and
(ii) a therapeutically effective amount of abiraterone acetate;
wherein the therapeutically effective amount of Compound A is about 5 mg to about 750 mg; and
wherein the therapeutically effective amount of abiraterone acetate is about 250 mg to about 1500 mg.
   BBBB. A method of treating prostate cancer in a subject comprising administering to a subject in need thereof:
(i) a therapeutically effective amount of Compound A,

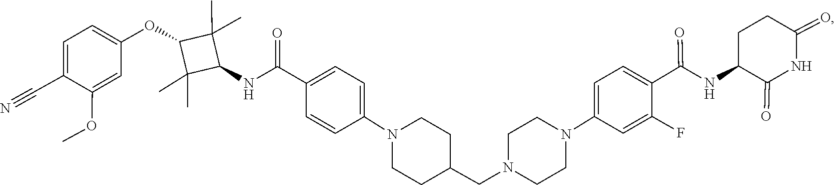 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

(ii) a therapeutically effective amount of abiraterone acetate;
wherein the therapeutically effective amount of Compound A is about 5 mg to about 750 mg; and
wherein the therapeutically effective amount of abiraterone acetate is about 250 mg to about 1500 mg.
   CCCC. The method of Embodiment RRR or SSS, wherein the prostate cancer is adenocarcinoma of the prostate.
   DDDD. The method of Embodiment RRR or SSS, wherein the prostate cancer is metastatic prostate cancer.

EEEE. The method of Embodiment RRR or SSS, wherein the prostate cancer is castrate-resistant prostate cancer.

FFFF. The method of Embodiment RRR or SSS, wherein the prostate cancer is metastatic castrate-resistant prostate cancer.

GGGG. The method of Embodiment RRR or SSS, wherein the prostate cancer is progressive metastatic castrate-resistant prostate cancer.
HHHH. The method of Embodiment RRR or SSS, wherein the prostate cancer is castrate-sensitive prostate cancer.
IIII. The method of Embodiment RRR or SSS, wherein the prostate cancer is metastatic castrate-sensitive prostate cancer.
JJJJ. A method of treating prostate cancer in a subject in need thereof, wherein the prostate cancer comprises at least one somatic AR tumor mutation, the method comprising administering to the subject in need thereof a therapeutically effective amount of Compound A,

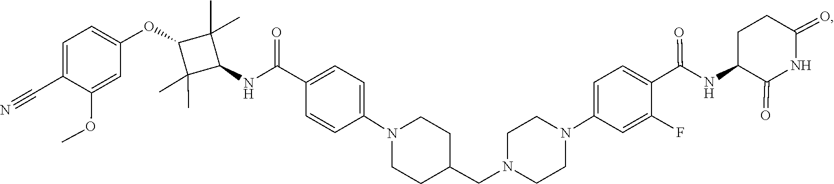 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

or a pharmaceutically acceptable salt thereof.
KKKK. A method of treating prostate cancer in a subject in need thereof, wherein the prostate cancer comprises at least one somatic AR tumor mutation, the method comprising administering to the subject in need thereof a therapeutically effective amount of Compound A,

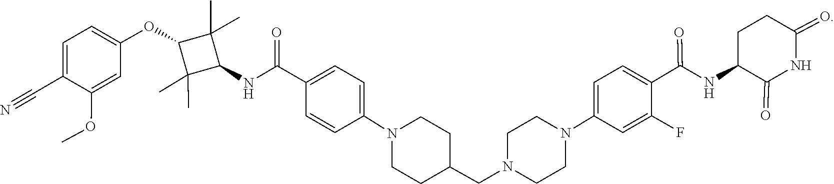 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

LLLL. A method of treating prostate cancer having at least one somatic AR tumor mutation in a subject comprising administering to the subject in need thereof:
(i) a therapeutically effective amount of Compound A,

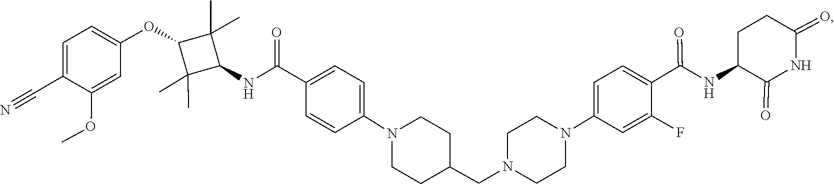 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

or a pharmaceutically acceptable salt thereof; and
(ii) a therapeutically effective amount of abiraterone acetate.
   MMMM. A method of treating prostate cancer having at least one somatic AR tumor mutation in a subject comprising administering to the subject in need thereof:
(i) a therapeutically effective amount of Compound A,

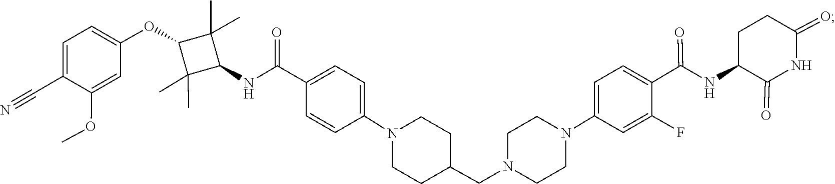 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]

and
(ii) a therapeutically effective amount of abiraterone acetate.
   OOOO. The method of Embodiment JJJJ, KKKK, LLLL, or MMMM, wherein the prostate cancer is adenocarcinoma of the prostate.
   PPPP. The method of Embodiment JJJJ, KKKK, LLLL, or MMMM, wherein the prostate cancer is metastatic prostate cancer.
   QQQQ. The method of Embodiment JJJJ, KKKK, LLLL, or MMMM, wherein the prostate cancer is castrate-resistant prostate cancer.
   RRRR. The method of Embodiment JJJJ, KKKK, LLLL, or MMMM, wherein the prostate cancer is metastatic castrate-resistant prostate cancer.
   SSSS. The method of Embodiment JJJJ, KKKK, LLLL, or MMMM, wherein the prostate cancer is progressive metastatic castrate-resistant prostate cancer.
   TTTT. The method of Embodiment JJJJ, KKKK, LLLL, or MMMM, wherein the prostate cancer is castrate-sensitive prostate cancer.
   UUUU. The method of any one of Embodiments JJJJ-TTTT, wherein the at least one somatic AR tumor mutation is AR tumor mutation comprises L702H.

1.-9. (canceled)

10. A method of treating prostate cancer in a subject comprising administering to the subject in need thereof a therapeutically effective amount of Compound A, 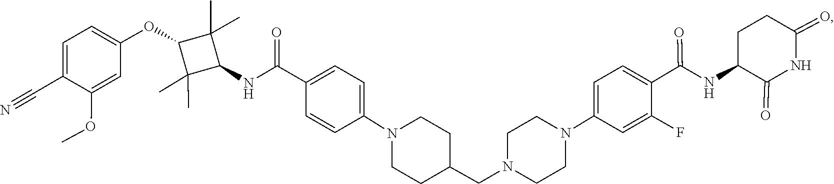 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]
or a pharmaceutically acceptable salt thereof;
wherein the prostate cancer is castrate-sensitive prostate cancer or metastatic castrate-sensitive prostate cancer; and
wherein the therapeutically effective amount of Compound A is about 5 mg to about 750 mg.

11. (canceled)

12. A method of treating prostate cancer in a subject comprising administering to the subject in need thereof:
(i) a therapeutically effective amount of Compound A, 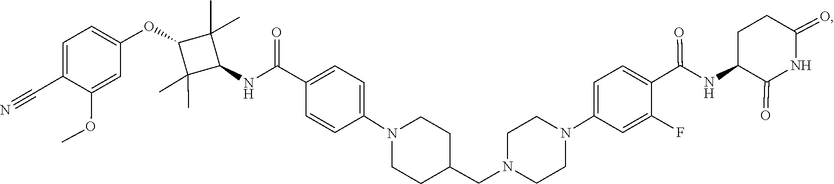 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]
or a pharmaceutically acceptable salt thereof; and
(ii) a therapeutically effective amount of abiraterone acetate;
wherein the prostate cancer is castrate-sensitive prostate cancer or metastatic castrate-sensitive prostate cancer; and
wherein the therapeutically effective amount of Compound A is about 5 mg to about 750 mg.

13.-15. (canceled)

16. A method of treating prostate cancer naïve to novel hormonal agents (NHAs) in a subject comprising administering to the subject in need thereof a therapeutically effective amount of Compound A, 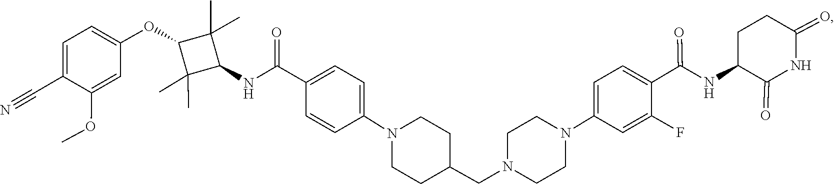 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]
or a pharmaceutically acceptable salt thereof; and
wherein the therapeutically effective amount of Compound A is about 5 mg to about 750 mg.

17. (canceled)

18. A method of treating prostate cancer naïve to novel hormonal agents (NHAs) in a subject comprising administering to the subject in need thereof:
(i) a therapeutically effective amount of Compound A, 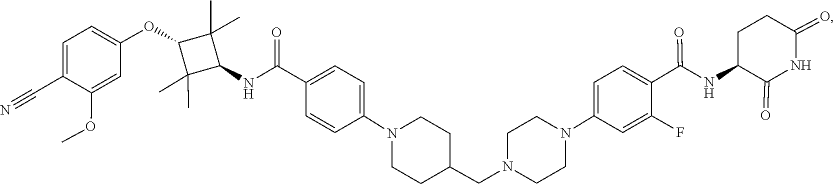 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]
or a pharmaceutically acceptable salt thereof; and
(ii) a therapeutically effective amount of abiraterone acetate; and
wherein the therapeutically effective amount of Compound A is about 5 mg to about 750 mg.

19. (canceled)

20. The method of claim 16, wherein the prostate cancer naïve to NHAs is metastatic prostate cancer naïve to NHAs, castrate-resistant prostate cancer naïve to NHAs, castrate-sensitive prostate cancer naïve to NHAs, metastatic castrate-resistant prostate cancer naïve to NHAs, or metastatic castrate-sensitive prostate cancer naïve to NHAs.

21. The method of claim 18, wherein the prostate cancer naïve to NHAs is metastatic prostate cancer naïve to NHAs, castrate-resistant prostate cancer naïve to NHAs, castrate-sensitive prostate cancer naïve to NHAs, metastatic castrate-resistant prostate cancer naïve to NHAs, or metastatic castrate-sensitive prostate cancer naïve to NHAs.

22.-71. (canceled)

72. A method of treating prostate cancer having at least one somatic AR tumor mutation in a subject comprising administering to the subject in need thereof a therapeutically effective amount of Compound A, 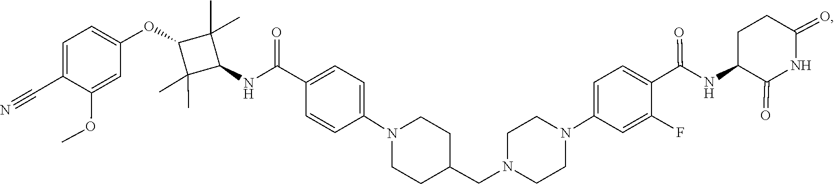 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]
or a pharmaceutically acceptable salt thereof;
wherein the prostate cancer having at least one somatic AR tumor mutation is adenocarcinoma of the prostate, metastatic prostate cancer, castrate-resistant prostate cancer, metastatic castrate-resistant prostate cancer, or progressive metastatic castrate-resistant prostate cancer; and
wherein the therapeutically effective amount of Compound A is about 5 mg to about 750 mg.

73. (canceled)

74. A method of treating prostate cancer having at least one somatic AR tumor mutation in a subject comprising administering to the subject in need thereof:
(i) a therapeutically effective amount of Compound A, 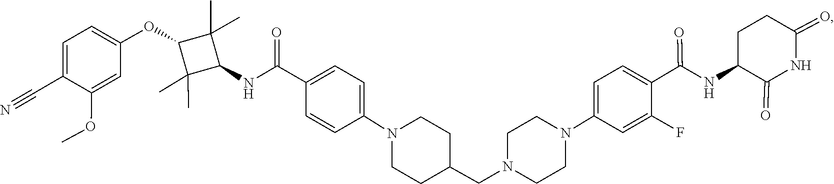 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]
or a pharmaceutically acceptable salt thereof; and
(ii) a therapeutically effective amount of abiraterone acetate;
wherein the prostate cancer having at least one somatic AR tumor mutation is adenocarcinoma of the prostate, metastatic prostate cancer, castrate-resistant prostate cancer, metastatic castrate-resistant prostate cancer, or progressive metastatic castrate-resistant prostate cancer; and
wherein the therapeutically effective amount of Compound A is about 5 mg to about 750 mg.

75.-80. (canceled)

81. A method of treating prostate cancer having at least one somatic AR tumor mutation in a subject comprising administering to the subject in need thereof a therapeutically effective amount of Compound A, 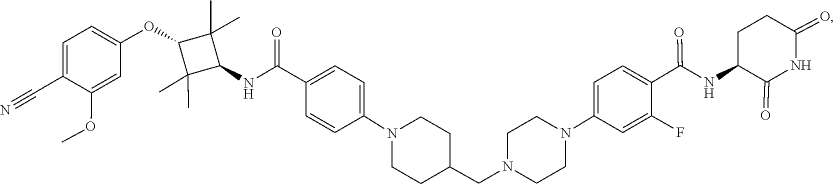 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]
or a pharmaceutically acceptable salt thereof;
wherein the prostate cancer having at least one somatic AR tumor mutation is castrate-sensitive prostate cancer or metastatic castrate-sensitive prostate cancer; and
wherein the therapeutically effective amount of Compound A is about 5 mg to about 750 mg.

82. (canceled)

83. A method of treating prostate cancer having at least one somatic AR tumor mutation in a subject comprising administering to the subject in need thereof:
(i) a therapeutically effective amount of Compound A, 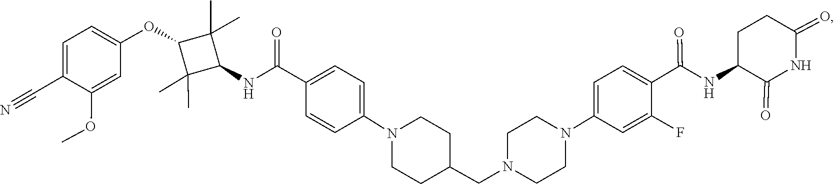 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]
or a pharmaceutically acceptable salt thereof; and
(ii) a therapeutically effective amount of abiraterone acetate;
wherein the prostate cancer having at least one somatic AR tumor mutation is castrate-sensitive prostate cancer or metastatic castrate-sensitive prostate cancer; and
wherein the therapeutically effective amount of Compound A is about 5 mg to about 750 mg.

84.-86. (canceled)

87. A method of treating prostate cancer naïve to novel hormonal agents (NHAs) having at least one somatic AR tumor mutation in a subject comprising administering to the subject in need thereof a therapeutically effective amount of Compound A, 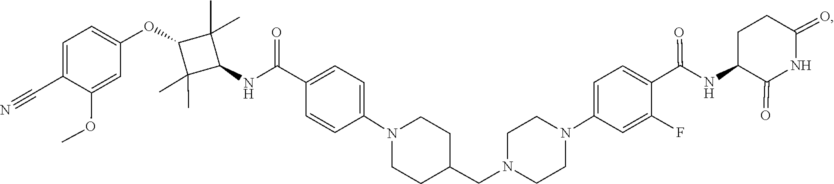 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]
or a pharmaceutically acceptable salt thereof; and
wherein the therapeutically effective amount of Compound A is about 5 mg to about 750 mg.

88. (canceled)

89. A method of treating prostate cancer naïve to novel hormonal agents (NHAs) having at least one somatic AR tumor mutation in a subject comprising administering to the subject in need thereof:
(i) a therapeutically effective amount of Compound A, 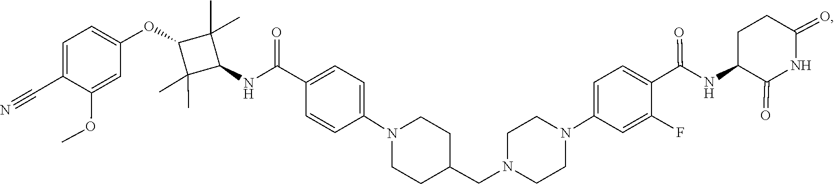 [SMILES: COc1cc(O[C@H]2C(C)(C)[C@H](NC(=O)c3ccc(N4CCC(CN5CCN(c6ccc(C(=O)N[C@H]7CCC(=O)NC7=O)c(F)c6)CC5)CC4)cc3)C2(C)C)ccc1C#N]
or a pharmaceutically acceptable salt thereof; and
(ii) a therapeutically effective amount of abiraterone acetate; and
wherein the therapeutically effective amount of Compound A is about 5 mg to about 750 mg.

90. (canceled)

91. The method of claim 87, wherein the prostate cancer naïve to NHAs having at least one somatic AR tumor mutation is metastatic prostate cancer naïve to NHAs, castrate-resistant prostate cancer naïve to NHAs, castrate-sensitive prostate cancer naïve to NHAs, metastatic castrate-resistant prostate cancer naïve to NHAs, or metastatic castrate-sensitive prostate cancer naïve to NHAs.

92. The method of claim 89, wherein the prostate cancer naïve to NHAs having at least one somatic AR tumor mutation is metastatic prostate cancer naïve to NHAs, castrate-resistant prostate cancer naïve to NHAs, castrate-sensitive prostate cancer naïve to NHAs, metastatic castrate-resistant prostate cancer naïve to NHAs, or metastatic castrate-sensitive prostate cancer naïve to NHAs.

93.-95. (canceled)

96. The method of claim 72, wherein the at least one somatic AR tumor mutation is an AR ligand binding domain missense mutation.

97.-113. (canceled)

114. The method of claim 74, wherein the at least one somatic AR tumor mutation is an AR ligand binding domain missense mutation.

115. The method of claim 81, wherein the at least one somatic AR tumor mutation is an AR ligand binding domain missense mutation.

116. The method of claim 83, wherein the at least one somatic AR tumor mutation is an AR ligand binding domain missense mutation.

117. The method of claim 87, wherein the at least one somatic AR tumor mutation is an AR ligand binding domain missense mutation.

118. The method of claim 89, wherein the at least one somatic AR tumor mutation is an AR ligand binding domain missense mutation.


SEQUENCE LISTING (1 sequence)

SEQ ID NO: 1 - protein, 920 aa, Homo sapiens
MEVQLGLGRVYPRPPSKTYRGAFQNLFQSVREVIQNPGPRHPEAASAAPPGASLLLLQQQQQQQQQQQQQQQQQQQQQQQETSPRQQQQQQGEDGSPQAHRRGPTGYLVLDEEQQPSQPQSALECHPERGCVPEPGAAVAASKGLPQQLPAPPDEDDSAAPSTLSLLGPTFPGLSSCSADLKDILSEASTMQLLQQQQQEAVSEGSSSGRAREASGAPTSSKDNYLGGTSTISDNAKELCKAVSVSMGLGVEALEHLSPGEQLRGDCMYAPLLGVPPAVRPTPCAPLAECKGSLLDDSAGKSTEDTAEYSPFKGGYTKGLEGESLGCSGSAAAGSSGTLELPSTLSLYKSGALDEAAAYQSRDYYNFPLALAGPPPPPPPPHPHARIKLENPLDYGSAWAAAAAQCRYGDLASLHGAGAAGPGSGSPSAAASSSWHTLFTAEEGQLYGPCGGGGGGGGGGGGGGGGGGGGGGGEAGAVAPYGYTRPPQGLAGQESDFTAPDVWYPGGMVSRVPYPSPTCVKSEMGPWMDSYSGPYGDMRLETARDHVLPIDYYFPPQKTCLICGDEASGCHYGALTCGSCKVFFKRAAEGKQKYLCASRNDCTIDKFRRKNCPSCRLRKCYEAGMTLGARKLKKLGNLKLQEEGEASSTTSPTEETTQKLTVSHIEGYECQPIFLNVLEAIEPGVVCAGHDNNQPDSFAALLSSLNELGERQLVHVVKWAKALPGFRNLHVDDQMAVIQYSWMGLMVFAMGWRSFTNVNSRMLYFAPDLVFNEYRMHKSRMYSQCVRMRHLSQEFGWLQITPQEFLCMKALLLFSIIPVDGLKNQKFFDELRMNYIKELDRIIACKRKNPTSCSRRFYQLTKLLDSVQPIARELHQFTFDLLIKSHMVSVDFPEMMAEIISVQVPKILSGKVKPIYFHTQ